SEQUENCE LISTING (41 sequences)

SEQ ID NO: 1 - protein, 93 aa, HOMO SAPIEN
PAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 2 - protein, 119 aa, HOMO SAPIEN
PFCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 3 - protein, 141 aa, HOMO SAPIEN
PSKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDT

SEQ ID NO: 4 - protein, 122 aa, HOMO SAPIEN
PSREPLRPWCHPINAILAVEKEGCPVCITVNTTICAGYCPTMMRVLQAVLPPLPQVVCTYRDVRFESIRLPGCPRGVDPVVSFPVALSCRCGPCRRSTSDCGGPKDHPLTCDHPQLSGLLFL

SEQ ID NO: 5 - protein, 110 aa, HOMO SAPIEN
PNSCELTNITIAIEKEECRFCISINTTWCAGYCYTRDLVYKDPARPKITCTFKELVYETVRVPGCAHHADSLYTYPVATQCHCGKCDSDSTDCTVRGLGPSYCSFGEMKE

SEQ ID NO: 6 - protein, 126 aa, HOMO SAPIEN
PSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTGRPRESGKKRKRKRLKPT

SEQ ID NO: 7 - protein, 161 aa, HOMO SAPIEN
PSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 8 - protein, 190 aa, HOMO SAPIEN
PAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 9 - protein, 121 aa, HOMO SAPIEN
PSSSHPIFHRGEFSVCDSVSVWVGDKTTATDIKGKEVMVLGEVNNINSVFKQYFFETKCRDPNPVDSGCRGIDSKHWNSYCTTTHTFVKAMLTDGKQAAWRFIRIDTACVCVLSRKAVRRA

SEQ ID NO: 10 - protein, 120 aa, HOMO SAPIEN
PHSDPARRGELSVCDSISEWVTAADKKTAVDMSGGTVTVLEKVSPVKGQLKQYFYETKCNPMGYTKEGCRGIDKRHWNSQCRTTQSYVRAMLTDSKKRIGWRFIRIDTSCVCILTIKRGR

SEQ ID NO: 11 - protein, 120 aa, HOMO SAPIEN
PYAEHKSHRGEYSVCDSESLWVTDKSSAIDIRGHQVTVLGEIGKTNSPVKQYFYETRCKEARPVKNGCRGIDDRHWNSQCKTSQTYVRASLTENNKLVGWRWIRIDTSCVCALSRKIGRT

SEQ ID NO: 12 - protein, 131 aa, HOMO SAPIEN
PGVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 13 - protein, 113 aa, HOMO SAPIEN
PALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 14 - protein, 113 aa, HOMO SAPIEN
PALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 15 - protein, 113 aa, HOMO SAPIEN
PALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 16 - protein, 371 aa, HOMO SAPIEN
PMWPLWLCWALWVLPLAGPGAALTEEQLLASLLRQLQLSEVPVLDRADMEKLVIPAHVRAQYVVLLRRDGDRSRGKRFSQSFREVAGRFLASEASTHLLVFGMEQRLPPNSELVQAVLRLFQEPVPQGALHRHGRLSPAAPKARVTVEWLVRDDGSNRTSLIDSRLVSVHESGWKAFDVTEAVNFWQQLSRPPEPLLVQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQHRPWCIH

SEQ ID NO: 17 - protein, 198 aa, HOMO SAPIEN
PMQRWKAAALASVLCSSVLSIWMCREGLLLSHRLGPALVPLHRLPRTLDARIARLAQYRALLQGAPDAMELRELTPWAGRPPGPRRRAGPRRRRARARLGARPCGLRELEVRVSELGLGYASDETVLFRYCAGACEAAARVYDLGLRRLRQRRRLRRERVRAQPCCRPTAYEDEVSFLDAHSRYHTVHELSARECACV

SEQ ID NO: 18 - protein, 367 aa, HOMO SAPIEN
PMVLHLLLFLLLTPQGGHSCQGLELARELVLAKVRALFLDALGPPAVTREGGDPGVRRLPRRHALGGFTHRGSEPEEEEDVSQAILFPATDASCEDKSAARGLAQEAEEGLFRYMFRPSQHTRSRQVTSAQLWFHTGLDRQGTAASNSSEPLLGLLALSPGGPVAVPMSLGHAPPHWAVLHLATSALSLLTHPVLVLLLRCPLCTCSARPEATPFLVAHTRTRPPSGGERARRSTPLMSWPWSPSALRLLQRPPEEPAAHANCHRVALNISFQELGWERWIVYPPSFIFHYCHGGCGLHIPPNLSLPVPGAPPTPAQPYSLLPGAQPCCAALPGTMRPLHVRTTSDGGYSFKYETVPNLLTQHCACI

SEQ ID NO: 19 - protein, 427 aa, HOMO SAPIEN
PMPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 20 - protein, 408 aa, HOMO SAPIEN
PMDGLPGRALGAACLLLLAAGWLGPEAWGSPTPPPTPAAPPPPPPPGSPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNMVEKRVDLKRSGWHTFPLTEAIQALFERGERRLNLDVQCDSCQELAVVPVFVDPGEESHRPFVVVQARLGDSRHRIRKRGLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 21 - protein, 427 aa, HOMO SAPIEN; identical to SEQ ID NO: 19

SEQ ID NO: 22 - protein, 408 aa, HOMO SAPIEN; identical to SEQ ID NO: 20

SEQ ID NO: 23 - protein, 561 aa, HOMO SAPIEN
PMRDLPLTSLALVLSALGALLGTEALRAEEPAVGTSGLIFREDLDWPPGIPQEPLCLVALGGDSNGSSSPLRVVGALSAYEQAFLGAVQRARWGPRDLATFGVCNTGDRQAALPSLRRLGAWLRDPGGQRLVVLHLEEVTWEPTPSLRFQEPPPGGAGPPELALLVLYPGPGPEVTVTRAGLPGAQSLCPSRDTRYLVLAVDRPAGAWRGSGLALTLQPRGEDSRLSTARLQALLFGDDHRCFTRMTPALLLLPRSEPAPLPAHGQLDTVPFPPPRPSAELEESPPSADPFLETLTRLVRALRVPPARASAPRLALDPDALAGFPQGLVNLSDPAALERLLDGEEPLLLLLRPTAATTGDPAPLHDPTSAPWATALARRVAAELQAAAAELRSLPGLPPATAPLLARLLALCPGGPGGLGDPLRALLLLKALQGLRVEWRGRDPRGPGRAQRSAGATAADGPCALRELSVDLRAERSVLIPETYQANNCQGVCGWPQSDRNPRYGNHVVLLLKMQARGAALARPPCCVPTAYAGKLLISLSEERISAHHVPNMVATECGCR

SEQ ID NO: 24 - protein, 397 aa, HOMO SAPIEN
PMVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 25 - protein, 473 aa, HOMO SAPIEN
PMAGASRLLFLWLGCFCVSLAQGERPKPPFPELRKAVPGDRTAGGGPDSELQPQDKVSEHMLRLYDRYSTVQAARTPGSLEGGSQPWRPRLLREGNTVRSFRAAAAETLERKGLYIFNLTSLTKSENILSATLYFCIGELGNISLSCPVSGGCSHHAQRKHIQIDLSAWTLKFSRNQSQLLGHLSVDMAKSHRDIMSWLSKDITQFLRKAKENEEFLIGFNITSKGRQLPKRRLPFPEPYILVYANDAAISEPESVVSSLQGHRNFPTGTVPKWDSHIRAALSIERRKKRSTGVLLPLQNNELPGAEYQYKKDEVWEERKPYKTLQAQAPEKSKNKKKQRKGPHRKSQTLQFDEQTLKKARRKQWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 26 - protein, 479 aa, HOMO SAPIEN
PMAHVPARTSPGPGPQLLLLLLPLFLLLLRDVAGSHRAPAWSALPAAADGLQGDRDLQRHPGDAAATLGPSAQDMVAVHMHRLYEKYSRQGARPGGGNTVRSFRARLEVVDQKAVYFFNLTSMQDSEMILTATFHFYSEPPRWPRALEVLCKPRAKNASGRPLPLGPPTRQHLLFRSLSQNTATQGLLRGAMALAPPPRGLWQAKDISPIVKAARRDGELLLSAQLDSEERDPGVPRPSPYAPYILVYANDLAISEPNSVAVTLQRYDPFPAGDPEPRAAPNNSADPRVRRAAQATGPLQDNELPGLDERPPRAHAQHFHKHQLWPSPFRALKPRPGRKDRRKKGQEVFMAASQVLDFDEKTMQKARRKQWDEPRVCSRRYLKVDFADIGWNEWIISPKSFDAYYCAGACEFPMPKIVRPSNHATIQSIVRAVGIIPGIPEPCCVPDKMNSLGVLFLDENRNVVLKVYPNMSVDTCACR

SEQ ID NO: 27 - protein, 409 aa, HOMO SAPIEN
PMIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEAISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 28 - protein, 455 aa, HOMO SAPIEN
PMHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 29 - protein, 112 aa, HOMO SAPIEN
PSSASDYNSSELKTACRKHELYVSFQDLGWQWIIAPKGYAANYCDGECSPPLNHTANHAIVQTLVHLMNPEYVPKPCCAPTKLNAISVLYFDDNSNVIKKYRNMVVRACGCH

SEQ ID NO: 30 - protein, 112 aa, HOMO SAPIEN
PANVAENSSSDQRQACKKHELYVSFRDLGWQWIIAPEGYAAYYCEGECAFPLNSATNHAIVQTLVHFINPETVPKPCCAPTQLNAISVLYFDDSSNVIKKYRNMVVRACGCH

SEQ ID NO: 31 - protein, 403 aa, HOMO SAPIEN
PMTALPGPLWLLGLALCALGGGGPGLRPPPGCPQRRLGARERRDVQREILAVLGLPGRPRPRAPPAASRLPASAPLFMLDLYHAMAGDDDEDGAPAERRLGRADLVMSFVNMVERDRALGHQEPHWKEFRFDLTQIPAGEAVTAAEFRIYKVPSIHLLNRTLHVSMFQVVQEQSNRESDLFFLDLQTLRAGDEGWLVLDVTAASDCWLLKRHKDLGLRLYVETEDGHSVDPGLAGLLGQRAPRSQQPFVVTFFRASPSPIRTPRAVRPLRRRQPKKSNELPQANRLPGIFDDVHGSHGRQVCRRHELYVSFQDLGWLDWVIAPQGYSAYYCEGECSFPLDSCMNATNHAILQSLVHLMKPNAVPKACCAPTKLSATSVLYYDSSNNVILRKHRNMVVKACGCH

SEQ ID NO: 32 - protein, 425 aa, HOMO SAPIEN
PMGSLVLTLCALFCLAAYLVSGSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRRNAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 33 - protein, 408 aa, HOMO SAPIEN
PMVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 34 - protein, 393 aa, HOMO SAPIEN
PMVLLSILRILFLCELVLFMEHRAQMAEGGQSFIALLAEAPTLPLIEEMLEESPGEQPRKPRLLGHSLRYMLELYRRSADSHGHPRENRTIGATMVRLVKPLTSVARPHRGTWHIQILGFPLRPNRGLYQLVRATVVYRHHLQLTRFNLSCHVEPWVQKNPTNHFPSSEGDSSKPSLMSNAWKEMDITQLVQQRFWNNKGHRILRLRFMCQQQKDSGGLELWHGTSSLDIAFLLLYFNDTHKSIRKAKFLPRGMEEFMERESLLRRTRQADGISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR

SEQ ID NO: 35 - protein, 134 aa, HOMO SAPIEN
PMRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 36 - protein, 373 aa, HOMO SAPIEN
PMPPPQQGPCGHHLLLLLALLLPSLPLTRAPVPPGPAAALLQALGLRDEPQGAPRLRPVPPVMWRLFRRRDPQETRSGSRRTSPGVTLQPCHVEELGVAGNIVRHIPDRGAPTRASEPVSAAGHCPEWTVVFDLSAVEPAERPSRARLELRFAAAAAAAPEGGWELSVAQAGQGAGADPGPVLLRQLVPALGPPVRAELLGAAWARNASWPRSLRLALALRPRAPAACARLAEASLLLVTLDPRLCHPLARPRRDAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMVVDECGCR

SEQ ID NO: 37 - protein, 502 aa, HOMO SAPIEN
PMRLPKLLTFLLWYLAWLDLEFICTVLGAPDLGQRPQGSRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAVPRSRRAAQLKLSSCPSGRQPAALLDVRSVPGLDGSGWEVFDIWKLFRNFKNSAQLCLELEAWERGRTVDLRGLGFDRAARQVHEKALFLVFGRTKKRDLFFNEIKARSGQDDKTVYEYLFSQRRKRRAPSATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 38 - protein, 376 aa, HOMO SAPIEN
PMQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 39 - protein, 455 aa, HOMO SAPIEN
PMARPNKFLLWFCCFAWLCFPISLGSQASGGEAQIAASAELESGAMPWSLLQHIDERDRAGLLPALFKVLSVGRGGSPRLQPDSRALHYMKKLYKTYATKEGIPKSNRSHLYNTVRLFTPCTRHKQAPGDQVTGILPSVELLFNLDRITTVEHLLKSVLLYNINNSVSFSSAVKCVCNLMIKEPKSSSRTLGRAPYSFTFNSQFEFGKKHKWIQIDVTSLLQPLVASNKRSIHMSINFTCMKDQLEHPSAQNGLFNMTLVSPSLILYLNDTSAQAYHSWYSLHYKRRPSQGPDQERSLSAYPVGEEAAEDGRSSHHRHRRGQETVSSELKKPLGPASFNLSEYFRQFLLPQNECELHDFRLSFSQLKWDNWIVAPHRYNPRYCKGDCPRAVGHRYGSPVHTMVQNIIYEKLDSSVPRPSCVPAKYSPLSVLTIEPDGSIAYKEYEDMIATKCTCR

SEQ ID NO: 40 - protein, 238 aa, HOMO SAPIEN
PMPGLISARGQPLLEVLPPQAHLGALFLPEAPLGLSAQPALWPTLAALALLSSVAEASLGSAPRSPAPREGPPPVLASPAGHLPGGRTARWCSGRARRPPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 41 - protein, 157 aa, HOMO SAPIEN
PMAVGKFLLGSLLLLSLQLGQGWGPDARGVPVADGEFSSEQVAKAGGTWLGTHRPLARLRRALSGPCQLWSLTLSVAELGLGYASEEKVIFRYCAGSCPRGARTQHGLALARLQGQGRAHGGPCCRPTRYTDVAFLDDRHRWQRLPQLSAAACGCGG

(19) United States
(12) Patent Application Publication
(10) Pub. No.: US 2002/0169292 A1
(43) Pub. Date: Nov. 14, 2002

(54) Cystine knot growth factor mutants

(76) Inventors: Bruce D. Weintraub, Rockville, MD (US); Mariusz W. Szkudlinski, Potomac, MD (US)

Correspondence Address: Steven B. Kelber, PIPER, MARBURY RUDNICK & WOLFE, L.L.P., 1200 Nineteenth Street N.W., Washington, DC, 20036-2412, US

(21) Appl. No.: 09/813,398

(22) Filed: Mar. 20, 2001

Related U.S. Application Data
(63) Continuation of application No. PCT/US99/05908, filed on Mar. 19, 1999.

(30) Foreign Application Priority Data
Sep. 22, 1998 (US) PCT/US98/19772

(51) Int. Cl. A61K 38/22; C07H 21/04; C12P 21/02; C12N 5/06; C07K 14/575

(52) U.S. Cl. 530/397; 514/8; 536/23.5; 435/69.4; 435/325; 435/320.1

(57) ABSTRACT

Compositions and methods based on mutant Cystine Knot Growth Factors (CKGFs) comprising amino acid substitutions relative to the wild type hormone/growth factor. Mutated glycoprotein hormones, including thyroid stimulating hormone (TSH) and chorionic gonadotropin (CG) are disclosed as exemplary mutant CKGFs. Mutant TSH heterodimers and hCH heterodimers possessed modified bioactivities, including superagonist activity. Accordingly, the present invention provides methods for using mutant CKGFs, CKGF analogs, fragments, and derivatives thereof for treating or preventing diseases. Pharmaceutical and diagnostic compositions, methods of using mutant TSH heterodimers and TSH analogs with utility for treatment and prevention of metabolic and reproductive diseases are also provided.

### RELATED APPLICATIONS

This application claims the benefit of priority from PCT/US99/05908, filed Mar. 19, 1999, which claims the benefit of priority from PCT/US98/19772, filed Sep. 22, 1998, each of which is hereby incorporated by reference in their entirety.

### FIELD OF THE INVENTION

The present invention relates generally to the field of protein growth factors. More specifically, the invention relates to cystine knot growth factor (CKGF) mutants having desirable pharmacological properties. The invention further relates to methods of producing these mutants, to pharmaceutical compositions and to methods of treatment and diagnosis based thereon.

### BACKGROUND OF THE INVENTION

Growth factors are a diverse group of proteins that regulate cell growth, differentiation and cell-cell communication. Although the molecular mechanisms governing growth factor-mediated processes remain largely unknown, it is clear that growth factors can be classified into one of several superfamilies based on structural and functional similarities.

Crystal structures of four different growth factors—nerve growth factor (NGF), transforming growth factor-β (TGF-β), platelet-derived growth factor (PDGF) and human chorionic gonadotropin (hCG)—representing four separate protein families revealed that family members were structurally related and shared a common overall topology. While these four proteins shared very little sequence homology, there was a characteristic arrangement of six cysteines linked in a “cystine-knot” conformation. The active forms of these proteins were dimers, either homodimers or heterodimers. Mutational analyses have indicated that mutation of any of the six conserved cysteine residues resulted in a loss of growth factor activity (Brunner et al., 1992, Mol. Endocrinol. 6:1691-1700; Glese et al., 1987, Science 236:1315-18).

The remarkable structural similarity shared among the cystine knot growth factors suggests evolution from a common ancestral gene. The structural and functional properties of the CKGF superfamily, and the crystal structures of TGF-β, NGF, PDGF and hCG have been reviewed by Sun and Davies (Annu. Rev. Biophys. Biomol. Struct. 1995, 24:269-291), McDonald and Hendrickson (Cell, 1993, 73:421-424), and Murray-Rust et al. (Structure, 1993, 1:153-159).

Glycoprotein Hormones

The glycoprotein hormones are a group of evolutionarily conserved hormones involved in the regulation of reproduction and metabolism (Pierce and Parsons, 1981, Endocr. Rev. 11:354-385). This family of hormones includes the follicle-stimulating hormone (FSH), luteinizing hormone (LH), thyroid stimulating hormone (TSH), and chorionic gonadotrophin (CG). Structurally, the glycoprotein hormones are heterodimers comprised of a common α-subunit and a hormone-specific β-subunit.

Structure-function relationships among the human glycoprotein hormones have been substantially based on models of gonadotropins, particularly hCG. Recently, the crystal structure of partially deglycosylated hCG revealed two key structural features that are relevant to the other glycoprotein hormones, (Lapthom et al., 1994, Nature 369:455-461; Wu et al., 1994, Structure 2:548-558). The common α-subunit contains an apoprotein core of 92 amino acids including 10 half-cystine residues, all of which are in disulfide linkage. The proposed pairs are 10-60, 28-82, 32-84, 7-31 and 59-87. Bonds 28-82 and 32-84 form a ring structure penetrated by a bond bridging cysteine residues 10 and 60 to result in a core—the cystine knot—that forms three hairpin loops. Both α-subunit and hCG β-subunit have a similar overall topology—each subunit has two β-hairpin loops (L1 and L3) on one side of the central cystine knot (formed by three disulfide bonds), and a long loop (L2) on the other.

TSH is a 28-30 kDa heterodimeric glycoprotein produced in the thyrotrophs of the anterior pituitary gland. This hormone controls thyroid function by interacting with the G protein-coupled TSH receptor (TSHR), (Vassant and Dumont, 1992, Endocr. Rev. 13:596-611) which leads to the stimulation of pathways involving secondary messenger molecules, such as, cyclic adenosine 3′5′-monophosphate (cAMP), and ultimately results in the modulation of thyroidal gene expression. Physiological roles of TSH include stimulation of differentiated thyroid functions, such as iodine uptake and the release of thyroid hormone from the gland, and promotion of thyroid growth (Wondisford et al., 1996, Thyrotropin. In: Braverman et al. (eds.), Werner and Ingbar's The Thyroid, Lippencott-Raven, Philadelphia, pp. 190-207).

Structurally, the glycoprotein hormones are related heterodimers comprised of a common α-subunit and a hormone-specific β-subunit. As indicated above, the common human α-subunit contains an apoprotein core of 92 amino acids including 10 half-cystine residues, all of which are in disulfide linkage. The α-subunit is encoded by a single gene which is located on chromosome 6 in humans, and is identical in amino acid sequence within a given species (Fiddes and Goodman, 1981, J. Mol. Appl. Gen. 1:3-18). The hormone specific β-subunit genes differ in length, structural organization and chromosomal localization (Shupnik et al., 1989, Endocr. Rev. 10:459-475). The human TSH β-subunit gene predicts a mature protein having 118 amino acid residues and is localized on chromosome 1 (Wondisford et al., supra). The various β-subunits can be aligned according to 12 invariant half-cystine residues forming 6 disulfide bonds. Despite a 30 to 80% amino acid sequence identity among the hormones, the β-subunits exhibit differential receptor binding with high specificity (Pierce and Parsons, supra).

Significantly, the carbohydrate moiety of the glycoprotein hormones constitutes 15-35% by weight of the hormone. The common α-subunit has two asparagine (N)-linked oligosaccharides, and the β-subunit one (in TSH and LH) or two (in CG and FSH). In addition, the CG β-subunit has a unique 32 residue carboxyl-terminal extension peptide (CTEP) with four serine (O)-linked glycosylation sites. (Baenziger, 1994, Glycosylation and glycoprotein hormone function, in Lustbander et al. (eds.) Glycoprotein Hormones: Structure, Function and Clinical Implications. Springer-Verlag, New York, pages 167-174).

Molecular studies on human TSH have been facilitated by the cloning of TSH β-subunit cDNA and gene (Joshi et al., 1995, Endocrinol. 136:3839-3848), the cloning of TSH receptor cDNA (Parmentier et al., 1989, Science 246:1620-1622; Nagayama et al., 1990, Biochem. Biophys. Res. Commun. 166:394-403), and the expression of recombinant TSH (Cole et al., 1993, Bio/Technol. 11:1014-1024; Grossmann et al., 1995, Mol. Endocrinol. 9:948-958; Szkudlinski et al., 1996 supra). Previous structure-function studies directed toward TSH focussed primarily on the highly conserved regions and the creation of chimeric subunits. However, these approaches did not result in mutant hormones having increased in vitro bioactivity (Grossmann et al., 1997, Endocr. Rev. 18:476-501).

Strategies for prolonging the half-life of glycoprotein hormones in circulation also have been developed. In gene fusion experiments, the carboxyl-terminal extension peptide (CTEP) of the hCG β-subunit, which contains several O-linked carbohydrates, was linked to the human TSH β subunit (Joshi et al., 1995, Endocrinol., 136:3839-3848; Grossmann et al., 1997, J. Biol. Chem. 272:21312-21316). Whereas the in vitro activity of these chimeras was not altered, their circulatory half-lives were prolonged to result in enhanced in vivo bioactivity. Additionally, expressing the β and α subunits as a single chain fusion protein enhanced stability and a prolonged plasma half-life compared to wild type glycoprotein hormone (Sugahara et al., 1995, Proc. Natl. Acad. Sci. USA 92:2041-2045; Grossmann et al., 1997, J. Biol. Chem. 272:21312-21316).

Use of TSH in the Diagnosis and Monitoring of Thyroid Carcinoma

Recombinant TSH has been tested for stimulating ¹³¹I uptake and thyroglobulin secretion in the diagnosis and follow up of 19 patients with differentiated thyroid carcinoma, thus avoiding the side effects of thyroid hormone withdrawal (Meier et al., J. Clin. Endocrinol. Metab. 78:188-196). Preliminary results from the first trial are highly encouraging. The incidence of thyroid carcinoma in the United States is approximately 14,000 cases per year. Most of these are differentiated, and papillary or-follicular cancers are the most common subtypes. As the 10- and 20-year survival rate of such differentiated thyroid carcinomas is 90% and 60% respectively, long term monitoring to detect local recurrence and distant metastases becomes essential in the management of such patients, especially since tumor can recur even decades after primary therapy. The principal methods used for follow-up are whole body radioiodine scanning and serum thyroglobulin measurements. For optimal sensitivity of these diagnostic procedures, stimulation of residual thyroid tissue by TSH to increase ¹³¹Iodine uptake or thyroglobulin secretion, respectively is required. However, post-thyroidectomy thyroid cancer patients are treated with thyroid hormone to suppress endogenous TSH to avoid potential stimulatory effects of TSH on residual thyroid tissue, as well as to maintain euthyroidism. Usually therefore, levo-T₄ or, less commonly used T₃ is withdrawn 4-6 and 2 weeks before radioiodine scanning and thyroglobulin determination in order to stimulate endogenous TSH secretion. The accompanying transient but severe hypothyroidism considerably impairs the quality of life, and may interfere with the ability to work. Further, since TSH can act as a growth factor for malignant thyroid tissue, prolonged periods of increased endogenous TSH secretion may pose a potential risk for such patients.

In the 1960s, bovine TSH (bTSH) was used to stimulate residual thyroid tissue to overcome the need for elevating endogenous TSH (Blahd et al., 1960, Cancer 13:745-756). However, several disadvantages led to the discontinuation of its use in clinical practice. Compared to hormone withdrawal, bTSH proved to be less efficacious in detecting residual malignant thyroid tissue and metastases. In addition, allergic reactions and the development of neutralizing antibodies limited the effects of subsequent bTSH administration and interfered with endogenous TSH determinations (Braverman et al., 1992, J. Clin. Endocrinol. Metab. 74:1135-1139).

Below there are described methods for making and using novel mutant CKGFs having desirable pharmacological properties. More particularly, the description presented below provides hormone compositions useful as agonists having prolonged hormonal half-lives or increased intrinsic activities. Alternative hormone compositions exhibit decreased hormonal activity and so represent potential antagonists.

### SUMMARY OF THE INVENTION

Compositions and methods based on mutant Cystine Knot Growth Factors (CKGFs) comprising amino acid substitutions relative to the wild type hormone/growth factor. Mutated glycoprotein hormones, including thyroid stimulating hormone (TSH) and chorionic gonadotropin (CG) are disclosed as exemplary mutant CKGFs. Mutant TSH heterodimers and hCH heterodimers possessed modified bioactivities, including superagonist activity. Additionally, a variety of mutant CKGF family proteins are disclosed. For example, mutant CKGF proteins disclosed include mutant platelet-derived growth factor (PDGF) family proteins such as mutant PDGF homo- and heterodimers, and mutant vascular epithelial cell growth factor (VEGF) proteins; mutant neurotrophin family proteins such as mutant nerve growth factor (NGF), mutant brain-derived neurotrophic factor (BDNF) proteins, and mutant neurotrophin-3 (NT-3) and mutant neurotrophin-4 (NT-4) proteins; mutant transforming growth factor-β (TGF-β) family proteins such as mutant TGF-β1, mutant TGF-β2, mutant TGF-β3, mutant TGF-β4/ebaf, mutant neurturin, mutant inhibin A, mutant inhibin B, mutant Activin A, mutant Activin B, mutant Activin AB, mutant Müllerian inhibitory substance (MIS), mutant bone morphogenic Protein-2 (BMP-2), mutant bone morphogenic protein-3 (BMP-3)/osteogenin, mutant bone morphogenic protein-3b (BMP-3b), mutant bone morphogenic protein-4 (BMP-4), mutant bone morphogenic protein-5 (BMP-5) (precursor only), mutant bone morphogenic protein-6 (BMP-6)/Vgrl, mutant bone morphogenic protein-7 (BMP-7)/osteogenic protein (OP)-1, mutant bone morphogenic protein-8 (BMP-8)/osteogenic protein (OP)-2, mutant bone morphogenic protein-10 (BMP-10), mutant bone morphogenic protein-11 (BMP-11), mutant bone morphogenic protein-15 (BMP-15), mutant Norrie Disease protein (NDP), mutant Growth/Differentiation Factor-1 (GDF-1), mutant Growth/Differentiation Factor-5 (GDF-5) (precursor only), mutant Growth/Differentiation Factor-8 (GDF-8), mutant Growth/Differentiation Factor-9 (GDF-9), mutant Glial Cell-Derived Neurotrophic Factor (GDNF)/Artemin, and mutant Glial Cell-Derived Neurotrophic Factor (GDNF)/Persephin proteins. Accordingly, the present invention provides methods for using mutant CKGFs, CKGF analogs, fragments, and derivatives thereof for treating or preventing diseases. Pharmaceutical and diagnostic compositions, methods of using mutant CKGF proteins, including TSH heterodimers and TSH analogs with utility for treatment and prevention of metabolic and reproductive diseases are also provided.

Definitions

As used herein, the following terms shall have the indicated meanings:

The term TSH means thyroid stimulating hormone.

The term TSHR means thyroid stimulating hormone receptor.

The term hCG means human chorionic gonadotropin.

The term CTEP refers to the carboxyl terminal extension peptide of hCG β subunit.

The term peripheral loops means the β-hairpin loops of the CKGF proteins that are composed of an antiparallel β-sheet and the actual loop. There are two peripheral loops in a typical CKGF subunit.

The term charge reversal technique means the generation of mutant CKGF proteins by introducing a charged residue of the opposite charge of the residue present in the wild type CKGF protein.

Conventional single letter codes are used to denote amino acid residues.

As used herein, mutations within the CKGF subunits, such as the TSH subunits are indicated by the wild type CKGF protein amino acid, followed by the amino acid position, and then mutant amino acid residue. For example, I58R shall mean a mutation from isoleucine to arginine at position 58.

### BRIEF DESCRIPTION OF THE DRAWINGS

FIG. 1 is a two dimensional representation of a cystine knot growth factor showing the cystine knot and the β hairpin loops, L1 and L3.

FIG. 2 shows the amino acid sequence (SEQ ID NO:1) of the human glycoprotein hormone common α subunit. The β hairpin L1 and L3 loops (positions 8-30 and positions 61-85 respectively) are indicated each by a line above or below the sequence.

FIG. 3 shows the amino acid sequence (SEQ ID NO:2) of the human TSH β subunit. The β hairpin L1 and L3 loops-(positions 1-30 and positions 53-87 respectively) are indicated each by a line above or below the sequence.

FIG. 4 shows the amino acid sequence (SEQ ID NO:3) of the human chorionic gonadotropin (hCG) β subunit. The β hairpin L1 and L3 loops (positions 8-33 and positions 58-87 respectively) are indicated each by a line above or below the sequence. The numbers above or below the sequence indicate the amino acid positions at which mutation is preferred.

FIG. 5 shows the amino acid sequence (SEQ ID NO:4) of the human luteinizng hormone (hLH) β subunit. The β hairpin L1 and L3 loops (positions 8-33 and positions 58-87 respectively) are indicated each by a line above or below the sequence.

FIG. 6 shows the amino acid sequence (SEQ ID NO:5) of the human follicle stimulating hormone (FSH). The β hairpin L1 and L3 loops (positions 4-7 and positions 65-81 respectively) are indicated each by a line above or below the sequence.

FIG. 7 shows the amino acid sequence (SEQ ID NO:6) of the human platelet-derived growth factor-A chain (PDGF A-Chain). The β hairpin L1 and L3 loops (positions 11-36 and positions 58-88 respectively) are indicated each by a line above or below the sequence.

FIG. 8 shows the amino acid sequence (SEQ ID NO:7) of the human platelet-derived growth factor-B chain (PDGF B-Chain). The β hairpin L1 and L3 loops (positions 17-42 and positions 64-94 respectively) are indicated each by a line above or below the sequence.

FIG. 9 shows the amino acid sequence (SEQ ID NO:8) of the human nerve vascular endothelial growth factor (VEGF). The β hairpin L1 and L3 loops (positions 27-50 and positions 73-99 respectively) are indicated each by a line above or below the sequence.

FIG. 10 shows the amino acid sequence (SEQ ID NO:9) of the human nerve growth factor (NGF). The β hairpin L1 and L3 loops (positions 16-57 and positions 81-107 respectively) are indicated each by a line above or below the sequence.

FIG. 11 shows the amino acid sequence (SEQ ID NO:10) of the human brain derived neurotrophic factor (BDNF). The β hairpin L1 and L3 loops (positions 14-57 and positions 81-108 respectively) are indicated each by a line above or below the sequence.

FIG. 12 shows the amino acid sequence (SEQ ID NO:11) of the human neurotrophin-3 (NT-3). The β hairpin L1 and L3 loops (positions 15-56 and positions 80-107 respectively) are indicated each by a line above or below the sequence.

FIG. 13 shows the amino acid sequence (SEQ ID NO:12) of the human neurotrophin-4 (NT-4). The β hairpin L1 and L3 loops (positions 18-60 and positions 91-118 respectively) are indicated each by a line above or below the sequence.

FIG. 14 shows the amino acid sequence (SEQ ID NO:13) of the human transforming growth factor B-1 (TGF-B1). The β hairpin L1 and L3 loops (positions 21-40 and positions 82-102 respectively) are indicated each by a line above or below the sequence.

FIG. 15 shows the amino acid sequence (SEQ ID NO:14) of the human transforming growth factor B-2 (TGF-B2). The β hairpin L1 and L3 loops (positions 21-40 and positions 82-102 respectively) are indicated each by a line above or below the sequence.

FIG. 16 shows the amino acid sequence (SEQ ID NO:15) of the human transforming growth factor B-3 (TGF-B3). The β hairpin L1 and L3 loops (positions 21-40 and positions 82-102 respectively) are indicated each by a line above or below the sequence.

FIG. 17 shows the amino acid sequence (SEQ ID NO:16) of the human transforming growth factor B-4 (TGF-B4). The β hairpin L1 and L3 loops (positions 267-287 and positions 319-337 respectively) are indicated each by a line above or below the sequence.

FIG. 18 shows the amino acid sequence (SEQ ID NO:17) of the human neurturin. The β hairpin L1 and L3 loops (positions 104-129 and positions 166-193 respectively) are indicated each by a line below the sequence.

FIG. 19 shows the amino acid sequence (SEQ ID NO:18) of the inhibin α. The β hairpin L1 and L3 loops (positions 266-286 and positions 332-359 respectively) are indicated each by a line below the sequence.

FIG. 20 shows the amino acid sequence (SEQ ID NO: 19) of the inhibin A β subunit. The β hairpin L1 and L3 loops (positions 326-346 and positions 395-419 respectively) are indicated each by a line below the sequence.

FIG. 21 shows the amino acid sequence (SEQ ID NO:20) of the human inhibin B β subunit. The β hairpin L1 and L3 loops (positions 307-328 and positions 376-400 respectively) are indicated each by a line below the sequence.

FIG. 22 shows the amino acid sequence (SEQ ID NO:21) of the human activin A subunit. The β hairpin L1 and L3 loops (positions 326-346 and positions 395-419 respectively) are indicated each by a line below the sequence.

FIG. 23 shows the amino acid sequence (SEQ ID NO:22) of the human activin B subunit. The β hairpin L1 and L3 loops (positions 308-328 and positions 376-400 respectively) are indicated each by a line below the sequence.

FIG. 24 shows the amino acid sequence (SEQ ID NO:23) of the human Müllerian inhibitory substance (MIS). The β hairpin L1 and L3 loops (positions 465-484 and positions 530-553 respectively) are indicated each by a line below the sequence.

FIG. 25 shows the amino acid sequence (SEQ ID NO:24) of the human bone morphogenic protein-2 (BMP-2). The β hairpin L1 and L3 loops (positions 302-321 and positions 365-389 respectively) are indicated each by a line below the sequence.

FIG. 26 shows the amino acid sequence (SEQ ID NO:25) of the human bone morphogenic protein-3 (BMP-3). The β hairpin L1 and L3 loops (positions 373-395 and positions 441-465 respectively) are indicated each by a line below the sequence.

FIG. 27 shows the amino acid sequence (SEQ ID NO:26) of the human bone morphogenic protein-3b (BMP-3b). The β hairpin L1 and L3 loops (positions 379-402 and positions 447-471 respectively) are indicated each by a line below the sequence.

FIG. 28 shows the amino acid sequence (SEQ ID NO:27) of the human bone morphogenic protein-4 (BMP-4). The β hairpin L1 and L3 loops (positions 312-333 and positions 377-401 respectively) are indicated each by a line below the sequence.

FIG. 29 shows the amino acid sequence (SEQ ID NO:28) of the human bone morphogenic protein-5 Precursor (BMP-5). The β hairpin L1 and L3 loops (positions 357-378 and positions 423-447 respectively) are indicated each by a line below the sequence.

FIG. 30 shows the amino acid sequence (SEQ ID NO:29) of the human bone morphogenic protein-6/Vgrl (BMR-6). The β hairpin L1 and L3 loops (positions 21-40 and positions 81-102 respectively) are indicated each by a line above the sequence.

FIG. 31 shows the amino acid sequence (SEQ ID NO:30) of the human bone morphogenic protein-7/osteogenic protein (OP)-1 (BMP-7). The β hairpin L1 and L3 loops (positions 21-40 and positions 81-102 respectively) are indicated each by a line above the sequence.

FIG. 32 shows the amino acid sequence (SEQ ID NO:31) of the human bone morphogenic protein-8/osteogenic protein (OP)-2 (BMP-8). The β hairpin L1 and L3 loops (positions 305-326 and positions 371-395 respectively) are indicated each by a line below the sequence.

FIG. 33 shows the amino acid sequence (SEQ ID NO:32) of the human bone morphogenic protein-10 (BMP-10). The β hairpin L1 and L3 loops (positions 327-353 and positions 393-416 respectively) are indicated each by a line below the sequence.

FIG. 34 shows the amino acid sequence (SEQ ID NO:33) of the human bone morphogenic protein-11 (BMP-11). The β hairpin L1 and L3 loops (positions 318-337 and positions 376-400 respectively) are indicated each by a line above or below the sequence.

FIG. 35 shows the amino acid sequence (SEQ ID NO:34) of the human bone morphogenic protein (BMP-15). The β hairpin L1 and L3 loops (positions 295-316 and positions 361-385 respectively) are indicated each by a line below the sequence.

FIG. 36 shows the amino acid sequence (SEQ ID NO:35) of the norrie disease protein (NDP). The β hairpin L1 and L3 loops (positions 43-62 and positions 100-123 respectively) are indicated each by a line above or below the sequence.

FIG. 37 shows the amino acid sequence (SEQ ID NO:36) of the human growth differentiation factor-1 (GDF-1). The β hairpin L1 and L3 loops (positions 271-292 and positions 341-365 respectively) are indicated each by a line below the sequence.

FIG. 38 shows the amino acid sequence (SEQ ID NO:37) of the human growth differentiation factor-5 Precursor (GDF-5). The β hairpin L1 and L3 loops (positions 404-425 and positions 470-494 respectively) are indicated each by a line below the sequence.

FIG. 39 shows the amino acid sequence (SEQ ID NO:38) of the human growth differentiation factor-8 (GDF-8). The β hairpin L1 and L3 loops (positions 286-305 and positions 344-368 respectively) are indicated each by a line below the sequence.

FIG. 40 shows the amino acid sequence (SEQ ID NO:39) of the human growth differentiation factor-9 (GDF-9). The β hairpin L1 and L3 loops (positions 357-378 and positions 423-447 respectively) are indicated each by a line below the sequence.

FIG. 41 shows the amino acid sequence (SEQ ID NO:40) of the human glial derived factor Artemin (GDNF). The β hairpin L1 and L3 loops (positions 144-163 and positions 209-229 respectively) are indicated each by a line below the sequence.

FIG. 42 shows the amino acid sequence (SEQ ID NO:41) of the human glial derived factor persephin (GDNF). The β hairpin L1 and L3 loops (positions 70-89 and positions 128-148 respectively) are indicated each by a line below the sequence.

### DETAILED DESCRIPTION OF THE INVENTION

The present invention relates to novel mutant cystine knot growth factor (CKGF) proteins comprising one or more mutant subunits. These mutant subunits contain amino acid substitutions, additions, or deletions that result in conveying to the novel mutant CFGF proteins altered binding characteristics. The invention further relates to polynucleotides encoding the mutant CKGF subunits, methods for making the proteins and polynucleotides and diagnostic and therapeutic methods based thereon.

The novel mutant CKGFs of the invention alternatively possess: (a) novel properties absent from naturally occurring or wild type CKGFs, or (b) improvements in desirable pharmacological properties that characterize wild type CKGFs. Preferably, when compared with wild type CKGFs, the novel mutant CKGFs disclosed herein have a higher affinity for their cognate receptors. Additionally, the novel mutant CKGFs can be either more active or less active in effecting receptor-mediated signal transduction. In certain embodiments, the novel mutant CKGFs have prolonged half-lives in vivo.

The novel properties possessed by the mutant CKGF proteins arise from the amino acid substitutions, additions, or deletions that alter the electrostatic interactions that occur between the CKGF protein as ligand and its biological receptor. Positively charged residues in the peripheral loops of the CKGF proteins play an important role in receptor interaction. By altering the electrostatic nature of the peripheral loop common to the CKGF superfamily of proteins, mutant CKGF proteins are produced that display increased biological activity as compared to the wild type form of the molecule. Those proteins are one aspect of the present invention.

The Cystine Knot Growth Factors

The CKGF superfamily comprises proteins that control cell proliferation, differentiation and survival. To date, four distinct families of proteins have been identified within the superfamily. These are the glycoprotein hormones, platelet derived growth factors and related proteins, the neurotrophins and related proteins, and the transforming growth factors type β (TGF-β) and related proteins (See Table 1).

The protein families within the CKGF superfamily of the invention differ from each other in function and polypeptide sequence. Within the CKGF superfamily, members of one family need not necessarily share significant sequence identity with members of the other families. Nevertheless, the three-dimensional structures of the superfamily members comprise the cystine knot topology. Furthermore, the cystine knot topology results in the creation of various hairpin loop structures within the CKGF superfamily members that play an important role in determining the ligand-receptor interactions of the CKGF superfamily members and their receptors. Thus, there are common structural features that link the CKGF superfamily members.

Interestingly, the superfamily members have differing numbers of cystine disulfides in their active dimer forms and act through different cell surface receptors. For example, NGF and PDGF each have receptors that function through tyrosine kinase domains, whereas TGF-β has a complex signalling system involves a serine/threonine kinase. The receptors for the glycoprotein hormones are coupled to G protein-mediated signalling pathways.

Identification of Loop Structures that Modulate Biological Activity

The present invention is based on the finding that mutations at certain positions in the CKGF hairpin loops significantly alter the biological activities of the assembled CKGFs. One class of mutations is directed toward altering the electrostatic nature of the hairpin loops of the CKGF proteins.

To chose the amino acids to be mutagenized, the amino acid sequences of various CKGF member proteins within a CKGF family were compared. This comparison examined the amino acid sequences from member proteins selected from a variety of animal species. The comparison discovered the presence of certain nonconservative amino acid substitutions existing between the members of the CKGF family. For example, human and bovine thyroid stimulating hormone (hTSH and bTSH, respectively) share 70% homology between their ox subunits and 89% homology between their β subunits. Yet, bTSH is 6-10 fold more potent than hTSH. (Yamazaki, et al., J. Clin. Endocrinol. Metab. 80:473-479 (1995)).

Further examination of these amino acid substitutions showed that a number of these nonconservative amino acid substitutions occurred in the hairpin loops of these proteins. Moreover, the changes in the amino acid sequence of examined proteins was found to have altered the electrostatic nature of the hairpin loops of these proteins. Using site-directed mutagenesis, the functional significance of the mutations appearing in these areas was studied. Key positions that influence biological activity of the CKGFs are located near or within segments of the polypeptides that constitute the β hairpin L1 loop and the β hairpin L3 loop of the CKGF subunits.

Accordingly, mutant subunits of CKGFs, CKGF derivatives, CKGF analogs, and fragments thereof, that have mutations in the amino acid sequences which constitute these β hairpin loops have been created and are described herein. The mutations may include, insertion and/or deletion of amino acid residues, and preferably, amino acid substitutions that alter the electrostatic character of the β hairpin L1 and/or L3 loops of the CKGF subunits so that certain desirable properties of the wild type CKGF subunit are enhanced.

It also has been discovered that the mutations described herein which increase bioactivity can synergize with each other so that mutant subunits having multiple mutations possess much higher bioactivity than would be expected from the sum of the additional activity conferred by each of the mutations individually.

The invention does not include mutations in subunits of CKGFs that are known in the art.

Process for Rationally Designing Mutant CKGFs

According to one aspect of the invention, the process of rationally designing a mutant CKGF subunit includes the steps of identifying one or more candidate positions in the amino acid sequence of a subunit of a CKGF, producing a mutant subunit that includes the mutation in the candidate position, and studying the functional characteristics of the mutant subunit and the assembled dimeric molecule using in vitro and in vivo assays to confirm that the mutant subunit possesses a modified biological activity. A protein data base provides the needed physical and chemical parameters that are used to create a three-dimensional model of the structure of a CKGF.

As disclosed herein, a set of design guidelines specifically applicable to methods of modifying CKGF subunits have been developed. In one embodiment, the design guidelines focus on the peripheral loops of CKGFs. One goal of these guidelines is to increase the affinity of a CKGF superfamily member for its respective receptor counterpart altering the electrostatic nature of the peripheral hairpin loops. Altering the electrostatic nature of the hairpin loops is accomplished by selecting amino acid residues in the selected hairpin loop regions and substituting or deleting the wild type residue with an amino acid residue with more desirable electrostatic characteristics.

Generally, CKGF proteins display increased biological activity when the electrostatic nature of the peripheral hairpin loops is changed from an acidic or neutral state to a more basic state. In view of this observation, amino acid substitutions in this region are made under the design guidelines of the present invention that increase the basic nature or positive charge of the mutagenized CKGF protein. For example, an acidic residue in the hairpin loop region can be mutagenized to a neutral or basic residue to alter the electrostatic character of the structural region. Also, the weak basic residue histidine can be mutagenized to a more basic residue. Additionally, a neutral amino acid can be mutagenized to a basic residue to alter the electrostatic character of the structural region. The guidelines further contemplate mutating the hairpin loop region by deleting residues in the general region of the hairpin loop so as to create a general increase in the positive electrostatic charge of the region of interest.

It should be noted that the present invention is not to be limited to mutagenesis guidelines that are directed toward increasing the basic or positive charge of the peripheral loops. The present invention further contemplates altering a peripheral hairpin loop from a basic electrostatic charge to an acidic one. Under such a design, amino acid substitutions in the hairpin loop region are made under design guidelines that increase the acidic nature or negative charge of the mutagenized CKGF protein. For example, a basic residue in the hairpin loop region can be mutagenized to a neutral or acidic residue to alter the electrostatic character of the structural region. Additionally, a neutral amino acid can be mutagenized to an acidic residue to alter the electrostatic character of the structural region. The guidelines further contemplate mutating the hairpin loop region by deleting residues in the general region of the hairpin loop so as to create a general increase in the negative electrostatic charge of the region of interest.

The residues chosen for substitution in the peripheral hairpin loops are selected using a number of factors. As discussed above, mutations in the amino acid sequence of a target CKGF protein are guided, in part, by an amino acid sequence alignment comparing the amino acid sequences from homologous CKGF proteins of a variety of different species.

The location of potential mutagenesis sites is preferably in the highly variable regions of the peripheral loops, however, conserved regions can also be mutagenized, provided the resulting mutant CKGF protein possesses the desired biological activity. Also, potential mutagenesis sites can be located in the solvent exposed residues of the peripheral loops, as residues in these regions are generally thought to be more tolerant of amino acid deletion or substitution. Amino acid residues that are “buried,” or not solvent exposed can be sites of mutagenesis, provided that the resulting mutant CKGF protein posesses the desired biological activity. Additionally, potential mutagenesis sites are preferably selected within the actual hairpin loop. Nevertheless, potential sites of mutagenesis can be located at the periphery of the hairpin loop.

The invention turther contemplates the introduction of multiple mutations that alter the electrostatic nature of the peripheral hairpin loops.

The mutagenesis guidelines of the present invention are implemented using the design process of the present invention. This process entails the selection of potential mutagenesis sites in a target CKGF protein as discussed above, and the evaluation of these potential mutation sites using a variety of computer modeling methods well known in the art. These methods are used to predict the structure and activity of each mutation in the subunit as modeled, evaluated and ranked by a human operator. Potential mutations that are evaluated as having potential utility are stored for future use, those mutations that are evaluated as detrimental are eliminated from consideration.

The information collected after each cycle of the design process is added to an evolving database of structural and functional data on the CKGF subunit. The process is reiterated to further refine the design of the mutant CKGF and to explore novel characteristics of the molecule.

Once the amino acid sequence for a mutant CKGF subunit has been designed by the above-described process, the mutant CKGF protein is generated. Standard molecular biological techniques well known to those having ordinary skill in the art are employed to prepare a polynucleotide sequence encoding the mutant subunit. In preparing this polynucleotide sequence, it is possible to utilize synthetic DNA by synthesizing the entire sequence de novo. Alternatively, it is possible to obtain the coding sequences encoding the wild type CKGF subunit and then generate nucleotide substitutions by site-directed mutagenesis. The resulting sequences are amplified by the polymerase chain reaction (PCR) and propagated utilizing well known and readily available cloning vectors and hosts. These vectors can be plasmid or viral vectors and the hosts can be prokaryotic or eukaryotic hosts.

In addition, an expression vector containing the mutated polynucleotide sequence encoding the mutant CKGF subunit can be generated. These expression vectors are constructed by inserting the mutated polynucleotide sequence into appropriate expression vectors, and transformed into hosts such as procaryotic or eukaryotic hosts. A variety of expression vectors are well known in the art and are readily available. Such vectors can express the mutant CKGF protein alone, or in the form of a fusion protein wherein the mutant CKGF protein and a fusion partner sequence are genetically linked within the expression vector. Bacteria, yeasts (or other fungi) or mammalian cells can be utilized as hosts for the expression constructs. Once an expression vector containing the mutated CKGF sequence is constructed and inserted into a host cell line, the mutant CKGF protein is expressed.

CKGF dimer formation is facilitated after the recombinant expression of the mutant CKGF protein. The recombinant protein, either as its native sequence or as a fusion polypeptide, is allowed to fold and assemble with a counterpart subunit to form a dimer. Generally, dimerization occurs in a physiological solution under appropriate conditions of pH, ionic strength, temperature, and redox potential. Thereafter the dimerized recombinant CKGF protein is recovered and optionally purified using standard separation procedures. Appropriate separation procedures include chromatography.

The thus obtained novel mutant CKGF protein comprising at least one mutant subunit can be utilized in a variety of forms. The mutant CKGF protein can be used by itself, in a detectably labelled form, in an immobilized form, or conjugated to drugs or other appropriate therapeutic agents. The novel mutant CKGF protein can be used in diagnostic, imaging, and therapeutic procedures and compositions. Fusion proteins, analogs, derivatives, and nucleic acid molecules encoding such proteins and analogs, and production of the foregoing proteins and analogs, e.g., by recombinant DNA methods, are also provided.

In particular aspects, the invention provides amino acid sequences of mutant subunits of CKGFs which are otherwise functionally active. “Functionally active” mutant subunits as used herein refers to material displaying one or more known functional activities associated with the wild-type subunit. These activities may include association with another subunit to form a homodimer or heterodimer, secretion as a subunit or as an assembled dimeric molecule, binding to its receptor, triggering receptor-mediated signal transduction, antigenicity and immunogenicity.

In specific embodiments, the invention provides fragments of mutant subunits of CKGFs consisting of at least 1 amino acid, 6 amino acids, 10 amino acids, 50 amino acids, or of at least 75 amino acids. In various embodiments, the mutant subunits comprise or consist essentially of a mutated L1 loop domain and/or a mutated L3 loop domain.

For clarity of disclosure, and not by way of limitation, the detailed description of the invention is divided into the subsections which follow.

TABLE 1
Examples of Cystine Knot Growth Factors and Their Receptors

| | Protein family | Bioactive form | Specific receptor |
|---|---|---|---|
| I. | Glycoprotein Hormones | | G protein coupled receptor |
| | TSH | α-TSHβ heterodimer | TSH-R |
| | CG | α-CGβ heterodimer | CG/LH-R |
| | LH | α-LHβ heterodimer | CG/LH-R |
| | FSH | α-FSHβ heterodimer | CG/LH-R |
| | α-Subunit | — | — |
| | CGβ-Subunit | — | — |
| II. | PDGF Family | | Tyrosine Receptor Kinase |
| | PDGF-AA | Homodimer | PDGF-Rα |
| | PDGF-BB | Homodimer | PDGF-Rβ |
| | PDGF-AB | Heterodimer | PDGF-Rα |
| | VEGF | Homodimer | Trk |
| | PDGF-B/v-sis | Heterdimer | PDGF-Rβ |
| III. | Neurotrophin Family | | Trk |
| | NGF | Homodimer | A |
| | BDNF | Homodimer | B |
| | NT-3 | Homodimer | C |
| | NT-4 | Homodimer | B |
| IV. | Transforming Growth | | Ser/Thr Receptor Kinase |
| | Factor-β Family | | |
| | TGF-β1 | Homodimer | I, II |
| | TGF-β2 | Homodimer | I, II |
| | TGF-β3 | Homodimer | I, II |
| | TGF-β4/ebaf | Homodimer | I, II |
| | Neurturin | Homodimer | Ret Ser/Thr rk |
| | Inhibin A | α-βA Heterodimer | I, II |
| | Inhibin B | α-βA Heterodimer | I, II |
| | Activin A | βA-βA Homodimer | I, II type I (Act-R I, Act-R IB) |
| | Activin B | βB-βB Homodimer | I, II type II(Act-R II Act-R IIB) |
| | Activin AB | βA-βB Heterodimer | I, II |
| | Müllerian Inhibitory | Homodimer | Ser/Thr rk |
| | Substance | | |
| | Bone Morphogenic Protein-2 | Homodimer or | Ser/Thr rk |
| | (BMP-2) | Heterodimer | |
| | Bone Morphogenic Protein-3 | Homodimer or | Ser/Thr rk |
| | (BMP-3)/Osteogenin | Heterodimer | |
| | Bone Morphogenic Protein-3 | Homodimer or | Ser/Thr rk |
| | (BMP-3b) | Heterodimer | |
| | Bone Morphogenic Protein-4 | Homodimer or | Ser/Thr rk |
| | (BMP-4) | Heterodimer | |
| | Bone Morphogenic Protein-5 | Homodimer or | Ser/Thr rk |
| | (BMP-5) (precursor only) | Heterodimer | |
| | Bone Morphogenic Protein-6 | Homodimer or | Ser/Thr rk |
| | (BMP-6)/Vgrl | Heterodimer | |
| | Bone Morphogenic Protein-7 | Homodimer or | Ser/Thr rk |
| | (BMP-7)/Osteogenic Protein | Heterodimer | |
| | (OP)-1 | | |
| | Bone Morphogenic Protein-8 | Homodimer or | Ser/Thr rk |
| | (BMP-8)/Osteogenic Protein | Heterodimer | |
| | (OP)-2 | | |
| | Bone Morphogenic Protein- | Homodimer or | Ser/Thr rk |
| | 10 (BMP-10) | Heterodimer | |
| | Bone Morphogenic Protein- | Homodimer or | Ser/Thr rk |
| | 11 (BMP-11) | Heterodimer | |
| | Bone Morphogenic Protein- | Homodimer or | Ser/Thr rk |
| | 15 (BMP-15) | Heterodimer | |
| | Norrie Disease Protein | Homodimer or | Ser/Thr rk |
| | (NDP) | Heterodimer | |
| | Growth/Differentiation | Homodimer or | Ser/Thr rk |
| | Factor (GDF)-1 | Heterodimer | |
| | Growth/Differentiation | Homodimer or | Ser/Thr rk |
| | Factor-5 (GDF-5) (precursor | Heterodimer | |
| | only) | | |
| | Growth/Differentiation | Homodimer or | Ser/Thr rk |
| | Factor-8 (GDF-8) | Heterodimer | |
| | Growth/Differentiation | Homodimer or | Ser/Thr rk |
| | Factor-9 (GDF-9) | Heterodimer | |
| | Glial Cell-Derived | Homodimer | Ret Ser/Thr rk |
| | Neurotrophic Factor | | |
| | (GDNF)/Artemin | | |
| | Glial Cell-Derived | Homodimer or | Ser/Thr rk |
| | Neurotrophic Factor | Heterodimer | |
| | (GDNF)/Persephin | | |

Structural Features of the Cystine Knot Growth Factors

As indicated above, the cystine knot growth factor (CKGF) superfamily comprises at least four families of growth factors: the glycoprotein hormones, the PDGF family, the neurotrophins, and the TGF-β family. Other proteins not belonging to the above-mentioned four families, but having structures that comprise the cystine knot topology and the β hairpin loops are also members of the CKGF superfamily, and fall within the scope of the invention.

The structural similarities among the four growth factor families were not predicted prior to the solution of the three-dimensional structures or representative family members. This conclusion is based upon the lack of homology among the polypeptide sequences of the individual CKGF superfamily members. Nevertheless, it is now clear that all four families of growth factors share a common fold or topological structure. The crystal structures of NGF (McDonald et al., 1991, Nature, 354:411-414), TGF-β₂ (Schlunegger et al., 1993, J. Mol. Biol., 231:445-458), PDGF-BB (Osfner et al., 1992, EMBO J. 11:3921-3926) and hCG (Lapthom et al., 1994, 369:455-461) demonstrate that each protein comprises a very similar cluster of three conserved intramolecular disulfide bonds. Moreover, the backbone conformations of the members of the CKGF superfamily are remarkably similar, especially in the regions near the cystine knot, including a conserved twist in the middle of the fourth strand.

Comparison of the cysteines of the cystine knot structure clearly shows that not only are the connectivities of these half cysteines identical among the resolved cystine structures, but the positions of the six Cα atoms of these cysteines are also readily superimposable, resulting in a root-mean-square (rms) agreement of 0.5 to 1.5 Å between different members of the superfamily. For example, pairwise superpositions of the equivalent Cα atoms give the following root mean square (rms) distance values; for NGF versus PDGF-BB, 0.88 Å; for PDBF-BB versus TGF-β2, 0.65 Å and for NGF versus TGF-β2, 0.93 Å.

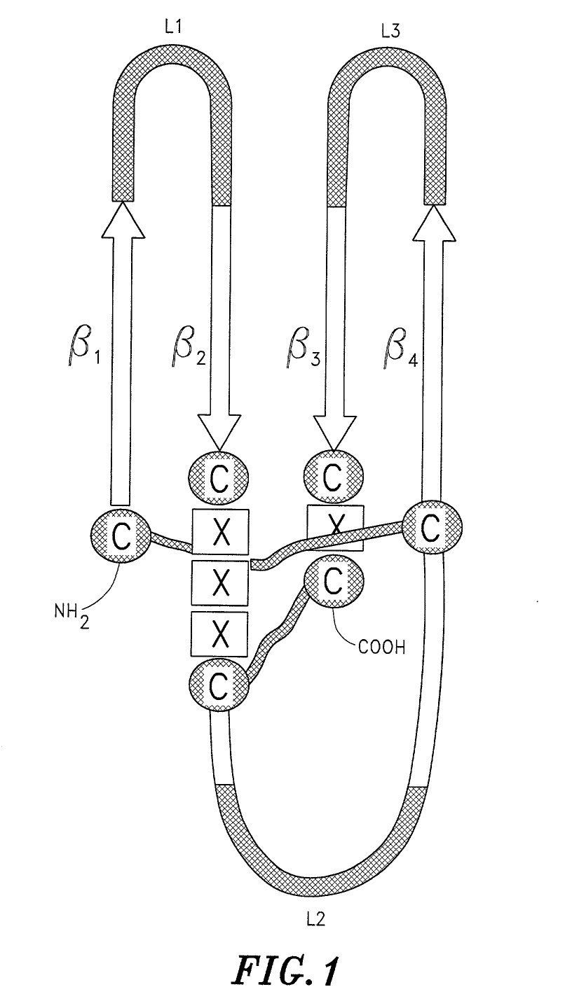

Each cystine knot structure is configured such that the three conserved cysteines are paired: I-IV, II-V, and III-VI (Table 2). Disulfide bonds II-V and III-VI, with their connecting residues, form a ring, through which the I-IV disulfide bond passes with the same topology, and approximately at right angles, thus forming a disulfide cluster (FIG. 1). The ring size is identical in TGF-β2 and PDGF-BB with sequences Cys(II)-X-Gly-X-Cys(III) and Cys(V)-Lys-cys(VI). In each case the glycine between Cys(II) and Cys(III) is in a positive φ conformation. This coupled with the lack of a side chain on glycine, facilitates the passing of disulfide bond I-IV through the ring. In NGF, the sequence between Cys(II) and Cys(III) consists of nine amino acids in a series of tight turns and, although a glycine occurs in a positive φ conformation in the position preceding Cys(III), the longer loop would in any case be sufficient to accommodate the Cys(I)-Cys(IV) bond.

Some general features emerge from the sequence alignment provided by the structural superpositions. For example, the spacing of the last two cysteines is always CXC—with only one residue between Cys V and Cys VI; and the size of the cystine ring depends on the spacing between Cys II and Cys III, which varies from 3 to 15. Among the five peptide chains in the structures of TGF-β2, PDGF-BB, β-NGF, and hCG, four have an 8-membered cystine ring and one, β-NGF, has a 14-membered cystine ring. Where only three residues lie between Cys II and Cys III, as is the case for all members of the TGF-β and PDGF families and glycoprotein hormones, the middle residue between the two cysteines is always a glycine to give a CXGXC (SEQ ID NO:5) pattern.

The cystine knot structure assumes a curled sheet-like nonglobular shape with overall dimensions of approximately 60×20×15 Å. The face of the sheet being formed by four irregular, distorted antiparallel β-strands. The three intramolecular disulfides form the center of a hydrophobic core which is the most rigid and least exposed part of the molecule. The β-strand loops connecting the cystine residues show considerable scope for size and sequence variation, providing different receptor-binding specificities without disturbing the basic structure of the core.

The similarity in overall topology shared among the CKGF member proteins also involves distorted β-hairpin loops between Cys(I) and Cys(II) and between Cys(IV) and Cys(V), and a more open connection between Cys(III) and Cys(VI). Although the three loops differ in length, the hydrogen bonding patterns, especially around the cluster of cysteines, are remarkably similar. In each member there are hydrogen bonds between the antiparallel strands around Cys(I) and Cys(II) such that the residue after Cys(I) (Asp16 in NGF) makes a hydrogen bond to the residue after Cys(II) (Arg59 in NGF). There is an extended β-hairpin ladder of hydrogen bonds between the two β-strands but the loop between them differs in length, conformation and hydrogen bonding patterns in the families.

The hydrogen bonding between the antiparallel β-strands around Cys(IV), Cys(V) and Cys(IV) is also similar. Hydrogen bonds exist between the residue before Cys(IV) (Tyr79 in NGF) and after Cys(VI) (e.g., Val111 in NGF); between the residue following cys(IV) (Thr81 in NGF); and the residue which lies between cys(V) and Cys(VI) (Val109 in NGF); and between the third residue from Cys(IV) (Thr83 in NGF) and that preceding Cys(V) (Ala107 in NGF). The β-ladders of the hairpins are much more extensive than in the first β-hairpin and there is always a β-bulge just before Cys(V). The twisted hairpins in NGF and PDGF-B are similar, but longer in the latter. In TGF-β2, this hairpin is further distorted by an insertion of two residues (Asn103 and Met104) which cause the hairpin to fold over to a greater extent. The connection between Cys(III) and Cys(IV) differs in length between NGF, TGF-β2 and PDGF-BB. The shortest loop occurs in PDGF-B. In NGF, it is replaced by a longer series of β-turns (a β-meander) and in TGF-β2 an even longer connection occurs, including a 12-residue α-helix. However, all are accommodated within the fixed framework of the strands forming the two hairpins and the disulfide cluster.

Members of the CKGF superfamily have been shown to have most if not all the above-desired topological and structural features. Other proteins possessing these features also are considered to be members of the CKGF superfamily. Methods of rational design applicable to CKGFs disclosed herein are also applicable to those proteins.

TABLE 2
List of Disulfide Bonds

| Cystine knot | β-NGF | TGF-β2 | PDGF-BB | hCG-α | hCG-β |
|---|---|---|---|---|---|
| I-IV | 15-18 | 15-78 | 16-60 | 10-60 | 9-57 |
| II-V | 58-108 | 44-109 | 49-97 | 28-82 | 34-88 |
| III-VI | 68-110 | 48-111 | 53-99 | 32-84 | 38-90 |
| Interchain | None | 77-77 | 43-52 | | |
| | | | 52-43 | | |
| Other | | 7-16 | | 7-31 | 23-72 |
| | | | | 59-87 | 26-110 |
| | | | | | 93-100 |

Structure and Function Analysis of CKGF Subunits

The present invention also provides a systematic approach for the rational design of novel mutant CKGF proteins comprising one or more mutant subunits. Described herein are methods for analyzing the structure of wild type and mutant CKGF subunits, CKGF dimers and CKGF analogs, and methods for determining the in vitro activities and in vivo biological functions of these molecules.

There are several considerations for specifying the amino acid position to be mutated in a CKGF protein. There are also a number of considerations for predicting the tolerance of specific residues in a particular region and for avoiding unwanted changes in analog specificity or stability. Sequence comparison of homologous proteins combined with three-dimensional structure modeling provide a rich source of information useful for interpreting structure-function relationships among proteins.

A molecular model of hTSH was constructed using as a template an hCG model derived from crystallographic data from Brookhaven Protein Data Bank (PDB). This model provides important leads for analog design limiting the number of necessary substitutions. Modeling of mutants is also invaluable for the interpretation of functional data. We have found that combined sequence-structure based predictions are often verified by functional changes observed in the analog.

First among the design considerations is that each protein contains functionally more important regions (such as the receptor binding site or the active site of an enzyme) and less important regions. It has been consistently found that the rate of evolution in the functionally more important parts of protein is considerably slower than in the functionally less constrained parts of molecules, such as for example peripheral β-hairpin loops of glycoprotein hormones. Consequently, solvent-exposed residues such as those in peripheral loops are less conserved than residues buried within the protein core. A conservative change of the most conserved amino acids is more likely to be deleterious. In contrast, a similar change in the less functionally constrained parts of the protein may have a higher chance of representing a type of “fine-tuning” improvement favored by natural selection. It is generally known that the overall fold of protein is usually highly conserved even after multiple amino acid substitutions. Thus, mutations located in the peripheral loops of hTSH are not expected to alter the overall fold of hTSH. Such prediction is supported by homology modeling of analogs as well as by the presence of “gain of function” mutations.

Second among the design considerations is the recent development of glycoprotein hormone superagonists supports a prediction that combination of domains with activity or receptor binding specificity maximized previously at a certain stage of protein evolution may provide a universal strategy for engineering human protein analogs. In the case of human glycoprotein hormones, selection of substitutions from the large library of homologous sequences in different vertebrate species largely reduces the probability of profoundly deleterious, nonconclusive mutations. This observation is consistent with the known ability of glycoprotein hormone subunits from different species to reassociate into functionally active hormones.

Third among the design considerations is that the regions known to confer protein specificity should be generally avoided in analog design, unless the change of hormone specificity is a part of intended modification. For example, recent studies involving β-subunit chimeras have shown that the “seat-belt” region is critical for conferring glycoprotein hormone specificity, probably by restricting heterologous ligand-receptor interactions and/or influencing the conformation of the composite binding domain. Furthermore, an unexpectedly high thyrotropic activity of hCG/hFSH chimeras suggested that specificity cannot reliably be predicted from the amino acid sequence and should be verified for all chimeras.

Fourth among the design considerations is that mammalian glycoprotein hormones have been shown to possess a low degree of species specificity. For example, mammalian TSH proteins have been shown to stimulate thyroid function in all vertebrates with the exception of certain fishes. Moreover, highly purified mammalian LH also has thyrotropic activity in other species, including species that are only as remotely related as teleosts. Moreover, we have found correlations between receptor binding affinity and biological activity of human TSH using TSH receptors from different mammalian species. Analogously, the introduction of residues and domains present in other species or homologous hormones is tolerated in many instances without alteration of hormone specificity.

Finally, the primary targets for site-detected mutagenesis are modification-permissive domains which can be predicted by sequence comparison. These domains are defined as regions of the molecule which allow introduction of nonconservative amino acid changes, enabling modulation of function without compromising subunit synthesis or assembly. Significantly, mutagenesis of the amino acid residue undergoing multiple and/or nonconservative changes during evolution does not ordinarily result in the loss of function or decrease of hormone expression.

The gain-of-function method for designing CKGF mutants involves first identifiing a “modification permissive domain” of the CKGF protein which tolerates introduction of nonconservative substitutions without compromising protein synthesis. Further mutagenesis in a modification permissive domain permits identification of substitutions which result in increased hormone bioactivity. Subsequent multiple residue replacements can be used to elucidate cooperative effects of individual residues and can be extended to the simultaneous mutagenesis of multiple hormone domains. The identification of gain-of-function mutations led to the finding that a partial or complete loss of hTSH activity caused by modifications in one domain can be completely compensated, thereby indicating that the TSH receptor is capable of accommodating ligands with significant structural modifications by means of an “analog induced fit”. It is even possible to create alternative contact domains of analog and receptor which are still able to transduce a signal.

Moreover, identification of cooperative, non-cooperative and mutually exclusive hormone domains can provide important leads for the development of therapeutically useful hormone analogs. With such approaches, it should ultimately be possible to individually modulate and dissociate biological properties of CKGFs.

Methods Based on Three-Dimensional Structure and Sequence Aligment

The methods for analyzing the structure of a CKGF subunit are based on analysis of polypeptide sequence data and three-dimensional protein structure data. One skilled in the art will readily appreciate that other biochemical data also can be used in the analysis.

The polypeptide sequence of a protein can be determined by methods well known in the art, such as standard techniques of protein sequencing, or hypothetical translation of the genetic sequence encoding the protein. Polypeptide sequences and polynucleotide sequences are generally available in sequence databases, such as GenBank. Computer programs, such as Entrez, can be used to browse the database and retrieve any amino acid sequence and genetic sequence data of interest for further analysis. Amino acid sequence and genetic sequence can be retrieved from a database by accession number. These databases can also be searched to identify sequences having various degrees of similarities to a query sequence using programs, such as FASTA and BLAST, which rank the similar sequences by alignment scores and statistics. Since the extent of sequence similarity between members of different families within the CKGF superfamily are low, searches with a query sequence are performed primarily to identify members within the same family.

The protein sequence of a CKGF subunit can also be characterized using a hydrophilicity analysis (Hopp, T. and Woods, K., 1981, Proc. Natl. Acad. Sci. U.S.A. 78:3824). A hydrophilicity profile can be used to identify the hydrophobic and hydrophilic regions of the subunit. Using this information and procedures that will be familiar to those having ordinary skill in the art, corresponding polynucleotide sequences encoding these regions can then be determined.

Secondary structural analysis (Chou, P. and Fasman, G., 1974, Biochemistry 13:222) can also be performed using the protein sequence of the CKGF subunit to identify regions of the subunit that assume specific secondary structures.

Methods of structural analysis that include X-ray crystallography (Engstom, A., 1974, Biochem. Exp. Biol. 11:7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.) can also be employed. Structure prediction, analysis of crystallographic data, sequence alignment, as well as homology modelling can be accomplished using commercially available computer software readily available in the art, such as BLAST, CHARMm release 21.2 for the Convex, and QUANTA v.3.3, (Molecular Simulations, Inc., York, United Kingdom).

Computer Assisted Methods

A computer model of the three-dimensional (3D) structure of a CKGF subunit can be constructed based on polypeptide sequence data. Other information, including the polypeptide sequence and 3D structure of other CKGFs subunits, also can be used in the computer modeling. A model of a CKGF or a CKGF subunit is constructed to represent a 3D structure of the molecule having the same connectivity of cystine residues.

The computer model can be elaborated using software algorithms known in the art for minimizing energy, optimizing the forces that determine intramolecular folding, such as hydrophobic, electrostatic, van der Waals, and hydrogen bond interactions. The disposition of each atom in the molecule relative to each other atom is optimized to conform to the overall cystine knot topology. The optimizing process can be formed automatically by computer software and/or a skilled human operator. Visual comparisons of hydrogen bonds and strand conformations within the topology can be carried out with the assistance of an interactive computer graphics display system.

Currently, there are publicly available at least five protein structures of CKGF subunits determined at 2.0 Å or higher resolution. The structures of these and other CKGFs can be determined or refined using techniques such as X-ray crystallography, neutron diffraction, and nuclear magnetic resonance (NMR).

Structure determination by X-ray crystallography produces a file of data for the protein. The Brookhaven Protein Data Bank (BPDB) exemplifies a repository of protein structural information, which is created and supplemented by the Brookhaven National Laboratory in Upton, Long Island, N.Y. Any other database which includes implicitly or explicitly the following data would be useful in connection with the methods described herein: (1) the amino acid sequence of each polypeptide chain; (2) the connectivity of disulfides; (3) the names and connectivities of any prosthetic groups; (4) the coordinates (x, y, z) of each atom in each observed configures; (5) the fractional occupancy of each atom; and (6) the temperature factors of the atoms. There is at least one record for each atom for which a coordinate was determined. Coordinates are given in angstrom units (100,000,000 Δ=1 cm) on a rectangular Cartesian grid. As some parts of a protein may adopt more than one spatial configuration, there may be two or more coordinates for some atoms. In such cases, fractional occupancies are given for each alternative position. X-ray crystallographic data can give an estimate of atomic motion which is reported as a temperature or “Debye-Waller” factor.

Although protein coordinates are most commonly determined for proteins in crystals, it is now generally accepted that the solution structure of a protein will differ from the crystal structure only in minor details. Thus, given the coordinates of the atoms one can calculate the solvent accessibility of each atom. The surface accessibility of molecules can also be determined and a score based on the hydrophobic residues in contact with the solvent can be determined. In addition, the coordinates implicitly give the charge distribution throughout the protein. This is of use in estimating whether a mutant subunit will fold and/or associate to form a dimer.

Certain steps of the rational design process of the present invention are carried out on conventional computer systems having storage devices capable of storing amino acid sequences, structure data bases, and various application programs used for conducting the sequence comparisons and structure modeling. An interactive computer graphics display system allows an operator to view the chemical structures being evaluated in the design process of the present invention. Graphics and software programs are used to model the wild type and mutant subunits and to rank candidates.

For example, the computer graphics interactive display system allows the human operator to visually display one or more structures or partial structures of members of the CKGF family. The visual representation of multiple polypeptide chains and side chains of the amino acids can be manipulated and superimposed as desired which increase the ability to perform the structural design process. The computer graphics display system can perform a set of functions such as but not limited to zooming, clipping, intensity depth queuing (where objects further away from the viewer are made dimmer so as to provide a desired depth effect in the image being displayed); and translation and rotation of the image in any of the three axes of the coordinate system. It is to be understood that the present invention can be carried out using other computer programs, operating systems and programming languages. Any suitable type of software and hardware can be used for displaying and manipulating the computer representation of the structure of these molecules.

Computer programs can be utilized to calculate the energy for each of the wild type and mutant structures and to make local adjustments in the hypothetical structures to minimize the energy. Finally, programs can be used to identify unstable parts of the molecule and to simulate the formation of a mutant CKGF dimer (structure of the other subunit may be required for a heterodimer) and the binding of the mutant CKGF dimer to its receptor (if the structure of the receptor is determined or predictable from existing data).

Structural data from the databases define a three-dimensional object. For many members of the CKGF superfamily, the cysteine residues involved in forming the three disulfide bonds of the cystine knot have been identified. If such information is not known, the cysteine residues that form the cystine knot can readily be identified by systematic mutagenesis of the cysteine residues in the molecule.

Once all of the cysteine residues that form the cystine knot are identified, these residues of the CKGF subunit can be aligned with those of the other CKGFs to predict which segments of the polypeptide most probably form the β hairpin L1 and L3 loops in the CKGF subunit.

A least-squares analysis is applied to fit the atoms from one CKGF subunit to the atoms from another. This least-squares fit allows degrees of freedom to superimpose two three-dimensional objects in space. If the Root-Mean-Square (RMS) error is less then some preset threshold, the structure is a good fit for the model being considered. The final step in the process involves ranking the plausible candidates from most plausible to least plausible, and eliminating those candidates that do not appear to be plausible based on criteria utilized by a skilled human operator and/or expert computer system.

For example, it is preferred that hydrogen bonds exist between the residue before cysIV and cysVI; between the residue following cysIV and the residue between cysV and cysVII; and between the third residue along from cysIV and that preceding cysV. It is preferable that a human expert refine the computer model by visual comparison of the human structures of CKGF subunits, and ranking of possible/optimal prediction of structures.

The candidates for substitution, insertion, or deletion are provided to the human operator, who displays them in three dimensions utilizing the computer graphics display system. The operator then can make decisions about the candidates based on knowledge concerning protein chemistry and the physical relationship of the altered amino acid residue with respect to the overall cystine knot topology and receptor binding. This analysis can be used to rank the candidates from most optimal/plausible to least optimal/plausible. Based on these rankings, the most optimal candidates can be selected for site-directed mutagenesis and production. It is also desired for the computer to assist a human operator in making the ranking selections and eliminating candidates based on prior experience that has been derived from previous modeling and/or actual genetic engineering experiments.

A candidate can be rejected if any atom of the mutant CKGF comes closer than a minimum allowed separation to any retained atom of the native protein structure. For example, the minimum allowed separation could be set at 2.0 angstroms. Note that any other value can be selected. This step can be automated, if desired, so that the human operator does not manually perform this elimination process.

A candidate can be penalized if the hydrophobic residues have high exposure to solvent. The side chains of phenylalanine, tryptophan, tyrosine, leucine, isoleucine, methionine, and valine are hydrophobic.

A candidate can be penalized when the hydrophilic residues have low exposure to solvent. The side chains of serine, threonine, aspartic acid, glutamic acid, asparagine, glutamine, lysine, arginine, and proline are hydrophilic.

A candidate can be penalized when the resulting mutant polypeptide fails to form hydrogen bonds that exist between residues near the six cysteines, or form hydrogen bonds that tend to disrupt the disulfide bonds between any of the six cysteines.

Another design rule penalizes candidates having sterically bulky side chains at undesirable positions along the mutant polypeptide. Furthermore, it is possible to switch a candidate with a bulky side chain by replacing the bulky side chain by a less bulky one. For example, a side chain carries a bulky substituent such as leucine or isoleucine, a possible design step replaces this amino acid by a glycine, which is the least bulky side chain.

Other rules and/or criteria can be utilized in the selection process and the present invention is not limited to the rules and/or criteria discussed.

In this way, the topology-based approach and method of the present invention can be utilized to engineer mutant CKGFs having a very significantly increased probability of having an increase bioactivity than would be obtained using a random selection process. This means that the genetic engineering aspect of creating the desired mutants is significantly reduced, since the number of candidates that have to be produced and tested is reduced. The most plausible candidate can be used to genetically engineer an actual molecule.

Mutants of the Glycoprotein Hormones

As elaborated more fully below, one aspect of the invention provides CKGFs that are glycoprotein hormones comprising at least one subunit having mutations at amino acid positions located within the β hairpin L1 loop and the β hairpin L3 loop of the α and/or β subunit. In the context of the invention, glycoprotein hormone β subunit include the hCG β subunit, LH β subunit, FSH β subunit and TSH β subunit.

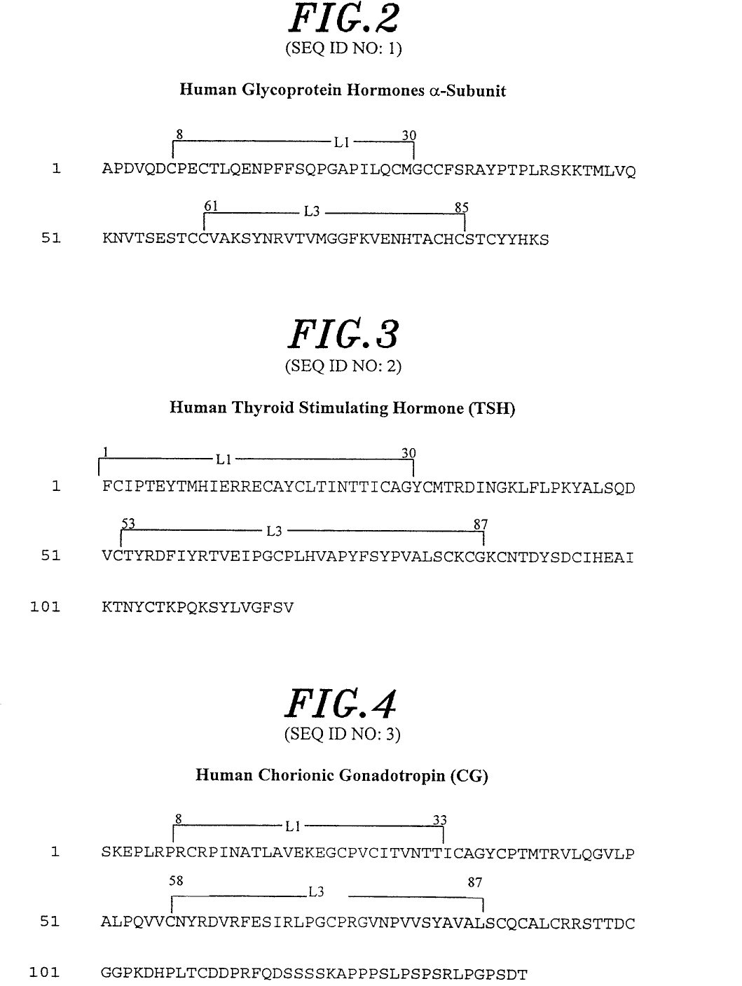

Mutant subunits can be created by combining individual mutations within a single subunit and by complexing mutant subunits to create doubly mutant heterodimers. In particular, the inventors have designed heterodimers that include mutuant α and mutant β mutant subunits, wherein the mutant subunits have mutations in specific domains. These domains include the β hairpin L1 and L3 loops of the common α subunit (as depicted in FIG. 2), and the β hairpin L1 and L3 loops of the glycoprotein hormone β subunit. In one embodiment, the present invention provides mutant a subunits, mutant TSH β subunits, mutant hCG β subunits, and TSH and hCG heterodimers comprising either one mutant α subunit or one mutant β subunit, wherein the mutant α subunit comprises single or multiple amino acid substitutions, preferably located within or near the β hairpin L1 and/or L3 loop of the α subunit, and wherein the mutant β subunit comprises single or multiple amino acid substitutions, preferably located within or near the β hairpin L1 and/or L3 loop of the β subunit. Preferably, these mutations increase bioactivity of the glycoprotein hormone heterodimer comprising the mutant subunit and the TSH heterodimer having the mutant subunit has also been modified to increase the serum half-life relative to the wild-type TSH heterodimer.

The α-subunit contains five disulfide bonds, three of which, Cys10-Cys60, Cys28-Cys82, and Cys32-Cys84, adopt the knotted configuration (Table 2). Except for a short three-turn α-helix located between residues 40 and 47, most of the secondary structures in the α-subunit are irregular β-strands and β-hairpin loops. The β-subunit contains six disulfide bonds; among them, Cys9-Cys57, Cys34-Cys88, and Cys38-Cys90 form the topological cystine knot.

The dimerization buries a total of 4525 square angstroms of surface area, according to Lapthorn et al. (Lapthorn et al., 1994, Nature, 369:455-61), and 3860 Å², according to Wu et al (1994, Structure, 2:545-58).

The present inventors have also found that one or more amino acid substitution that alter the electrostatic charge of the L1 and L3 β hairpin loop regions of the human α subunit (as depicted in FIG. 2 (SEQ ID NO: 1), results in an increase in the bioactivity of the mutant protein as compared to the wild type form of the molecule. In one embodiment, a substitution of a basic amino acid, such as lysine or arginine, more preferably arginine, increases the bioactivity of TSH relative to wild type TSH.

In another embodiment, the present invention provides a mutant CKGF subunit that is a mutant TSH β subunit having an amino acid substitution at position 6 as depicted in FIG. 3 (SEQ ID NO:2). The present invention also provides a mutant CKGF subunit that is a mutant hCG β subunit having an amino acid substitution at position 75 and/or 77 as depicted in FIG. 4 (SEQ ID NO:3).

In a preferred embodiment, the present invention provides a mutant CKGF that is a heterodimeric glycoprotein hormone, such as a mutant hCG or a mutant TSH, comprising at least one of the above-described mutant glycoprotein hormone α and/or β subunits.

According to the invention, a mutant βsubunit comprising single or multiple amino acid substitutions, preferably located in or near the β hairpin L3 loop of the β subunit, can be fused at its carboxyl terminal to the CTEP. Such a mutant β subunit-CTEP subunit may be coexpressed and/or assembled with either a wild type or mutant α subunit to form a functional TSH heterodimer which has a bioactivity and a serum half life greater than wild type TSH.

In another embodiment, a mutant β subunit comprising single or multiple amino acid substitutions preferably located in or near the β hairpin L3 loop of the β subunit, and mutant α subunit comprising single or multiple amino acid substitutions preferably located in or near the β hairpin L1 loop of the α subunit, are fused to form a single chain TSH analog. Such a mutant β subunit-mutant α subunit fusion has a bioactivity and serum half-life greater than wild type TSH.

In yet another embodiment, mutant β subunit comprising single or multiple amino acid substitutions preferably located in or near the β hairpin L3 loop of the β subunit and further comprising the CTEP in the carboxyl terminus, and mutant α subunit comprising single or multiple amino acid substitutions preferably located in or near the β hairpin L1 loop of the α subunit, are fused to form a single chain TSH analog.

Mutants of the Common α Subunit

The common human α subunit of glycoprotein hormones contains 92 amino acids. This amino acid sequence includes 10 half-cysteine residues, all of which are in disulfide linkages. The invention relates to mutants of the α subunit of human glycoprotein hormones wherein the subunit comprises single or multiple amino acid substitutions, preferably located in or near the β hairpin L1 loop of the α subunit. The amino acid residues located in or near the αL1 loop, starting from position 8-30 as depicted in FIG. 2 are found to be important in effecting receptor binding and signal transduction. Amino acid residues located in the αL1 loop, such as those at positions 11-22, form a cluster of basic residues in all vertebrates except hominoids, and have the ability to promote receptor binding and signal transduction.

According to the invention, the mutant α subunits have substitutions, deletions or insertions of one, two, three, four or more amino acid residues in the wild type protein.

Mutants of the Human Glycoprotein β Subunit

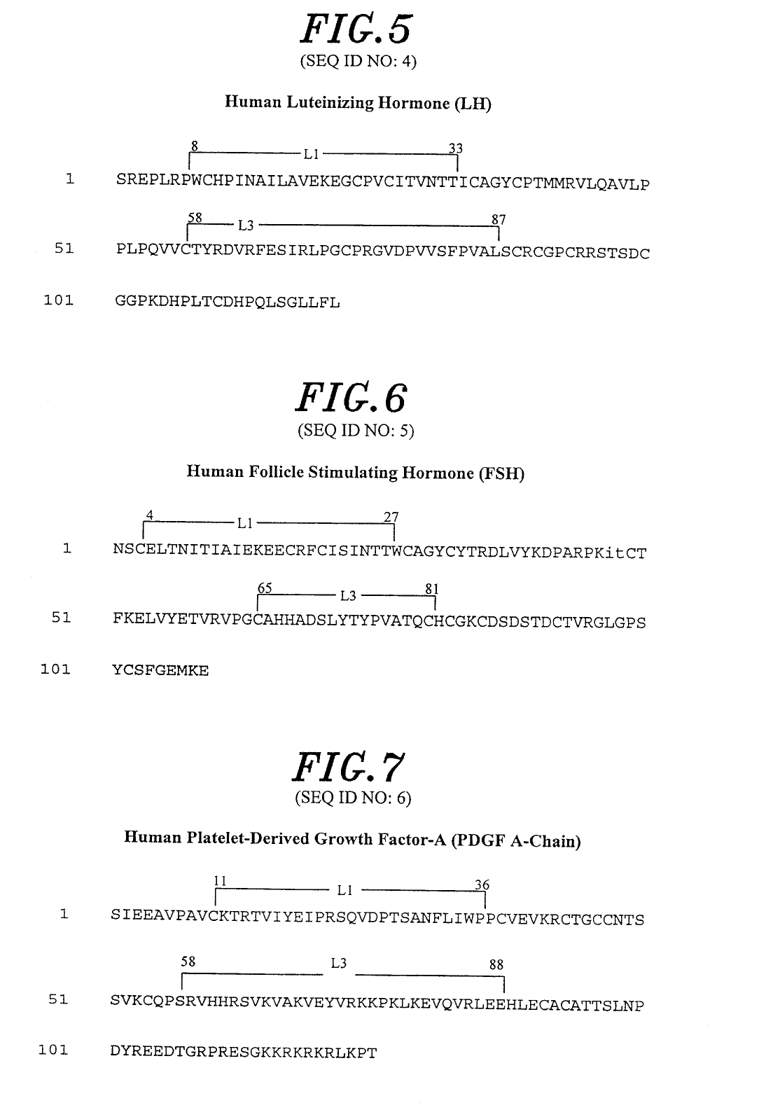

The number of amino acids in the β subunits of the human glycoprotein hormones range from 109 in FSH, depicted in FIG. 6 (SEQ ID No: 5)) to 140 amino acids in hCG, depicted in FIG. 4 (SEQ ID No: 3). The invention relates to mutants of the β subunit of the human glycoproteins which include TSH, CG, LH and FSH, wherein a mutant subunit of one of these protein hormones comprises single or multiple amino acid substitutions, preferably located in or near the β hairpin L1 and/or L3 loops of these β subunits, where such mutant β subunits are fused to CTEP of the β subunit of another human glycoprotein such as hCG or are part of a CKGF heterodimer having a mutant α subunit with an amino acid substitution at position 22 (as depicted in FIG. 2 (SEQ ID NO: 1)), or being an α subunit-β subunit fusion. The mutant β subunits of the present invention have substitutions, deletions or insertions, of one, two, three, four or more amino acid residues when compared with the wild type subunit.

Mutants of the PDGF Family

Platelet-derived growth factor (PDGF) is a major mitogenic factor for cells of mesenchymal origin. It promotes the growth and differentiation of fibroblasts and smooth muscle cells during development and embryogenesis. It also functions as a chemotactic reagent for inflammatory cells during wound healing (Heldin, 1992, EMBO J., 11:4251-59). Two forms of the PDGF gene are expressed, PDGF-A and PDGF-B, resulting in three isoforms of the dimeric growth factor, PDGF-AA, PDGF-AB, and PDGF-BB. Other members of the PDGF family include the vascular endothelial growth factor (VEGF) and the v-sis oncogene product of p28^{v-sis}, a transforming protein of simian sarcoma virus (SSV) which binds to and activates both the α and β PDGF receptors (Lee and Donoghue, 1991, J. Cell. Biol., 113:361-70).

Oefner et al. (1992, EMBO J. 11:3921-26) determined the crystal structure of the mature homodimeric isoform of human platelet-derived growth factor, PDGF-BB, at 3.0-Å resolution. The cystine knot structure comprises 109 amino acids and consists of four irregular anti-parallel β-strands and a 17-residue N-terminal tail. Of the eight disulfide-bonded cysteines, six, Cys16-Cys60, Cys49-Cys97, and Cys53-Cys99, form the knotted arrangement and two, Cys43-Cys52, form two interchain disulfide bonds (Table 2). The edges of the four-stranded β-sheet form the dimer, which results in the majority of inter-subunit contacts being between the first two strands of the β-sheet and the N-terminal tail. The total surface area buried is estimated to be 2200 square angstroms, and most of the buried residues are hydrophobic in nature.

The platelet-derived growth factor (PDGF) family is composed of proteins possessing varying numbers of amino acids as depicted in FIGS. 7-9 (SEQ ID Nos: 6-8). Often, the active form of members of this family of proteins are dimers, either homo- or heterodimers. The invention relates to mutations in the monomeric subunits of these proteins wherein a mutant monomer comprises a single or multiple amino acid substitutions, deletions or insertions, preferably located in or near the β hairpin L1 or L3 loops. Mutations outside of these hairpin loop regions that alter the structure of the hairpin loops such that the electrostatic interaction between the ligand and its cognate receptor are increased, are also contemplated. Fusion proteins and chimeric monomeric subunits are also contemplated by the present invention. The mutant PDGF monomers of the invention have amino acid substitutions, deletions or insertions, of one, two, three, four or more amino acid residues when compared with the wild type subunit.

Mutants of the Neurotrophin Family

The neurotrophins represent a family of growth factors that control the development and survival of certain neurons in both the peripheral (PNS) and the central nervous systems (CNS). The members of this family include nerve growth factor (NGF) (Levi-Montalcini, 1987, EMBO J. 6:1145-54), brain-derived neurotrophic factor (BDNF) (Hohn et al., 1990, Nature, 344:339-41; and Leibrock et al., 1989, Nature, 341:149-52), neurotrophin-3 (NT-3), neurotrophin-4 (NT-4), and neurotrophin-5 (NT-5) (Barde, 1989, Neuron, 2:1525-34; Berkemeier et al., 1991, Neuron, 7:857-66; and Hallbook et al., 1991, Neuron, 6:845-58).

The cystine knot structure of the prototype member of the neurotrophin family, β-NGF, consists mainly of four irregular anti-parallel β-strands (McDonald et al., 1991, Nature, 354:411-14; and Holland et al., 1994, J. Mol. Biol. 239:385-400) with an insertion of two shorter strands between the first and the second strand. The overall dimension of the molecule is roughly 60×25×15 Å. Six cystines in each monomer form the knotted disulfide bonds (Cys15-Cys80, Cys58-Cys108, and Cys68-Cys110, see Table 2) clustered at the one end of all the β-strands. The dimer is formed between the two flat faces of the four-stranded β-sheets, burying a total of 2300 square angstroms of surface area. The interface is characterized as largely hydrophobic.

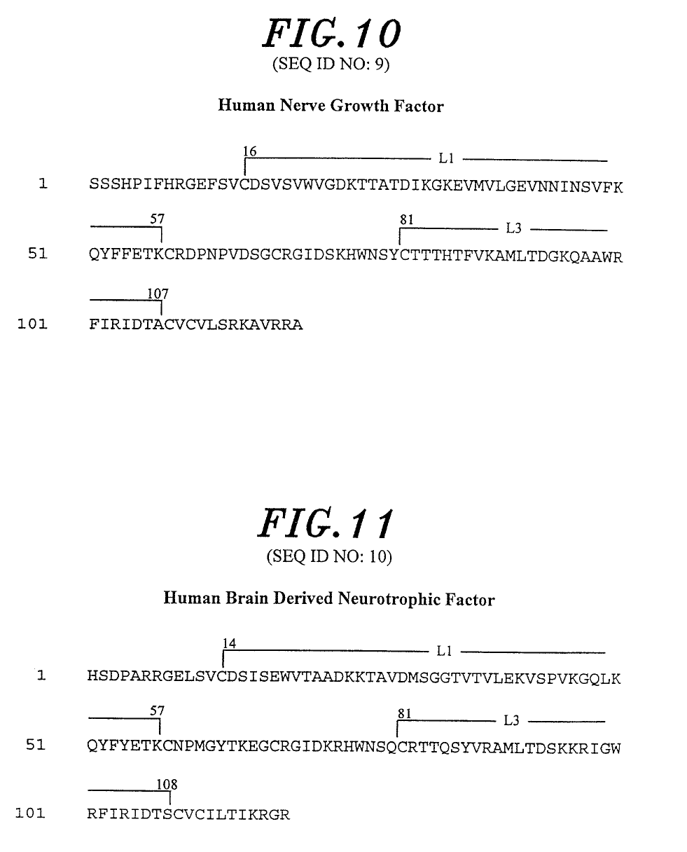

The neurotrophin family is composed of proteins possessing varying numbers of amino acids as depicted in FIGS. 10-13 (SEQ ID Nos: 9-12). Often, the active form of members this family of proteins are dimers, either homo- or heterodimers. The invention relates to mutations in the monomeric subunits of these proteins wherein a mutant monomer comprises a single or multiple amino acid substitutions, deletions or insertions, preferably located in or near the β hairpin L1 or L3 loops. Mutations outside of these hairpin loop regions that alter the structure of the hairpin loops such that the electrostatic interaction between the ligand and its cognate receptor are increased, are also contemplated. Fusion proteins and chimeric monomeric subunits are also contemplated by the present invention. The mutant neurotrophin monomers of the invention have amino acid substitutions, deletions or insertions, of one, two, three, four or more amino acid residues when compared with the wild type subunit.

Mutants of the TGF-β Family

The TGF-β family consists of a set of growth factors that share at least 25% sequence identity in their mature amino acid sequence. Members in this gene family include but are not limited to the transforming growth factors, TGF-β1, TGF-β2, TGF-β3, TGF-β4 and TGF-β5 (Assoan et al., 1983, J. Biol. Chem., 258:7155-60; Cheifetz et al., 1987, Cell, 48:409-15; Derynck et al., 1988, EMBO J., 7:3737-43; Jakowlew et al., 1988, J. Mol. Biol., 239:385-400; Jakowlew et al., 1988, Mol. Endocrinol., 2:1186-95; Kondaiah et al., 1990, J. Biol. Chem., 265:1089-93; and Ten Dikje et al., 1988, Proc. Natl. Acad. Sci., USA, 85:4715-19); inhibins and activins (inhibin A, inhibin B, activin A, and activin B) (Forage et al., 1986, Proc. Natl. Acad. Sci., USA, 83:301-95; Ling et al., 1986, Nature, 321:779-82; Mason et al., 1985, Nature, 318:659-63; and Vale et al., 1986, Nature, 321:776-79); bone morphogenic proteins, BMP-2, BMP-3, BMP-4, BMP-5, BMP-6 and BMP-7 (Celeste et al., 1990, Proc. Natl. Acad. Sci., USA, 87:9843-47; Ozkaynak et al., 1992, J. Biol. Chem., 267:25220-27; and Wozney et al., 1988, Science, 242:1528-34); the decapentaplegic gene complex, DPP-C (Padgett et al., 1987, Nature, 325:81-84); Vgl (Weeks and Melton, 1987, Cell, 51:861-67); vgr-1 (Lyons et al., 1989, Proc. Natl. Acad. Sci., USA, 86:4554-58); Müllerian inhibiting substance (MIS)(Cate et al., 1986, Cell, 45:685-98); a growth-differentiation factor, GDF-1 (Lee, 1991, Proc. Natl. Acad. Sci., USA, 88:4250-54); and dorsalin-1, dsl-1 (Centrella et al., 1988, FASEB J., 2:3066-73). Most proteins in this family exist as homo- or heterodimers.

The diverse biological activities of TGF-β in cell growth and regulation include: (a) its ability to interrupt the cell cycle during late G₁ phase, and to prevent induction of DNA synthesis and progression into S phase (Thompson et al., 1989, J. Cell Biol., 108:661-69; Centrella et al., 1988, FASEB J., 2:3066-73; and Heine et al., 1987, J. Cell Biol., 105:2861-76), (b) cell accumulation and their response to extracellular-matrix components, including type I, III, IV, and V collagen; tenascin; and elastin (Liu and Davidson, 1988, Biochem. Biophys. Res. Commun., 154:895-901; Pearson et al., 1988, EMBO J., 7:2677-81; and Varga et al., 1987, Biochem J., 247:597-604) and (c) promote or inhibit cell growth by modulating the secretion of other growth factors, for example, PDGF (Roberts et al., 1985, Proc. Natl. Acad. Sci., USA, 82:119-23).

The cystine knot structure of TGF-β2 consists mainly of four irregular anti-parallel β-strands and an 11-residue α-helix between the second and the third strand. Of the nine cystines in each monomer, eight form four intrachain disulfides. The three intrachain disulfide bonds Cys15-Cys78, Cys44-Cys109, and Cys48-Cys111, define a topological cystine knot in which the Cys15-Cys78 disulfide passes through a ring bounded by the Cys44-Cys109 and Cys48-Cys11 disulfides together with the connecting polypeptide backbone, residues 44-48 and 109-111.

The two monomers form a head-to-tail dimer with the residues on the long helix (residues 58-68) packed against the residues near the end of the β-sheets. The TGF-β2 growth factor exists as a disulfide-linked dimer in which the overall dimensions of each monomer are 60×20×15 Å.

The transforming growth factor-β family is composed of proteins possessing varying numbers of amino acids as depicted in FIGS. 14-42 (SEQ ID Nos: 13-41). Often, the active form of the members of the TGF-β family of proteins are dimers, either homo- or heterodimers. The invention relates to mutations in the monomeric subunits of these proteins wherein a mutant monomer comprises a single or multiple amino acid substitutions, deletions or insertions, preferably located in or near the β hairpin L1 or L3 loops. Mutations outside of these hairpin loop regions that alter the structure of the hairpin loops such that the electrostatic interaction between the ligand and its cognate receptor are increased, are also contemplated. Fusion proteins and chimeric monomeric subunits are also contemplated by the present invention. The mutant TGF-β monomers of the invention have amino acid substitutions, deletions or insertions, of one, two, three, four or more amino acid residues when compared with the wild type subunit.

Polynucleotides Encoding Mutant CKGF and Analogs

The present invention also relates to nucleic acids molecules comprising polynucleotide sequences encoding mutant subunits of CKGFs and CKGF analogs, wherein the sequences contain at least one base insertion, deletion or substitution, or combinations thereof that result in single or multiple amino acid additions, deletions and substitutions relative to the wild type CKGF. As used herein, when two coding regions are said to be fused, the 3′ end of one nucleic acid molecule is ligated to the 5′ end of the other nucleic acid molecule such that translation proceeds from the coding region of one nucleic acid molecule into the other without a frameshift.

Due to the degeneracy of nucleotide coding sequences, any other DNA sequences that encode the same amino acid sequence for a mutant subunit may be used in the practice of the present invention. These include but are not limited to nucleotide sequences comprising all or portions of the coding region of a CKGF subunit which are altered by the substitution of different codons that encode the same amino acid residue within the sequence, thus producing a silent change.

In yet another embodiment, the invention provides nucleic acid molecules comprising sequences encoding single chain glycoprotein hormone analogs, wherein the coding region of a mutant α subunit comprising single or multiple amino acid substitutions, preferably located in or near the β hairpin L1 and/or L3 loop of the common α subunit, is fused with the coding region of a mutant glycoprotein hormone β subunit comprising single or multiple amino acid substitutions, preferably located in or near the β hairpin L1 and/or L3 loop of the β subunit. Also provided are nucleic acid molecules encoding a single chain glycoprotein hormone analog wherein the carboxyl terminus of the mutant glycoprotein hormone β subunit is linked to the amino terminus of the mutant common α subunit through the CTEP of the β subunit of hCG. In a preferred embodiment, the nucleic acid molecule encodes a single chain glycoprotein hormone analog, wherein the carboxyl terminus of a mutant β subunit is covalently bound to the amino terminus of CTEP, and the carboxyl terminus of the CTEP is covalently bound to the amino terminus of a mutant α subunit without the signal peptide.

The single chain glycoprotein hormone analogs of the invention can be made by ligating the nucleic acid sequences encoding the mutant α and β subunits to each other by methods known in the art, in the proper coding frame, and expressing the fusion protein by methods commonly known in the art. Alternatively, such a fusion protein may be made by protein synthetic techniques that employ a peptide synthesizer.

The production and use of the mutant subunits, mutant dimers, single chain glycoprotein hormone analogs, derivatives and fragments thereof of the invention are within the scope of the present invention.

CKGF Gene Cloning

Polynucleotides encoding the CKGF subunits can be obtained by standard procedures from sources of cloned DNA, as would be represented by a “library” of biological clones, by chemical synthesis, by cDNA cloning, or by the cloning of genomic DNA purified from a desired cell type. Methods useful for conducting these procedures have been detailed by Sambrook et al., in Molecular Cloning, A Laboratory Manual, 2d Ed., Cold Spring Harbor Laboratory Press, Cold Spring Harbor, N.Y. (1989); and by Glover, D. M. (ed.), in DNA Cloning: A Practical Approach, MRL Press, Ltd., Oxford, U.K. (1985). Polymerase chain reaction (PCR) can be used to amplify sequences encoding a CKGF subunit in a genomic or cDNA library. Synthetic oligonucleotides can be utilized as primers in a PCR protocol using RNA or DNA, preferably a cDNA library, as a source of polynucleotide templates. The DNA being amplified can include cDNA or genomic DNA from any human. After successful isolation or amplification of a polynucleotide encoding a segment of a CKGF subunit, that segment can be molecularly cloned and sequenced, and utilized as a probe to isolate a complete cDNA or genomic clone. This, in turn, will permit characterization of the nucleotide sequence of the CKGF-encoding polynucleotide, and the production of the CKGF protein product for functional analysis and/or therapeutic or diagnostic use.

Alternatives to isolating the coding regions for the subunits include chemically synthesizing the gene sequence itself from the published sequence. Other methods are possible and within the scope of the invention. The above-methods are not meant to limit the following general description of methods by which mutants of the hormone subunits may be obtained.

The identified and isolated polynucleotide can be inserted into an appropriate cloning vector for amplification of the gene sequence. A large number of vector-host systems known in the art may be used for this purpose. Possible vectors include, but are not limited to, plasmids or modified viruses. Of course, the vector system must be compatible with the host cell used in these procedures. Such vectors include, but are not limited to, bacteriophages such as lambda derivatives, or plasmids such as pBR322 or pUC plasmid derivatives or the pBLUESCRIPT vector (Stratagene). The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and mutant subunit gene may be modified by homopolymeric tailing. Recombinant molecules can be introduced into host cells via transformation, transfection, infection or electroporation so that many copies of the gene sequence are generated.

In an alternative method, the desired gene may be identified and isolated after insertion into a suitable cloning vector in a “shot gun” approach. Enrichment for the desired gene, for example, by size fractionation, can be done before insertion into the cloning vector.

In specific embodiments, transformation of host cells with recombinant DNA molecules that comprise the mutant subunit gene, cDNA, or synthesized DNA sequence enables generation of multiple copies of the gene. Thus, the CKGF-encoding polynucleotide may be obtained in large quantities by growing transformants, isolating the recombinant DNA molecules from the transformants and, when necessary, retrieving the inserted gene from the isolated recombinant DNA. Copies of the gene are used in mutagenesis experiments to study the structure and function of mutant CKGF subunits, mutant dimers and CKGF analogs.

Mutagenesis

The mutations present in mutant CKGF subunits, mutant dimers, analogs, fragments and derivatives of the invention can be produced by various methods known in the art. The manipulations which result in their production can occur at the gene or protein level. For example, the cloned coding region of the subunits can be modified by any of numerous strategies known in the art (see Sambrook et al., 1990, Molecular Clonings A Laboratory Manual, 2d ed., Cold Spring Harbor Laboratory, Cold Spring Harbor, N.Y.). The polynucleotide sequence can be cleaved at appropriate sites using restriction endonucleases, followed by further enzymatic modification if desired, isolated, and ligated in vitro. In the production of a mutant subunit, care should be taken to ensure that the modified gene remains within the same translational reading frame, uninterrupted by translational stop signals in the gene region where the subunit is encoded.

Additionally, the polynucleotide sequence encoding the subunits can be mutated in vitro or in vivo, to create variations in coding regions (e.g. amino acid substitutions), and/or to create and/or destroy translation, initiation, and/or termination sequences, and/or form new restriction endonuclease sites or destroy preexisting ones, to facilitate further in vitro modification. Any technique for mutagenesis known in the art can be used, including but not limited to, chemical mutagenesis, in vitro site-directed mutagenesis (Hutchinson, C., et al., 1978, J. Biol. Chem 253:6551), PCR-based overlap extension (Ho et al., 1989, Gene 77:51-59), PCR-based megaprimer mutagenesis (Sarkar et al., 1990, Biotechniques, 8:404-407), or similar methods. The presence of mutations can be confirmed by doublestranded dideoxy DNA sequencing.

One or more amino acid residue within a subunit can be substituted by another amino acid, preferably with different properties, in order to generate a range of functional differentials. Substitutes for an amino acid within the sequence may be selected from members of a different class to which the amino acid belongs. The nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Manipulations of the mutant subunit sequence may also be made at the protein level. Included within the scope of the invention are mutant CKGF subunits, mutant dimers, CKGF analogs which are differentially modified during or after translation, e.g., by glycosylation, acetylation, phosphorylation, amidation, derivatization by known protecting/blocking groups, proteolytic cleavage, linkage to an antibody molecule or other cellular ligand. Any of numerous chemical modifications may be carried out by known techniques, including but not limited to specific chemical cleavage by cyanogen bromide, trypsin, chymotrypsin, papain, V8 protease, NaBH₄; acetylation, formylation, oxidation, reduction; or metabolic synthesis in the presence of tunicamycin.

In addition, mutant CKGF subunits and analogs can be chemically synthesized. For example, a peptide corresponding to a portion of a mutant subunit which comprises the desired mutated domain can be synthesized using an automated peptide synthesizer. Optionally, nonclassical amino acids or chemical amino acid analogs can be introduced as a substitution or addition into the mutant subunit sequence. Non-classical amino acids include but are not limited to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, omithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

Expression of Mutant CKGF Subunit-Encoding Polynucleotides

The polynucleotide sequence encoding a mutant subunit of a CKGF or a functionally active analog or fragment or other derivative thereof can be inserted into an appropriate expression vector. In the context of the invention, appropriate expression vectors will contain the necessary elements for the transcription and translation of the inserted protein-coding sequence. The necessary transcriptional and translational signals can also be supplied by the native CKGF subunit cDNA or gene, and/or genomic sequences flanking each of the subunit genes. A variety of host-vector systems may be utilized to express the protein-coding sequence. These include mammalian cell systems infected with a recombinant virus such as a vaccinia virus or adenovirus; insect cell systems infected with a virus such as a recombinant baculovirus; and microorganisms such as yeast containing vectors capable of replication in yeast.

The expression elements of vectors vary in their strengths and specificities. Depending on the host-vector system utilized, any one of a number of suitable transcription and translation elements may be used. In specific embodiments, a mutant subunit coding region or a sequence encoding a mutated and functionally active portion of the respective mutant subunit is expressed.

Any of the methods previously described for the insertion of DNA fragments into a vector may be used to construct expression vectors containing a chimeric gene consisting of appropriate transcriptional/translational control signals and the protein coding sequences. These methods may include in vitro recombinant DNA synthetic techniques as well as in vivo recombination. Expression of polynucleotide sequences encoding mutant CKGF subunits or peptide fragments thereof may be regulated by a second polynucleotide sequence so that the mutant subunit(s) or peptide is expressed in a host transformed with the recombinant DNA molecule. For example, expression of a mutant CKGF subunit or peptide fragments thereof may be controlled by any promoter/enhancer element known in the art. Promoters which may be used include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), and the regulatory sequences of the metallothionein gene (Brinster et al., 1982, Nature 296:39-42).

In a specific embodiment, a vector is used that comprises one or more promoters operably linked to the coding region of a mutant CKGF subunit, one or more origins of replication, and, optionally, one or more selectable markers (e.g., an antibiotic resistance gene). For those CKGFs that exist naturally as heterodimers, expression of the two subunits within the same eukaryotic host cell is preferred as such coexpression favors proper assembly and glycosylation of a functional heterodimeric CKGF. Thus, in a preferred embodiment, such vectors are used to express both a first mutant subunit and a second mutant subunit in a host cell. The coding region of each of the mutant subunits may be cloned into separate vectors; the vectors being introduced into a host cell sequentially or simultaneously. Alternatively, the coding regions of both subunits may be inserted in one vector to which the appropriate promoters are operably linked.

A host cell strain may be chosen which modulates the expression of the inserted sequences, or modifies and processes the gene product in the specific fashion desired. Expression from certain promoters can be elevated in the presence of certain inducers. In this matter, expression of the genetically engineered mutant subunits may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. Expression in mammalian cells can be used to ensure “native” glycosylation of a heterologous protein. Furthermore, different vector/host expression systems may effect processing reactions to different extents.

Once a recombinant host cell which expresses the mutant subunit gene sequence(s) is identified, the gene product(s) can be analyzed. This is achieved by assays based on the physical or functional properties of the product, including radioactive labelling of the product followed by analysis by gel electrophoresis, immunoassay or other techniques useful for detecting the biological activity of the mutant subunit.

Production of Antibodies to Mutant Subunits and Analogs Thereof

According to the invention, mutant CKGF subunits, mutant CKGF dimers, single chain glycoprotein hormone analogs, its fragments or other derivatives thereof may be used as an immunogen to generate antibodies which immunospecifically bind such an immunogen. Preferably, the antibodies do not bind the wild type subunit or a dimer comprising the wild type subunit. Such antibodies include but are not limited to polyclonal, monoclonal, chimeric, single chain, Fab fragments, and an Fab expression library. In another embodiment, antibodies to a domain of a mutant subunit are produced. In a specific embodiment, antibodies to a mutant glycoprotein hormone, such as TSH, are produced.

Various procedures known in the art may be used for the production of polyclonal antibodies directed against mutant CKGF subunits, mutant CKGF dimers, analogs, single chain glycoprotein hormone analogs, its fragments or other derivatives thereof. For the production of antibodies, various host animals can be immunized by injection with the subunits, heterodimer, single chain analog, and derivatives thereof. Appropriate host animals include rabbits, mice, rats, other mammals as well as birds such as chickens. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies directed against mutant CKGF subunits, mutant CKGF dimers, analogs, single chain glycoprotein hormone analogs, its fragments or other derivatives thereof, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for the epitope together with genes from a human antibody molecule of appropriate biological activity can be used. The antibody products of these techniques fall within the scope of this invention.

According to the invention, techniques described for the production of single chain antibodies (U.S. Pat. No. 4,946,778) can be adapted to produce specific single chain antibodies against CKGF subunits, heterodimers, single chain analogs, or fragments or derivatives thereof. An additional embodiment of the invention utilizes the techniques described for the construction of Fab expression libraries (Huse et al., 1989, Science 246:1275-1281) to allow rapid and easy identification of monoclonal Fab fragments with the desired specificity.

Antibody fragments which contain the idiotype of the molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, the Fab fragments which can be generated by treating the antibody molecule with papain and a reducing agent, and Fv fragments.

In the production of antibodies, screening for the desired antibody can be accomplished using standard techniques known in the art. For example, the ELISA (enzyme-linked immunosorbent assay) would be an appropriate screening technique. For example, to select antibodies which recognize a specific domain of a mutant subunit, one may assay hybridomas for a product which binds to a fragment of a mutant subunit containing such domain. For selection of an antibody that specifically binds a mutant CKGF subunit, mutant CKGF dimer or a single chain analog but which does not specifically bind the wild type protein, one can select on the basis of positive binding to the mutant and a lack of binding to the wild type protein. Antibodies specific for a domain of a mutant CKGF subunit, mutant CKGF dimer or a single chain analog are also provided by the present invention.

The foregoing antibodies can be used in methods known in the art relating to the localization and activity of the mutant CKGF subunits, mutant CKGFs or single chain glycoprotein hormone analogs of the invention. These methods can involve imaging of the proteins, measuring levels thereof in appropriate physiological samples in diagnostic methods.

Structure and Function Analysis of Mutant CKGF Subunits

Described herein are methods for determining the structure of mutant CKGF subunits, mutant CKGF dimers and CKGF analogs, and for analyzing the in vitro activities and in vivo biological functions of the foregoing.

Once a mutant CKGF subunit is identified, it may be isolated and purified by standard methods including chromatography (e.g., ion exchange, affinity, and sizing column chromatography), centrifligation, differential solubility, or by any other standard technique useful for purifying proteins. Functional properties of the protein can be evaluated using any suitable assay, including immunoassays or biological assays that detect a product that it produced by a cell in response to stimulation by wild type or mutant CKGF protein.

Alternatively, once a mutant CKGF subunit produced by a recombinant host cell is identified, the amino acid sequence of the subunit(s) can be determined using standard techniques for protein sequencing, including the use of an automated amino acid sequencer.

The functional activity of mutant CKGF subunits, mutant CKGF dimers analogs, single chain glycoprotein hormone analogs, derivatives and fragments thereof can be assayed by various methods known in the art.

For example, where a mutant CKGF subunit or mutant CKGF dimer is assayed for its ability to bind or compete with the corresponding wild-type CKGF, or CKGF subunits are assayed for antibody binding, various immunoassays known in the art can be used. These immunoassays include competitive and non-competitive assay systems using techniques such as radio-immunoassays, ELISA, “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays. Antibody binding can be detected by detecting a label on the primary antibody. Alternatively, the primary antibody can be detected by detecting binding of a secondary antibody or reagent to the primary antibody, particularly where the secondary antibody is labeled.

Diagnostic and Therapeutic Uses of Mutant CKGFs

The invention provides for treatment or prevention of various diseases and disorders by administration of therapeutic compounds (termed herein “Therapeutic”) of the invention.

Disorders involving absence or decreased CKGF receptor signal transduction are treated or prevented by administration of a Therapeutic that promotes CKGF signal transduction. Disorders in which constitutive or increased CKGF receptor signal transduction is deficient or is desired are treated or prevented by administration of a Therapeutic that antagonizes or inhibits CKGF receptor signal transduction.

Pharmaceutical Compositions

The invention provides methods of diagnosis and methods of treatment by administration to a subject of an effective amount of a Therapeutic of the invention. In a preferred aspect, the Therapeutic is substantially purified. The subject is preferably an animal, including but not limited to animals such as cows, pigs, horses, chickens, cats, dogs, etc., and is preferably a mammal, and most preferably human. In a specific embodiment, a non-human mammal is the subject. Thus, in a particularly preferred embodiment, a mutant and/or modified human CKGF homodimer, heterodimer, derivative or analog, or nucleic acid, is therapeutically or prophylactically or diagnostically administered to a human patient.

The CKGF mutants, derivatives or analogs of the invention are preferably tested in vitro, and then in vivo for the desired, prior to use in humans. In various specific embodiments, in vitro assays can be carried out with representative cells of cell types (e.g., thyroid cells) involved in a patient's disorder, to determine if a mutant protein has a desired effect upon such cell types.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used.

Various delivery systems are known and can be used to administer a CKGF mutant, derivative or analog of the invention, e.g., encapsulation in liposomes, microparticles, microcapsules, recombinant cells capable of expressing the CKGF mutant, derivative or analog, receptor-mediated endocytosis (see, e.g., Wu and Wu, 1987, J. Biol. Chem. 262:4429-4432), etc. Methods of administration include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, local infusion during surgery, by means of a catheter, by means of a suppository, or by means of an implant, the implant being of a porous, non-porous, or gelatinous material, including membranes, such as sialastic membranes or fibers.

In another embodiment, the CKGF mutant, derivative or analog can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327.

In yet another embodiment, the CKGF mutant, derivative or analog can be delivered using a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

In a specific embodiment, a nucleic acid encoding the CKGF mutant, derivative or analog can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid molecule encoding a CKGF mutant, derivative or analog can be introduced intracellularly and incorporated within host cell DNA for expression, by homologous recombination.

The present invention also provides pharmaceutical compositions. Such compositions comprise a therapeutically effective amount of a CKGF mutant, derivative or analog and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the CKGF mutant, derivative or analog, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The CKGF mutants, derivatives or analogs of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylarnine, triethylamine, 2-ethylamino ethanol, histidine, procaine, etc.

The amount of the CKGF mutant, derivative or analog of the invention which will be effective in the treatment of a particular disorder or condition will depend on the nature of the disorder or condition, and can be determined by standard clinical techniques. In addition, in vitro assays and animal models may optionally be employed to help identify optimal dosage ranges. The precise dose to be employed in the formulation will also depend on the route of administration, and the seriousness of the disease or disorder, and should be decided according to the judgment of the practitioner and each patient's circumstances.

In specific embodiments, the Therapeutics of the invention are administered intramuscularly. Suitable dosage ranges for the intramuscular administration are generally about 10 μg to 1 mg per dose, preferably about 10 μg to 100 μg per dose. Generally, for diagnostic and therapeutic methods in which a CKGF mutant, for example a mutant TSH heterodimer, is administered, for example to stimulate iodine uptake, the mutant protein can be administered in a regimen of 1-3 injections. In one embodiment, the Therapeutic is administered in two doses, where the second dose is administered 24 hours after the first dose; in another embodiment, the Therapeutic is administered in three doses, with one dose being administered on days 1, 4 and 7 of a 7 day regimen.

Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pack or kit for therapeutic or diagnostic use comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or diagnostic products, which notice reflects approval by the agency of manufacture, use or sale for human administration.

Mutants of Thyroid Stimulating Hormone

As indicated above, one aspect of the invention particularly relates to novel mutant TSH proteins, mutant TSH protein-encoding polynucleotides, and methods of making these proteins and polynucleotides, and diagnostic and therapeutic methods based thereon. The present inventors have particularly designed and made mutant thyroid stimulating hormones (TSH), TSH derivatives, TSH analogs, and fragments thereof, that both have mutations (preferably amino acid substitutions) in the α and β subunits that increase the bioactivity of the TSH heterodimer comprised of these subunits relative to the bioactivity of wild type TSH and that are modified to increase the hormonal half life in circulation. The present inventors have found that these mutations to increase bioactivity and the strategies to increase hormonal half life synergize such that TSH heterodimers that have both the superactive mutations and the long acting modifications have much higher bioactivity than would be expected from the sum of the additional activity conferred by the superactive mutations and the long acting modifications individually.

The present inventors have also found that an amino acid substitution at amino acid 22 of the human α subunit, preferably a substitution of a basic amino acid, such as lysine or arginine, more preferably arginine, increases the bioactivity of TSH relative to wild type TSH.

The present inventors have designed mutant subunits by combining individual mutations within a single subunit and modifying the subunits and heterodimers to increase the half-life of the heterodimer in vivo. In particular, the inventors have designed mutuant α, mutant β mutant TSH heterodimers having mutations, particularly mutations in specific domains. These domains include the β hairpin L1 loop of the common α subunit, and the β hairpin L3 loop of the TSH β subunit. In one embodiment, the present invention provides mutant α subunits, mutant TSH β subunits, and TSH heterodimers comprising either one mutant α subunit or one mutant β subunit, wherein the mutant α subunit comprises single or multiple amino acid substitutions, preferably located within or near the β hairpin L1 loop of the α subunit, and wherein the mutant β subunit comprises single or multiple amino acid substitutions, preferably located in or near the β hairpin L3 loop of the β subunit (preferably, these mutations increase bioactivity of the TSH heterodimer comprising the mutant subunit and the TSH heterodimer having the mutant subunit has also been modified to increase the serum half-life relative to the wild-type TSH heterodimer).

According to the invention, a mutant β subunit comprising single or multiple amino acid substitutions, preferably located in or near the β hairpin L3 loop of the β subunit, can be fused at its carboxyl terminal to the CTEP. Such a mutant β subunit-CTEP subunit may be coexpressed and/or assembled with either a wild type or mutant α subunit to form a functional TSH heterodimer which has a bioactivity and a serum half life greater than wild type TSH.

In another embodiment, a mutant β subunit comprising single or multiple amino acid substitutions, preferably located in or near the β hairpin L3 loop of the β subunit, and mutant α subunit comprising single or multiple amino acid substitutions, preferably located in or near the β hairpin L1 loop of the α subunit, are fused to form a single chain TSH analog. Such a mutant β subunit-mutant α subunit fusion has a bioactivity and serum half-life greater than wild type TSH.

In yet another embodiment, mutant β subunit comprising single or multiple amino acid substitutions, preferably located in or near the β hairpin L3 loop of the β subunit, and further comprising the CTEP in the carboxyl terminus, and mutant α subunit comprising single or multiple amino acid substitutions, preferably located in or near the β hairpin L1 loop of the α subunit, are fused to form a single chain TSH analog.

Fusion proteins, analogs, and nucleic acid molecules encoding such proteins and analogs, and production of the foregoing proteins and analogs, e.g., by recombinant DNA methods, are also provided.

In particular aspects, the invention provides amino acid sequences of mutant α and β subunits, and fragments and derivatives thereof which are otherwise functionally active. “Functionally active” mutant TSH α and β subunits as used herein refers to that material displaying one or more known functional activities associated with the wild-type subunit, e.g., binding to the TSHR, triggering TSHR signal transduction, antigenicity (binding to an anti-TSH antibody), immunogenicity, etc.

In specific embodiments, the invention provides fragments of mutant α and TSH β subunits consisting of at least 6 amino acids, 10 amino acids, 50 amino acids, or of at least 75 amino acids. In various embodiments, the mutant α subunits comprise or consist essentially of a mutated αL1 loop domain; the mutant β subunits comprise or consist essentially of a mutated βL3 loop domain.

The present invention further provides nucleic acid sequences encoding mutant α and mutant β subunits and modified mutant α and β subunits (e.g mutant β subunit-CTEP fusions or mutant β subunit-mutant α subunit fusions), and methods of using the nucleic acid sequences.

The present invention also relates to therapeutic and diagnostic methods and compositions based on mutant α subunits, mutant β subunits, mutant TSH heterodimers, and TSH analogs, derivatives, and fragments thereof. The invention provides for the use of mutant TSH and analogs of the invention in the diagnosis and treatment of thyroid cancer by administering mutant TSH and analogs that are more active and have a longer half life in circulation than the wild type TSH. The invention further provides methods of diagnosing diseases and disorders characterized by the presence of autoantibodies against the TSH receptor using the mutant TSH heterodimers and analogs of the invention in TSH receptor binding inhibition assays. Diagnostic kits are also provided by the invention.

The invention particularly provides methods of treatment of disorders of the thyroid gland, such as thyroid cancer.

For clarity of disclosure, and not by way of limitation, the detailed description of the invention related to mutants of TSH and derivatives and analogs thereof is divided into the subsections which follow.

Mutants of the TSH α Subunit

As indicated above, the common human α subunit of glycoprotein hormones contains 92 amino acids as depicted in FIG. 2 (SEQ ID NO: 1), including 10 half-cysteine residues, all of which are in disulfide linkages. In one embodiment, the invention relates to mutants of the α subunit of human glycoprotein hormones wherein the subunit comprises single or multiple amino acid substitutions, preferably located in or near the β hairpin L1 and/or L3 loops of the α subunit. The amino acid residues located in or near the αL1 loop, starting from position 8-30 and the αL3 loop, starting from positions 61-85, as depicted in FIG. 2 have been found to be important in effecting receptor binding and signal transduction. Amino acid residues located in the αL1 loop, such as those at position 11-22, form a cluster of basic residues in all vertebrates except hominoids, and have the ability to promote receptor binding and signal transduction. In particular, the amino acid residue at position 22 is found to be one of the residues that influence the potency of TSH. According to the invention, the mutant α subunits have substitutions, deletions or insertions, of one, two, three, four, or more amino acid residues in the wild type protein.

In one embodiment, the mutant α subunits have one or more substitutions of amino acid residues relative to the wild type α subunit of the present invention, preferably, one or more amino acid substitutions in the amino acid residues selected from among residues at position 8-30 and 61-85.

In one aspect of this embodiment, a series of mutations in the α subunit of TSH are generated using the methods of the present invention. The goal of the mutation procedure is to yield a mutant TSH protein α subunit that will convey increased bioactivity relative to wild type TSH dimer. These mutant TSH proteins possess the amino acid sequence of SEQ ID NO: 1 concerning the α L1 subunit with at least one of the following amino acid substitutions: P8X, E9X, T11X, L12X, Q13X, E14X, N15X, P16X, F17X, F18X, S19X, Q20X, P21X, G22X, A23X, P24X, I25X, Q26 X M28X, or G30X. “X” represents the amino acid used to replace the wild type residue.

As with all of the mutations described herein, the amino acids to which “X” corresponds will depend on the nature of the electrostatic charge alteration sought by the artisan utilizing the method of the present invention. When an increase in the overall positive or basic electrostatic charge of the peripheral loop is sought, “X” will correspond to basic residues such as lysine (K), arginine (R) or histidine (H). When an increase in the overall negative or acidic electrostatic charge of the peripheral loop is sought, “X” will correspond to acidic residues such as aspartic acid (D) or glutamic acid (E). Other amino acids, such as aliphatic amino acids, are contemplated for use with the method described here.

In one aspect of this invention, neutral or acidic amino acid residues in the α subunit of TSH are mutated to alter the electrostatic charge of the L1 loop. The change in electrostatic charge is designed to yield an increased bioactivity for the mutant relative to a wild type TSH. These mutant TSH proteins possess the amino acid sequence of SEQ ID NO: 1 concerning the α L1 subunit with at least one of the following amino acid substitutions: E9B, T11B, Q13B, E14B, N15B, P16B, F17B, F18B, S19B, Q20B, G22B, P24B, or Q26B. “B” represents the basic amino acid used to replace the wild type residue. Basic amino acid residues are selected from the group consisting of lysine (K), arginine (R), and histidine (H).

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at E9U and E14U, wherein “U” is a neutral amino acid.

Mutant human glycoprotein hormone common alpha-subunit monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include P8Z, C10Z, T11Z, L12Z, Q13Z, N15Z, P16Z, F17Z, F18Z, S19Z, Q20Z, P21Z, G22Z, A23Z, P24Z, I25Z, L26Z, Q27Z, C28Z, M29Z, G30Z, P8B, C10B, T11B, L12B, Q13B, N15B, P16B, F17B, F18B, S19B, Q20B, P21B, G22B, A23B, P24B, I25B, L26B, Q27B, C28B, M29B, and G30B, wherein “Z” is an acidic amino acid and “B” is a basic amino acid.

In another embodiment, the present invention provides a mutant CKGF subunit that is a mutant human glycoprotein hormone α subunit L3 hairpin loop having an amino acid substitution at any of the positions from 61 to 85, inclusive, excluding Cys residues (excluding Cys residues). This sequence is also depicted in FIG. 2. These mutant TSH proteins possess the amino acid sequence of SEQ ID NO: 1 concerning the α L3 subunit with at least one of the following amino acid substitutions: V61X, A62X, K63X, S64X, Y65X, N66X, R67X, V68X, T69X, V70X, M71X, G72X, G73X, F74X, K75X, V76X, E77X, N78X H79X, T80X, A81X, H83X, or S85X. “X” represents the amino acid used to replace the wild type residue.

In one aspect of this embodiment, neutral or acidic amino acid residues in the α subunit of TSH are mutated. The resulting mutated subunits contain at least one mutation in the amino acid sequence of SEQ ID NO: 1 at the following amino acid positions: S64B, N66B, M71B, G72B, G73B, V76B, E77B, or A81B.

The invention further contemplates introducing one or more acidic residues into the amino acid sequence of the human glycoprotein hormone common alpha-subunit L3 hairpin loop. For example, one or more acidic amino acids can be introduced in the described above, wherein the variable “X” corresponds to an acidic amino acid. Specific examples of such mutations include K63Z, R67Z, K75Z, H79Z, and H83Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K63U, R67U, K75U, E77U, H79U, and H83U, wherein “U” is a neutral amino acid.

Mutant human glycoprotein hormone common alpha-subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include, V61Z, A62Z, S64Z, Y65Z, N66Z, V68Z, T69Z, V70Z, M71Z, G72Z, G73Z, F74Z, V76Z, N78Z, T80Z, A81Z, C82Z, C84Z, S85Z, V61B, A62B, S64B, Y65B, N66B, V68B, T69B, V70B, M71B, G72B, G73B, F74B, V76B, N78B, T80B, A81B, C82B, C84B, and S85B, wherein “Z” is an acidic amino acid and “B” is a basic amino acid.

The present invention also contemplate human glycoprotein hormone common alpha-subunit containing mutations outside of said β hairpin loop structures that alter the structure or conformation of those hairpin loops. These structural alterations in turn serve to increase the electrostatic interactions between regions of the β hairpin loop structures of human glycoprotein hormone common alpha-subunit contained in a dimeric molecule, and a receptor having affinity for the dimeric protein. These mutations are found at positions selected from the group consisting of positions 1-7, 31-60, and 86-92 of the human glycoprotein hormone common alpha-subunit monomer.

Specific examples of these mutation outside of the β hairpin L1 and L3 loop structures include, A1J, P2J, D3J, V4J, Q5J, D6J, C7J, C31J, C32J, F33J, S34J, R35J, A36J, Y37J, P38J, T39J, P40J, L41J, R42J, S43J, K44J, K45J, T46J, M47J, L48J, V49J, Q50J, K51J, N52J, V53J, T54J, S55J, E56J, S57J, T58J, C59J, C60J, T86J, C87J, Y88J, Y89J, H90J, K91J, and S92J. The variable “J” is any amino acid whose introduction results in an increase in the electrostatic interaction between the L1 and L3 β hairpin loop structures of the human glycoprotein hormone common alpha-subunit and a receptor with affinity for a dimeric protein containing the mutant human glycoprotein hormone common alpha-subunit monomer.

The invention also contemplates a number of human glycoprotein hormone common alpha-subunit in modified forms. These modified forms include human glycoprotein hormone common alpha-subunit linked to another cystine knot growth factor or a fraction of such a monomer.

In specific embodiments, the mutant human glycoprotein hormone common alpha-subunit heterodimer comprising at least one mutant subunit or the single chain human glycoprotein hormone common alpha-subunit analog as described above is functionally active, i.e., capable of exhibiting one or more functional activities associated with the wild-type human glycoprotein hormone common alpha-subunit, such as human glycoprotein hormone common alpha-subunit receptor binding, human glycoprotein hormone common alpha-subunit protein family receptor signalling and extracellular secretion. Preferably, the mutant human glycoprotein hormone common alpha-subunit heterodimer or single chain human glycoprotein hormone common alpha-subunit analog is capable of binding to the human glycoprotein hormone common alpha-subunit receptor, preferably with affinity greater than the wild type human glycoprotein hormone common alpha-subunit. Also it is preferable that such a mutant human glycoprotein hormone common alpha-subunit heterodimer or single chain human glycoprotein hormone common alpha-subunit analog triggers signal transduction. Most preferably, the mutant human glycoprotein hormone common alpha-subunit heterodimer comprising at least one mutant subunit or the single chain human glycoprotein hormone common alpha-subunit analog of the present invention has an in vitro bioactivity and/or in vivo bioactivity greater than the wild type human glycoprotein hormone common alpha-subunit and has a longer serum half-life than wild type BMP-11. Mutant human glycoprotein hormone common alpha-subunit heterodimers and single chain human glycoprotein hormone common alpha-subunit analogs of the invention can be tested for the desired activity by procedures known in the art.

In a preferred embodiment, the mutant α subunit of the invention has a single amino acid substitution at position 22, wherein a glycine residue is substituted with an arginine, i.e., αG22R. A mutant α subunit having the αG22R mutation may have at least one or more additional amino acid substitutions, such as but not limited to αT11K, αQ13K, αE14K, αP16K, αF17R, and αQ20K. In other preferred embodiments, the mutant α subunit has one, two, three, four, or more of the amino acid substitutions selected from the group consisting of αT11K, αQ13K, αE14K, αP16K, αF17R, αQ20K, and αG22R. For example, one of the preferred mutant α subunit (to be used in conjunction with a modification to increase the serum half-life of the TSH heterodimer having the mutant α subunit), also referred to herein as α4K, comprises four mutations: αQ13K+αE14K+αP16K+αQ20K.

The mutant α subunits of the invention are functionally active, i.e., capable of exhibiting one or more functional activities associated with the wild-type α subunit. Preferably, the mutant α subunit is capable of noncovalently associating with a wild type or mutant β subunit to form a TSH heterodimer that binds to the TSHR. Preferably, such a TSH heterodimer also triggers signal transduction. Most preferably, such a TSH heterodimer comprising a mutant α subunit has an in vitro bioactivity and/or in vivo bioactivity greater than the wild type TSH. It is contemplated in the present invention that more than one mutation can be combined within a mutant α subunit to make a superactive α mutant, which in association with a wild type or mutant β subunit, forms a TSH heterodimer, that has a significant increase in bioactivity relative to the wild type TSH. It is also contemplated that the α subunit mutations will be combined with strategies to increase the serum half-life of the TSH heterodimer having the mutant α subunit (i.e. a TSH heterodimer having a β subunit-CTEP fusion or a β subunit-α subunit fusion). The mutations within a subunit and the long acting modifications act synergistically to produce an unexpected increase in the bioactivity.

As another example, such mutant α subunits which have the desired immunogenicity or antigenicity can be used, for example, in immunoassays, for immunization and for inhibition of TSH receptor (TSHR) signal transduction.

Mutants of the TSH β Subunit

The common human β subunit of glycoprotein hormones contains 118 amino acids as depicted in FIG. 3 (SEQ ID No: 2). The invention relates to mutants of the β subunit of TSH wherein the subunit comprises single or multiple amino acid substitutions, preferably located in or near the β hairpin L3 loop of the β subunit, where such mutant β subunits are fused to another CKGF protein or polypeptide to increase the half-life of the protein, such as the CTEP of the β subunit of hCG or are part of a TSH heterodimer having a mutant α subunit with an amino acid substitution at position 22 (as depicted in FIG. 2 (SEQ ID NO: 1)), or being an α subunit-β subunit fusion. The amino acid residues located in or near the βL3 loop at positions 53-87 of the human TSH β subunits are mapped to amino acid residues in hCG that are located peripherally and appear to be exposed to the surface in the crystal structure. Of particular interest is a cluster of basic residues in hCG which is not present in TSH (starting from position 58-69). Substitution of basic or positively charged residues into this domain of human TSH leads to an additive and substantial increase in TSHR binding affinity as well as intrinsic activity.

The present invention provides a series of mutations in the TSH β subunit, generated using the methods of the present invention. The mutant TSH heterodimers of the invention have β subunits having substitutions, deletions or insertions, of one, two, three, four, or more amino acid residues in the wild type subunit. Mutations in the L1 loop of this subunit are contemplated in the amino acid residues between 1-30, inclusive, excluding Cys residues. The goal of the mutation procedure is to yield a mutant TSH protein β subunit that, when in a dimer, will convey increased bioactivity relative to wild type TSH dimer.

One embodiment of the present invention contemplates mutant TSH α subunit L1 hairpin loop subunits encoded by the amino acid sequence of SEQ ID NO: 2 with at least one of the following amino acid substitutions: F1X, I3X, P4X, T5X, E6X, Y7X, T8X, M9X, H10X, I11X, E12X, R13X, R14X, E15X, A17X, Y18X, L20X, T21X, I22X, N23X, T24X, T25X, I26X, A28X, G29X, or Y30X. “X” represents any amino acid residue, the substitution of which alters the electrostatic character of the L1 loop.

In an aspect of this embodiment, neutral or acidic amino acid residues in the α subunit L1 hairpin loop subunit are mutated to increase the positive electrostatic nature of this protein domain. The resulting mutated subunits contain at least one mutation in the amino acid sequence of SEQ ID NO: 2 at the following amino acid positions: F1B, I3B, T5B, E6B, T8B, M9B, E12B, E15B, A17B, T21B, N23B, T24B, T25B, I26B, A28B, G29B, and Y30B. “B” represents a basic amino acid reside.

Introducing acidic amino acid residues where basic residues are present in the hTSH beta-subunit monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following H10Z, R13Z, and R14Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at E6U, H10U, E12U, R13U, R14U and E15U, wherein “U” is a neutral amino acid.

Mutant hTSH beta-subunit monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues of I1Z, C2Z, I3Z, P4Z, T5Z, Y7Z, T8Z, M9Z, I11Z, C16Z, A17Z, Y18Z, C19Z, L20Z, T21Z, I22Z, N23Z, T24Z, T25Z, I26Z, C27Z, A28Z, G29Z, Y30Z, I1B, C2B, I3B, P4B, T5B, Y7B, T8B, M9B, I11B, C16B, A17B, Y18B, C19B, L20B, T21B, I22B, N23B, T24B, T25B, I26B, C27B, A28B, G29B, and Y30B, wherein “Z” is an acidic amino acid and “B” is a basic amino acid.

Mutations in the L3 loop of the β subunit are also contemplated in the amino acid residues between 53-87, inclusive, excluding Cys residues. These mutant TSH proteins possess the amino acid sequence of SEQ ID NO: 2 with at least one of the following amino acid substitutions: T53X, Y54X, R55X, D56X, F57X, I58X, Y59X, R60X, T61X, V62X, E63X, I64X, P65X, G66X, P68X, L69X, H70X, V71X, A72X, P73X, Y74X, F75X, S76X, Y77X, P78X, V79X, A80X, L81X, S82X, K84X, G86X, or K87X.

In an aspect of this embodiment, neutral or acidic amino acid residues in the β subunit of TSH are mutated. The resulting subunit contains at least one mutation in the amino acid sequence of SEQ ID NO: 2 at the following amino acid positions: I58B, Y59B, T61B, V62B, E63B, S64B, P65B, G66B, P68B, L69B, V71B, and A72B. Wherein “B” is a basic amino acid residue.

The invention further contemplates introducing one or more acidic residues into the amino acid sequence of the hTSH beta-subunit L3 hairpin loop. For example, one or more acidic amino acids can be introduced in the sequence described above, wherein the variable “X” corresponds to an acidic amino acid. Specific examples of such mutations include R55Z, R60Z, H70Z, K84Z, and K87Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at R55U, D56U, R60U, E63U, H70U, K84U, and K87U, wherein “U” is a neutral amino acid.

Mutant hTSH beta-subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include, T53Z, Y54Z, F57Z, I58Z, Y59Z, T61Z, V62Z, I64Z, P65Z, G66Z, C67Z, P68Z, L69Z, V71Z, A72Z, P73Z, Y74Z, F75Z, S76Z, Y77Z, P78Z, V79Z, A80Z, L81Z, S82Z, C83Z, C85Z, G86Z, T53B, Y54B, F57B, I58B, Y59B, T61B, V62B, I64B, P65B, G66B, C67B, P68B, L69B, V71B, A72B, P73B, Y74B, F75B, S76B, Y77B, P78B, V79B, A80B, L81B, S82B, C83B, C85B, and G86B, wherein “Z” is an acidic amino acid and “B” is a basic amino acid.

The present invention also contemplate hTSH beta-subunit containing mutations outside of said β hairpin loop structures that alter the structure or conformation of those hairpin loops. These structural alterations in turn serve to increase the electrostatic interactions between regions of the β hairpin loop structures of hTSH beta-subunit contained in a dimeric molecule, and a receptor having affinity for the dimeric protein. These mutations are found at positions selected from the group consisting of positions 31-52 and 88-118 of the hTSH beta-subunit monomer.

Specific examples of these mutation outside of the β hairpin L1 and L3 loop structures include, C31J, M32J, T33J, R34J, D35J, I36J, N37J, G38J, K39J, L40J, F41J, L42J, P43J, K44J, Y45J, A46J, L47J, S48J, Q49J, D50J, V51J, C52J, C88J, N89J, T90J, D91J, Y92J, S93J, D94J, C95J, I96J, H97J, E98J, A99J, I100J, K101J, T102J, N103J, Y104J, C105J, T106J, K107J, P108J, Q109J, K110J, S111J, Y112J, L113J, V114J, G115J, F116J, S117J, and V118J. The variable “J” is any amino acid whose introduction results in an increase in the electrostatic interaction between the L1 and L3 β hairpin loop structures of the hTSH beta-subunit and a receptor with affinity for a dimeric protein containing the mutant hTSH beta-subunit monomer.

The invention also contemplates a number of hTSH beta-subunit in modified forms. These modified forms include hTSH beta-subunit linked to another cystine knot growth factor or a fraction of such a monomer.

In specific embodiments, the mutant hTSH beta-subunit heterodimer comprising at least one mutant subunit or the single chain hTSH beta-subunit analog as described above is functionally active, i.e., capable of exhibiting one or more functional activities associated with the wild-type hTSH beta-subunit, such as hTSH beta-subunit receptor binding, hTSH beta-subunit protein family receptor signalling and extracellular secretion. Preferably, the mutant hTSH beta-subunit heterodimer or single chain hTSH beta-subunit analog is capable of binding to the hTSH beta-subunit receptor, preferably with affinity greater than the wild type hTSH beta-subunit. Also it is preferable that such a mutant hTSH beta-subunit heterodimer or single chain hTSH beta-subunit analog triggers signal transduction. Most preferably, the mutant hTSH beta-subunit heterodimer comprising at least one mutant subunit or the single chain hTSH beta-subunit analog of the present invention has an in vitro bioactivity and/or in vivo bioactivity greater than the wild type hTSH beta-subunit and has a longer serum half-life than wild type hTSH beta-subunit. Mutant hTSH beta-subunit heterodimers and single chain hTSH beta-subunit analogs of the invention can be tested for the desired activity by procedures known in the art.

In one embodiment, the mutant β subunit has one or more substitutions of amino acid residues relative to the wild type β subunit, preferably, one or more amino acid substitutions in the amino acid residues selected from among residues at position 53-87 of the β subunit as depicted in FIG. 3 (SEQ ID NO:2).

In a preferred embodiment, the mutant β subunit has one, two, three, or more of the amino acid substitutions selected from the group consisting of βI58R, βE63R, and βL69R. For example, one of the preferred mutant β subunit, also referred to herein as β3R, comprises three mutations: βI58R+βE63R+βL69R.

The mutant TSH, TSH analogs, derivatives, and fragments thereof of the invention having mutant β subunits either also have a mutant α subunit with an amino acid substitution at position 22 (as depicted in FIG. 2 (SEQ ID NO: 1)) and/or have a serum half life that is greater than wild type TSH. In one embodiment, a mutant β subunit comprising one or more substitutions of amino acid residues relative to the wild type β subunits is covalently bound to a carboxyl terminal portion of another CKGF protein, one example of which is the carboxyl terminal portion extension peptide (CTEP) of hCG. The CTEP, which comprises the carboxyl terminus 32 amino acids of the hCG β subunit (as depicted in FIG. 4), is covalently bound to the mutant β subunit, preferably the carboxyl terminus of the mutant β subunit is covalently bound to the amino terminus of CTEP. The β subunit and the CTEP may be covalently bound by any method known in the art, e.g. by a peptide bond or by a heterobifunctional reagent able to form a covalent bond between the amino terminus and carboxyl terminus of a protein, for example but not limited to, a peptide linker. In a preferred embodiment, the mutant β subunit and CTEP are linked via a peptide bond. In various preferred embodiments, the mutant β subunit-CTEP fusions may comprise one, two, three, or more of the amino acid substitutions selected from the group consisting of βI58R, βE63R, and βL69R.

In another embodiment, a mutant β subunit is fused, i.e. covalently bound, to an α subunit, preferably a mutant α subunit.

The mutant β subunits of the invention are functionally active, i.e., capable of exhibiting one or more functional activities associated with the wild-type β subunit. Preferably, the mutant β subunit is capable of noncovalently associating with a wild type or mutant α subunit to form a TSH heterodimer that binds to the TSHR. Preferably, such a TSH heterodimer also triggers signal transduction. Most preferably, such a TSH heterodimer comprising a mutant β subunit has an in vitro bioactivity and/or in vivo bioactivity greater than the bioactivity of wild type TSH. It is contemplated in the present invention that more than one mutation can be combined within a mutant β subunit to make a mutant TSH heterodimer having a significant increase in bioactivity relative to the wild type TSH. The inventors discovered that multiple mutations within a subunit and modifications to increase the half-life of the TSH heterodimer (i.e. the β subunit-CTEP fusion and/or the β subunit-α subunit fusion) can act synergistically to achieve bioactivity that is greater than the sum of the increase of the mutations and the long acting modifications.

Mutant β subunit can be tested for the desired activity by procedures that will be familiar to those having ordinary skill in the art.

Mutant TSH Heterodimers and TSH Analogs

The present invention provides mutant human TSH heterodimers and human TSH analogs comprising a mutant α subunit and a mutant β subunit, wherein the mutant α subunit comprises single or multiple amino acid substitutions, often located in or near the β hairpin L1 and/or L3 loops of the α subunit, and the mutant β subunit comprises single or multiple amino acid substitutions, preferably located in or near the β hairpin L1 and/or L3 loops of the β subunit, which heterodimer or analog also is modified to increase the serum half-life (e.g. by β subunit-CKGF fusion, such as a CTEP fusion or by α subunit-β subunit fusion). The single or multiple amino acid substitutions in the mutant α subunit can be made in amino acid residues selected from among positions 8-30 and 61-85, of the amino acid sequence of human α subunit. The single or multiple amino acid substitutions in the mutant TSH β subunit can be made in amino acid residues selected from among positions 1-30 and positions 53-87, of the amino acid sequence of human TSH β subunit. A non-limiting example of such a mutant TSH comprises a heterodimer of the mutant α subunit, α4K, and the mutant β subunit, β3R, as described above.

In one embodiment, the invention provides TSH heterodimers comprising an α subunit, preferably a mutant α subunit, and a β subunit, preferably a mutant β subunit, wherein either the mutant α or mutant β subunit is fused to a portion of another CKGF protein such as the CTEP of the β subunit of hCG. The term fusion protein refers herein to a protein which is the product of the covalent bonding of two peptides. The fusion may be to another CKGF protein as a whole, or a portion of that protein. Covalent bonding includes any method known in the art to bond two peptides covalently at their amino- and carboxyl-termini, respectively, such methods include but are not limited to, joining via a peptide bond or via a heterobifunctional reagent, for example but not by way of limitation, a peptide linker. In a preferred embodiment, the mutant TSH heterodimer may comprise a mutant human α subunit and a mutant human TSH β subunit, wherein the mutant human TSH β subunit is covalently bound at its carboxyl terminus to the amino terminus of CTEP.

The present invention also relates to single chain human TSH analogs comprising a mutant human α subunit covalently bound (as described above for the β subunit-CTEP fusion) to a mutant human TSH β subunit wherein the mutant α subunit and the mutant human TSH β subunit each comprise at least one amino acid substitution in the amino acid sequence of the respective subunit. In a preferred embodiment, the mutant β subunit is joined via a peptide linker to a mutant α subunit. In a more preferred embodiment, the CTEP of hCG, which has a high serine/proline content and lacks significant secondary structure, is the peptide linker.

Preferably, the mutant α subunit comprising single or multiple amino acid substitutions, preferably located in or near the β hairpin L1 and/or L3 loops of the α subunit is covalently bound to a mutant β subunit comprising single or multiple amino acid substitutions, preferably located in or near the β hairpin L1 and/or L3 loop of the β subunit.

In one embodiment, the mutant human TSH β subunit comprising at least one amino acid substitution in amino acid residues selected from among positions 1-30, preferably positions 53-87, of the amino acid sequence of human TSH β subunit is covalently bound at its carboxyl terminus with the amino terminus of a wild type human TSH α subunit or a mutant TSH α subunit comprising at least one amino acid substitution, wherein the one or more substitutions are in amino acid residues selected from among positions 8-30 and 61-85, of the amino acid sequence of human α subunit.

The mutant α subunit or mutant human TSH β subunit may each lack its signal sequence.

The present invention also provides a human TSH analog comprising a mutant human TSH β subunit covalently bound to CTEP which is, in turn, covalently bound to a mutant α subunit, wherein the mutant α subunit and the mutant human TSH β subunit each comprise at least one amino acid substitution in the amino acid sequence of each of the subunits.

In a specific embodiment, a mutant β subunit-CTEP fusion is covalently bound to a mutant α subunit, such that the carboxyl terminus of the mutant β subunit is linked to the amino terminal of the mutant α subunit through the CTEP of hCG. Preferably, the carboxyl terminus of a mutant β subunit is covalently bound to the amino terminus of CTEP, and the carboxyl terminus of the CTEP is covalently bound to the amino terminal of a mutant α subunit without the signal peptide.

Accordingly, in a specific embodiment, the human TSH analog comprises a mutant human TSH β subunit comprising at least one amino acid substitution in amino acid residues selected from among positions 1-30 and 53-87 of the amino acid sequence of human TSH β subunit covalently bound at the carboxyl terminus of the mutant human TSH β subunit with the amino terminus of CTEP which is joined covalently at the carboxyl terminus of said carboxyl terminal extension peptide with the amino terminus of a mutant α subunit comprising at least one amino acid substitution, wherein the one or more substitutions are in amino acid residues selected from among positions 8-30 and 61-85 of the amino acid sequence of human α subunit.

In another preferred embodiment, the mutant TSH heterodimer comprises a mutant α subunit having an amino acid substitution at position 22 of the human α subunit sequence (as depicted in FIG. 2 (SEQ ID NO:1)), preferably a substitution with a basic amino acid (such as arginine, lysine, and less preferably, histidine), more preferably with arginine.

In specific embodiments, the mutant TSH heterodimer comprising at least one mutant subunit or the single chain TSH analog as described above is functionally active, i.e., capable of exhibiting one or more functional activities associated with the wild-type TSH, such as TSHR binding, TSHR signalling and extracellular secretion. Preferably, the mutant TSH heterodimer or single chain TSH analog is capable of binding to the TSHR, preferably with affinity greater than the wild type TSH. Also it is preferable that such a mutant TSH heterodimer or single chain TSH analog triggers signal transduction. Most preferably, the mutant TSH heterodimer comprising at least one mutant subunit or the single chain TSH analog of the present invention has an in vitro bioactivity and/or in vivo bioactivity greater than the wild type TSH and has a longer serum half-life than wild type TSH. Mutant TSH heterodimers and single chain TSH analogs of the invention can be tested for the desired activity by procedures known in the art.

Polynucleotides Encoding Mutant TSH and Analogs

The present invention also relates to nucleic acids molecules comprising sequences encoding mutant subunits of human TSH and TSH analogs of the invention, wherein the sequences contain at least one base insertion, deletion or substitution, or combinations thereof that results in single or multiple amino acid additions, deletions and substitutions relative to the wild type TSH. Base mutation that does not alter the reading frame of the coding region is preferred. As used herein, when two coding regions are said to be fused, the 3′ end of one nucleic acid molecule is ligated to the 5′ (or through a nucleic acid encoding a peptide linker) end of the other nucleic acid molecule such that translation proceeds from the coding region of one nucleic acid molecule into the other without a frameshift.

Due to the degeneracy of the genetic code, any other DNA sequences that encode the same amino acid sequence for a mutant α or β subunit may be used in the practice of the present invention. These include but are not limited to nucleotide sequences comprising all or portions of the coding region of the α or β subunit which are altered by the substitution of different codons that encode the same amino acid residue within the sequence, thus producing a silent change.

In one embodiment, the present invention provides nucleic acid molecules comprising sequences encoding mutant α subunits, wherein the mutant α subunits comprise single or multiple amino acid substitutions, preferably located in or near the β hairpin L1 loop of the α subunit. In a specific embodiment, the invention provides nucleic acids encoding mutant α subunits having an amino acid substitution at position 22 of the amino acid sequence of the α subunit as depicted in FIG. 2 (SEQ ID NO:1), preferably substitution with a basic amino acid, more preferably substitution with arginine. The present invention further provides nucleic acids molecules comprising sequences encoding mutant β subunits comprising single or multiple amino acid substitutions, preferably located in or near the β hairpin L3 loop of the β subunit, and/or covalently joined to CTEP.

In yet another embodiment, the invention provides nucleic acid molecules comprising sequences encoding single chain TSH analogs, wherein the coding region of a mutant α subunit comprising single or multiple amino acid substitutions, preferably located in or near the β hairpin L1 loop of the α subunit, is fused with the coding region of a mutant β subunit comprising single or multiple amino acid substitutions, preferably located in or near the β hairpin L3 loop of the β subunit. Also provided are nucleic acid molecules encoding a single chain TSH analog wherein the carboxyl terminus of the mutant β subunit is linked to the amino terminus of the mutant α subunit through the CTEP of the β subunit of hCG. In a preferred embodiment, the nucleic acid molecule encodes a single chain TSH analog, wherein the carboxyl terminus of a mutant β subunit is covalently bound to the amino terminus of CTEP, and the carboxyl terminus of the CTEP is covalently bound to the amino terminus of a mutant α subunit without the signal peptide.

The single chain analogs of the invention can be made by ligating the nucleic acid sequences encoding the mutant α and β subunits to each other by methods known in the art, in the proper coding frame, and expressing the fusion protein by methods commonly known in the art. Alternatively, such a fusion protein may be made by protein synthetic techniques, e.g., by use of a peptide synthesizer.

Preparation of Mutant TSH Subunits and Analogs

The production and use of the mutant α subunits, mutant β subunits, mutant TSH heterodimers, TSH analogs, single chain analogs, derivatives and fragments thereof of the invention are within the scope of the present invention. In specific embodiments, the mutant subunit or TSH analog is a fusion protein either comprising, for example, but not limited to, a mutant β subunit and the CTEP of the β subunit of hCG or a mutant β subunit and a mutant α subunit. In one embodiment, such a fusion protein is produced by recombinant expression of a nucleic acid encoding a mutant or wild type subunit joined in-frame to the coding sequence for another protein, such as but not limited to toxins, such as ricin or diphtheria toxin. Such a fusion protein can be made by ligating the appropriate nucleic acid sequences encoding the desired amino acid sequences to each other by methods known in the art, in the proper coding frame, and expressing the fusion protein by methods commonly known in the art. Alternatively, such a fusion protein may be made by protein synthetic techniques, e.g., by use of a peptide synthesizer. Chimeric genes comprising portions of mutant α and/or β subunit fused to any heterologous protein-encoding sequences may be constructed. A specific embodiment relates to a single chain analog comprising a mutant α subunit fused to a mutant β subunit, preferably with a peptide linker between the mutant α subunit and the mutant β subunit.

Structure and Function Analysis of Mutant TSH Subunits

Described herein are methods for determining the structure of mutant TSH subunits, mutant heterodimers and TSH analogs, and for analyzing the in vitro activities and in vivo biological functions of the foregoing.

Once a mutant α or TSH β subunit is identified, it may be isolated and purified by standard methods including chromatography (e.g., ion exchange, affinity, and sizing column chromatography), centrifugation, differential solubility, or by any other standard technique for the purification of proteins. The functional properties may be evaluated using any suitable assay (including immunoassays as described infra).

Alternatively, once a mutant α subunit and/or TSH β subunit produced by a recombinant host cell is identified, the amino acid sequence of the subunit(s) can be determined by standard techniques for protein sequencing, e.g., with an automated amino acid sequencer.

The mutant subunit sequence can be characterized by a hydrophilicity analysis (Hopp, T. and Woods, K., 1981, Proc. Natl. Acad. Sci. U.S.A. 78:3824). A hydrophilicity profile can be used to identify the hydrophobic and hydrophilic regions of the subunit and the corresponding regions of the gene sequence which encode such regions.

Secondary structural analysis (Chou, P. and Fasman, G., 1974, Biochemistry 13:222) can also be done, to identify regions of the subunit that assume specific secondary structures.

Other methods of structural analysis can also be employed. These include but are not limited to X-ray crystallography (Engstom, A., 1974, Biochem. Exp. Biol. 11:7-13) and computer modeling Fletterick, R. and Zoller, M. (eds.), 1986, Computer Graphics and Molecular Modeling, in Current Communications in Molecular Biology, Cold Spring Harbor Laboratory, Cold Spring Harbor, N.Y.). Structure prediction, analysis of crystallographic data, sequence alignment, as well as homology modelling, can also be accomplished using computer software programs available in the art, such as BLAST, CHARMMm release 21.2 for the Convex, and QUANTA v.3.3, (Molecular Simulations, Inc., York, United Kingdom).

The functional activity of mutant α subunits, mutant β subunits, mutant TSH heterodimers, TSH analogs, single chain analogs, derivatives and fragments thereof can be assayed by various methods known in the art.

For example, where one is assaying for the ability of a mutant subunit or mutant TSH to bind or compete with wild-type TSH or its subunits for binding to an antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. Antibody binding can be detected by detecting a label on the primary antibody. Alternatively, the primary antibody is detected by detecting binding of a secondary antibody or reagent to the primary antibody, particularly where the secondary antibody is labelled. Many means are known in the art for detecting binding in an immunoassay and are within the scope of the present invention.

The binding of mutant α subunits, mutant β subunits, mutant TSH heterodimers, TSH analogs, single chain analogs, derivatives and fragments thereof, to the thyroid stimulating hormone receptor (TSHR) can be determined by methods well-known in the art, such as but not limited to in vitro assays based on displacement from the TSHR of a radiolabelled TSH of another species, such as bovine TSH, for example, but not limited to, as described by Szkudlinski et al. (1993, Endocrinol. 133:1490-1503). The bioactivity of mutant TSH heterodimers, TSH analogs, single chain analogs, derivatives and fragments thereof, can also be measured, for example, by assays based on cyclic AMP stimulation in cells expressing TSHR, such as those disclosed by Grossmann et al. (1995, Mol. Endocrinol. 9:948-958); and stimulation of thymidine uptake in thyroid cells, for example but not limited to as described by Szkudlinski et al. (1993, Endocrinol. 133:1490-1503).

In vivo bioactivity can be determined by physiological correlates of TSHR binding in animal models, such as measurements of T4 secretion in mice after injection of the mutant TSH heterodimer, TSH analog, or single chain analog, e.g. as described by East-Palmer et al. (1995, Thyroid 5:55-59). For example, wild type TSH and mutant TSH are injected intraperitoneally into male albino Swiss Crl:CF-1 mice with previously suppressed endogenous TSH by administration of 3 μg/ml T₃ in drinking water for 6 days. Blood samples are collected 6 hours later from orbital sinus and the serum T₄ and TSH levels are measured by respective chemiluminescence assays (Nichols Institute).

The half-life of a protein is a measurement of protein stability and indicates the time necessary for a one-half reduction in the concentration of the protein. The half life of a mutant TSH can be determined by any method for measuring TSH levels in samples from a subject over a period of time, for example but not limited to, immunoassays using anti-TSH antibodies to measure the mutant TSH levels in samples taken over a period of time after administration of the mutant TSH or detection of radiolabelled mutant TSH in samples taken from a subject after administration of the radiolabelled mutant TSH.

Other methods will be known to the skilled atisan and are within the scope of the invention.

Diagnostic and Therapeutic Uses

The invention provides for treatment or prevention of various diseases and disorders by administration of therapeutic compound (termed herein “Therapeutic”) of the invention. Such Therapeutics include TSH heterodimers having a mutant α subunit having at least an amino acid substitution at position 22 of the α subunit as depicted in FIG. 2 (SEQ ID NO:1) and either a mutant or wild type β subunit; TSH heterodimers having a mutant α subunit, preferably with one or more amino acid substitutions in or near the L1 loop (amino acids 8-30 as depicted in FIG. 2 (SEQ ID NO:1)) and a mutant β subunit, preferably with one or more amino acid substitutions in or near the L3 loop (amino acids 52-87 as depicted in FIG. 3 (SEQ ID NO:2)) and covalently bound to the CTEP of the β subunit of hCG; TSH heterodimers having a mutant α subunit, preferably with one or more amino acid substitutions in or near the L1 loop, and a mutant β subunit, preferably with one or more amino acid substitutions in or near the L3 loop, where the mutant α subunit and the mutant β subunit are covalently bound to form a single chain analog, including a TSH heterodimer where the mutant α subunit and the mutant β subunit and the CTEP of the β subunit of hCG are covalently bound in a single chain analog, other derivatives, analogs and fragments thereof (e.g. as described hereinabove) and nucleic acids encoding the mutant TSH heterodimers of the invention, and derivatives, analogs, and fragments thereof.

The subject to which the Therapeutic is administered is preferably an animal, including but not limited to animals such as cows, pigs, horses, chickens, cats, dogs, etc., and is preferably a mammal. In a preferred embodiment, the subject is a human. Generally, administration of products of a species origin that is the same species as that of the subject is preferred. Thus, in a preferred embodiment, a human mutant and/or modified TSH heterodimer, derivative or analog, or nucleic acid, is therapeutically or prophylactically or diagnostically administered to a human patient.

In a preferred aspect, the Therapeutic of the invention is substantially purified.

A number of disorders which manifest as hypothyroidism can be treated by the methods of the invention. Disorders in which TSH is absent or decreased relative to normal or desired levels are treated or prevented by administration of a mutant TSH heterodimer or TSH analog of the invention. Disorders in which TSH receptor is absent or decreased relative to normal levels or unresponsive or less responsive than normal TSHR to wild type TSH, can also be treated by administration of a mutant TSH heterodimer or TSH analog. Constitutively active TSHR can lead to hyperthyroidism, and it is contemplated that mutant TSH heterodimers and TSH analogs can be used as antagonists.

In specific embodiments, mutant TSH heterodimers or TSH analogs that are capable of stimulating differentiated thyroid functions are administered therapeutically, including prophylactically. Diseases and disorders that can be treated or prevented include but are not limited to hypothyroidism, hyperthyroidism, thyroid development, thyroid cancer, benign goiters, enlarged thyroid, protection of thyroid cells from apoptosis, etc.

The absence of decreased level in TSH protein or function, or TSHR protein and function can be readily detected, e.g., by obtaining a patient tissue sample (e.g., from biopsy tissue) and assaying it in vitro for RNA or protein levels, structure and/or activity of the expressed RNA or protein of TSH or TSHR. Many methods standard in the art can be thus employed, including but not limited to immunoassays to detect and/or visualize TSH or TSHR protein (e.g., Western blot, immunoprecipitation followed by sodium dodecyl sulfate polyacrylamide gel electrophoresis, immunocytochemistry, etc.) and/or hybridization assays to detect TSH or TSHR expression by detecting and/or visualizing TSH or TSHR mRNA (e.g., Northern assays, dot blots, in situ hybridization, etc.), etc.

In specific embodiments, Therapeutics of the invention are used to treat cancer of the thyroid. The mutant TSH heterodimers and analogs are useful in the stimulation of thyroidal and metastatic tissue prior to therapeutic ablation with radioactive iodine. For example, the mutant TSH heterodimers of the invention can be administered to a patient suffering from thyroidal cancer prior to administration of radiolabelled iodine for radioablation. The Therapeutics of the invention can also be used to stimulate iodine uptake by benign multinodular goiters prior to radioablation for treatment of the multinodular goiters, or to stimulate iodine uptake by thyroid tissue prior to radioablation for treatment of enlarged thyroid.

Specifically, the radioablation therapy is carried out by administering the Therapeutic of the invention, preferably administering the Therapeutic intramuscularly, in a regimen of one to three doses, for example but not limited to, one dose per day for two days, or one dose on the first, fourth and seventh days of a seven day regimen. The dosage is any appropriate dose, for example but not limited to a dose of approximately 10 μg to 1 mg, preferably a dose of approximately 10 μg to 100 μg. One day after the last dose of the regimen, radiolabelled iodine, preferably ¹³¹I, is administered to the subject in an amount sufficient to treat the cancer, noncancerous goiter or enlarged thyroid. The amount of radiolabelled iodine to be administered will depend upon the type and severity of the disease. In general, 30 to 300 mCi of ¹³¹I is administered to treat thyroid carcinoma.

In other specific embodiments, the mutant TSH heterodimers of the invention can be used for targeted delivery of therapeutics to the thyroid or to thyroid cancer cells, e.g. for targeted delivery of nucleic acids for gene therapy (for example targeted delivery of tumor suppressor genes to thyroid cancer cells) or for targeted delivery of toxins such as, but not limited to, ricin, diphtheria toxin, etc.

The invention further provides methods of diagnosis, prognosis, screening for thyroid cancer, preferably thyroid carcinoma, and of monitoring treatment of thyroid cancer, for example, by administration of the TSH heterodimers of the invention. In specific embodiments, Therapeutics of the invention are administered to a subject to stimulate uptake of iodine (preferably radiolabelled iodine such as, but not limited to, ¹³¹I or ¹²⁵I) by thyroid cells (including thyroid cancer cells) and/or to stimulate secretion of thyroglobulin from thyroid cells (including thyroid cancer cells). Subsequent to administration of the Therapeutic, radiolabelled iodine can be administered to the patient, and then the presence and localization of the radiolabelled iodine (i.e. the thyroid cells) can be detected in the subject (for example, but not by way of limitation, by whole body scanning) and/or the levels of thyroglobulin can be measured or detected in the subject, wherein increased levels of radioactive iodine uptake or increased levels of thyroglobulin secretion, as compared to levels in a subject not suffering from a thyroid cancer or disease or to a standard level, indicates that the subject has thyroid cancer. Certain subjects may have undergone thyroidectomy or thyroid tissue ablation therapy and have little or no residual thyroid tissue. In these subjects, or any other subject lacking noncancerous thyroid cells, detection of any iodine uptake or thyroglobulin secretion (above any residual levels remaining after the thyroidectomy or ablation therapy or after the loss of thyroid tissue for any other reason) indicates the presence of thyroid cancer cells. The localization of the incorporated radiolabelled iodine in the subject can be used to detect the spread or metastasis of the disease or malignancy. Additionally, the diagnostic methods of the invention can be used to monitor treatment of thyroid cancer by measuring the change in radiolabelled iodine or thyroglobulin levels in response to a course of treatment or by detecting regression or growth of thyroid tumor or metastasis.

Specifically, the diagnostic methods are carried out by administering the Therapeutic of the invention, preferably intramuscularly, in a regimen of one to three doses, for example but not limited to, one dose per day for two days, or one dose on the first, fourth and seventh days of a seven day regimen. The dosage is any appropriate dose, for example but not limited to a dose of approximately 10 μg to 1 mg, preferably a dose of approximately 10 μg to 100 μg. One day after the last dose of the regimen, radiolabelled iodine, preferably ¹³¹I, is administered to the subject in an amount sufficient for the detection of thyroid cells (including cancer cells), in general, 1-5 mCi of ¹³¹I is administered to diagnose thyroid carcinoma. Two days after administration of the radiolabelled iodine, the uptake of radiolabelled iodine in the patient is detected and/or localized in the patient, for example but not limited to, by whole body radioiodine scanning. Alternatively, in cases where all or most of the thyroid tissue has been removed (e.g. in patients with prior thyroidectomy or thyroid tissue ablation therapy), levels of thyroglobulin can be measured from 2 to 7 days after administration of the last dose of the Therapeutic of the invention. Thyroglobulin can be measured by any method well known in the art, including use of a immunoradiometric assay specific for thyroglobulin, which assay is well known in the art.

The mutant TSH heterodimers of the invention can also be used in TSH binding inhibition assays for TSH receptor autoantibodies, e.g. as described in Kakinuma et al. (1997, J. Clin. Endo. Met. 82:2129-2134). Antibodies against the TSH receptor are involved in certain thyroid diseases, such as but not limited to Graves' disease and Hashimoto's thyroiditis; thus, these binding inhibition assays can be used as a diagnostic for diseases of the thyroid such as Graves' disease and Hashimoto's thyroiditis. Briefly, cells or membrane containing the TSH receptor are contacted with the sample to be tested for TSHR antibodies and with radiolabelled mutant TSH of the invention, inhibition of the binding of the radiolabelled mutant TSH of the invention relative to binding to cells or membranes contacted with the radiolabelled mutant TSH but not with the sample to be tested indicates that the sample to be tested has antibodies which bind to the TSH receptor. The binding inhibition assay using the mutant TSH heterodimers of the invention, which have a greater bioactivity than the wild type TSH, has greater sensitivity for the anti-TSH receptor antibodies than does a binding inhibition assay using wild type TSH.

Accordingly, an embodiment of the invention provides methods of diagnosing or screening for a disease or disorder characterized by the presence of antibodies to the TSHR, preferably Graves' disease, comprising contacting cultured cells or isolated membrane containing TSH receptors with a sample putatively containing the antibodies from a subject and with a diagnostically effective amount of a radiolabelled mutant TSH heterodimer of the invention; measuring the binding of the radiolabelled mutant TSH to the cultured cells or isolated membrane, wherein a decrease in the binding of the radiolabelled TSH relative to the binding in the absence of said sample or in the presence of an analogous sample not having said disease or disorder, indicates the presence of said disease or disorder.

The mutant heterodimers and analogs may also be used in diagnostic methods to detect suppressed, but functional thyroid tissue in patients with autonomous hyperfunctioning thyroid nodules or exogenous thyroid hormone therapy. The mutant TSH heterodimers and TSH analogs may have other applications such as but not limited to those related to the diagnosis of central and combined primary and central hypothyroidism, hemiatrophy of the thyroid, and resistance to TSH action.

Mutants of the hCG β Subunit

The human β subunit of chorionic gonadotropin contains 145 amino acids as shown in FIG. 4 (SEQ ID No: 2). The invention contemplates mutants of the β subunit of hCG wherein the subunit comprises single or multiple amino acid substitutions, located in or near the β hairpin L1 and/or L3 loops of the β subunit, where such mutants are fused another CKGF protein, in whole or in part, for example fusion to TSH or are part of a hCG heterodimer. The mutant hCG heterodimers of the invention have β subunits having substitutions, deletions or insertions, of one, two, three, four or more amino acid residues when compared with the wild type subunit.

The present invention also provides a mutant hCG β subunit with an L1 hairpin loop having one or more amino acid substitutions between positions 1 and 37, inclusive, excluding Cys residues, as depicted in FIG. 4 (SEQ ID NO:3). The amino acid substitutions include: S1X, K2X, E3X, P4X, L5X, R6X, P7X, R8X, R10X, P11X, I12X, N13X, A14X, T15X, L16X, A17X, V18X, E19X, K20X, E21X, G22X, P24X, V25X, I27X, T28X, V29X, N30X, T31X, T32X, I33X, A35X, G36X, and Y37X.

In another aspect of this embodiment, neutral or acidic amino acid residues in the hCG β subunit, L1 hairpin loop are mutated. The resulting mutated subunits contain at least one mutation in the amino acid sequence of SEQ ID NO: 3 at the following amino acid positions: S1B, E3B, P4B, L5B, P7B, R8B, R10B, P11B, I12B, N13B, A14B, T15B, L16B, A17B, V18B, E19B, E21B, G22B, P24B, V25B, I27B, T28B, V29B, N30B, T31B, T32B, I33B, A35B, G36B, and Y37B.

Introducing acidic amino acid residues where basic residues are present in the hCG beta-subunit monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following K2Z, K6Z, K8Z, K10Z, and K20Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at K2U, E3U, R6U, R8U, R10U, E19U, K20U and E21U, wherein “U” is a neutral amino acid.

Mutant hCG beta-subunit monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues S1Z, P4Z, L5Z, P7Z, C9Z, P11Z, I12Z, N13Z, A14Z, T15Z, L16Z, A17Z, V18Z, G22Z, C23Z, P24Z, V25Z, C26Z, I27Z, T28Z,

V29Z, N30Z, T31Z, T32Z, I33Z, C34Z, A35Z, G36Z, Y37Z, S1B, P4B, L5B, P7B, C9B, P11B, I12B, N13B, A14B, T15B, L16B, A17B, V18B, G22B, C23B, P24B, V25B, C26B, I27B, T28B, V29B, N30B, T31B, T32B, I33B, C34B, A35B, G36B, and Y37B, wherein “Z” is an acidic amino acid and “B” is a basic amino acid.

The present invention also provides a mutant CKGF subunit that is a mutant hCG β subunit, L3 hairpin loop having one or more amino acid substitutions between positions 58 and 87, inclusive, excluding Cys residues, as depicted in FIG. 4 (SEQ ID NO:3). The amino acid substitutions include: N58X, Y59X, R60X, D61X, V62X, R63X, F64X, E65X, S66X, I67X, R68X, L69X, P70X, G71X, C72X, P73X, R74X, G75X, V76X, N77X, P78X, V79X, V80X, S81X,

Y82X, A83X, V84X, A85X, L86X, and S87X. “X” is any amino acid residue, the substitution with which alters the electrostatic character of the hairpin loop.

In another aspect of this embodiment, neutral or acidic amino acid residues in the hCG β subunit, L3 hairpin loop are mutated. The resulting mutated subunits contain at least one mutation in the amino acid sequence of SEQ ID NO: 3 at the following amino acid positions: N58B, Y59B, D61B, V62B, F64B, E65B, S66B, I67B, L69B, P70B, G71B, P73B, G75B, V76B, N77B, P78B,

G79B, V80B, S81B, Y82B, A83B, V84B, A85B, L86B, and S87B. “B” is a basic amino acid.

The invention further contemplates introducing one or more acidic residues into the amino acid sequence of the hCG beta-subunit L3 hairpin loop. For example, one or more acidic amino acids can be introduced in the sequence described above, wherein the variable “X” corresponds to an acidic amino acid. Specific examples of such mutations R60Z, R63Z, R68Z, and R73Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at R60U, D61U, R63U, E65U, R68U, and R74U, wherein “U” is a neutral amino acid.

Mutant hCG beta-subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include of N58Z, Y59Z, V62Z, F64Z, S66Z, I67Z, L69Z, P70Z, G71Z, C72Z, P73Z, G75Z, V76Z, N77Z, P78Z, V79Z, V80Z, S81Z, Y82Z, A83Z, V84Z, A85Z, L86Z, S87Z, N58B, Y59B, V62B, F64B, S66B, I67B, L69B, P70B, G71B, C72B, P73B, G75B, V76B, N77B, P78B, V79B, V80B, S81B, Y82B, A83B, V84B, A85B, L86B, and S87B, wherein “Z” is an acidic amino acid and “B” is a basic amino acid.

The present invention also contemplate hCG beta-subunit containing mutations outside of said β hairpin loop structures that alter the structure or conformation of those hairpin loops. These structural alterations in turn serve to increase the electrostatic interactions between regions of the β hairpin loop structures of hCG beta-subunit contained in a dimeric molecule, and a receptor having affinity for the dimeric protein. These mutations are found at positions selected from the group consisting of positions 38-57, and 88-140 of the hCG beta-subunit monomer.

Specific examples of these mutation outside of the β hairpin L1 and L3 loop structures include, C38J, P39J, T40J, M41J, T42J, R43J, V44J, L45J, Q46J, G47J, V48J, L49J, P50J, A51J, L52J, P53J, Q54J, V55J, V56J, C57J, C88J, Q89J, C90J, A91J, L92J, C93J, R94J, R95J, S96J, T97J, T98J, D99J, C100J, G101J, G102J, P103J, K104J, D105J, H106J, P107J, L108J,

T109J, C110J, D111J, D112J, P113J, R114J, F115J, Q116J, D117J, S118J, S119J, S120J, S121J, K122J, A123J, P124J, P125J, P126J, S127J, L128J, P129J, S130J, P131J, S132J, R133J, L134J,

P135J, G136J, P137J, S138J, D139J, and T140J. The variable “J” is any amino acid whose introduction results in an increase in the electrostatic interaction between the L1 and L3 β hairpin loop structures of the hCG beta-subunit and a receptor with affinity for a dimeric protein containing the mutant hCG beta-subunit monomer.

The invention also contemplates a number of hCG beta-subunit in modified forms.

These modified forms include hCG beta-subunit linked to another cystine knot growth factor or a fraction of such a monomer.

In specific embodiments, the mutant hCG beta-subunit heterodimer comprising at least one mutant subunit or the single chain hCG beta-subunit analog as described above is functionally active, i.e., capable of exhibiting one or more functional activities associated with the wild-type hCG beta-subunit, such as hCG beta-subunit receptor binding, hCG beta-subunit protein family receptor signalling and extracellular secretion. Preferably, the mutant hCG beta-subunit heterodimer or single chain hCG beta-subunit analog is capable of binding to the hCG beta-subunit receptor, preferably with affinity greater than the wild type hCG beta-subunit. Also it is preferable that such a mutant hCG beta-subunit heterodimer or single chain hCG beta-subunit analog triggers signal transduction. Most preferably, the mutant hCG beta-subunit heterodimer comprising at least one mutant subunit or the single chain hCG beta-subunit analog of the present invention has an in vitro bioactivity and/or in vivo bioactivity greater than the wild type hCG beta-subunit and has a longer serum half-life than wild type hCG beta-subunit. Mutant hCG beta-subunit heterodimers and single chain hCG beta-subunit analogs of the invention can be tested for the desired activity by procedures known in the art.

In one embodiment, the present invention provides a mutant hCG that is a heterodimeric protein, such as a mutant TSH or a mutant hCG, comprising at least one of the above-described mutant α and/or β subunits. The mutant subunits comprise one or more amino acid substitutions.

In specific embodiments, the mutant hCG heterodimer comprising at least one mutant subunit or the single chain hCG analog as described above is functionally active, i.e., capable of exhibiting one or more functional activities associated with the wild-type hCG, such as hCGR binding, hCGR signalling and extracellular secretion. Preferably, the mutant hCG heterodimer or single chain hCG analog is capable of binding to the hCGR, preferably with affinity greater than the wild type hCG. Also it is preferable that such a mutant hCG heterodimer or single chain hCG analog triggers signal transduction. Most preferably, the mutant hCG heterodimer comprising at least one mutant subunit or the single chain hCG analog of the present invention has an in vitro bioactivity and/or in vivo bioactivity greater than the wild type hCG and has a longer serum half-life than wild type hCG. Mutant hCG heterodimers and single chain hCG analogs of the invention can be tested for the desired activity by procedures known in the art.

Polynucleotides Encoding Mutant hCG β Subunit and Analogs

The present invention also relates to nucleic acids molecules comprising sequences encoding mutant subunits of human hCG β Subunit and hCG β subunit and analogs of the invention, wherein the sequences contain at least one base insertion, deletion or substitution, or combinations thereof that results in single or multiple amino acid additions, deletions and substitutions relative to the wild type protein. Base mutation that does not alter the reading frame of the coding region are preferred. As used herein, when two coding regions are said to be fused, the 3′ end of one nucleic acid molecule is ligated to the 5′ (or through a nucleic acid encoding a peptide linker) end of the other nucleic acid molecule such that translation proceeds from the coding region of one nucleic acid molecule into the other without a frameshift.

Due to the degeneracy of the genetic code, any other DNA sequences that encode the same amino acid sequence for a mutant subunit or monomer may be used in the practice of the present invention. These include but are not limited to nucleotide sequences comprising all or portions of the coding region of the subunit or monomer that are altered by the substitution of different codons that encode the same amino acid residue within the sequence, thus producing a silent change.

In one embodiment, the present invention provides nucleic acid molecules comprising sequences encoding mutant hCG β subunits, wherein the mutant hCG β Subunit subunits comprise single or multiple amino acid substitutions, preferably located in or near the β hairpin L1 and/or L3 loops of the target protein. The invention also provides nucleic acids molecules encoding mutant hCG β Subunit subunits having an amino acid substitution outside of the L1 and/or L3 loops such that the electrostatic interaction between those loops and the cognate receptor of the hCG β Subunit holo-protein are increased. The present invention further provides nucleic acids molecules comprising sequences encoding mutant hCG β Subunit subunits comprising single or multiple amino acid substitutions, preferably located in or near the β hairpin L1 and/or L3 loops of the hCG β Subunit subunit, and/or covalently joined to CTEP or another CKGF protein.

In yet another embodiment, the invention provides nucleic acid molecules comprising sequences encoding hCG β Subunit analogs, wherein the coding region of a mutant hCG β Subunit subunit comprising single or multiple amino acid substitutions, is fused with the coding region of its corresponding dimeric unit, which can be a wild type subunit or another mutagenized monomeric subunit. Also provided are nucleic acid molecules encoding a single chain hCG β Subunit analog wherein the carboxyl terminus of the mutant hCG β Subunit monomer is linked to the amino terminus of another CKGF protein, such as the CTEP of the β subunit of hCG. In still another embodiment, the nucleic acid molecule encodes a single chain hCG β Subunit analog, wherein the carboxyl terminus of the mutant hCG β Subunit monomer is covalently bound to the amino terminus another CKGF protein such as the amino terminus of CTEP, and the carboxyl terminus of bound amino acid sequence is covalently bound to the amino terminus of a mutant hCG β Subunit monomer without the signal peptide.

The single chain analogs of the invention can be made by ligating the nucleic acid sequences encoding monomeric subunits of hCG β Subunit to each other by methods known in the art, in the proper coding frame, and expressing the fusion protein by methods commonly known in the art. Alternatively, such a fusion protein may be made by protein synthetic techniques, e.g., by use of a peptide synthesizer.

Preparation of Mutant hCG β Subunit and Analogs

The production and use of mutant hCG β subunits, mutant hCG heterodimers, hCG analogs, single chain analogs, derivatives and fragments thereof of the invention are within the scope of the present invention. In specific embodiments, the mutant subunit or hCG analog is a fusion protein either comprising, for example, but not limited to, a mutant β subunit and another CKGF protein or fragment thereof or a mutant β subunit and a mutant α subunit. In one embodiment, such a fusion protein is produced by recombinant expression of a nucleic acid encoding a mutant or wild type subunit joined in-frame to the coding sequence for another protein, such as but not limited to toxins, such as ricin or diphtheria toxin. Such a fusion protein can be made by ligating the appropriate nucleic acid sequences encoding the desired amino acid sequences to each other by methods known in the art, in the proper coding frame, and expressing the fusion protein by methods commonly known in the art. Alternatively, such a fusion protein may be made by protein synthetic techniques, e.g., by use of a peptide synthesizer. Chimeric genes comprising portions of mutant α and/or β subunit fused to any heterologous protein-encoding sequences may be constructed. A specific embodiment relates to a single chain analog comprising a mutant α subunit fused to a mutant β subunit, preferably with a peptide linker between the mutant α subunit and the mutant β subunit.

Structure and Function Analysis of Mutant hCG Subunits

Described herein are methods for determining the structure of mutant hCG subunits, mutant heterodimers and hCG analogs, and for analyzing the in vitro activities and in vivo biological functions of the foregoing.

Once a mutant hCG β subunit is identified, it may be isolated and purified by standard methods including chromatography (e.g., ion exchange, affinity, and sizing column chromatography), centrifugation, differential solubility, or by any other standard technique for the purification of proteins. The functional properties may be evaluated using any suitable assay (including immunoassays as described infra).

Alternatively, once a mutant hCG subunit produced by a recombinant host cell is identified, the amino acid sequence of the subunit(s) can be determined by standard techniques for protein sequencing, e.g., with an automated amino acid sequencer.

The mutant subunit sequence can be characterized by a hydrophilicity analysis (Hopp, T. and Woods, K., 1981, Proc. Natl. Acad. Sci. U.S.A. 78:3824). A hydrophilicity profile can be used to identify the hydrophobic and hydrophilic regions of the subunit and the corresponding regions of the gene sequence which encode such regions.

Secondary structural analysis (Chou, P. and Fasman, G., 1974, Biochemistry 13:222) can also be done, to identify regions of the subunit that assume specific secondary structures.

Other methods of structural analysis can also be employed. These include but are not limited to X-ray crystallography (Engstom, A., 1974, Biochem. Exp. Biol. 11:7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.). Structure prediction, analysis of crystallographic data, sequence alignment, as well as homology modelling, can also be accomplished using computer software programs available in the art, such as BLAST, CHARMM release 21.2 for the Convex, and QUANTA v.3.3, (Molecular Simulations, Inc., York, United Kingdom).

The functional activity of mutant hCG β subunits, mutant hCG heterodimers, hCG analogs, single chain analogs, derivatives and fragments thereof can be assayed by various methods known in the art.

For example, where one is assaying for the ability of a mutant hCG β subunit or mutant hCG to bind or compete with wild-type hCG or its subunits for binding to an antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel difflusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. Antibody binding can be detected by detecting a label on the primary antibody. Alternatively, the primary antibody is detected by detecting binding of a secondary antibody or reagent to the primary antibody, particularly where the secondary antibody is labelled. Many means are known in the art for detecting binding in an immunoassay and are within the scope of the present invention.

The binding of mutant hCG β subunits, mutant hCG heterodimers, hCG analogs, single chain analogs, derivatives and fragments thereof, to the human chorionic gonadotropin receptor (hCGR) can be determined by methods well-known in the art, such as but not limited to in vitro assays based on displacement from the hCGR of a radiolabelled mutant hCG by wild type hCG, for example. The bioactivity of mutant hCG heterodimers, hCG analogs, single chain analogs, derivatives and fragments thereof, can also be measured in a cell-based assay. For example, the transformed Leydig tumor cell line, MA-10, (Dr. Mario Ascoli, University of Iowa, Iowa City, Iowa) is used to measure the bioactivity of the mutant hCG proteins of the present invention. The cells are grown in Waymouth's MB 752/1 medium supplemented with 15% equine serum (Hyclone Laboratory, Park City, Utah), 4.77 g/L Hepes, 2.24 g/L NaHCO₃, 100 U/ml penicillin, 100 μg/ml streptomycin, 50 μg/ml gentamycin and 1.0 μg/ml amphotercin B (growth medium). Cells are maintained at 37° C. in 5% CO₂ and used for assays between passages 5 and 15. Cells are plated in 24-well plates at a density of 2.5×105 cells per well in 1 ml of growth medium. Following the first 48 hours of culture, the medium is replaced with 1 ml of growth medium containing 1 mg/ml BSA in place of equine serum. Approximately 18 hours later the level of hCG or LH induced progesterone production is measured in a 2 hour assay.

A standard line of wild type hCG proteins are included with each assay to determine the concentration at which progesterone production is stimulated at 50% of maximum (EC₅₀). The EC₅₀ for hCG is 0.125 nM. Each 24-well plate contains three control wells that consist of 450 μl of modified growth medium (10 μg/ml BSA without equine serum) and 50 μl sterile deionized and distilled water. Each plate also has 2 wells with the same medium as the control wells containing a final concentration of 0.125 mM hCG wild type proteins in 500 μl. The test wells contained 0.125 nM mutant hCG proteins in a volume of 500 μl. Two hours after the addition of hormone, medium is harvested and stored frozen for later analysis of progesterone. The cell monolayer are then washed once with saline, incubated with 500 μl of detergent (Triton X-100) and stored frozen for analysis of protein content. Progesterone concentrations are determined with a radioimmunoassay kit (Diagnostic Products, Los Angeles, Calif.). Protein levels are determined if large variations in progesterone values are due to differences in cell numbers.

The amount of progesterone production is compared between the wells containing the wild type forms of the proteins being tested and those wells containing mutant proteins. The bioactivity of the mutant proteins tested is expressed as the percentage of wild type progesterone production displayed by the mutant proteins. An example of this assay is found in Morbeck, et al., Mole. and Cell. Endocrinol., 97:173-181 (1993).

The half-life of a protein is a measurement of protein stability and indicates the time necessary for a one-half reduction in the concentration of the protein. The half life of a mutant hCG can be determined by any method for measuring hCG levels in samples from a subject over a period of time, for example but not limited to, immunoassays using anti-hCG antibodies to measure the mutant hCG levels in samples taken over a period of time after administration of the mutant hCG or detection of radiolabelled mutant hCG in samples taken from a subject after administration of the radiolabelled mutant hCG.

Other methods will be known to the skilled artisan and are within the scope of the invention.

Diagnostic and Therapeutic Uses

The invention provides for treatment or prevention of various diseases and disorders by administration of therapeutic compound (termed herein “Therapeutic”) of the invention. Such Therapeutics include hCG heterodimers having a mutant α and either a mutant or wild type hCG β subunit; hCG heterodimers having a mutant α subunit, preferably with one or more amino acid substitutions in or near the L1 and/or L3 loops and a mutant β subunit, preferably with one or more amino acid substitutions in or near the L1 and/or L3 loops and covalently bound to another CKGF protein, in whole or in part; hCG heterodimers having a mutant α subunit, and a mutant β subunit, where the mutant α subunit and the mutant β subunit are covalently bound to form a single chain analog, including a hCG heterodimer where the mutant α subunit and the mutant β subunit and another CKGF protein covalently bound in a single chain analog, other derivatives, analogs and fragments thereof (e.g. as described hereinabove) and nucleic acids encoding the mutant hCG heterodimers of the invention, and derivatives, analogs, and fragments thereof.

The subject to which the Therapeutic is administered is preferably an animal, including but not limited to animals such as cows, pigs, horses, chickens, cats, dogs, etc., and is preferably a mammal. In a preferred embodiment, the subject is a human. Generally, administration of products of a species origin that is the same species as that of the subject is preferred. Thus, in a preferred embodiment, a human mutant and/or modified hCG heterodimer, derivative or analog, or nucleic acid, is therapeutically or prophylactically or diagnostically administered to a human patient.

In a preferred aspect, the Therapeutic of the invention is substantially purified.

Human chorionic gonadotropin is secreted in large quatities by the placenta during pregnancy. This hormone stimulates the formation of Leydig cells in the testes of the fetus and causes testosterone secretion. Since testosterone secretion during fetal development is important for promoting formation of the male sexual organs, an insufficient amount of hCG may result in hypogonadism in the male. One form of this condition is hypogonadotropic hypogonadism. Disorders such as hypogonadotropic hypogonadism in which hCG is absent or decreased relative to normal or desired levels are treated or prevented by administration of a mutant hCG heterodimer or hCG analog of the invention. Disorders in which hCG receptor is absent or decreased relative to normal levels or unresponsive or less responsive than normal hCGR to wild type hCG, can also be treated by administration of a mutant hCG heterodimer or hCG analog. Constitutively active hCGR can lead to hypergonadism, and it is contemplated that mutant hCG heterodimers and hCG analogs can be used as antagonists.

The administration of hCG has also been shown to be effective in treating luteal phase defect. Blumenfeld & Nahhas, Fertil. Steril., 50(3):403-7 (1988). Accordingly, the mutant hCG proteins of the present invention can be used to treat luteal phase defects.

The invention further provides methods of diagnosis, prognosis, screening for ovarian, pancreatic, gastric and hepatocellular carcinoma, and of monitoring treatment of testicular cancer.

Mutants of the hLH β Subunit

The human β subunit of human luteinizing hormone (hLH) contains 121 amino acids as shown in FIG. 5 (SEQ ID No:4). The invention contemplates mutants of the β subunit of hLH wherein the subunit comprises single or multiple amino acid substitutions, located in or near the β hairpin L1 and/or L3 loops of the β subunit, where such mutants are fused to TSH, or another CKGF protein, or are part of a hLH heterodimer.

The mutant hLH heterodimers of the invention have β subunits having substitutions, deletions or insertions, of one, two, three, four or more amino acid residues when compared with the wild type subunit. The present invention further provides a mutant hLH β subunit having an L1 hairpin loop having one or more amino acid substitutions between positions 1 and 33, inclusive, excluding Cys residues, as depicted in FIG. 5 (SEQ ID NO:4). The amino acid substitutions include: W8X, H10X, P11X, I12X, N13X, A14X, I15X, L16X, A17X, V18X, E19X, K20X, E21X, G22X, P24X, V25X, I27X, T28X, V29X, N30X, T31X, T32X, and I33X.

In another aspect of this embodiment, neutral or acidic amino acid residues in the hLH β subunit, L1 hairpin loop are mutated. The resulting mutated subunits contain at least one mutation in the amino acid sequence of SEQ ID NO: 4 at the following amino acid positions: W8B, P11B, I12B, N13B, A14B, I15B, L16B, A17B, V18B, E19B, E21B, G22B, P24B, V25B, I27B, T28B,

V29B, N30B, T31B, T32B, and I33B.

Introducing acidic amino acid residues where basic residues are present in the hLH beta-subunit monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following R2Z, R6Z, H10Z, and K20Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at R2U, E3U, R6U, E19U, K20U and E21U, wherein “U” is a neutral amino acid.

Mutant hLH beta-subunit monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues S1Z, P4Z, L5Z, P7Z, W8Z, C9Z, P11Z, I12Z, N13Z, A14Z, I15Z, L16Z, A17Z, V18Z, G22Z, C23Z, P24Z, V25Z, C26Z, I27Z, T28Z, V29Z, N30Z, T31Z, T32Z, I33Z, S1B, P4B, L5B, P7B, W8B, C9B, P11B, I12B, N13B, A14B, I15B, L16B, A17B, V18B, G22B, C23B, P24B, V25B, C26B, I27B, T28B, V29B, N30B, T31B, T32B, and I33B, wherein “Z” is an acidic amino acid and “B” is a basic amino acid.

The present invention also provides a mutant CKGF subunit that is a mutant hLH β subunit, L3 hairpin loop having one or more amino acid substitutions between positions 58 and 87, inclusive, excluding Cys residues, as depicted in FIG. 5 (SEQ ID NO:4). The amino acid substitutions include: N58X, Y59X, R60X, D61X, V62X, R63X, F64X, E65X, S66X, I67X, R68X, L69X, P70X, G71X, C72X, P73X, R74X, G75X, V76X, N77X, P78X, V79X, V80X, S81X, Y82X, A83X, V84X, A85X, L86X, or S87X.

In another aspect of this embodiment, neutral or acidic amino acid residues in the hLH β subunit, L3 hairpin loop are mutated. The resulting mutated subunits contain at least one mutation in the amino acid sequence of SEQ ID NO: 4 at the following amino acid positions: N58B, Y59B, D61B, V62B, F64B, E65B, S66B, I67B, L69B, P70B, G71B, P73B, G75B, V76B, N77B, P78B, G79B, V79B, V80B, S81B, Y82B, A83B, V84B, A85B, L86B, and S87B.

The invention further contemplates introducing one or more acidic residues into the amino acid sequence of the hLH beta-subunit L3 hairpin loop. For example, one or more acidic amino acids can be introduced in the sequence described above, wherein the variable “X” corresponds to an acidic amino acid. Specific examples of such mutations include R60Z, R63Z, R68Z, and R74Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at R60U, D61U, R63U, E65U, R68U, R74U, and D77U, wherein “U” is a neutral amino acid.

Mutant hLH beta-subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include, T58Z, Y59Z, V62Z, I64Z, S66Z, I67Z, L69Z, P70Z, G71Z, C72Z, P73Z, G75Z, V76Z, P78Z, V79Z, V80Z, S81Z, F82Z, P83Z, V84Z, A85Z, L86Z, S87Z, T58B, Y59B, V62B, I64B, S66B, I67B, L69B, P70B, G71B, C72B, P73B, G75B, V76B, P78B, V79B, V80B, S81B, F82B, P83B, V84B, A85B, L86B, and S87B, wherein “Z” is an acidic amino acid and “B” is a basic amino acid.

The present invention also contemplate hLH beta-subunit containing mutations outside of said β hairpin loop structures that alter the structure or conformation of those hairpin loops.

These structural alterations in turn serve to increase the electrostatic interactions between regions of the β hairpin loop structures of hLH beta-subunit contained in a dimeric molecule, and a receptor having affinity for the dimeric protein. These mutations are found at positions selected from the group consisting of positions 34-57, and 88-121 of the hLH beta-subunit monomer.

Specific examples of these mutation outside of the β hairpin L1 and L3 loop structures include, A35J, G36J, Y37J, C38J, P39J, T40J, M41J, M42J, R43J, V44J, L45J, Q46J, A47J, V48J, L49J, P50J, P51J, L52J, P53J, Q54J, V55J, V56J, C57J, C88J, R89J, C90J, G91J, P92J, C93J, R94J, R95J, S96J, T97J, S98J, D99J, C100J, G101J, G102J, P103J, K104J, D105J, H106J, P107J, L108J, T109J, C110J, D111J, H112J, P113J, Q114J, L115J, S116J, G117J, L118J, J, L119J, F120J, and L121J. The variable “J” is any amino acid whose introduction results in an increase in the electrostatic interaction between the L1 and L3 β hairpin loop structures of the hLH beta-subunit and a receptor with affinity for a dimeric protein containing the mutant hLH beta-subunit monomer.

The invention also contemplates a number of hLH beta-subunit in modified forms. These modified forms include hLH beta-subunit linked to another cystine knot growth factor or a fraction of such a monomer.

In specific embodiments, the mutant hLH beta-subunit heterodimer comprising at least one mutant subunit or the single chain hLH beta-subunit analog as described above is functionally active, i.e., capable of exhibiting one or more functional activities associated with the wild-type hLH beta-subunit, such as hLH beta-subunit receptor binding, hLH beta-subunit protein family receptor signalling and extracellular secretion. Preferably, the mutant hLH beta-subunit heterodimer or single chain hLH beta-subunit analog is capable of binding to the hLH beta-subunit receptor, preferably with affinity greater than the wild type hLH beta-subunit. Also it is preferable that such a mutant hLH beta-subunit heterodimer or single chain hLH beta-subunit analog triggers signal transduction. Most preferably, the mutant hLH beta-subunit heterodimer comprising at least one mutant subunit or the single chain hLH beta-subunit analog of the present invention has an in vitro bioactivity and/or in vivo bioactivity greater than the wild type hLH beta-subunit and has a longer serum half-life than wild type hLH beta-subunit. Mutant hLH beta-subunit heterodimers and single chain hLH beta-subunit analogs of the invention can be tested for the desired activity by procedures known in the art.

In one embodiment, the present invention provides a mutant CKGF that is a heterodimeric protein, such as a mutant TSH or a mutant hLH, comprising at least one of the above-described mutant α and/or β subunits. The mutant subunits comprise one or more amino acid substitutions.

In specific embodiments, the mutant LH heterodimer comprising at least one mutant subunit or the single chain LH analog as described above is functionally active, i.e., capable of exhibiting one or more functional activities associated with the wild-type LH, such as LHR binding, LHR signalling and extracellular secretion. Preferably, the mutant LH heterodimer or single chain LH analog is capable of binding to the LHR, preferably with affinity greater than the wild type LH. Also it is preferable that such a mutant LH heterodimer or single chain LH analog triggers signal transduction. Most preferably, the mutant LH heterodimer comprising at least one mutant subunit or the single chain LH analog of the present invention has an in vitro bioactivity and/or in vivo bioactivity greater than the wild type LH and has a longer serum half-life than wild type LH. Mutant LH heterodimers and single chain LH analogs of the invention can be tested for the desired activity by procedures known in the art.

Polynucleotides Encoding Mutant LH β Subunit and Analogs

The present invention also relates to nucleic acids molecules comprising sequences encoding mutant subunits of human LH β subunit and LH analogs of the invention, wherein the sequences contain at least one base insertion, deletion or substitution, or combinations thereof that results in single or multiple amino acid additions, deletions and substitutions relative to the wild type protein. Base mutation that does not alter the reading frame of the coding region are preferred. As used herein, when two coding regions are said to be fused, the 3′ end of one nucleic acid molecule is ligated to the 5′ (or through a nucleic acid encoding a peptide linker) end of the other nucleic acid molecule such that translation proceeds from the coding region of one nucleic acid molecule into the other without a frameshift.

Due to the degeneracy of the genetic code, any other DNA sequences that encode the same amino acid sequence for a mutant subunit or monomer may be used in the practice of the present invention. These include but are not limited to nucleotide sequences comprising all or portions of the coding region of the subunit or monomer that are altered by the substitution of different codons that encode the same amino acid residue within the sequence, thus producing a silent change.

In one embodiment, the present invention provides nucleic acid molecules comprising sequences encoding mutant LH β subunits, wherein the mutant LH β subunits comprise single or multiple amino acid substitutions, preferably located in or near the β hairpin L1 and/or L3 loops of the target protein. The invention also provides nucleic acids molecules encoding mutant LH β subunits having an amino acid substitution outside of the L1 and/or L3 loops such that the electrostatic interaction between those loops and the cognate receptor of the LH β subunit holo-protein are increased. The present invention further provides nucleic acids molecules comprising sequences encoding mutant LH β subunits comprising single or multiple amino acid substitutions, preferably located in or near the β hairpin L1 and/or L3 loops of the LH β subunit, and/or covalently joined to CTEP or another CKGF protein.

In yet another embodiment, the invention provides nucleic acid molecules comprising sequences encoding LH β subunit analogs, wherein the coding region of a mutant LH β subunit comprising single or multiple amino acid substitutions, is fused with the coding region of its corresponding dimeric unit, which can be a wild type subunit or another mutagenized monomeric subunit. Also provided are nucleic acid molecules encoding a single chain LH β subunit analog wherein the carboxyl terminus of the mutant LH subunit monomer is linked to the amino terminus of another CKGF protein, such as the CTEP of the β subunit of LH. In still another embodiment, the nucleic acid molecule encodes a single chain LH β subunit analog, wherein the carboxyl terminus of the mutant LH β subunit monomer is covalently bound to the amino terminus another CKGF protein such as the amino terminus of CTEP, and the carboxyl terminus of bound amino acid sequence is covalently bound to the amino terminus of a mutant LH β subunit monomer without the signal peptide.

The single chain analogs of the invention can be made by ligating the nucleic acid sequences encoding monomeric subunits of LH β subunit to each other by methods known in the art, in the proper coding frame, and expressing the fusion protein by methods commonly known in the art. Alternatively, such a fusion protein may be made by protein synthetic techniques, e.g. by use of a peptide synthesizer.

Preparation of Mutant LH β Subunit and Analogs

The production and use of the mutant o, subunits, mutant LH β subunits, mutant LH heterodimers, LH analogs, single chain analogs, derivatives and fragments thereof of the invention are within the scope of the present invention. In specific embodiments, the mutant subunit or LH analog is a fusion protein either comprising, for example, but not limited to, a mutant LH β subunit and another CKGF protein or fragment thereof, or a mutant β subunit and a mutant α subunit. In one embodiment, such a fusion protein is produced by recombinant expression of a nucleic acid encoding a mutant or wild type subunit joined in-frame to the coding sequence for another protein, such as but not limited to toxins, such as ricin or diphtheria toxin. Such a fusion protein can be made by ligating the appropriate nucleic acid sequences encoding the desired amino acid sequences to each other by methods known in the art, in the proper coding frame, and expressing the fusion protein by methods commonly known in the art. Alternatively, such a fusion protein may be made by protein synthetic techniques, e.g., by use of a peptide synthesizer. Chimeric genes comprising portions of mutant α and/or β subunit fused to any heterologous protein-encoding sequences may be constructed. A specific embodiment relates to a single chain analog comprising a mutant α subunit fused to a mutant β subunit, preferably with a peptide linker between the mutant α subunit and the mutant β subunit.

Structure and Function Analysis of Mutant LH Subunits

Described herein are methods for determining the structure of mutant LH subunits, mutant heterodimers and LH analogs, and for analyzing the in vitro activities and in vivo biological functions of the foregoing.

Once a mutant LH β subunit is identified, it may be isolated and purified by standard methods including chromatography (e.g., ion exchange, affinity, and sizing column chromatography), centrifugation, differential solubility, or by any other standard technique for the purification of proteins. The functional properties may be evaluated using any suitable assay (including immunoassays as described infra).

Alternatively, once a mutant LH subunit produced by a recombinant host cell is identified, the amino acid sequence of the subunit(s) can be determined by standard techniques for protein sequencing, e.g., with an automated amino acid sequencer.

The mutant subunit sequence can be characterized by a hydrophilicity analysis (Hopp, T. and Woods, K., 1981, Proc. Natl. Acad. Sci. U.S.A. 78:3824). A hydrophilicity profile can be used to identify the hydrophobic and hydrophilic regions of the subunit and the corresponding regions of the gene sequence which encode such regions.

Secondary structural analysis (Chou, P. and Fasman, G., 1974, Biochemistry 13:222) can also be done, to identify regions of the subunit that assume specific secondary structures.

Other methods of structural analysis can also be employed. These include but are not limited to X-ray crystallography (Engstom, A., 1974, Biochem. Exp. Biol. 11:7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.). Structure prediction, analysis of crystallographic data, sequence alignment, as well as homology modelling, can also be accomplished using computer software programs available in the art, such as BLAST, CHARMM release 21.2 for the Convex, and QUANTA v.3.3, (Molecular Simulations, Inc., York, United Kingdom).

The functional activity of mutant LH β subunits, mutant LH heterodimers, LH analogs, single chain analogs, derivatives and fragments thereof can be assayed by various methods known in the art.

For example, where one is assaying for the ability of a mutant LH β subunit or mutant LH to bind or compete with wild-type LH or its subunits for binding to an antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. Antibody binding can be detected by detecting a label on the primary antibody. Alternatively, the primary antibody is detected by detecting binding of a secondary antibody or reagent to the primary antibody, particularly where the secondary antibody is labeled. Many means are known in the art for detecting binding in an immunoassay and are within the scope of the present invention.

The binding of mutant LH β subunits, mutant LH heterodimers, LH analogs, single chain analogs, derivatives and fragments thereof, to the human chorionic gonadotropin receptor (LHR) can be determined by methods well-known in the art, such as but not limited to in vitro assays based on displacement from the LHR of a radiolabelled mutant LH by wild type LH, for example. The bioactivity of mutant LH heterodimers, LH analogs, single chain analogs, derivatives and fragments thereof, can also be measured in the cell based assay used for hCG bioactivity that is modeled on work by in Morbeck, et al., Mole. and Cell. Endocrinol., 97:173-181 (1993).

The half-life of a protein is a measurement of protein stability and indicates the time necessary for a one-half reduction in the concentration of the protein. The half life of a mutant LH can be determined by any method for measuring LH levels in samples from a subject over a period of time, for example but not limited to, immunoassays using anti-LH antibodies to measure the mutant LH levels in samples taken over a period of time after administration of the mutant LH or detection of radiolabelled mutant LH in samples taken from a subject after administration of the radiolabelled mutant LH.

Other methods will be known to the skilled artisan and are within the scope of the invention.

Diagnostic and Therapeutic Uses

The invention provides for treatment or prevention of various diseases and disorders by administration of therapeutic compound (termed herein “Therapeutic”) of the invention. Such Therapeutics include LH heterodimers having a mutant α and either a mutant or wild type LH β subunit; LH heterodimers having a mutant α subunit, preferably with one or more amino acid substitutions in or near the L1 and/or L3 loops and a mutant β subunit, preferably with one or more amino acid substitutions in or near the L1 and/or L3 loops and covalently bound to another CKGF protein, in whole or in part; LH heterodimers having a mutant α subunit, and a mutant β subunit, where the mutant α subunit and the mutant β subunit are covalently bound to form a single chain analog, including a LH heterodimer where the mutant α subunit and the mutant β subunit and another CKGF protein covalently bound in a single chain analog, other derivatives, analogs and fragments thereof (e.g. as described hereinabove) and nucleic acids encoding the mutant LH heterodimers of the invention, and derivatives, analogs, and fragments thereof.

The subject to which the Therapeutic is administered is preferably an animal, including but not limited to animals such as cows, pigs, horses, chickens, cats, dogs, etc., and is preferably a mammal. In a preferred embodiment, the subject is a human. Generally, administration of products of a species origin that is the same species as that of the subject is preferred. Thus, in a preferred embodiment, a human mutant and/or modified LH heterodimer, derivative or analog, or nucleic acid, is therapeutically or prophylactically or diagnostically administered to a human patient.

In a preferred aspect, the Therapeutic of the invention is substantially purified.

A reproductive disorder known as luteal phase disorder effects the development of the corpus luteum. Administration of LH can restore the ovulation mechanism, which has the luteal phase as a step, to normal functioning. Conditions in which LH is absent or decreased relative to normal or desired levels are treated or prevented by administration of a mutant LH heterodimer or LH analog of the invention. Disorders in which the LH receptor is absent or decreased relative to normal levels or unresponsive or less responsive than normal LHR to wild type LH, can also be treated by administration of a mutant LH heterodimer or LH analog. Constitutively active LHR can lead to hyperthyroidism, and it is contemplated that mutant LH heterodirners and LH analogs can be used as antagonists.

In specific embodiments, mutant LH heterodimers or LH analogs that are capable of stimulating ovulatory or sexual characteristic development functions are administered therapeutically, including prophylactically. Diseases and disorders that can be treated or prevented include but are not limited to hypogonadism, hypergonadism, luteal phase disorder, unexplained infertility, etc.

The absence of decreased level in LH protein or function, or LHR protein and function can be readily detected, e.g., by obtaining a patient tissue sample (e.g., from biopsy tissue) and assaying it in vitro for RNA or protein levels, structure and/or activity of the expressed RNA or protein of LH or LH R. Many methods standard in the art can be thus employed, including but not limited to immunoassays to detect and/or visualize LH or LH R protein (e.g., Western blot, immunoprecipitation followed by sodium dodecyl sulfate polyacrylamide gel electrophoresis, immunocytochemistry, etc.) and/or hybridization assays to detect LH or LHR expression by detecting and/or visualizing LH or LHR mRNA (e.g., Northern assays, dot blots, in situ hybridization, etc.), etc.

Mutants of the FSH β Subunit

The human β subunit of human follicle stimulating hormone (FSH) contains 109 amino acids as shown in FIG. 6 (SEQ ID No: 5). The invention contemplates mutants of the β subunit of hFSH wherein the subunit comprises single or multiple amino acid substitutions, located in or near the β hairpin L1 and/or L3 loops of the β subunit, where such mutants are fused to another CKGF protein, in whole or in part, such as TSH or are part of a hFSH heterodimer. The mutant hFSH heterodimers of the invention have β subunits having substitutions, deletions or insertions, of one, two, three, four or more amino acid residues when compared with the wild type subunit.

The present invention further provides a mutant hFSH β subunit having an L1 hairpin loop with one or more amino acid substitutions between positions 4 and 27, inclusive, excluding Cys residues, as depicted in FIG. 6 (SEQ ID NO:5). The amino acid substitutions include: E4X, L5X, T6X, N7X, I8X, T9X, I10X, A11X, I12X, E13X, K14X, E15X, E16X, R18X, F19X, I21X, S22X, I23X, N24X, T25X, T26X, and W27X.

In another aspect of this embodiment, neutral or acidic amino acid residues in the hFSH β subunit, L1 hairpin loop are mutated. The resulting mutated subunits contain at least one mutation in the amino acid sequence of SEQ ID NO: 5 at the following amino acid positions: E4B, L5B, T6B, N7B, I8B, T9B, I10B, A11B, I12B, E13B, E15B, E16B, F19B, I21B, S22B, I23B, N24B, T25B, T26B, and W27B.

Introducing acidic amino acid residues where basic residues are present in the hFSH beta-subunit monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following K14Z and R18Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at E4U, E13U, K14U, E15U, E16U and R18U, wherein “U” is a neutral amino acid.

Mutant hFSH beta-subunit monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include L5Z, T6Z, N7Z, I8Z, T9Z, I10Z, A11Z, I12Z, C17Z, F19Z, C20Z, I21Z, S22Z, I23Z, N24Z, T25Z, T26Z, W27Z, L5B, T6B, N7B, I8B, T9B, I10B, A11B, I12B, C17B, F19B, C20B, I21B, S22B, I23B, N24B, T25B, T26B, and W27B, wherein “Z” is an acidic amino acid and “B” is a basic amino acid.

The present invention also provides a mutant CKGF subunit that is a mutant hFSH β subunit, L3 hairpin loop having one or more amino acid substitutions between positions 65 and 81, inclusive, excluding Cys residues, as depicted in FIG. 6 (SEQ ID NO: 5). The amino acid substitutions include: A65X, H66X, H67X, A68X, D69X, S70X, L71X, Y72X, T73X, Y74X, P75X, V76X, A77X, T78X, Q79X, and H81X.

In another aspect of this embodiment, neutral or acidic amino acid residues in the hFSH β subunit, L3 hairpin loop are mutated. The resulting mutated subunits contain at least one mutation in the amino acid sequence of SEQ ID NO: 5 at the following amino acid positions: A65B, A68B, D69B, S70B, L71B, Y72B, T73B, Y74B, P75B, V76B, A77B, T78B, and Q79B.

The invention further contemplates introducing one or more acidic residues into the amino acid sequence of the hFSH beta-subunit L3 hairpin loop. For example, one or more acidic amino acids can be introduced in the sequence described above, wherein the variable “X” corresponds to an acidic amino acid. Specific examples of such mutations include H66Z, H67Z, and H81Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at H66U, H67U, D69U, and H81U, wherein “U” is a neutral amino acid.

Mutant hFSH beta-subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include A66Z, H67Z, H68Z, A69Z, D70Z, S71Z, L72Z, Y73Z, T74Z, Y75Z, P76Z, V77Z, A78Z, T79Z, Q80Z, A66B, H67B, H68B, A69B, D70B, S71B, L72B, Y73B, T74B, Y75B, P76B, V77B, A78B, T79B, and Q80B, wherein “Z” is an acidic amino acid and “B” is a basic amino acid.

The present invention also contemplate hFSH beta-subunit containing mutations outside of said β hairpin loop structures that alter the structure or conformation of those hairpin loops. These structural alterations in turn serve to increase the electrostatic interactions between regions of the β hairpin loop structures of hFSH beta-subunit contained in a dimeric molecule, and a receptor having affinity for the dimeric protein. These mutations are found at positions selected from the group consisting of positions 1-3, 28-64, and 82-109 of the hFSH beta-subunit monomer.

Specific examples of these mutation outside of the β hairpin L1 and L3 loop structures include, N1J, S2J, C3J, A29J, G30J, Y31J, C32J, Y33J, T34J, R35J, D36J, L37J, V38J, Y39J, K40J, D41J, P42J, A43J, R44J, P45J, K46J, i47J, t48J, C49J, T50J, F51J, K52J, E53J, L54J, V55J, Y56J, E57J, T58J, V59J, R60J, V61J, P62J, G63J, C64J, C82J, G83J, K84J, C85J, D86J, S87J, D88J, S89J, T90J, D91J, C92J, T93J, V94J, R95J, G96J, L97J, G98J, P99J, S100J, Y101J, C102J, S103J, F104J, G105J, E106J, M107J, K108J, and E109J. The variable “J” is any amino acid whose introduction results in an increase in the electrostatic interaction between the L1 and L3 β hairpin loop structures of the hFSH beta-subunit and a receptor with affinity for a dimeric protein containing the mutant hFSH beta-subunit monomer.

The invention also contemplates a number of hFSH beta-subunit in modified forms. These modified forms include hFSH beta-subunit linked to another cystine knot growth factor or a fraction of such a monomer.

In specific embodiments, the mutant hFSH beta-subunit heterodimer comprising at least one mutant subunit or the single chain hFSH beta-subunit analog as described above is functionally active, i.e., capable of exhibiting one or more functional activities associated with the wild-type hFSH beta-subunit, such as hFSH beta-subunit receptor binding, hFSH beta-subunit protein family receptor signalling and extracellular secretion. Preferably, the mutant hFSH beta-subunit heterodimer or single chain hFSH beta-subunit analog is capable of binding to the hFSH beta-subunit receptor, preferably with affinity greater than the wild type hFSH beta-subunit. Also it is preferable that such a mutant hFSH beta-subunit heterodimer or single chain hFSH beta-subunit analog triggers signal transduction. Most preferably, the mutant hFSH beta-subunit heterodimer comprising at least one mutant subunit or the single chain hFSH beta-subunit analog of the present invention has an in vitro bioactivity and/or in vivo bioactivity greater than the wild type hFSH beta-subunit and has a longer serum half-life than wild type hFSH beta-subunit. Mutant hFSH beta-subunit heterodimers and single chain hFSH beta-subunit analogs of the invention can be tested for the desired activity by procedures known in the art.

In one embodiment, the present invention provides a mutant CKGF that is a heterodimeric protein, such as a mutant hFSH or a mutant hFSH, comprising at least one of the above-described mutant α and/or β subunits. The mutant subunits comprise one or more amino acid substitutions.

In specific embodiments, the mutant FSH heterodimer comprising at least one mutant subunit or the single chain FSH analog as described above is functionally active, i.e., capable of exhibiting one or more functional activities associated with the wild-type FSH, such as FSHR binding, FSHR signalling and extracellular secretion. Preferably, the mutant FSH heterodimer or single chain FSH analog is capable of binding to the FSHR, preferably with affinity greater than the wild type FSH. Also it is preferable that such a mutant FSH heterodimer or single chain FSH analog triggers signal transduction. Most preferably, the mutant FSH heterodimer comprising at least one mutant subunit or the single chain FSH analog of the present invention has an in vitro bioactivity and/or in vivo bioactivity greater than the wild type FSH and has a longer serum half-life than wild type FSH. Mutant FSH heterodimers and single chain FSH analogs of the invention can be tested for the desired activity by procedures known in the art.

Polynucleotides Encoding Mutant FSH and Analogs

The present invention also relates to nucleic acids molecules comprising sequences encoding mutant subunits of human FSH and FSH analogs of the invention, wherein the sequences contain at least one base insertion, deletion or substitution, or combinations thereof that results in single or multiple amino acid additions, deletions and substitutions relative to the wild type protein. Base mutation that does not alter the reading frame of the coding region are preferred. As used herein, when two coding regions are said to be fused, the 3′ end of one nucleic acid molecule is ligated to the 5′ (or through a nucleic acid encoding a peptide linker) end of the other nucleic acid molecule such that translation proceeds from the coding region of one nucleic acid molecule into the other without a frameshift.

Due to the degeneracy of the genetic code, any other DNA sequences that encode the same amino acid sequence for a mutant subunit or monomer may be used in the practice of the present invention. These include but are not limited to nucleotide sequences comprising all or portions of the coding region of the subunit or monomer that are altered by the substitution of different codons that encode the same amino acid residue within the sequence, thus producing a silent change.

In one embodiment, the present invention provides nucleic acid molecules comprising sequences encoding mutant FSH subunits, wherein the mutant FSH subunits comprise single or multiple amino acid substitutions, preferably located in or near the β hairpin L1 and/or L3 loops of the target protein. The invention also provides nucleic acids molecules encoding mutant FSH subunits having an amino acid substitution outside of the L1 and/or L3 loops such that the electrostatic interaction between those loops and the cognate receptor of the FSH dimer are increased. The present invention further provides nucleic acids molecules comprising sequences encoding mutant FSH subunits comprising single or multiple amino acid substitutions, preferably located in or near the β hairpin L1 and/or L3 loops of the FSH subunit, and/or covalently joined to CTEP or another CKGF protein.

In yet another embodiment, the invention provides nucleic acid molecules comprising sequences encoding FSH analogs, wherein the coding region of a mutant FSH subunit comprising single or multiple amino acid substitutions, is fused with the coding region of its corresponding dimeric unit, which can be a wild type subunit or another mutagenized monomeric subunit. Also provided are nucleic acid molecules encoding a single chain FSH analog wherein the carboxyl terminus of the mutant FSH monomer is linked to the amino terminus of another CKGF protein, such as the CTEP of the β subunit of hLH. In still another embodiment, the nucleic acid molecule encodes a single chain FSH analog, wherein the carboxyl terminus of the mutant FSH monomer is covalently bound to the amino terminus another CKGF protein such as the amino terminus of CTEP, and the carboxyl terminus of bound amino acid sequence is covalently bound to the amino terminus of a mutant FSH monomer without the signal peptide.

The single chain analogs of the invention can be made by ligating the nucleic acid sequences encoding monomeric subunits of FSH to each other by methods known in the art, in the proper coding frame, and expressing the fusion protein by methods commonly known in the art. Alternatively, such a fusion protein may be made by protein synthetic techniques, e.g. by use of a peptide synthesizer.

Preparation of Mutant FSH Subunits and Analogs

The production and use of the mutant α subunits, mutant FSH β subunits, mutant FSH heterodimers, FSH analogs, single chain analogs, derivatives and fragments thereof of the invention are within the scope of the present invention. In specific embodiments, the mutant subunit or FSH analog is a fusion protein either comprising, for example, but not limited to, a mutant FSH β subunit and the CTEP of the β subunit of hLH or a mutant β subunit and a mutant α subunit. In one embodiment, such a fusion protein is produced by recombinant expression of a nucleic acid encoding a mutant or wild type subunit joined in-frame to the coding sequence for another protein, such as but not limited to toxins, such as ricin or diphtheria toxin. Such a fusion protein can be made by ligating the appropriate nucleic acid sequences encoding the desired amino acid sequences to each other by methods known in the art, in the proper coding frame, and expressing the fusion protein by methods commonly known in the art. Alternatively, such a fusion protein may be made by protein synthetic techniques, e.g., by use of a peptide synthesizer. Chimeric genes comprising portions of mutant α and/or β subunit fused to any heterologous protein-encoding sequences may be constructed. A specific embodiment relates to a single chain analog comprising a mutant α subunit fused to a mutant β subunit, preferably with a peptide linker between the mutant α subunit and the mutant β subunit.

Structure and Function Analysis of Mutant FSH Subunits

Described herein are methods for determining the structure of mutant FSH subunits, mutant heterodimers and FSH analogs, and for analyzing the in vitro activities and in vivo biological functions of the foregoing.

Once a mutant α or FSH β subunit is identified, it may be isolated and purified by standard methods including chromatography (e.g., ion exchange, affinity, and sizing column chromatography), centrifugation, differential solubility, or by any other standard technique for the purification of proteins. The functional properties may be evaluated using any suitable assay (including immunoassays as described infra).

Alternatively, once a mutant α subunit and/or FSH β subunit produced by a recombinant host cell is identified, the amino acid sequence of the subunit(s) can be determined by standard techniques for protein sequencing, e.g., with an automated amino acid sequencer.

The mutant subunit sequence can be characterized by a hydrophilicity analysis (Hopp, T. and Woods, K., 1981, Proc. Natl. Acad. Sci. U.S.A. 78:3824). A hydrophilicity profile can be used to identify the hydrophobic and hydrophilic regions of the subunit and the corresponding regions of the gene sequence which encode such regions.

Secondary structural analysis (Chou, P. and Fasman, G., 1974, Biochemistry 13:222) can also be done, to identify regions of the subunit that assume specific secondary structures.

Other methods of structural analysis can also be employed. These include but are not limited to X-ray crystallography (Engstom, A., 1974, Biochem. Exp. Biol. 11:7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.). Structure prediction, analysis of crystallographic data, sequence alignment, as well as homology modelling, can also be accomplished using computer software programs available in the art, such as BLAST, CHARMM release 21.2 for the Convex, and QUANTA v.3.3, (Molecular Simulations, Inc., York, United Kingdom).

The functional activity of mutant α subunits, mutant β subunits, mutant FSH heterodimers, FSH analogs, single chain analogs, derivatives and fragments thereof can be assayed by various methods known in the art.

For example, where one is assaying for the ability of a mutant subunit or mutant FSH to bind or compete with wild-type FSH or its subunits for binding to an antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. Antibody binding can be detected by detecting a label on the primary antibody. Alternatively, the primary antibody is detected by detecting binding of a secondary antibody or reagent to the primary antibody, particularly where the secondary antibody is labeled. Many means are known in the art for detecting binding in an immunoassay and are within the scope of the present invention.

The binding of mutant α subunits, mutant FSH β subunits, mutant FSH heterodimers, FSH analogs, single chain analogs, derivatives and fragments thereof, to the follicle stimulating hormone receptor (FSHR) can be determined by methods well-known in the art, such as but not limited to in A vitro assays based on displacement from the FSHR of a radiolabelled FSH of another species, such as bovine FSH. The bioactivity of mutant FSH heterodimers, FSH analogs, single chain analogs, derivatives and fragments thereof, can also be measured, for example, by assays based on measurements taken in Chinese hamster ovary (CHO) cells that stably express the human FSH receptor and a cAMP responsive human glycoprotein hormone α subunit luciferase reporter construct. In this assay, the bioactivity of a mutant FSH protein is determined by establishing the amount of luciferase activity induced from a test cell population and comparing that value to the luciferase activity induce by the wild type form of the protein.

Chinese hamster ovary cells (American Type Culture Collection, Rockville, Md.) are transfected with the human FSH receptor as described by Albanese, et al., Mole. Cell. Endocrinol., 101:211-219 (1994). These cells are also transfected with the reporter gene construct described by Albanese et al. Briefly, Exponentially dividing CHO cells are transfected at 30% confluency using 10 μg of the FSH receptor expressing construct and 2 μg of the reporter gene construct per 100-mm plate using a calcium phosphate precipitation method. Stable transformants are selected using Geneticin (GIBCO/BRL, Grand Island, N.Y.). Resistant cells are subcloned and a cell line, CHO/FSH-R, are selected by virtue of FSH stimulation of the luciferase reporter activity. Receptor stimulation assay are carried out by dispensing 5×105 cells per well in 24-well tissue culture plates or 4×104 cells per well in 96-well culture plates. After 16-20 hours, cells were incubated at 37° C. in 300 μl or 100 μl, respectively, of culture medium containing 0.25 mM 3-isobutyl-1-methylzanthine, IBMX (Sigma, St. Louis, Mo.) along with the indicated additions.

Luciferase assays are carried out as described by Albanese et al., Mol. Endocrinol., 5:693-702 (1991). Briefly, after incubation, the tissue culture media is aspirated and 200 μl of lysis solution, containing 25 mM EGTA, 1% Triton X-100 and 1 mM DTT, is added to each well and allowed to sit for 10 minutes. After agitation, the cell lysate is added to 365 μl of assay buffer containing 25 mM glycylglycine pH 7.8, 15 mM MgSO₄, 4 mM EGTA, 16.5 mM KPO₄, 1 mM DTT and 2.2 mM ATP. Luciferase activity is assayed by injection of 100 μl of 250 μM luciferin and 10 mM DTT at room temperature and measuring the light emitted during the first 10 seconds of the reaction with a luminometer (Monolight 2010, Analytical Luminescensce Laboratory, San Diego, Calif.). An example of this assay is found in Albanese, et al., Mole. Cell. Endocrinol., 101:211-219 (1994).

The half-life of a protein is a measurement of protein stability and indicates the time necessary for a one-half reduction in the concentration of the protein. The half life of a mutant FSH can be determined by any method for measuring FSH levels in samples from a subject over a period of time, for example but not limited to, immunoassays using anti-FSH antibodies to measure the mutant FSH levels in samples taken over a period of time after administration of the mutant FSH or detection of radiolabelled mutant FSH in samples taken from a subject after administration of the radiolabelled mutant FSH.

Other methods will be known to the skilled artisan and are within the scope of the invention.

Diagnostic and Therapeutic Uses

The invention provides for treatment or prevention of various diseases and disorders by administration of therapeutic compound (termed herein “Therapeutic”) of the invention. Such Therapeutics include FSH heterodimers having a mutant α subunit and either a mutant or wild type β subunit; FSH heterodimers having a mutant α subunit and a mutant β subunit and covalently bound to another CKGF protein, in whole or in part, such as the CTEP of the β subunit of hLH; FSH heterodimers having a mutant α subunit and a mutant β subunit, where the mutant α subunit and the mutant β subunit are covalently bound to form a single chain analog, including a FSH heterodimer where the mutant α subunit and the mutant β subunit and the CKGF protein or fragment are covalently bound in a single chain analog, other derivatives, analogs and fragments thereof (e.g. as described hereinabove) and nucleic acids encoding the mutant FSH heterodimers of the invention, and derivatives, analogs, and fragments thereof.

The subject to which the Therapeutic is administered is preferably an animal, including but not limited to animals such as cows, pigs, horses, chickens, cats, dogs, etc., and is preferably a mammal. In a preferred embodiment, the subject is a human. Generally, administration of products of a species origin that is the same species as that of the subject is preferred. Thus, in a preferred embodiment, a human mutant and/or modified FSH heterodimer, derivative or analog, or nucleic acid, is therapeutically or prophylactically or diagnostically administered to a human patient.

In a preferred aspect, the Therapeutic of the invention is substantially purified.

A number of disorders which manifest as infertility or sexual disfunction can be treated by the methods of the invention. Disorders in which FSH is absent or decreased relative to normal or desired levels are treated or prevented by administration of a mutant FSH heterodimer or FSH analog of the invention. Disorders in which FSH receptor is absent or decreased relative to normal levels or unresponsive or less responsive than normal FSHR to wild type FSH, can also be treated by administration of a mutant FSH heterodimer or FSH analog. Mutant FSH heterodimers and FSH analogs for use as antagonists are contemplated by the present invention.

In specific embodiments, mutant FSH heterodimers or FSH analogs with bioactivity are administered therapeutically, including prophylactically to treat ovulatory dysfunction, luteal phase defect, unexplained infertility, time-limited conception, and in assisted reproduction.

The absence of or a decrease in FSH protein or function, or FSHR protein and function can be readily detected, e.g., by obtaining a patient tissue sample (e.g., from biopsy tissue) and assaying it in vitro for RNA or protein levels, structure and/or activity of the expressed RNA or protein of FSH or FSHR. Many methods standard in the art can be thus employed, including but not limited to immunoassays to detect and/or visualize FSH or FSHR protein (e.g., Western blot, immunoprecipitation followed by sodium dodecyl sulfate polyacrylamide gel electrophoresis, immunocytochemistry, etc.) and/or hybridization assays to detect FSH or FSHR expression by detecting and/or visualizing FSH or FSHR mRNA (e.g., Northern assays, dot blots, in situ hybridization, etc.), etc.

Mutants of the PDGF Family

The present invention contemplates introducing mutations throughout the platelet-derived growth factor sequence of the β hairpin L1 and/or L3 loops of the PDGF monomers such that the eletrostatic charge of these structures are altered. The invention contemplates mutants of the PDGF monomeric chains comprising single or multiple amino acid substitutions, or amino acid deletions or insertions, located in or near the β hairpin L1 and/or L3 loops of the PDGF monomeric chains that result in a change in the electrostatic character of the β hairpin loops of these proteins. The invention further contemplates mutations to the PDGF monomeric chains that alter the conformation of the β hairpin loops of the protein such that the interaction between the PDGF dimer and its cognate receptor or receptors is increased. Furthermore, the invention contemplates mutant PDGF monomers that are linked to another CKGF protein.

Mutants of the PDGF-A (PDGF A-Chain)

The human A-chain of human platelet-derived growth factor-A (PDGF-A) contains 125 amino acids as shown in FIG. 7 (SEQ ID NO: 6). The invention contemplates mutants of the PDGF A-Chain comprises amino acid substitutions, deletions or insertions, of one, two, three, four or more amino acid residues when compared with the wild type subunit. Furthermore, the invention contemplates mutant PDGF A-Chain molecules that are linked to another CKGF protein.

The present invention provides mutant PDGF A-chain L1 hairpin loops having one or more amino acid substitutions between positions 11 and 36, inclusive, excluding Cys residues, as depicted in FIG. 7 (SEQ ID NO: 6). The amino acid substitutions include: K11X, T12X, R13X, T14X, V15X, I16X, Y17X, E18X, I19X, P20X, R21X, S22X, Q23X, V24X, D25X, P26X, T27X, S28X, A29X, N30X, F31X, L32X, I33X, W34X, P35X, and P36X. “X” represent any amino acid residue.

Specific examples of the mutagenesis regime of the present invention include the introduction of basic amino acid residues where acidic amino acid residues are present. The introduction of these basic residues alters the electrostatic charge of the L1 hairpin loop to have a more positive character for each basic amino acid introduced. For example, when introducing basic residues into the L1 loop of the PDGF A monomer, the variable “X” would correspond to a basic amino acid residue selected from the group consisting of lysine (K) or arginine (R). Specific examples of electrostatic charge altering mutations where a basic residue is introduced into the PDGF A monomer include one or more of the following: E18B and D25B, wherein “B” is a basic amino acid residue.

Introducing acidic amino acid residues where basic residues are present in the PDGF A monomer sequence is also contemplated. In this embodiment, the variable “X” corresponds to an acidic amino acid such as aspartic acid (D) or glutamic acid (E). The introduction of these amino acids serves to alter the electrostatic character of the L1 hairpin loops to a more negative state. Examples of such amino acid substitutions include one or more of the following: K11Z, R13Z and R21Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at K11U, R13U, E18U, R21U and D25U, wherein “U” is a neutral amino acid. For the purposes of the invention, a neutral amino acid is any amino acid other than D, E, K, R, or H. Accordingly, neutral amino acids are selected from the group consisting of A, N, C, Q, G, I, L, M, F, P, S, T, W, Y, and V.

Mutant PDGF A-chain proteins are provided containing one or more electrostatic charge altering mutations in the L1 hairpin loop amino acid sequence that convert non-charged or neutral amino acid residues to charged residues. Examples of mutations converting neutral amino acid residues to charged residues include: T12Z, T14Z, V15Z, I16Z, Y17Z, I19Z, P20Z, S22Z, Q23Z, V24Z, P26Z, T27Z, S28Z, A29Z, N30Z, F31Z, L32Z, I33Z, W34Z, P35Z, P36Z, T12B, T14B, V15B, I16B, Y17B, I19B, P20B, S22B, Q23B, V24B, P26B, T27B, S28B, A29B, N30B, F31B, L32B, I33B, W34B, P35B, and P36B, wherein “Z” is an acidic amino acid and “B” is a basic amino acid.

Mutant PDGF A-chain monomers containing mutants in the L3 hairpin loop are also described. These mutant proteins have one or more amino acid substitutions, deletion or insertions, between positions 58 and 88, inclusive, excluding Cys residues, of the L3 hairpin loop, as depicted in FIG. 7 (SEQ ID NO: 6). The amino acid substitutions include: R58X, V59X, H60X, H61X, R62X, S63X, V64X, K65X, V66X, A67X, K68X, V69X, E70X, Y71X, V72X, R73X, K74X, K75X, P76X, K77X, L78X, K79X, E80X, V81X, Q82X, V83X, R84X, L85X, E86X, E87X, and H88X, wherein “X” is any amino acid residue, the substitution of which alters the electrostatic character of the L3 loop.

One set of mutations of the L3 hairpin loop includes introducing a basic amino acid into PDGF A-chain L3 hairpin loops amino acid sequence replacing acidic amino acid residues. For example, when introducing basic residues into the L3 loop of the PDGF A monomer, the variable “X” would corresponds to a basic amino acid residue. Specific examples of electrostatic charge altering mutations where a basic residue is introduced into the PDGF A monomer include one or more of the following E70B, E80B, E86B and E87B, wherein “B” is a basic amino acid residue.

The invention further contemplates introducing one or more acidic residues into the amino acid sequence of the PDGF L3 hairpin loop where a basic amino acid residue is positioned. For example, one or more acidic amino acids can be introduced in the sequence of 58-88 described above, wherein the variable “X” corresponds to an acidic amino acid. Specific examples of such mutations include R58Z, H60Z, H61Z, R62Z, K65Z, K68Z, R73Z, K74Z, K75Z, K77Z, K79Z, R84Z, and H88Z.

The invention also contemplates reducing a positive or negative charge in the L3 hairpin loop by mutating a charged residue to a neutral residue. For example, one or more neutral amino acids can be introduced into the L3 hairpin loop amino acid sequence described above where the variable “X” corresponds to a neutral amino acid. For example, one or more neutral residues can be introduced at R58U, H60U, H61U, R62U, K65U, K68U, E70U, R73U, K74U, K75U, K77U, K79U, E80U, R84U, E86U, E87U, and H88U, wherein “U” is a neutral amino acid.

Mutant PDGF A-chain proteins are provided containing one or more electrostatic charge altering mutations in the L3 hairpin loop amino acid sequence that convert non-charged or neutral amino acid residues to charged residues. Examples of mutations converting neutral amino acid residues to charged residues include, V59Z, S63Z, V64Z, V66Z, A67Z, V69Z, Y71Z, V72Z, P76Z, L78Z, V81Z, Q82Z, V83Z, L85Z, V59B, S63B, V64B, V66B, A67B, V69B, Y71B, V72B, P76B, L78B, V81B, Q82B, V83B, and L85B, wherein “Z” is an acidic amino acid and “B” is a basic amino acid.

The present invention also contemplate PDGF A-chain monomers containing mutations outside of said β hairpin loop structures that alter the structure or conformation of those hairpin loops. These structural alterations in turn serve to increase the electrostatic interactions between regions of the β hairpin loop structures of PDGF A-chain monomer contained in a dimeric molecule, and a receptor having affinity for the dimeric protein. These mutations are found at positions selected from the group consisting of positions 1-9, 38-57, and 89-125 of the PDGF A-chain monomer.

Specific examples of these mutation outside of the β hairpin L1 and L3 loop structures include, S1J, I2J, E3J, E4J, A5J, V6J, P7J, A8J, V9J, V38J, E39J, V40J, K41J, R42J, C43J, T44J, G45J, C46J, C47J, N48J, T49J, S50J, S51J, V52J, K53J, C54J, Q55J, P56J, S57J, L89J, E90J, C91J, A92J, C93J, A94J, T95J, T96J, S97J, L98J, N99J, P100J, D101J, Y102J, R103J, E104J, E105J, D106J, T107J, G108J, R109J, P110J, R111J, E112J, S113J, G114J, K115J, K116J, R117J, K118J, R119J, K120J, R121J, L122J, K123J, P124J, and T125J. The variable “J” is any amino acid whose introduction results in an increase in the electrostatic interaction between the L1 and L3 β hairpin loop structures of the PDGF A-chain and a receptor with affinity for a dimeric protein containing the mutant PDGF A-chain monomer.

The invention also contemplates a number of PDGF A-chain monomers in modified forms. These modified forms include PDGF-A monomers linked to another cystine knot growth factor monomer or a fraction of such a monomer.

Mutants of the PDGF-B (PDGF B-Chain)

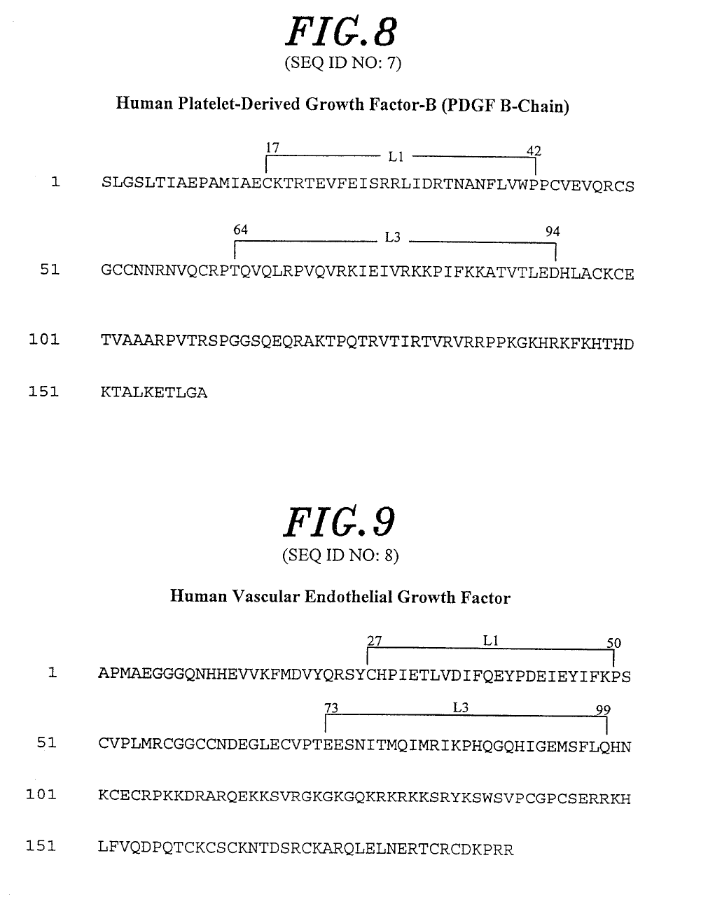

The human B-chain of human platelet-derived growth factor-B (PDGF-B) contains 160 amino acids as shown in FIG. 8 (SEQ ID No: 7). The invention contemplates mutants of the PDGF B-Chain comprising single or multiple amino acid substitutions, deletions or insertions, of one, two, three, four or more amino acid residues when compared with the wild type subunit. Furthermore, the invention contemplates mutant PDGF B-chain molecules that are linked to another CKGF protein.

The present invention provides mutant PDGF B-chain L1 hairpin loops having one or more amino acid substitutions between positions 17 and 42, inclusive, excluding Cys residues, as depicted in FIG. 8 (SEQ ID NO: 7). The amino acid substitutions include: K17X, T18X, R19X, T20X, E21X, V22X, F23X, E24X, I25X, S26X, R27X, R28X, L29X, I30X, D31X, R32X, T33X, N34X, A35X, N36X, F37X, L38X, V39X, W40X, P41X, and P42X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the PDGF “B” monomer, the variable “X” would correspond to a basic amino acid residue. Specific examples of electrostatic charge altering mutations where a basic residue is introduced into the PDGF “B” monomer include one or more of the following: E21B, E24B, and D31B, wherein “B” is a basic amino acid residue.

Introducing acidic amino acid residues where basic residues are present in the PDGF “B” monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: K17Z, R19Z, R27Z, R28Z, and R32Z, wherein “Z” is an acidic amino acid.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at K17U, R19U, E21U, E24U, R27U, R28U, D31U, and R32U, wherein “U” is a neutral amino acid.

Mutant PDGF B-chain proteins are provided containing one or more electrostatic charge altering mutations in the L1 hairpin loop amino acid sequence that convert non-charged or neutral amino acid residues to charged residues. Examples of mutations converting neutral amino acid residues to charged residues include: T18Z, T20Z, V22Z, F23Z, I25Z, S26Z, L29Z, I30Z, T33Z, N34Z, A35Z, N36Z, F37Z, L38Z, V39Z, W40Z, P41Z, P42Z, T18B, T20B, V22B, F23B, I25B, S26B, L29B, I30B, T33B, N34B, A35B, N36B, F37B, L38B, V39B, W40B, P41B, and P42B, wherein “Z” is an acidic amino acid and “B” is a basic amino acid.

Mutant PDGF B-chain monomers containing mutants in the L3 hairpin loop are also described. These mutant proteins have one or more amino acid substitutions, deletion or insertions, between positions 64 and 94, inclusive, excluding Cys residues, of the L3 hairpin loop, as depicted in FIG. 8 (SEQ ID NO: 7). The amino acid substitutions include: Q64X, V65X, Q66X, L67X, R68X, P69X, V70X, Q71X, V72X, R73X, K74X, I75X, E76X, I77X, V78X, R79X, K80X, K81X, P82X, I83X, F84X, K85X, K86X, A87X, T88X, V89X, T90X, L91X, E92X, D93X, and H94X, wherein “X” is any amino acid residue, the substitution of which alters the electrostatic character of the L3 loop.

One set of mutations of the L3 hairpin loop includes introducing one or more basic amino acid residues into the PDGF B-chain L3 hairpin loop amino acid sequence. For example, when introducing basic residues into the L3 loop of the PDGF “B” monomer, the variable “X” of the sequence described above corresponds to a basic amino acid residue. Specific examples of electrostatic charge altering mutations where a basic residue is introduced into the PDGF “B” monomer where an acidic residue resides include one or more of the following: E76B, E92B, and D93B, wherein “B” is a basic amino acid residue.

The invention further contemplates introducing one or more acidic residues into the amino acid sequence of the PDGF L3 hairpin loop. For example, one or more acidic amino acids can be introduced in the sequence of 64-94 described above where a basic residue resides, wherein the variable “X” corresponds to an acidic amino acid. Specific examples of such mutations include R73Z, K74Z, R79Z, K80Z, K81Z, K85Z, K86Z, and H94Z, wherein “Z” is the acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at R68U, R73U, K74U, E76U, R79U, K80U, K81U, K85U, K86U, E92U, D93U, and H94U, wherein “U” is a neutral amino acid.

Mutant PDGF B-chain proteins are provided containing one or more electrostatic charge altering mutations in the L3 hairpin loop amino acid sequence that convert non-charged or neutral amino acid residues to charged residues. Examples of mutations converting neutral amino acid residues to charged residues include, Q64Z, V65Z, Q66Z, L67Z, P69Z, V70Z, Q71Z, V72Z, I75Z, I77Z, V78Z, P82Z, I83Z, F84Z, A87Z, T88Z, V89Z, T90Z, L91Z, Q64B, V65B, Q66B, L67B, P69B, V70B, Q71B, V72B, I75B, I77B, V78B, P82B, I83B, F84B, A87B, T88B, V89B, T90B, and L91B, wherein “Z” is an acidic amino acid and “B” is a basic amino acid.

The present invention also contemplate PDGF B-chain monomers containing mutations outside of said β hairpin loop structures that alter the structure or conformation of those hairpin loops. These structural alterations in turn serve to increase the electrostatic interactions between regions of the β hairpin loop structures of PDGF B-chain monomer contained in a dimeric molecule, and a receptor having affinity for the dimeric protein. These mutations are found at positions selected from the group consisting of positions 1-15, 44-63, and 95-160 of the PDGF B-chain monomer.

Specific examples of these mutation outside of the β hairpin L1 and L3 loop structures include, S1J, L2J, G3J, S4J, L5J, T6J, I7J, A8J, E9J, P10J, A11J, M12J, I13J, A14J, E15J, V44J, E45J, V46J, Q47J, R48J, C49J, S50J, G51J, C52J, C53J, N54J, N55J, R56J, N57J, V58J, Q59J, C60J, R61J, P62J, T63J, L95J, A96J, C97J, K98J, C99J, E100J, T101J, V102J, A103J, A104J, A105J, R106J, P107J, V108J, T109J, R110J, S111J, P112J, G113J, G114J, S115J, Q116J, E117J, Q118J, R119J, A120J, K121J, T122J, P123J, Q124J, T125J, R126J, V127J, T128J, I129J, R130J, T131J, V132J, R133J, V134J, R135J, R136J, P137J, P138J, K139J, G140J, K141J, H142J, R143J, K144J, F145J, K146J, H147J, T148J, H149J, D150J, K151J, T152J, A153J, L154J, K155J, E156J, T157J, L158J, G159J, and A160J. The variable “J” is any amino acid whose introduction results in an increase in the electrostatic interaction between the L1 and L3 β hairpin loop structures of the PDGF B-chain and a receptor with affinity for a dimeric protein containing the mutant PDGF B-chain monomer.

The invention also contemplates a number of PDGF B-chain monomers in modified forms. These modified forms include PDGF-B monomers linked to another cystine knot growth factor monomer or a fraction of such a monomer.

In specific embodiments, the mutant PDGF (A or B-chain) heterodimer comprising at least one mutant subunit or the single chain PDGF analog as described above is functionally active, i.e., capable of exhibiting one or more functional activities associated with the wild-type PDGF, such as PDGFR binding, PDGFR signalling and extracellular secretion. Preferably, the mutant PDGF heterodimer or single chain PDGF analog is capable of binding to the PDGFR, preferably with affinity greater than the wild type PDGF. Also it is preferable that such a mutant PDGF heterodimer or single chain PDGF analog triggers signal transduction. Most preferably, the mutant PDGF heterodimer comprising at least one mutant subunit or the single chain PDGF analog of the present invention has an in vitro bioactivity and/or in vivo bioactivity greater than the wild type PDGF and has a longer serum half-life than wild type PDGF. Mutant PDGF heterodimers and single chain PDGF analogs of the invention can be tested for the desired activity by procedures known in the art.

Mutants of the Human Vascular Endothelial Growth Factor (VEGF)

The human VEGF protein contains 197 amino acids as shown in FIG. 9 (SEQ ID No: 8). The invention contemplates mutants of the human VEGF protein comprising single or multiple amino acid substitutions, deletions or insertions, of one, two, three, four or more amino acid residues when compared with the wild type monomer. Furthermore, the invention contemplates mutant human VEGF proteins linked to another CKGF protein.

The present invention provides mutant VEGF protein L1 hairpin loops having one or more amino acid substitutions between positions 27-50, inclusive, excluding Cys residues, as depicted in FIG. 9 (SEQ ID NO: 8). The amino acid substitutions H27X, P28X, I29X, E30X, T31X, L32X, V33X, D34X, I35X, F36X, Q37X, E38X, Y39X, P40X, D41X, E42X, I43X, E44X, Y45X, I46X, F47X, K48X, P49X, and S50X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the VEGF protein where an acidic residue is present, the variable “X” would correspond to a basic amino acid residue. Specific examples of electrostatic charge altering mutations where a basic residue is introduced into the VEGF protein include one or more of the following: of E30B, D34B, E38B, D41B, E42B, and E44B, wherein “B” is a basic amino acid residue.

Introducing acidic amino acid residues where basic residues are present in the VEGF protein sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following H27Z and K48Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at H27U, E30U, D34U, E38U, D41U, E42U, E44U, and K48U, wherein “U” is a neutral amino acid.

Mutant VEGF protein proteins are provided containing one or more electrostatic charge altering mutations in the L1 hairpin loop amino acid sequence that convert non-charged or neutral amino acid residues to charged residues. Examples of mutations converting neutral amino acid residues to charged residues include: P28Z, I29Z, T31Z, L32Z, V33Z, I35Z, F36Z, Q37Z, Y39Z, P40Z, I43Z, Y45Z, I46Z, F47Z, P49Z, S50Z, P28B, I29B, T31B, L32B, V33B, I35B, F36B, Q37B, Y39B, P40B, I43B, Y45B, I46B, F47B, P49B, and S50B, wherein “Z” is an acidic amino acid and “B” is a basic amino acid.

Mutant VEGF protein containing mutants in the L3 hairpin loop are also described. These mutant proteins have one or more amino acid substitutions, deletion or insertions, between positions 73 and 99, inclusive, excluding Cys residues, of the L3 hairpin loop, as depicted in FIG. 9 (SEQ ID NO: 8). The amino acid substitutions include: E73X, S74X, N75X, I76X, T77X, M78X, Q79X, I80X, M81X, R82X, I83X, K84X, P85X, H86X, Q87X, G88X, Q89X, H90X, I91X, G92X, E93X, M94X, S95X, F96X, L97X, Q98X, and H99X, wherein “X” is any amino acid residue, the substitution of which alters the electrostatic character of the L3 loop.

One set of mutations of the L3 hairpin loop includes introducing one or more basic amino acid residues into the VEGF protein L3 hairpin loop amino acid sequence. For example, when introducing basic residues into the L3 loop of the VEGF protein, the variable “X” of the sequence described above corresponds to a basic amino acid residue. Specific examples of electrostatic charge altering mutations where a basic residue is introduced into the VEGF protein include one or more of the following: E73B and E93B, wherein “B” is a basic amino acid residue.

The invention further contemplates introducing one or more acidic residues into the amino acid sequence of the VEGF protein L3 hairpin loop. For example, one or more acidic amino acids can be introduced in the sequence of 166-3193 described above, wherein the variable “X” corresponds to an acidic amino acid. Specific examples of such mutations include R82Z, K84Z, H86Z, H90Z, and H99Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at E73U, R82U, K84U, H86U, H90U, E93B, and H99U, wherein “U” is a neutral amino acid.

Mutant VEGF protein proteins are provided containing one or more electrostatic charge altering mutations in the L3 hairpin loop amino acid sequence that convert non-charged or neutral amino acid residues to charged residues. Examples of mutations converting neutral amino acid residues to charged residues include S74Z, N75Z, I76Z, T77Z, M78Z, Q79Z, I80Z, M81Z, I83Z, P85Z, Q87Z, G88Z, Q89Z, I91Z, G92Z, M94Z, S95Z, F96Z, L97Z, Q98Z, S74B, N75B, I76B, T77B, M78B, Q79B, I80B, M81B, I83B, P85B, Q87B, G88B, Q89B, I91B, G92B, M94B, S95B, F96B, L97B, and Q98B, wherein “Z” is an acidic amino acid and “B” is a basic amino acid.

The present invention also contemplate VEGF protein containing mutations outside of said β hairpin loop structures that alter the structure or conformation of those hairpin loops. These structural alterations in turn serve to increase the electrostatic interactions between regions of the β hairpin loop structures of VEGF protein contained in a dimeric molecule, and a receptor having affinity for the dimeric protein. These mutations are found at positions selected from the group consisting of positions 1-26, 51-72, and 100-189 of the VEGF protein.

Specific examples of these mutation outside of the β hairpin L1 and L3 loop structures include, A1J, P2J, M3J, A4J, E5J, G6J, G7J, G8J, Q9J, N10J, H11J, H12J, E13J, V14J, V15J, K16J, F17J, M18J, D19J, V20J, Y21J, Q22J, R23J, S24J, Y25J, V52J, P53J, L54J, M55J, R56J, C57J, G58J, G59J, C60J, C61J, N62J, D63J, E64J, G65J, L66J, E67J, C68J, V69J, P70J, T71J, E72J, N100J, K101J, C102J, E103J, C104J, R105J, P106J, K107J, K108J, D109J, R110J, A111J, R112J, Q113J, E114J, K115J, K116J, S117J, V118J, R119J, G120J, K121J, G122J, K123J, G124J, Q125J, K126J, R127J, K128J, R129J, K130J, K131J, S132J, R133J, Y134J, K135J, S136J, W137J, S138J, V139J, P140J, C141J, G142J, P143J, C144J, S145J, E146J, R147J, R148J, K149J, H150J, L151J, F152J, V153J, Q154J, D155J, P156J, Q157J, T158J, C159J, K160J, C161J, S162J, C163J, K164J, N165J, T166J, D167J, S168J, R169J, C170J, K171J, A172J, R173J, Q174J, L175J, E176J, L177J, N178J, E179J, R180J, T181J, C182J, R183J, C184J, D185J, K186J, P187J, R188J, and R189J. The variable “J” is any amino acid whose introduction results in an increase in the electrostatic interaction between the L1 and L3 β hairpin loop structures of the VEGF protein and a receptor with affinity for a dimeric protein containing the mutant VEGF protein monomer.

The invention also contemplates a number of VEGF proteins in modified forms. These modified forms include VEGF proteins linked to another cystine knot growth factor monomer or a fraction of such a monomer.

In specific embodiments, the mutant VEGF protein heterodimer comprising at least one mutant subunit or the single chain VEGF protein analog as described above is functionally active, i.e., capable of exhibiting one or more functional activities associated with the wild-type VEGF protein, such as VEGF protein receptor binding, VEGF protein protein family receptor signalling and extracellular secretion. Preferably, the mutant VEGF protein heterodimer or single chain VEGF protein analog is capable of binding to the VEGF protein receptor, preferably with affinity greater than the wild type VEGF protein. Also it is preferable that such a mutant VEGF protein heterodimer or single chain VEGF protein analog triggers signal transduction. Most preferably, the mutant VEGF protein heterodimer comprising at least one mutant subunit or the single chain VEGF protein analog of the present invention has an in vitro bioactivity and/or in vivo bioactivity greater than the wild type VEGF protein and has a longer serum half-life than wild type VEGF protein. Mutant VEGF protein heterodimers and single chain VEGF protein analogs of the invention can be tested for the desired activity by procedures known in the art.

Polynucleotides Encoding Mutant PDGF Family Proteins and Analogs

The present invention also relates to nucleic acids molecules comprising sequences encoding mutant subunits of human PDGF family proteins and PDGF family protein analogs of the invention, wherein the sequences contain at least one base insertion, deletion or substitution, or combinations thereof that results in single or multiple amino acid additions, deletions and substitutions relative to the wild type protein. Base mutation that does not alter the reading frame of the coding region are preferred. As used herein, when two coding regions are said to be fused, the 3′ end of one nucleic acid molecule is ligated to the 5′ (or through a nucleic acid encoding a peptide linker) end of the other nucleic acid molecule such that translation proceeds from the coding region of one nucleic acid molecule into the other without a frameshift.

Due to the degeneracy of the genetic code, any other DNA sequences that encode the same amino acid sequence for a mutant subunit or monomer may be used in the practice of the present invention. These include but are not limited to nucleotide sequences comprising all or portions of the coding region of the subunit or monomer that are altered by the substitution of different codons that encode the same amino acid residue within the sequence, thus producing a silent change.

In one embodiment, the present invention provides nucleic acid molecules comprising sequences encoding mutant PDGF family protein subunits, wherein the mutant PDGF family protein subunits comprise single or multiple amino acid substitutions, preferably located in or near the β hairpin L1 and/or L3 loops of the target protein. The invention also provides nucleic acids molecules encoding mutant PDGF family protein subunits having an amino acid substitution outside of the L1 and/or L3 loops such that the electrostatic interaction between those loops and the cognate receptor of the PDGF family protein dimer are increased. The present invention further provides nucleic acids molecules comprising sequences encoding mutant PDGF family protein subunits comprising single or multiple amino acid substitutions, preferably located in or near the β hairpin L1 and/or L3 loops of the PDGF family protein subunit, and/or covalently joined to another CKGF protein, in whole or in part.

In yet another embodiment, the invention provides nucleic acid molecules comprising sequences encoding PDGF family protein analogs, wherein the coding region of a mutant PDGF family protein subunit comprising single or multiple amino acid substitutions, is fused with the coding region of its corresponding dimeric unit, which can be a wild type subunit or another mutagenized monomeric subunit. Also provided are nucleic acid molecules encoding a single chain PDGF family protein analog wherein the carboxyl terminus of the mutant PDGF family protein monomer is linked to the amino terminus of another CKGF protein. In still another embodiment, the nucleic acid molecule encodes a single chain PDGF family protein analog, wherein the carboxyl terminus of the mutant PDGF family protein monomer is covalently bound to the amino terminus a another CKGF protein, and the carboxyl terminus of bound amino acid sequence is covalently bound to the amino terminus of a mutant PDGF family protein monomer without the signal peptide.

The single chain analogs of the invention can be made by ligating the nucleic acid sequences encoding monomeric subunits of a PDGF family protein to each other by methods known in the art, in the proper coding frame, and expressing the fusion protein by methods commonly known in the art. Alternatively, such a fusion protein may be made by protein synthetic techniques, e.g., by use of a peptide synthesizer.

Preparation of Mutant PDGF Family Protein Subunits and Analogs

The production and use of the mutant α subunits, mutant PDGF family protein subunits, mutant PDGF family protein heterodimers, PDGF family protein analogs, single chain analogs, derivatives and fragments thereof of the invention are within the scope of the present invention. In specific embodiments, the mutant subunit or PDGF analog is a fusion protein either comprising, for example, but not limited to, a mutant PDGF family protein subunit and another CKGF protein or two mutant PDGF family protein subunits, or a mutant PDGF family protein subunit and a corresponding wild PDGF family protein subunit. In one embodiment, such a fusion protein is produced by recombinant expression of a nucleic acid encoding a mutant or wild type subunit joined in-frame to the coding sequence for another protein, such as but not limited to toxins, such as ricin or diphtheria toxin. Such a fusion protein can be made by ligating the appropriate nucleic acid sequences encoding the desired amino acid sequences to each other by methods known in the art, in the proper coding frame, and expressing the fusion protein by methods commonly known in the art. Alternatively, such a fusion protein may be made by protein synthetic techniques, e.g., by use of a peptide synthesizer. Chimeric genes comprising portions of mutant PDGF family protein subunits fused to any heterologous protein-encoding sequences may be constructed. A specific embodiment relates to a single chain analog comprising a mutant PDGF family protein subunit fused to another PDGF family protein subunit, preferably with a peptide linker between the two subunits.

Structure and Function Analysis of Mutant PDGF Family Protein Subunits

Described herein are methods for determining the structure of mutant PDGF family protein subunits, mutant family protein heterodimers and PDGF family protein analogs, and for analyzing the in vitro activities and in vivo biological functions of the foregoing.

Once a mutant PDGF family protein subunit is identified, it may be isolated and purified by standard methods including chromatography (e.g., ion exchange, affinity, and sizing column chromatography), centrifugation, differential solubility, or by any other standard technique for the purification of proteins. The functional properties may be evaluated using any suitable assay (including immunoassays as described infra).

Alternatively, once a mutant PDGF family protein subunit produced by a recombinant host cell is identified, the amino acid sequence of the subunit(s) can be determined by standard techniques for protein sequencing, e.g., with an automated amino acid sequencer.

The mutant subunit sequence can be characterized by a hydrophilicity analysis (Hopp, T. and Woods, K., 1981, Proc. Natl. Acad. Sci. U.S.A. 78:3824). A hydrophilicity profile can be used to identify the hydrophobic and hydrophilic regions of the subunit and the corresponding regions of the gene sequence which encode such regions.

Secondary structural analysis (Chou, P. and Fasman, G., 1974, Biochemistry 13:222) can also be done, to identify regions of the subunit that assume specific secondary structures.

Other methods of structural analysis can also be employed. These include but are not limited to X-ray crystallography (Engstom, A., 1974, Biochem. Exp. Biol. 11:7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.). Structure prediction, analysis of crystallographic data, sequence alignment, as well as homology modeling, can also be accomplished using computer software programs available in the art, such as BLAST, CHARMM release 21.2 for the Convex, and QUANTA v.3.3, (Molecular Simulations, Inc., York, United Kingdom).

The functional activity of mutant PDGF family protein subunits, mutant PDGF family protein heterodimers, PDGF family protein analogs, single chain analogs, derivatives and fragments thereof can be assayed by various methods known in the art.

For example, where one is assaying for the ability of a mutant PDGF family protein or subunits to bind or compete with wild-type PDGF family protein or its subunits for binding to an antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. Antibody binding can be detected by detecting a label on the primary antibody. Alternatively, the primary antibody is detected by detecting binding of a secondary antibody or reagent to the primary antibody, particularly where the secondary antibody is labeled. Many means are known in the art for detecting binding in an immunoassay and are within the scope of the present invention.

The binding of mutant PDGF family protein subunits, mutant PDGF family protein heterodimers, PDGF family protein analogs, single chain analogs, derivatives and fragments thereof, to a platelet-derived growth factor family protein receptor (PDGFR) can be determined by methods well-known in the art, such as but not limited to in vitro assays based on displacement from the PDGFR of a radiolabelled PDGF family protein of another species, such as bovine PDGF. The bioactivity of a mutant PDGF family protein heterodimers, PDGF family protein analogs, single chain analogs, derivatives and fragments thereof, can also be measured by a variety of bioassays The platelet derived growth factor family of protein (PDGF) effect the growth of a variety of cell types. The PDGF proteins exert their stimulatory effects on cell growth by activating a number of cellular systems by binding to protein tyrosine kinase receptors. Cellular response assays (e.g., cell growth and DNA synthesis assays), hormone stimulated protein expression assays, and binding assays are all examples of assay systems available to measure the bioactivity of the mutant PDGF proteins described by the present invention.

Androgen Metabolism Bioassay

Human gingival fibroblasts derived from chronically inflamed gingival tissue are used to measure and compare the bioactivity of PDGF mutant proteins with wild type forms of the molecules. In one embodiment of this assay, carbon 14 (¹⁴C) labeled precursor molecules are used to measure the bioactivity of mutant PDGF growth factors of the present invention. In fibroblasts, testosterone is metabolized to DHT and 4-androstenedione. Fibroblasts also metabolize 4-androstenedione to DHT and testosterone. The rate of product synthesis in these two metabolic pathways is sensitive to PDGF stimulation. Therefore, radiolabeled substrate molecules can be used to measure the amount of labeled product generated as a result of stimulation by a mutant PDGF family protein as compared to the level of product generation stimulated by the wild type form of the PDGF family protein.

In one embodiment of this assay system, ¹⁴C-testosterone and ¹⁴C-4-androstenedione are used to determine the bioactivity of a mutant PDGF family protein. These reagents are commercially available from Amersham International (Princeton, N.J.). A sufficient concentration of radiolabeled substrate is prepared for use in the assay. For example, 50 μCi/ml of testosterone can be used in the assay. The mutant and wild type PDGF family proteins are expressed and purified according to the methods described by the present invention. A range of serial dilutions is prepared to establish the stimulatory concentrations for androgen metabolism for each mutant PDGF family protein. For example, wild type PDGF at 0.5 ng/ml has been reported to be a stimulatory concentration. (Kasasa et al., J. Clin. Periodontal., 25: 640-646 (1998)).

Human gingival fibroblasts of the 5^{th}-9^{th} passage are derived from chronically-inflamed gingival tissue from periodontal pockets of 3-7 patients after completion of an initial phase of treatment and are isolated during periodental surgery for pocket elimination (no bleeding on probing and depths of 6-8 mm). Fibroblasts derived from an inflamed source have been reported to have an elevated metabolic response to androgens at baseline and in response to inflammatory stimuli compared with healthy controls. Accordingly, cells from this type of source are to be used in the assay.

Confluent gingival fibroblasts in monolayer culture derived from 3-7 cell-lines were incubated in duplicate in multi-well dishes in Eagle's MEM with the androgen substrates 14C-testosterone/14C-4-androstenedione and growth factors to be tested for activity. Optimal stimulatory concentrations for androgen metabolism, in response to individual PDGF family protein incubations are established using a range of concentrations close to the ED50 values of the wild type form of the protein.

Incubations are performed for 24 hours at 37° C. in a humidified tissue culture incubator with 5% CO2. At the end of the incubation period, the metabolites are extracted from the medium using ethyl acetate (2 ml×3), evaporated in a rotary evaporator (Gyrovap, V. A. Howe Ltd., Banbury, Oxon, UK) and separated by thin layer chromatography in a benzene:acetone solvent system (4:1 v/v). The separated metabolites were quantified using a radioisotope scanner (Berthold linear analyzer, Victoria, Australia). The biologically-active metabolite DHT is characterized to determine the bioactivity of the mutant PDGF family proteins.

DHT is characterized after extraction using standard techniques such as gas chromatography and mass spectrometry. These techniques are described in Soory, M., J. Peridontal Res., 30:124-131 (1995).

DNA Synthesis Assay

In another embodiment, the bioactivity of a mutant PDGF family protein is assayed by measuring the amount of ³H-thymidine incorporated into growing fibroblasts in the presence of the mutant protein. The assay is performed by taking keloid fibroblasts obtained from patients with keloids on the upper chest. These cells are cultured in fetal calf serum (FCS) containing minimum essential medium (MEM) in T75 flasks at 37° C. in 95% air and 5% CO₂. Cells at the fifth passage are used for the assay. Prepared cells (2×10⁴/well) are placed in 24-well plates in MEM with 10% FCS and grown to confluence. The cells are washed with phosphate-buffered saline once and followed by a 24-hour incubation in MEM with 0.1% bovine serum albumin (serum-free medium). the cells are then stimulated with growth factors for 24 hours in the absence of serum. The cells are then grown for 2 hours in the presence of ³H-thymidine (NEN, Boston, Mass.) at a final concentration of 1 μCi/ml and then washed 3 times with cold phosphate-buffered saline and 4 times with 5% trichloroacetic acid. Five hundred microliters of 0.1 N NaOH/0.1% sodium dodecyl sulfate were added, and the radioactivity was measured in 5 ml of ACS II (Amersham Corp., Arlington Heights, Ill.), using a liquid scintillation system. All experiments are performed in triplicate.

By comparing the amount of ³H-thymidine incorporation in cells stimulated with a mutant PDGF family protein with cells that are stimulated with the wild type form of PDGF family protein, it is possible to determine which mutations to the PDGF amino acid sequence result in elevated bioactivity. An example of this assay is found in Kikuchi et al., Dermatology, 190:4-8 (1995).

Extracellular P1CP Assay

In another embodiment, the bioactivity of a mutant PDGF family protein is compared to the bioactivity of the wild type form of the protein by measuring the amount of procollagen type I carboxy terminal peptide (P1CP) produced by cultured fibroblasts in response to PDGF family protein stimulation. The production of P1CP reflects type I collagen metabolism, which is stimulated by exposure to PDGF family proteins and other types of growth factors. In this assay, fibroblasts cultured using the method described in the ³H-thymidine assay, are placed in 24-well culture plates at 1×10⁴ cells/well. After overnight incubation, the wells are washed and fresh serum-free medium is added with or without PDGF family proteins. After 72 hours of incubation, the supernatants are collected and stored at 4° C. The amount of P1CP in the supernatant is determined using an enzyme-linked immunosorbent assay kit obtainable from Takara Shuzo (Kyoto, Japan), as described in Ryan, et al., Hum. Pathol., 4:55-67 (1974). All experiments are performed in duplicate. The values for the amount of P1CP are expressed per 2×10⁴ fibroblasts. An example of this assay is found in Kikuchi et al., Dermatology, 190:4-8 (1995).

VEGF Bioassay System

The vascular endothelial growth factor subfamily of proteins are members of the PDGF family. Nevertheless, there are particular bioassay systems available for analyzing the binding characteristics and bioactivity of the mutant VEGF proteins described by the present invention. Two such systems are direct binding studies performed with the mutant VEGF proteins and measurements of cell growth induced by the mutant VEGF proteins.

VEGF Receptor Binding Assay

Binding assays are performed in 96-well immunoplates (Immunlon-1, DYNEX TECHNOLOGIES, Chantilly, Va.); each well is coated with 100 μl of a solution containing 10 μg/ml of rabbit IgG anti-human IgG (F_{c}-specific) in 50 mM sodium carbonate buffer, pH 9.6, overnight at 4° C. After the supernatant is discarded, the wells are washed 3 times in washing buffer (0.01% Tween 80 in PBS). The plates are blocked (300 μl/well) for one hour in assay buffer (0.5% BSA, 0.03% Tween 80, 0.01% Thimerosal in PBS). The supernatant is then discarded, and the wells are washed. A mixture is prepared with conditioned media containing either a wild type or mutant VEGF family protein at varying concentration (100 μl) and ¹²⁵I-radiolabeled wild type VEGF family protein (˜5×103 cpm in 50 μl), which is mixed with VEGF receptor specific antibody at 3-15 ng/ml, final concentration, 50 μl in micronic tubes. An irrelevant antibody is used as a control for nonspecific binding of radiolabeled VEGF family proteins. Aliquots of these solutions (100 μl) are added to precoated microtiter plates and incubated for 4 hours at 25° C. The supernatant is discarded, the plates are washed, and individual wells are counted by γ scintigraphy (LKB model 1277,). The competitive binding between unlabeled wild type or mutant VEGF family proteins and the labeled wild type VEGF family protein to the VEGF family protein receptor are plotted and analyzed by four parameter fitting (Kaleidagraph, Abelbeck Software,). The apparent dissociation constant for each mutant VEGF family protein is estimated from the concentration required for 50% inhibition (IC₅₀). An example of this assay is found in Keyt, et al., J. Biol. Chem., 271(10):5638-5646 (1996).

VEGF Induced Vascular Endothelial Cell Growth Assay

In another embodiment, the mitogenic activity of mutant VEGF family proteins is determined by using bovine adrenal cortical endothelial cells as target cells as described in Ferra & Henzel, Biochem. Biophys. Res. Commun., 161:851-859 (1989). Briefly, cells are plated sparsely (7000 cells/well) in 12-well plates and incubated overnight in Dulbecco's modified Eagle's medium with 10% calf serum, 2 mM glutarnine, and antibiotics. The medium is exchanged on the following day, and wild type or mutant VEGF family proteins diluted in culture media from 100 ng/ml to 10 pg/ml are layered in duplicate onto the seeded cells. After 5 days of incubation at 37° C., the cells are dissociated with trypsin and quantified using a Coulter counter. An example of this assay is found in Keyt, et al., J. Biol. Chem., 271(10):5638-5646 (1996).

VEGF Mitogenic Activity

The effect of mutant VEGF family proteins on the mitogenic activity of target cells is an additional assay to measure the bioactivity of these proteins as compared to the wild type form of the molecule. Mitogenic assays are performed as described by Mizazono et al., J. Biol. Chem., 262:4098-4103 (1987). Briefly, human umbilical vein endothelial (HUVE) cells are seeded at 1×104 cells/well in 24-well plates in endothelial growth medium from BTS. Cells are allowed to attach overnight at 37° C. Medium is replaced with endothelial basal medium (BTS) supplemented with 5% fetal calf serum and 1.5 μM thymidine and wild type or mutant VEGF family proteins are added 24 hours later. Incubation is continued for an additional 18 hours, after which time 1 μCi [³H]-methylthymidine (56.7 Ci/mmol, NEN, Boston, Mass.) is added. Cells are kept at 37° C. for an additional 6 hours. Cell monolayers are fixed with methanol, washed with 5% trichloroacetic acid, solubilized in 0.3M NaOH, and counted by liquid scintillation. Levels of [³H]-methylthymidine incorporation are compared between cell populations treated with wild type or mutant VEGF family proteins. An example of this assay is found at Fiebich, et al., Eur. J. Biochem. 211:19-26 (1993).

The half life of a protein is a measurement of protein stability and indicates the time necessary for a one-half reduction in the concentration of the protein. The half life of a mutant PDGF family protein can be determined by any method for measuring PDGF family protein levels in samples from a subject over a period of time, for example but not limited to, immunoassays using anti-PDGF family protein antibodies to measure the mutant PDGF family protein levels in samples taken over a period of time after administration of the mutant PDGF family protein or detection of radiolabeled mutant PDGF family proteins in samples taken from a subject after administration of the radiolabeled mutant PDGF family proteins.

Other methods will be known to the skilled artisan and are within the scope of the invention.

Diagnostic and Therapeutic Uses

The invention provides for treatment or prevention of various diseases and disorders by administration of therapeutic compound (termed herein “Therapeutic”) of the invention. Such Therapeutics include PDGF family protein heterodimers having a mutant subunit and either a wild type or mutant subunit; PDGF family protein heterodimers having a mutant subunit and either a mutant or wild type subunit and covalently bound to another CKGF protein, in whole or in part; PDGF family protein heterodimers having a mutant subunit and a wild type subunit, where the mutant subunits are covalently bound to form a single chain analog, including a PDGF family protein heterodimer where the mutant subunit and the wild type or mutant subunit and the CKGF protein or fragment are covalently bound in a single chain analog, other derivatives, analogs and fragments thereof (e.g. as described hereinabove) and nucleic acids encoding the mutant PDGF family protein heterodimers of the invention, and derivatives, analogs, and fragments thereof.

The subject to which the Therapeutic is administered is preferably an animal, including but not limited to animals such as cows, pigs, horses, chickens, cats, dogs, etc., and is preferably a mammal. In a preferred embodiment, the subject is a human. Generally, administration of products of a species origin that is the same species as that of the subject is preferred. Thus, in a preferred embodiment, a human mutant and/or modified PDGF family protein heterodimer, derivative or analog, or nucleic acid, is therapeutically or prophylactically or diagnostically administered to a human patient.

In a preferred aspect, the Therapeutic of the invention is substantially purified.

The PDGF family of proteins play an active role in stimulating cell growth. The isoforms of PDGF specifically play an important role in wound healing. This wound healing function can be enhanced by by the methods of the invention. Disorders in which a PDGF family protein is absent or decreased relative to normal or desired levels are treated or prevented by administration of a mutant PDGF family protein heterodimer or PDGF family protein analog of the invention. Disorders in which a PDGF family protein receptor is absent or decreased relative to normal levels or unresponsive or less responsive than normal PDGF family protein receptor to the wild type PDGF family protein, can also be treated by administration of a mutant PDGF family protein heterodimer or PDGF family protein analog. Mutant PDGF family protein heterodimers and PDGF family protein analogs for use as antagonists are contemplated by the present invention.

In specific embodiments, mutant PDGF family protein heterodimers or PDGF family protein analogs with bioactivity are administered therapeutically, including prophylactically to treat a number of cellular growth and development conditions, including promoting wound healing.

The absence of or a decrease in PDGF family protein or function, or PDGF family protein receptor and function can be readily detected, e.g., by obtaining a patient tissue sample (e.g., from biopsy tissue) and assaying it in vitro for RNA or protein levels, structure and/or activity of the expressed RNA or protein of PDGF family protein or PDGF family protein receptor. Many methods standard in the art can be thus employed, including but not limited to immunoassays to detect and/or visualize PDGF family protein or PDGF family protein receptor protein (e.g., Western blot, immunoprecipitation followed by sodium dodecyl sulfate polyacrylamide gel electrophoresis, immunocytochemistry, etc.) and/or hybridization assays to detect PDGF family protein or PDGF family protein receptor expression by detecting and/or visualizing PDGF family protein or PDGF family protein receptor mRNA (e.g., Northern assays, dot blots, in situ hybridization, etc.), etc.

Mutants of the Human Nerve Growth Factor Monomer

The human nerve growth factor monomer contains 120 amino acids as shown in FIG. 10 (SEQ ID No: 9). The invention contemplates mutants of the human nerve growth factor monomer comprising single or multiple amino acid substitutions, deletions or insertions, of one, two, three, four or more amino acid residues when compared with the wild type monomer. Furthermore, the invention contemplates mutant human nerve growth factor monomers that are linked to another CKGF protein.

The present invention provides mutant nerve growth factor monomer L1 hairpin loops having one or more amino acid substitutions between positions 16 and 57, inclusive, excluding Cys residues, as depicted in FIG. 10 (SEQ ID NO: 9). The amino acid substitutions include: D16X, S17X, V18X, S19X, V20X, W21X, V22X, G23X, D24X, K25X, T26X, T27X, A28X, T29X, D30X, I31X, K32X, G33X, K34X, E35X, V36X, M37X, V38X, L39X, G40X, E41X, V42X, N43X, N44X, I45X, N46X, S47X, V48X, F49X, K50X, Q51X, Y52X, F53X, F54X, E55X, T56X, and K57X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the nerve growth factor monomer, the variable “X” would correspond to a basic amino acid residue. Specific examples of electrostatic charge altering mutations where a basic residue is introduced into the nerve growth factor monomer include one or more of the following: D16B, D24B, D30B, E35B, E41B, and E55B, wherein “B” is a basic amino acid residue.

Introducing acidic amino acid residues where basic residues are present in the nerve growth factor monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: K25Z, K32Z, K34Z, K50Z, and K57Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D16U, D24U, K25U, D30U, K32U, K34U, E35U, E41U, K50U, E55U, and K57U, wherein “U” is a neutral amino acid.

Mutant nerve growth factor monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: S17Z, V18Z, S19Z, V20Z, W21Z, V22Z, G23Z, T26Z, T27Z, A28Z, T29Z, I31Z, G33Z, V36Z, M37Z, V38Z, L39Z, G40Z, V42Z, N43Z, N44Z, I45Z, N46Z, S47Z, V48Z, F49Z, Q51Z, Y52Z, F53Z, F54Z, T56Z, S17B, V18B, S19B, V20B, W21B, V22B, G23B, T26B, T27B, A28B, T29B, I31B, G33B, V36B, M37B, V38B, L39B, G40B, V42B, N43B, N44B, I45B, N46B, S47B, V48B, F49B, Q51B, Y52B, F53B, F54B, and T56B, wherein “Z” is an acidic amino acid and “B” is a basic amino acid.

Mutant nerve growth factor monomers containing mutants in the L3 hairpin loop are also described. These mutant proteins have one or more amino acid substitutions, deletion or insertions, between positions 81 and 107, inclusive, excluding Cys residues, of the L3 hairpin loop, as depicted in FIG. 10 (SEQ ID NO: 9). The amino acid substitutions include, T81X, T82X, T83X, H84X, T85X, F86X, V87X, K88X, A89X, M90X, L91X, T92X, D93X, G94X, K95X, Q96X, A97X, A98X, W99X, R100X, F101X, I102X, R103X, I104X, D105X, T106X, and A107X, wherein “X” is any amino acid residue, the substitution of which alters the electrostatic character of the L3 loop.

One set of mutations of the L3 hairpin loop includes introducing one or more basic amino acid residues into the nerve growth factor L3 hairpin loop amino acid sequence where acidic amino acid residues reside. For example, when introducing basic residues into the L3 loop of the nerve growth factor monomer, the variable “X” of the sequence described above corresponds to a basic amino acid residue. Specific examples of electrostatic charge altering mutations where a basic residue is introduced into the nerve growth factor monomer include one or more of the following: D93B and D105B, wherein “B” is a basic amino acid residue.

The invention further contemplates introducing one or more acidic residues into the amino acid sequence of the nerve growth factor L3 hairpin loop. For example, one or more acidic amino acids can be introduced in the sequence of 81-107 described above, wherein the variable “X” corresponds to an acidic amino acid. Specific examples of such mutations include H84Z, K88Z, K95Z, R100Z, and R103Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at H84U, K88U, D93U, K95U, R100U, R103U, and D105U, wherein “U” is a neutral amino acid.

Mutant nerve growth factor monomers are provided containing one or more electrostatic charge altering mutations in the L3 hairpin loop amino acid sequence that convert non-charged or neutral amino acid residues to charged residues. Examples of mutations converting neutral amino acid residues to charged residues include, T81Z, T82Z, T83Z, T85Z, F86Z, V87Z, A89Z, M90Z, L91Z, T92Z, G94Z, Q96Z, A97Z, A98Z, W99Z, F101Z, I102Z, I104Z, T106Z, A107Z, T81B, T82B, T83B, T85B, F86B, V87B, A89B, M90B, L91B, T92B, G94B, Q96B, A97B, A98B, W99B, F101B, I102B, I104B, T106B, and A107B, wherein “Z” is an acidic amino acid and “B” is a basic amino acid.

The present invention also contemplate nerve growth factor monomers containing mutations outside of said β hairpin loop structures that alter the structure or conformation of those hairpin loops. These structural alterations in turn serve to increase the electrostatic interactions between regions of the β hairpin loop structures of nerve growth factor monomer contained in a dimeric molecule, and a receptor having affinity for the dimeric protein. These mutations are found at positions selected from the group consisting of positions 1-14, 59-79, and 109-120 of the nerve growth factor monomer.

Specific examples of these mutation outside of the β hairpin L1 and L3 loop structures include, S1J, S2J, S3J, H4J, P5J, I6J, F7J, H8J, R9J, G10J, E11J, D12J, S13J, V14J, R59J, D60J, P61J, N62J, P63J, V64J, D65J, S66J, G67J, C68J, R69J, G70J, I71J, D72J, S73J, K74J, H75J, W76J, N77J, S78J, Y79J, V109J, C110J, V111J, L112J, S113J, R114J, K115J, A116J, V117J, R118J, R119J, and A120J. The variable “J” is any amino acid whose introduction results in an increase in the electrostatic interaction between the L1 and L3 β hairpin loop structures of the nerve growth factor and a receptor with affinity for a dimeric protein containing the mutant nerve growth factor monomer.

The invention also contemplates a number of nerve growth factor monomers in modified forms. These modified forms include nerve growth factor monomers linked to another cystine knot growth factor monomer or a fraction of such a monomer.

In specific embodiments, the mutant nerve growth factor heterodimer comprising at least one mutant subunit or the single chain nerve growth factor analog as described above is functionally active, i.e., capable of exhibiting one or more functional activities associated with the wild-type nerve growth factor, such as nerve growth factor receptor binding, nerve growth factor receptor signalling and extracellular secretion. Preferably, the mutant nerve growth factor heterodimer or single chain nerve growth factor analog is capable of binding to the nerve growth factor receptor, preferably with affinity greater than the wild type nerve growth factor. Also it is preferable that such a mutant nerve growth factor heterodimer or single chain nerve growth factor analog triggers signal transduction. Most preferably, the mutant nerve growth factor heterodimer comprising at least one mutant subunit or the single chain nerve growth factor analog of the present invention has an in vitro bioactivity and/or in vivo bioactivity greater than the wild type nerve growth factor and has a longer serum half-life than wild type nerve growth factor. Mutant nerve growth factor heterodimers and single chain nerve growth factor analogs of the invention can be tested for the desired activity by procedures known in the art.

Mutants of the Human Brain Derived Neurotrophic Factor

The human brain-derived neurotrophic factor monomer contains 119 amino acids as shown in FIG. 11 (SEQ ID No: 10). The invention contemplates mutants of the human brain-derived neurotrophic factor monomer comprising single or multiple amino acid substitutions, deletions or insertions, of one, two, three, four or more amino acid residues when compared with the wild type monomer. Furthermore, the invention contemplates mutant human brain-derived neurotrophic factor monomers that are linked to another CKGF protein.

The present invention provides mutant brain-derived neurotrophic factor monomer L1 hairpin loops having one or more amino acid substitutions between positions 14 and 57, inclusive, excluding Cys residues, as depicted in FIG. 11 (SEQ ID NO: 10). The amino acid substitutions include D14X, S15X, I16X, S17X, E18X, W19X, V20X, T21X, A22X, A23X, D24X, K25X, K26X, T27X, A28X, V29X, D30X, M31X, S32X, G33X, G34X, T35X, V36X, T37X, V38X, L39X, E40X, K41X, V42X, S43X, P44X, V45X, K46X, G47X, Q48X, L49X, K50X, Q51X, Y52X, F53X, Y54X, E55X, T56X, and K57X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the brain-derived neurotrophic factor monomer, the variable “X” would correspond to a basic amino acid residue. Specific examples of electrostatic charge altering mutations where a basic residue is introduced into the brain-derived neurotrophic factor monomer include one or more of the following: D14B, E18B, D24B, D30B, E40B, E55B, and E57B, wherein “B” is a basic amino acid residue.

Introducing acidic amino acid residues where basic residues are present in the brain-derived neurotrophic factor monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: K25Z, K26Z, K41Z, K46Z, K50Z, and K57Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D14U, E18U, D24U, K25U, K26U, D30U, E40U, K41U, K46U, K50U, E55U, and K57U, wherein “U” is a neutral amino acid.

Mutant brain-derived neurotrophic factor monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: S15Z, I16Z, S17Z, W19Z, V20Z, T21Z, A22Z, A23Z, T27Z, A28Z, V29Z, M31Z, S32Z, G33Z, G34Z, T35Z, V36Z, T37Z, V38Z, L39Z, V42Z, S43Z, P44Z, V45Z, G47Z, Q48Z, L49Z, Q51Z, Y52Z, F53Z, Y54Z, T56Z, S15B, I16B, S17B, W19B, V20B, T21B, A22B, A23B, T27B, A28B, V29B, M31B, S32B, G33B, G34B, T35B, V36B, T37B, V38B, L39B, V42B, S43B, P44B, V45B, G47B, Q48B, L49B, Q51B, Y52B, F53B, Y54B, and T56B, wherein “Z” is an acidic amino acid and “B” is a basic amino acid.

Mutant brain-derived neurotrophic factor monomers containing mutants in the L3 hairpin loop are also described. These mutant proteins have one or more amino acid substitutions, deletion or insertions, between positions 81 and 108, inclusive, excluding Cys residues, of the L3 hairpin loop, as depicted in FIG. 11 (SEQ ID NO: 10). The amino acid substitutions include: R81X, T82X, T83X, Q84X, S85X, Y86X, V87X, R88X, A89X, M90X, L91X, T92X, D93X, S94X, K95X, K96X, R97X, I98X, G99X, W100X, R101X, F102X, I103X, R104X, I105X, D106X, T107X, and S108X, wherein “X” is any amino acid residue, the substitution of which alters the electrostatic character of the L3 loop.

One set of mutations of the L3 hairpin loop includes introducing one or more basic amino acid residues into the brain-derived neurotrophic factor L3 hairpin loop amino acid sequence. For example, when introducing basic residues into the L3 loop of the brain-derived neurotrophic factor monomer, the variable “X” of the sequence described above corresponds to a basic amino acid residue. Specific examples of electrostatic charge altering mutations where a basic residue is introduced into the brain-derived neurotrophic factor monomer include one or more of the following: D93B and D106B, wherein “B” is a basic amino acid residue.

The invention further contemplates introducing one or more acidic residues into the amino acid sequence of the brain-derived neurotrophic factor L3 hairpin loop. For example, one or more acidic amino acids can be introduced in the sequence of 81-108 described above, wherein the variable “X” corresponds to an acidic amino acid. Specific examples of such mutations include R81Z, R88Z, K95Z, K96Z, R97Z, R101Z, and R104Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at R81U, R88U, D93B, K95U, K96U, R97U, R101U, and R104Z, wherein “U” is a neutral amino acid.

Mutant brain-derived neurotrophic factor proteins are provided containing one or more electrostatic charge altering mutations in the L3 hairpin loop amino acid sequence that convert non-charged or neutral amino acid residues to charged residues. Examples of mutations converting neutral amino acid residues to charged residues include, T82Z, T83Z, Q84Z, S85Z, Y86Z, V87Z, A89Z, M90Z, L91Z, T92Z, S94Z, I98Z, G99Z, W100Z, F102Z, I103Z, I105Z, T107Z, S108Z, C109Z, V110Z, T82B, T83B, Q84B, S85B, Y86B, V87B, A89B, M90B, L91B, T92B, S94B, I98B, G99B, W100B, F102B, I103B, I105B, T107B, S108B, and V110B, wherein “Z” is an acidic amino acid and “B” is a basic amino acid.

The present invention also contemplate brain-derived neurotrophic factor monomers containing mutations outside of said β hairpin loop structures that alter the structure or conformation of those hairpin loops. These structural alterations in turn serve to increase the electrostatic interactions between regions of the β hairpin loop structures of brain-derived neurotrophic factor monomer contained in a dimeric molecule, and a receptor having affinity for the dimeric protein. These mutations are found at positions selected from the group consisting of positions 1-12, 59-79, and 110-119 of the brain-derived neurotrophic factor monomer.

Specific examples of these mutation outside of the β hairpin L1 and L3 loop structures include, H1J, S2J, D3J, P4J, A5J, R6J, R7J, G8J, E9J, L10J, S11J, V12J, N59J, P60J, M61J, G62J, Y63J, T64J, K65J, E66J, G67J, C68J, R69J, G70J, I71J, D72J, K73J, R74J, H75J, W76J, N77J, S78J, Q79J, V110J, C111J, I112J, L113J, T114J, I115J, K116J, R117J, G118J, and E119J. The variable “J” is any amino acid whose introduction results in an increase in the electrostatic interaction between the L1 and L3 β hairpin loop structures of the brain-derived neurotrophic factor and a receptor with affinity for a dimeric protein containing the mutant brain-derived neurotrophic factor monomer.

The invention also contemplates a number of brain-derived neurotrophic factor monomers in modified forms. These modified forms include brain-derived neurotrophic factor monomers linked to another cystine knot growth factor monomer or a fraction of such a monomer.

In specific embodiments, the mutant brain-derived neurotrophic factor heterodimer comprising at least one mutant subunit or the single chain brain-derived neurotrophic factor analog as described above is functionally active, i.e., capable of exhibiting one or more functional activities associated with the wild-type brain-derived neurotrophic factor, such as brain-derived neurotrophic factor receptor binding, brain-derived neurotrophic factor receptor signalling and extracellular secretion. Preferably, the mutant brain-derived neurotrophic factor heterodimer or single chain brain-derived neurotrophic factor analog is capable of binding to the brain-derived neurotrophic factor receptor, preferably with affinity greater than the wild type brain-derived neurotrophic factor. Also it is preferable that such a mutant brain-derived neurotrophic factor heterodimer or single chain brain-derived neurotrophic factor analog triggers signal transduction. Most preferably, the mutant brain-derived neurotrophic factor heterodimer comprising at least one mutant subunit or the single chain brain-derived neurotrophic factor analog of the present invention has an in vitro bioactivity and/or in vivo bioactivity greater than the wild type brain-derived neurotrophic factor and has a longer serum half-life than wild type brain-derived neurotrophic factor. Mutant brain-derived neurotrophic factor heterodimers and single chain brain-derived neurotrophic factor analogs of the invention can be tested for the desired activity by procedures known in the art.

Mutants of the Human Neurotrophin-3 Monomer

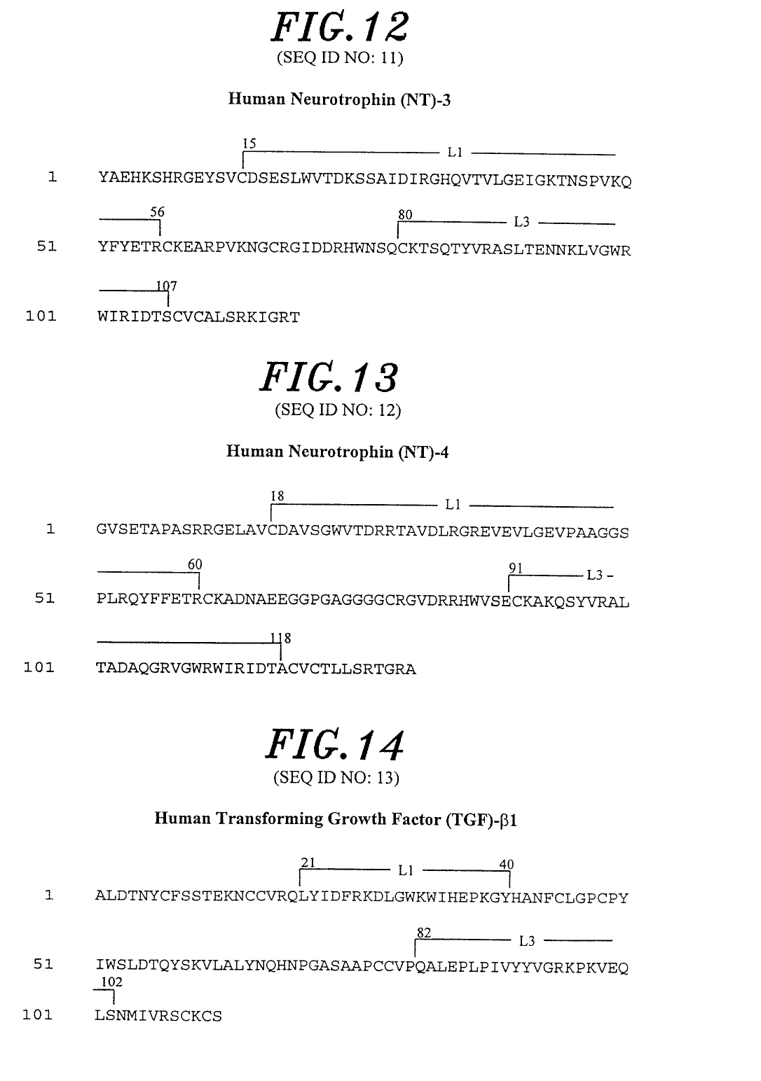

The human neutrophin-3 monomer contains 119 amino acids as shown in FIG. 12 (SEQ ID No: 11). The invention contemplates mutants of the human neutrophin-3 monomer comprising single or multiple amino acid substitutions, deletions or insertions, of one, two, three, four or more amino acid residues when compared with the wild type monomer. Furthermore, the invention contemplates mutant human neutrophin-3 monomers that are linked to another CKGF protein.

The present invention provides mutant neutrophin-3 monomer L1 hairpin loops having one or more amino acid substitutions between positions 15 and 56, inclusive, excluding Cys residues, as depicted in FIG. 12 (SEQ ID NO: 11). The amino acid substitutions include: D15X, S16X, E17X, S18X, L19X, W20X, V21X, T22X, D23X, K24X, S25X, S26X, A27X, I28X, D29X, I30X, R31X, G32X, H33X, Q34X, V35X, T36X, V37X, L38X, G39X, E40X, I41X, G42X, K43X, T44X, N45X, S46X, P47X, V48X, K49X, Q50X, Y51X, F52X, Y53X, E54X, T55X, and R56X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the neutrophin-3 monomer, the variable “X” would correspond to a basic amino acid residue. Specific examples of electrostatic charge altering mutations where a basic residue is introduced into the neutrophin-3 monomer include one or more of the following: D15B, E17B, D23B, D29B, E40B, and E54B, wherein “B” is a basic amino acid residue.

Introducing acidic amino acid residues where basic residues are present in the neutrophin-3 monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: K24Z, R31Z, H33Z, K43Z, K49Z, and R56Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D15U, E17U, D23U, K24U, D29U, R31U, H33U, E40U, K43U, K49U, E54U, and R56U, wherein “U” is a neutral amino acid.

Mutant neutrophin-3 monomers are provided containing one or more electrostatic charge altering mutations in the L1 hairpin loop amino acid sequence that convert non-charged or neutral amino acid residues to charged residues. Examples of mutations converting neutral amino acid residues to charged residues include: S16Z, S18Z, L19Z, W20Z, V21Z, T22Z, S25Z, S26Z, A27Z, I28Z, I30Z, G32Z, Q34Z, V35Z, T36Z, V37Z, L38Z, G39Z, I41Z, G42Z, T44Z, N45Z, S46Z, P47Z, V48Z, Q50Z, Y51Z, F52Z, Y53Z, T55Z, R56Z, S16B, S18B, L19B, W20B, V21B, T22B, S25B, S26B, A27B, I28B, I30B, G32B, Q34B, V35B, T36B, V37B, L38B, G39B, I41B, G42B, T44B, N45B, S46B, P47B, V48B, Q50B, Y51B, F52B, Y53B, and T55B, wherein “Z” is an acidic amino acid and “B” is a basic amino acid.

Mutant neutrophin-3 monomers containing mutants in the L3 hairpin loop are also described. These mutant proteins have one or more amino acid substitutions, deletion or insertions, between positions 80 and 107, inclusive, excluding Cys residues, of the L3 hairpin loop, as depicted in FIG. 12 (SEQ ID NO: 11). The amino acid substitutions include, K80X, T81X, S82X, Q83X, T84X, Y85X, V86X, R87X, A88X, S89X, L90X, T91X, E92X, N93X, N94X, K95X, L96X, V97X, G98X, W99X, R100X, W101X, I102X, R103X, I104X, D105X, T106X, and S107X, wherein “X” is any amino acid residue, the substitution of which alters the electrostatic character of the L3 loop.

One set of mutations of the L3 hairpin loop includes introducing one or more basic amino acid residues into the neutrophin-3 L3 hairpin loop amino acid sequence. For example, when introducing basic residues into the L3 loop of the neutrophin-3 monomer, the variable “X” of the sequence described above corresponds to a basic amino acid residue. Specific examples of electrostatic charge altering mutations where a basic residue is introduced into the neutrophin-3 monomer include one or more of the following: E92B and D105B, wherein “B” is a basic amino acid residue.

The invention further contemplates introducing one or more acidic residues into the amino acid sequence of the neutrophin-3 L3 hairpin loop. For example, one or more acidic amino acids can be introduced in the sequence of 80-107 described above, wherein the variable “X” corresponds to an acidic amino acid. Specific examples of such mutations include K80Z, R87Z, N93Z, K95Z, L96Z, R100Z, and R103Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K80U, R87U, E92U, K95U, R100U, R103U, and D105U, wherein “U” is a neutral amino acid.

Mutant neutrophin-3 proteins are provided containing one or more electrostatic charge altering mutations in the L3 hairpin loop amino acid sequence that convert non-charged or neutral amino acid residues to charged residues. Examples of mutations converting neutral amino acid residues to charged residues include, T81Z, S82Z, Q83Z, T84Z, Y85Z, V86Z, A88Z, S89Z, L90Z, T91Z, N93Z, N94Z, L96Z, V97Z, G98Z, W99Z, W101Z, I102Z, I104Z, T106Z, S107Z, T81B, S82B, Q83B, T84B, Y85B, V86B, A88B, S89B, L90B, T91B, N93B, N94B, L96B, V97B, G98B, W99B, W101B, I102B, I104B, T106B, and S107B, wherein “Z” is an acidic amino acid and “B” is a basic amino acid.

The present invention also contemplate neutrophin-3 monomers containing mutations outside of said β hairpin loop structures that alter the structure or conformation of those hairpin loops. These structural alterations in turn serve to increase the electrostatic interactions between regions of the β hairpin loop structures of neutrophin-3 monomer contained in a dimeric molecule, and a receptor having affinity for the dimeric protein. These mutations are found at positions selected from the group consisting of positions 1-13, 58-78, and 109-119 of the neutrophin-3 monomer.

Specific examples of these mutation outside of the β hairpin L1 and L3 loop structures include, Y1J, A2J, E3J, H4J, K5J, S6J, H7J, R8J, G9J, E10J, Y11J, S12J, V13J, K58J, E59J, A60J, R61J, P62J, V63J, K64J, N65J, G66J, C67J, R68J, G69J, I70J, D71J, R73J, H74J, W75J, N76J, S77J, Q78J, V109J, C110J, A111J, L112J, S113J, R114J, K115J, I116J, G117J, R118J, and T119J. The variable “J” is any amino acid whose introduction results in an increase in the electrostatic interaction between the L1 and L3 β hairpin loop structures of the neutrophin-3 and a receptor with affinity for a dimeric protein containing the mutant neutrophin-3 monomer.

The invention also contemplates a number of neutrophin-3 monomers in modified forms. These modified forms include neutrophin-3 monomers linked to another cystine knot growth factor monomer or a fraction of such a monomer.

In specific embodiments, the mutant neutrophin-3 heterodimer comprising at least one mutant subunit or the single chain neutrophin-3 analog as described above is functionally active, i.e., capable of exhibiting one or more functional activities associated with the wild-type neutrophin-3, such as neutrophin-3 receptor binding, neutrophin-3 receptor signalling and extracellular secretion. Preferably, the mutant neutrophin-3 heterodimer or single chain neutrophin-3 analog is capable of binding to the neutrophin-3 receptor, preferably with affinity greater than the wild type neutrophin-3. Also it is preferable that such a mutant neutrophin-3 heterodimer or single chain neutrophin-3 analog triggers signal transduction. Most preferably, the mutant neutrophin-3 heterodimer comprising at least one mutant subunit or the single chain neutrophin-3 analog of the present invention has an in vitro bioactivity and/or in vivo bioactivity greater than the wild type neutrophin-3 and has a longer serum half-life than wild type neutrophin-3. Mutant neutrophin-3 heterodimers and single chain neutrophin-3 analogs of the invention can be tested for the desired activity by procedures known in the art.

Mutants of the Human Neurotrophin-4 Monomer

The human neutrophin-4 monomer contains 130 amino acids as shown in FIG. 13 (SEQ ID No: 12). The invention contemplates mutants of the human neutrophin-4 monomer comprising single or multiple amino acid substitutions, deletions or insertions, of one, two, three, four or more amino acid residues when compared with the wild type monomer. Furthermore, the invention contemplates mutant human neutrophin-4 monomers that are linked to another CKGF protein.

The present invention provides mutant neutrophin-4 monomer L1 hairpin loops having one or more amino acid substitutions between positions 18 and 60, inclusive, excluding Cys residues, as depicted in FIG. 13 (SEQ ID NO: 12). The amino acid substitutions include: D18X, A19X, V20X, S21X, G22X, W23X, V24X, T25X, D26X, R27X, R28X, T29X, A30X, V31X, D32X, L33X, R34X, G35X, R36X, E37X, V38X, E39X, V40X, L41X, G42X, E43X, V44X, P45X, A46X, A47X, G48X, G49X, S50X, P51X, L52X, R53X, Q54X, Y55X, F56X, F57X, E58X, T59X, and R60X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the neutrophin-4 monomer, the variable “X” would correspond to a basic amino acid residue. Specific examples of electrostatic charge altering mutations where a basic residue is introduced into the neutrophin-4 monomer include one or more of the following: D18B, D26B, D32B, E37B, E39B, E43B, and E58B, wherein “B” is a basic amino acid residue.

Introducing acidic amino acid residues where basic residues are present in the neutrophin-4 monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: R27Z, R28Z, R34Z, R36Z, R53Z, and R60Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D18U, D26U, R27U, R28U, D32U, R34U, R36U, E37U, E39U, E43U, R53U, E58U, and R60U, wherein “U” is a neutral amino acid.

Mutant neutrophin-4 monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: A19Z, V20Z, S21Z, G22Z, W23Z, V24Z, T25Z, T29Z, A30Z, V31Z, L33Z, G35Z, V38Z, V40Z, L41Z, G42Z, V44Z, P45Z, A46Z, A47Z, G48Z, G49Z, S50Z, P51Z, L52Z, Q54Z, Y55Z, F56Z, F57Z, T59Z, A19B, V20B, S21B, G22B, W23B, V24B, T25B, T29B, A30B, V31B, L33B, G35B, V38B, V40B, L41B, G42B, V44B, P45B, A46B, A47B, G48B, G49B, S50B, P51B, L52B, Q54B, Y55B, F56B, F57B, and T59B, wherein “Z” is an acidic amino acid and “B” is a basic amino acid.

Mutant neutrophin-4 monomers containing mutants in the L3 hairpin loop are also described. These mutant proteins have one or more amino acid substitutions, deletion or insertions, between positions 91 and 118, inclusive, excluding Cys residues, of the L3 hairpin loop, as depicted in FIG. 13 (SEQ ID NO: 12). The amino acid substitutions include: K91X, A92X, K93X, Q94X, S95X, Y96X, V97X, R98X, A99X, L100X, T101X, A102X, D103X, A104X, Q105X, G106X, R107X, V108X, G109X, W110X, R111X, W112X, I113X, R114X, I115X, D116X, T117X, and A118X, wherein “X” is any amino acid residue, the substitution of which alters the electrostatic character of the L3 loop.

One set of mutations of the L3 hairpin loop includes introducing one or more basic amino acid residues into the neutrophin-4 L3 hairpin loop amino acid sequence where an acidic residue resides. For example, when introducing basic residues into the L3 loop of the neutrophin-4 monomer, the variable “X” of the sequence described above corresponds to a basic amino acid residue. Specific examples of electrostatic charge altering mutations where a basic residue is introduced into the neutrophin-4 monomer include one or more of the following: D103B and D116B, wherein “B” is a basic amino acid residue.

The invention further contemplates introducing one or more acidic residues into the amino acid sequence of the neutrophin-4 L3 hairpin loop. For example, one or more acidic amino acids can be introduced in the sequence of 91-118 described above, wherein the variable “X” corresponds to an acidic amino acid. Specific examples of such mutations include K91Z, K93Z, Q94Z, R98Z, A104Z, Q105Z, G106Z, R107Z, V108Z, R111Z, and R114Z, wherein “Z”is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K91U, K93U, R98U, D103U, R107U, R111U, R114U, and D116U, wherein “U” is a neutral amino acid.

Mutant neutrophin-4 proteins are provided containing one or more electrostatic charge altering mutations in the L3 hairpin loop amino acid sequence that convert non-charged or neutral amino acid residues to charged residues. Examples of mutations converting neutral amino acid residues to charged residues include, A92Z, Q94Z, S95Z, Y96Z, V97Z, A99Z, L100Z, T101Z, A102Z, A104Z, Q105Z, G106Z, V108Z, G109Z, W110Z, W112Z, I113Z, I115Z, T117Z, A118Z, A92B, Q94B, S95B, Y96B, V97B, A99B, L100B, T101B, A102B, A104B, Q105B, G106B, V108B, G109B, W110B, W112B, I113B, I115B, T117B, and A118B, wherein “Z” is an acidic amino acid and “B” is a basic amino acid.

The present invention also contemplate neutrophin-4 monomers containing mutations outside of said β hairpin loop structures that alter the structure or conformation of those hairpin loops. These structural alterations in turn serve to increase the electrostatic interactions between regions of the β hairpin loop structures of neutrophin-4 monomer contained in a dimeric molecule, and a receptor having affinity for the dimeric protein. These mutations are found at positions selected from the group consisting of positions 1-16, 62-89, and 120-130 of the neutrophin-4 monomer.

Specific examples of these mutation outside of the β hairpin L1 and L3 loop structures include, G1J, V2J, S3J, E4J, T5J, A6J, P7J, A8J, S9J, R10J, R11J, G12J, E13J, L14J, A15J, V16J, K62J, A63J, D64J, N65J, A66J, E67J, E68J, G69J, G70J, P71J, G72J, A73J, G74J, G75J, G76J, G77J, C78J, R79J, G80J, V81J, D82J, R83J, R84J, H85J, W86J, V87J, S88J, E89J, V120J, C121J, T122J, L123J, L124J, S125J, R126J, T127J, G128J, R129J, and A130. The variable “J” is any amino acid whose introduction results in an increase in the electrostatic interaction between the L1 and L3 β hairpin loop structures of the neutrophin-4 and a receptor with affinity for a dimeric protein containing the mutant neutrophin-4 monomer.

The invention also contemplates a number of neutrophin-4 monomers in modified forms. These modified forms include neutrophin-4 monomers linked to another cystine knot growth factor monomer or a fraction of such a monomer.

In specific embodiments, the mutant neutrophin-4 heterodimer comprising at least one mutant subunit or the single chain neutrophin-4 analog as described above is functionally active, i.e., capable of exhibiting one or more functional activities associated with the wild-type neutrophin-4, such as neutrophin-4 receptor binding, neutrophin-4 receptor signalling and extracellular secretion. Preferably, the mutant neutrophin-4 heterodimer or single chain neutrophin-4 analog is capable of binding to the neutrophin-4 receptor, preferably with affinity greater than the wild type neutrophin-4. Also it is preferable that such a mutant neutrophin-4 heterodimer or single chain neutrophin-4 analog triggers signal transduction. Most preferably, the mutant neutrophin-4 heterodimer comprising at least one mutant subunit or the single chain neutrophin-4 analog of the present invention has an in vitro bioactivity and/or in vivo bioactivity greater than the wild type neutrophin-4 and has a longer serum half-life than wild type neutrophin-4. Mutant neutrophin-4 heterodimers and single chain neutrophin-4 analogs of the invention can be tested for the desired activity by procedures known in the art.

Polynucleotides Encoding Mutant Neutrotrophin Family Proteins and Analogs

The present invention also relates to nucleic acids molecules comprising sequences encoding mutant subunits of human neurotrophin family protein and neurotrophin family protein analogs of the invention, wherein the sequences contain at least one base insertion, deletion or substitution, or combinations thereof that results in single or multiple amino acid additions, deletions and substitutions relative to the wild type protein. Base mutations that do not alter the reading frame of the coding region are preferred. As used herein, when two coding regions are said to be fused, the 3′ end of one nucleic acid molecule is ligated to the 5′ (or through a nucleic acid encoding a peptide linker) end of the other nucleic acid molecule such that translation proceeds from the coding region of one nucleic acid molecule into the other without a frameshift.

Due to the degeneracy of the genetic code, any other DNA sequences that encode the same amino acid sequence for a mutant subunit or monomer may be used in the practice of the present invention. These include but are not limited to nucleotide sequences comprising all or portions of the coding region of the subunit or monomer that are altered by the substitution of different codons that encode the same amino acid residue within the sequence, thus producing a silent change.

In one embodiment, the present invention provides nucleic acid molecules comprising sequences encoding mutant neurotrophin family protein subunits, wherein the mutant neurotrophin family protein subunits comprise single or multiple amino acid substitutions, preferably located in or near the β hairpin L1 and/or L3 loops of the target protein. The invention also provides nucleic acids molecules encoding mutant neurotrophin family protein subunits having an amino acid substitution outside of the L1 and/or L3 loops such that the electrostatic interaction between those loops and the cognate receptor of the neurotrophin family protein dimer are increased. The present invention further provides nucleic acids molecules comprising sequences encoding mutant neurotrophin family protein subunits comprising single or multiple amino acid substitutions, preferably located in or near the β hairpin L1 and/or L3 loops of the neurotrophin family protein subunit, and/or covalently joined to another CKGF protein.

In yet another embodiment, the invention provides nucleic acid molecules comprising sequences encoding neurotrophin family protein analogs, wherein the coding region of a mutant neurotrophin family protein subunit comprising single or multiple amino acid substitutions, is fused with the coding region of its corresponding dimeric unit, which can be a wild type subunit or another mutagenized monomeric subunit. Also provided are nucleic acid molecules encoding a single chain neurotrophin family protein analog wherein the carboxyl terminus of the mutant neurotrophin family protein monomer is linked to the amino terminus of another CKGF protein. In still another embodiment, the nucleic acid molecule encodes a single chain neurotrophin family protein analog, wherein the carboxyl terminus of the mutant neurotrophin family protein monomer is covalently bound to the amino terminus another CKGF protein such as the amino terminus of CTEP, and the carboxyl terminus of bound amino acid sequence is covalently bound to the amino terminus of a mutant neurotrophin family protein monomer without the signal peptide.

The single chain analogs of the invention can be made by ligating the nucleic acid sequences encoding monomeric subunits of neurotrophin family protein to each other by methods known in the art, in the proper coding frame, and expressing the fusion protein by methods commonly known in the art. Alternatively, such a fusion protein may be made by protein synthetic techniques, e.g., by use of a peptide synthesizer.

Preparation of Mutant Nerve Growth Factor Subunits and Analogs

The production and use of the mutant neurotrophin family protein, mutant neurotrophin family protein heterodimers, neurotrophin family protein analogs, single chain analogs, derivatives and fragments thereof of the invention are within the scope of the present invention. In specific embodiments, the mutant subunit or neurotrophin family protein analog is a fusion protein either comprising, for example, but not limited to, a mutant neurotrophin family protein subunit and another CKGF, in whole or in part, two mutant nerve growth subunits. In one embodiment, such a fusion protein is produced by recombinant expression of a nucleic acid encoding a mutant or wild type subunit joined in-frame to the coding sequence for another protein, such as but not limited to toxins, such as ricin or diphtheria toxin. Such a fusion protein can be made by ligating the appropriate nucleic acid sequences encoding the desired amino acid sequences to each other by methods known in the art, in the proper coding frame, and expressing the fusion protein by methods commonly known in the art. Alternatively, such a fusion protein may be made by protein synthetic techniques, e.g., by use of a peptide synthesizer. Chimeric genes comprising portions of mutant neurotrophin family protein subunits fused to any heterologous protein-encoding sequences may be constructed. A specific embodiment relates to a single chain analog comprising a mutant neurotrophin family protein subunit fused to another mutant neurotrophin family protein subunit, preferably with a peptide linker between the two mutant.

Structure and Function Analysis of Mutant Neurotrophin Family Protein Subunits

Described herein are methods for determining the structure of mutant neurotrophin family protein subunits, mutant heterodimers and neurotrophin family protein analogs, and for analyzing the in vitro activities and in vivo biological functions of the foregoing.

Once a mutant neurotrophin family protein subunit is identified, it may be isolated and purified by standard methods including chromatography (e.g., ion exchange, affnity, and sizing column chromatography), centrifugation, differential solubility, or by any other standard technique for the purification of protein. The functional properties may be evaluated using any suitable assay (including immunoassays as described infra).

Alternatively, once a mutant neurotrophin family protein subunit produced by a recombinant host cell is identified, the amino acid sequence of the subunit(s) can be determined by standard techniques for protein sequencing, e.g., with an automated amino acid sequencer.

The mutant subunit sequence can be characterized by a hydrophilicity analysis (Hopp, T. and Woods, K., 1981, Proc. Natl. Acad. Sci. U.S.A. 78:3824). A hydrophilicity profile can be used to identify the hydrophobic and hydrophilic regions of the subunit and the corresponding regions of the gene sequence which encode such regions.

Secondary structural analysis (Chou, P. and Fasman, G., 1974, Biochemistry 13:222) can also be done, to identify regions of the subunit that assume specific secondary structures.

Other methods of structural analysis can also be employed. These include but are not limited to X-ray crystallography (Engstom, A., 1974, Biochem. Exp. Biol. 11:7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.). Structure prediction, analysis of crystallographic data, sequence alignment, as well as homology modelling, can also be accomplished using computer software programs available in the art, such as BLAST, CHARMM release 21.2 for the Convex, and QUANTA v.3.3, (Molecular Simulations, Inc., York, United Kingdom).

The functional activity of mutant neurotrophin family protein subunits, mutant neurotrophin family protein heterodimers, neurotrophin family protein analogs, single chain analogs, derivatives and fragments thereof can be assayed by various methods known in the art.

For example, where one is assaying for the ability of a mutant subunit or mutant neurotrophin family protein to bind or compete with wild-type neurotrophin family protein or its subunits for binding to an antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. Antibody binding can be detected by detecting a label on the primary antibody. Alternatively, the primary antibody is detected by detecting binding of a secondary antibody or reagent to the primary antibody, particularly where the secondary antibody is labeled. Many means are known in the art for detecting binding in an immunoassay and are within the scope of the present invention.

The binding of mutant neurotrophin family protein subunits, mutant neurotrophin family protein heterodimers, neurotrophin family protein analogs, single chain analogs, derivatives and fragments thereof, to the neurotrophin family protein receptor can be determined by methods well-known in the art, such as but not limited to in vitro assays based on displacement from the neurotrophin family protein receptor of a radiolabeled neurotrophin family protein of another species, such as bovine neurotrophin family protein. The bioactivity of mutant neurotrophin family protein heterodimers, neurotrophin family protein analogs, single chain analogs, derivatives and fragments thereof, can also be measured, by a variety of bioassays are known in the art to determine the functionality of mutant neurotrophin protein. For example, auto-phosphorylation studies, cross-linking studies and ligand binding studies are well-known in the art and are used to evaluate the functional aspects of the mutant neurotrophin protein of the present invention. Further, bioassays that compare mutant and wild type activities in inducing phenotypic changes in a population of test cells.

Autophosphorylation

To determine whether or not a mutant neurotrophin protein demonstrates biological activity, a receptor molecule for the neurotrophin protein of interest is created. In one assay system, the cDNA for trkC is generated and subcloned into expression vectors, transfected, and stably expressed in NIH 3T3 fibroblasts, cells that do not normally express any trk family protein. Expression of the transfected receptor is confirmed using standard techniques known in the art. (See, Tsoulfas et al., Neuron, 10:975-990 (1993)).

Following the transfection procedure, the modified NIH 3T3 cells are tested for their ability to respond to the mutant neurotrophin protein of the present invention. The transfected fibroblasts are subsequently exposed to various amounts of purified, partially purified, or crude recombinant mutant neurotrophins and assayed for the results. In one assay, mutant NT-3 protein over a range of concentrations from about 0 to 1000 ng/ml are applied to a trkC expressing cell line for a period of time sufficient to elicit a biological response from the test cell. In one example, this time period is approxinately five (5) minutes. Following exposure to the mutant protein, the cells are lysed and the lysates are immunoprecipitated with an antiserum that recognizes the highly conserved C-terminus of all Trk family receptors. One example of such an antibody is rabbit antiserum 443. (See Soppet, et al., Cell May 31, 1991, 65:5 895-903). After gel electrophoresis and transfer to nitrocellulose, the filters were probed with another antibody to detect to presence of phosphorylated tyrosine residues. The monoclonal antibody 4G10 is a monoclonal antibody specific for such phosphorylated residues. (See Kaplan et al., Tsoulfas et al.). The phosphorylation of TrkC tyrosine residues indicates catalytic activation of the receptor and also indicates the functionality of the tested mutant neurotrophin protein.

Affinity Cross-Linking

Chemical cross-linking experiments are performed to determine binding affinities for the various mutant neurotrophin protein of the present invention. One example of this technique involves the preparation of cell membranes isolated from neurotrophin receptor expressing cell lines. These membranes are incubated with ¹²⁵I-labled neurotrophins, either mutant or wild type forms, and are then treated with a chemical cross-linking agent such as EDAC. The neurotrophin receptors present in the cell membranes are then isolated and examined for the presence of bound and crosslinked neurotrophin. For example, antisera 443 can be used to immunoprecipitate Trk receptors from cell solutions. The immunoprecipitated material is then applied to a polyacrylamide gel and an autoradiograph is prepared using standard techniques. Only receptors that bound and are cross-linked to a labeled ligand will be detected on the autoradiograph. The assay provides a simple method to determine which mutant neurotrophin protein are capable of binding to their respective cognate receptors.

Ligand Binding Kinetics

Equilibrium binding experiments using radiolabled mutant neurotrophin protein are performed to determine the ligand binding kinetics of cells expressing a neurotrophin receptor. An example of such a methodology utilizes a group of mutant NT-3 protein that contain at least one electrostatic charge altering mutation in either the L1 or L3 loops, or both. These protein are radioiodinated and are the ligands in the study.

The mutant neurotrophin protein are prepared and purified according to the methods described herein. A purified preparation of the mutant neurotrophin protein is radioiodinated according to standard techniques well known in the art. To illustrate, mutant neurotrophin protein are labeled with ¹²⁵I using lactoperoxidase treatment using a modification of the Enzymobead radioiodination reagent (Bio-Rad, Hercules, Calif.) procedure. Routinely, 2 μg amounts of the ligands are iodinated to specific activities ranging from 2500 to 3500 cpm/fmmol. The ¹²⁵I-labeled factors are stored at 4° C. and used within 2 weeks of preparation. Often the bioactivity of the radiolabeled mutant neurotrophin protein is tested before binding studies are performed to determine that the iodination procedure did not damage the ligand.

One series of experiments performed involves using fixed concentrations of iodinated ligand and membrane preparations. In these displacement studies, unlabeled wild type neurotrophin displaces the labeled mutant neurotrophin at a particular concentration or concentrations, depending on the binding characteristics of the protein. The concentration at which half of the labeled protein is displaced is known as the inhibition constant or IC₅₀. By calculating the IC₅₀, of a mutant neurotrophin protein and comparing that value to the wild type protein, it is possible to determine which mutations taught by the present invention result in an increased affinity for the receptor by the mutant ligand protein.

The data gathered from this type of experiment also permit the preparation of a Scathard plot and from this a disassociation constant for the mutant neurotrophin protein can be determined. This value further indicates the affinity of the mutant neurotrophin ligand for its receptor and the determined value can be compared to the wild type value in order to evaluate the desirability of a mutation or combination of mutations.

PC12 Cell Bioassays

PC12 cells are transiently transfected with a neurotrophin receptor expression vector using standard techniques well known in the art. The expression vector encodes a neurotrophin receptor with activity for the wild type neurotrophin protein of interest. This receptor is used to determine the effect mutations introduced into the amino acid sequence of the wild type neurotrophin protein of interest have on the biological activity of the mutant protein as compared to that of the wild type protein. For example, the PC12 bioassay has been applied to NGF analysis, (Patterson & Childs, Endocrinology, 135:1697-1704(1994)); BDNF, (Suter, et al., J. Neuroscience, 12:306-318(1992)); NT-3, (Tsoulfas, et al., Neuron, 10:975-990 (1993)); and NT-4, (Tsoulfas, et al., Neuron, 10:975-990 (1993)).

To compare wild type and mutant neurotrophin protein bioactivity, PC12 cells are grown on collagen-coated dishes and resuspended in PC12 growth medium by gentle trituration and plated at 10%-20% density on 10 cm collagen-coated dishes. The following day cells are washed 4 times with DMEM and 5 ml of DMEM, 3 μg/ml insulin, 100 μg of Li pofectin (GIBCO-BRL, Gaithersburg, Md.) and 50 μg of an expression vector containing the neurotrophin receptor. The lipofectin mixture is replaced with fresh PC12 medium after eight (8) hours. The following day, cells are fed with PC12 medium with or without 10 ng/ml of neurotrophin mutant protein or wild type protein. Three days following treatment, the plates are scored for cells exhibiting neurite processes >2 cell diameters in length. Scoring is performed by counting >1000 random 1.2 mm2 fields. The results are reported as the number of neurite-bearing cells multiplied by 100/the number of fields counted. Neurite induction is compared between mutant protein and wild type neurotrophin protein.

The half-life of a protein is a measurement of protein stability and indicates the time necessary for a one-half reduction in the concentration of the protein. The half life of a mutant neurotrophin family protein can be determined by any method for measuring neurotrophin family protein levels in samples from a subject over a period of time, for example but not limited to, immunoassays using anti-neurotrophin family protein antibodies to measure the mutant neurotrophin family protein levels in samples taken over a period of time after administration of the mutant neurotrophin family protein or detection of radiolabelled mutant neurotrophin family protein in samples taken from a subject after administration of the radiolabelled mutant neurotrophin family protein.

Other methods will be known to the skilled artisan and are within the scope of the invention.

Diagnostic and Therapeutic Uses

The invention provides for treatment or prevention of various diseases and disorders by administration of therapeutic compound (termed herein “Therapeutic”) of the invention. Such Therapeutics include neurotrophin family protein heterodimers having a mutant α subunit and either a mutant or wild type β subunit; neurotrophin family protein heterodimers having a mutant α subunit and a mutant β subunit and covalently bound to another CKGF protein, in whole or in part, such as the CTEP of the β subunit of hLH; neurotrophin family protein heterodimers having a mutant α subunit and a mutant β subunit, where the mutant α subunit and the mutant β subunit are covalently bound to form a single chain analog, including a neurotrophin family protein heterodimer where the mutant α subunit and the mutant β subunit and the CKGF protein or fragment are covalently bound in a single chain analog, other derivatives, analogs and fragments thereof (e.g. as described hereinabove) and nucleic acids encoding the mutant neurotrophin family protein heterodimers of the invention, and derivatives, analogs, and fragments thereof.

The subject to which the Therapeutic is administered is preferably an animal, including but not limited to animals such as cows, pigs, horses, chickens, cats, dogs, etc., and is preferably a mammal. In a preferred embodiment, the subject is a human. Generally, administration of products of a species origin that is the same species as that of the subject is preferred. Thus, in a preferred embodiment, a human mutant and/or modified neurotrophin family protein heterodimer, derivative or analog, or nucleic acid, is therapeutically or prophylactically or diagnostically administered to a human patient.

In a preferred aspect, the Therapeutic of the invention is substantially purified.

A number of disorders which manifest as neurodegenerative diseases or disorders can be treated by the methods of the invention. Neurodegenerative disease in which neurotrophin family protein is absent or decreased relative to normal or desired levels are treated or prevented by administration of a mutant neurotrophin family protein heterodimer or neurotrophin family protein analog of the invention. Examples of these diseases or disorders include: parkinson's disease and alzheimer's disease. Disorders in which neurotrophin family protein receptor is absent or decreased relative to normal levels or unresponsive or less responsive than normal neurotrophin family protein receptor to wild type neurotrophin family protein, can also be treated by administration of a mutant neurotrophin family protein heterodimer or neurotrophin family protein analog. Mutant neurotrophin family protein heterodimers and neurotrophin family protein analogs for use as antagonists are contemplated by the present invention.

In specific embodiments, mutant neurotrophin family protein heterodimers or neurotrophin family protein analogs with bioactivity are administered therapeutically, including prophylactically to accelerate angiogenesis. For example, VEGF, PDGF and TGF-β are all endothelial mitogens. In situations where angiogenesis is to be promoted, the application of mutant PDGF family proteins that have increased bioactivity would be beneficial.

In another embodiment, the application of PDGF family receptors antagonists would inhibit angiogenesis. Angiogenesis inhibition is useful in conditions where one of skill in the art would want to inhibit novel or increased vascularization. Examples of such conditions include: tumors, where tumor growth corresponds to an increased rate of angiogenic activity; diabetic retinopathy, which is neovascularization into the vitreous humor of the eye; prolonged menstal bleed; infertility; and hemangiomas.

The absence of or a decrease in neurotrophin family protein protein or function, or neurotrophin family protein receptor protein and function can be readily detected, e.g., by obtaining a patient tissue sample (e.g., from biopsy tissue) and assaying it in vitro for RNA or protein levels, structure and/or activity of the expressed RNA or protein of neurotrophin family protein or neurotrophin family protein receptor. Many methods standard in the art can be thus employed, including but not limited to immunoassays to detect and/or visualize neurotrophin family protein or neurotrophin family protein receptor protein (e.g., Western blot, immunoprecipitation followed by sodium dodecyl sulfate polyacrylamide gel electrophoresis, immunocytochemistry, etc.) and/or hybridization assays to detect neurotrophin family protein or neurotrophin family protein receptor expression by detecting and/or visualizing neurotrophin family protein or neurotrophin family protein receptor mRNA (e.g., Northern assays, dot blots, in situ hybridization, etc.), etc.

Mutants of the TGF-β Protein Family

As discussed above, the TGF-β protein family encompasses a multitude of protein subfamilies. Mutants of the TGF-β protein family are discussed below.

Mutants of the Human Transforming Growth Factor β1 Monomer

The human transforming growth factor β1 monomer contains 112 amino acids as shown in FIG. 14 (SEQ ID No: 13). The invention contemplates mutants of the human transforming growth factor β1 monomer comprising single or multiple amino acid substitutions, deletions or insertions, of one, two, three, four or more amino acid residues when compared with the wild type monomer. Furthermore, the invention contemplates mutant human transforming growth factor β1 monomers that are linked to another CKGF protein.

The present invention provides mutant transforming growth factor β1 monomer L1 hairpin loops having one or more amino acid substitutions between positions 21 and 40, inclusive, excluding Cys residues, as depicted in FIG. 14 (SEQ ID NO: 13). The amino acid substitutions include: Y21X, I22X, D23X, F24X, R25X, K26X, D27X, L28X, G29X, W30X, K31X, W32X, I33X, H34X, E35X, P36X, K37X, G38X, Y39X, and H40X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the transforming growth factor β1 monomer where an acidic residue is present, the variable “X” would correspond to a basic amino acid residue. Specific examples of electrostatic charge altering mutations where a basic residue is introduced into the transforming growth factor β1 monomer include one or more of the following: D23B, D27B, and E35B wherein “B” is a basic amino acid residue.

Introducing acidic amino acid residues where basic residues are present in the transforming growth factor β1 monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: R25Z, K26Z, K31Z, H34Z, K37Z, and H40Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D23U, R25U, K26U, D27U, K31U, H34U, E35U, K37U, and H40U, wherein “U” is a neutral amino acid.

Mutant transforming growth factor β1 monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: Y21Z, I22Z, F24Z, L28Z, G29Z, W30Z, W32Z, I33Z, P36Z, G38Z, Y39Z, Y21B, I22B, F24B, L28B, G29B, W30B, W32B, I33B, P36B, G38B, and Y39B, wherein “Z” is an acidic amino acid and “B” is a basic amino acid.

Mutant transforming growth factor β1 monomers containing mutants in the L3 hairpin loop are also described. These mutant proteins have one or more amino acid substitutions, deletion or insertions, between positions 82 and 102, inclusive, excluding Cys residues, of the L3 hairpin loop, as depicted in FIG. 14 (SEQ ID NO: 13). The amino acid substitutions include: A82X, L83X, E84X, P85X, L86X, P87X, I88X, V89X, Y90X, Y91X, V92X, G93X, R94X, K95, P96X, K97X, V98X, E99X, Q100X, L101X, and S102X, wherein “X” is any amino acid residue, the substitution of which alters the electrostatic character of the L3 loop.

One set of mutations of the L3 hairpin loop includes introducing one or more basic amino acid residues into the transforming growth factor β1 L3 hairpin loop amino acid sequence. For example, when introducing basic residues into the L3 loop of the transforming growth factor β1 monomer, the variable “X” of the sequence described above corresponds to a basic amino acid residue. Specific examples of electrostatic charge altering mutations where a basic residue is introduced into the transforming growth factor β1 monomer include one or more of the following: E84B and E99B, wherein “B” is a basic amino acid residue.

The invention further contemplates introducing one or more acidic residues into the amino acid sequence of the transforming growth factor β1 L3 hairpin loop. For example, one or more acidic amino acids can be introduced in the sequence of 82-102 described above, wherein the variable “X” corresponds to an acidic amino acid. Specific examples of such mutations include R94Z, K95Z, and K97Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at E84U, R94U, K95U, K97U, and E99U, wherein “U” is a neutral amino acid.

Mutant transforming growth factor β1 proteins are provided containing one or more electrostatic charge altering mutations in the L3 hairpin loop amino acid sequence that convert non-charged or neutral amino acid residues to charged residues. Examples of mutations converting neutral amino acid residues to charged residues include, A82Z, L83Z, P85Z, L86Z, P87Z, I88Z, V89Z, Y90Z, Y91Z, V92Z, G93Z, P96Z, V98Z, Q100Z, L101Z, S102Z, A82B, L83B, P85B, L86B, P87B, I88B, V89B, Y90B, Y91B, V92B, G93B, P96B, V98B, Q100B, L101B, and S102B, wherein “Z” is an acidic amino acid and “B” is a basic amino acid.

The present invention also contemplate transforming growth factor β1 monomers containing mutations outside of said β hairpin loop structures that alter the structure or conformation of those hairpin loops. These structural alterations in turn serve to increase the electrostatic interactions between regions of the β hairpin loop structures of transforming growth factor β1 monomer contained in a dimeric molecule, and a receptor having affinity for the dimeric protein. These mutations are found at positions selected from the group consisting of positions 1-20, 41-81, and 103-112 of the transforming growth factor β1 monomer.

Specific examples of these mutation outside of the β hairpin L1 and L3 loop structures include, A1J, L2J, D3J, T4J, N5J, Y6J, C7J, F8J, S9J, S10J, T11J, E12J, K13J, N14J, C15J, C16J, V17J, R18J, Q19J, L20J, A41J, N42J, F43J, C44J, L45J, G46J, P47J, C48J, P49J, Y50J, I51J, W52J, S53J, L54J, D55J, T56J, Q57J, Y58J, S59J, K60J, V61J, L62J, A63J, L64J, Y65J, N66J, Q67J, H68J, N69J, P70J, G71J, A72J, S73J, A74J, A75J, P76J, C77J, C78J, V79J, P80J, Q81J, N103J, M104J, I105J, V106J, R107J, S108J, C109J, K110J, C111J, and S112J. The variable “J” is any amino acid whose introduction results in an increase in the electrostatic interaction between the L1 and L3 β hairpin loop structures of the transforming growth factor β1 and a receptor with affinity for a dimeric protein containing the mutant transforming growth factor β1 monomer.

The invention also contemplates a number of transforming growth factor β1 monomers in modified forms. These modified forms include transforming growth factor β1 monomers linked to another cystine knot growth factor monomer or a fraction of such a monomer.

In specific embodiments, the mutant TGF-β1 heterodimer comprising at least one mutant subunit or the single chain TGF-β1 analog as described above is functionally active, i.e., capable of exhibiting one or more functional activities associated with the wild-type TGF-β1,such as TGF-β1 receptor binding, TGF-β1 protein family receptor signalling and extracellular secretion. Preferably, the mutant TGF-β1 heterodimer or single chain TGF-β1 analog is capable of binding to the TGF-β1 receptor, preferably with affinity greater than the wild type TGF-β1. Also it is preferable that such a mutant TGF-β1 heterodimer or single chain TGF-β1 analog triggers signal transduction. Most preferably, the mutant TGF-β1 heterodimer comprising at least one mutant subunit or the single chain TGF-β1 analog of the present invention has an in vitro bioactivity and/or in vivo bioactivity greater than the wild type TGF-β1 and has a longer serum half-life than wild type TGF-β1. Mutant TGF-β1 heterodimers and single chain TGF-β1 analogs of the invention can be tested for the desired activity by procedures known in the art.

Mutants of the Human Transforming Growth Factor β2 Monomer

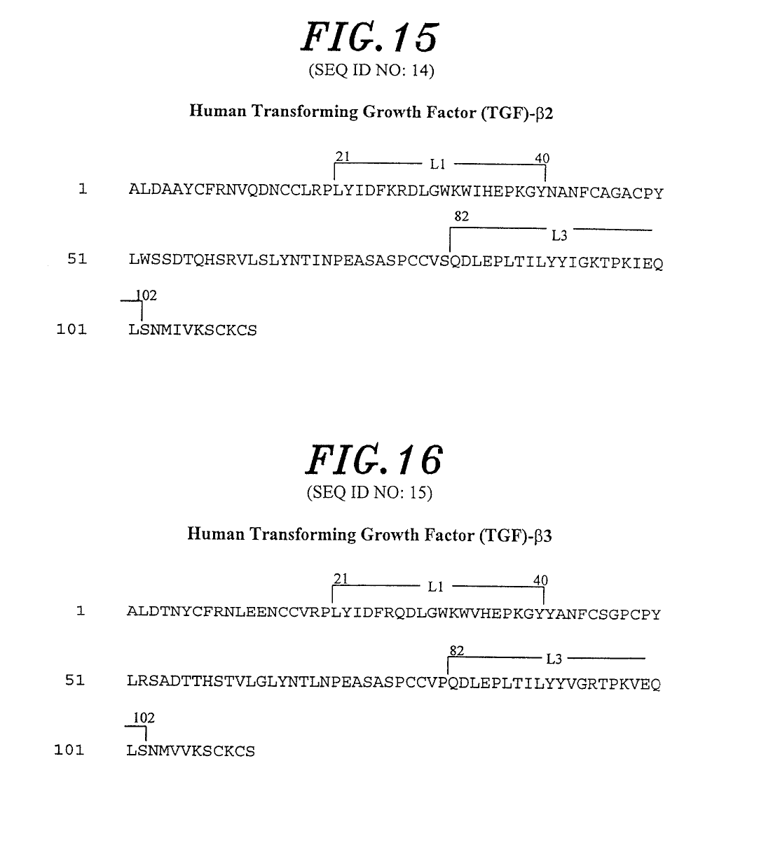

The human transforming growth factor β2 monomer contains 112 amino acids as shown in FIG. 15 (SEQ ID No: 14). The invention contemplates mutants of the human transforming growth factor β2 monomer comprising single or multiple amino acid substitutions, deletions or insertions, of one, two, three, four or more amino acid residues when compared with the wild type monomer. Furthermore, the invention contemplates mutant human transforming growth factor β2 monomers that are linked to another CKGF protein.

The present invention provides mutant transforming growth factor β2 monomer L1 hairpin loops having one or more amino acid substitutions between positions 21 and 40, inclusive, excluding Cys residues, as depicted in FIG. 15 (SEQ ID NO: 14). The amino acid substitutions include: Y21X, I22X, D23X, F24X, K25X, R26X, D27X, L28X, G29X, W30X, K31X, W32X, I33X, H34X, E35X, P36X, K37X, G38X, Y39X, and N40X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the transforming growth factor β2 monomer, the variable “X” would correspond to a basic amino acid residue. Specific examples of electrostatic charge altering mutations where a basic residue is introduced into the transforming growth factor β2 monomer include one or more of the following: D23B, D27B, and E35B, wherein “B” is a basic amino acid residue.

Introducing acidic amino acid residues where basic residues are present in the transforming growth factor β2 monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: K25Z, R26Z, K31Z, H34Z, and K37Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D23U, K25U, R26U, D27U, K31U, H34U, E35U, and K37U, wherein “U” is a neutral amino acid.

Mutant Transforming growth factor β2 monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: Y21Z, I22Z, F24Z, L28Z, G29Z, W30Z, W32Z, I33Z, P36Z, G38Z, Y39Z, N40Z, Y21B, I22B, F24B, L28B, G29B, W30B, W32B, I33B, P36B, G38B, Y39B, and N40B, wherein “Z” is an acidic amino acid and “B” is a basic amino acid.

Mutant transforming growth factor β2 monomers containing mutants in the L3 hairpin loop are also described. These mutant proteins have one or more amino acid substitutions, deletion or insertions, between positions 82 and 102, inclusive, excluding Cys residues, of the L3 hairpin loop, as depicted in FIG. 15 (SEQ ID NO: 14). The amino acid substitutions include D82X, L83X, E84X, P85X, L86X, T87X, I88X, L89X, Y90X, Y91X, I92X, G93X, K94X, T95X, P96X, K97X, I98X, E99X, Q100X, L101X, and S102X, wherein “X” is any amino acid residue, the substitution of which alters the electrostatic character of the L3 loop.

One set of mutations of the L3 hairpin loop includes introducing one or more basic amino acid residues into the transforming growth factor β2 L3 hairpin loop amino acid sequence. For example, when introducing basic residues into the L3 loop of the transforming growth factor β2 monomer, the variable “X” of the sequence described above corresponds to a basic amino acid residue. Specific examples of electrostatic charge altering mutations where a basic residue is introduced into the transforming growth factor β2 monomer include one or more of the following: D82B, E84B, and E99B, wherein “B” is a basic amino acid residue.

The invention further contemplates introducing one or more acidic residues into the amino acid sequence of the transforming growth factor β1 L3 hairpin loop. For example, one or more acidic amino acids can be introduced in the sequence of 82-102 described above, wherein the variable “X” corresponds to an acidic amino acid. Specific examples of such mutations include K94Z and K97Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at D82U, E84U, K94U, K97U, and E99U, wherein “U” is a neutral amino acid.

Mutant transforming growth factor β2 proteins are provided containing one or more electrostatic charge altering mutations in the L3 hairpin loop amino acid sequence that convert non-charged or neutral amino acid residues to charged residues. Examples of mutations converting neutral amino acid residues to charged residues include, L83Z, P85Z, L86Z, T87Z, I88Z, L89Z, Y90Z, Y91Z, I92Z, G93Z, T95Z, P96Z, I98Z, Q100Z, L101Z, S102Z, L83B, P85B, L86B, T87B, I88B, L89B, Y90B, Y91B, I92B, G93B, T95B, P96B, I98B, Q100B, L101B, and S102B, wherein “Z” is an acidic amino acid and “B” is a basic amino acid.

The present invention also contemplate transforming growth factor β2 monomers containing mutations outside of said β hairpin loop structures that alter the structure or conformation of those hairpin loops. These structural alterations in turn serve to increase the electrostatic interactions between regions of the β hairpin loop structures of transforming growth factor β2 monomer contained in a dimeric molecule, and a receptor having affinity for the dimeric protein. These mutations are found at positions selected from the group consisting of positions 1-20, 41-81, and 103-112 of the transforming growth factor β2 monomer.

Specific examples of these mutation outside of the β hairpin L1 and L3 loop structures include, A1J, L2J, D3J, A4J, A5J, Y6J, C7J, F8J, R9J, N10J, V11J, Q12J, D13J, N14J, C15J, C16J, L17J, R18J, P19J, L20J, A41J, N42J, F43J, C44J, A45J, G46J, A47J, C48J, P49J, Y50J, L51J, W52J, S53J, S54J, D55J, T56J, Q57J, H58J, S59J, R60J, V61J, L62J, S63J, L64J, Y66J, N66J, T67J, I68J, N69J, P70J, E71J, A72J, S73J, A74J, S75J, P76J, C77J, C78J, V79J, S80J, Q81J, N103J, M104J, I105J, V106J, K107J, S108J, C109J, K110J, C111J, and S112J. The variable “J” is any amino acid whose introduction results in an increase in the electrostatic interaction between the L1 and L3 β hairpin loop structures of the transforming growth factor β2 and a receptor with affinity for a dimeric protein containing the mutant transforming growth factor β2 monomer.

The invention also contemplates a number of transforming growth factor β2 monomers in modified forms. These modified forms include transforming growth factor β2 monomers linked to another cystine knot growth factor monomer or a fraction of such a monomer.

In specific embodiments, the mutant TGF-β2 heterodimer comprising at least one mutant subunit or the single chain TGF-β2 analog as described above is functionally active, i.e., capable of exhibiting one or more functional activities associated with the wild-type TGF-β2, such as TGF-β2 receptor binding, TGF-β2 protein family receptor signalling and extracellular secretion. Preferably, the mutant TGF-β2 heterodimer or single chain TGF-β2 analog is capable of binding to the TGF-β2 receptor, preferably with affinity greater than the wild type TGF-β2. Also it is preferable that such a mutant TGF-β2 heterodimer or single chain TGF-β2 analog triggers signal transduction. Most preferably, the mutant TGF-β2 heterodimer comprising at least one mutant subunit or the single chain TGF-β2 analog of the present invention has an in vitro bioactivity and/or in vivo bioactivity greater than the wild type TGF-β2 and has a longer serum half-life than wild type TGF-β2. Mutant TGF-β2 heterodimers and single chain TGF-β2 analogs of the invention can be tested for the desired activity by procedures known in the art.

Mutants of the Human Transforming Growth Factor β3 Monomer

The human transforming growth factor β3 monomer contains 112 amino acids as shown in FIG. 16 (SEQ ID No: 15). The invention contemplates mutants of the human transforming growth factor β3 monomer comprising single or multiple amino acid substitutions, deletions or insertions, of one, two, three, four or more amino acid residues when compared with the wild type monomer. Furthermore, the invention contemplates mutant human transforming growth factor β3 monomers that are linked to another CKGF protein.

The present invention provides mutant transforming growth factor β3 monomer L1 hairpin loops having one or more amino acid substitutions between positions 21 and 40, inclusive, excluding Cys residues, as depicted in FIG. 16 (SEQ ID No: 15). The amino acid substitutions include: Y21X, I22X, D23X, F24X, R25X, Q26X, D27X, L28X, G29X, W30X, K31X, W32X, V33X, H34X, E35X, P36X, K37X, G38X, Y39X, and Y40X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the transforming growth factor β3 monomer, the variable “X” would correspond to a basic amino acid residue. Specific examples of electrostatic charge altering mutations where a basic residue is introduced into the transforming growth factor β3 monomer include one or more of the following: D23B, D27B, and E35B wherein “B” is a basic amino acid residue.

Introducing acidic amino acid residues where basic residues are present in the transforming growth factor β3 monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: R25Z, K31Z, H34Z, and K37Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D23U, R25U, D27U, K31U, H34U, E35U, and K37U, wherein “U” is a neutral amino acid.

Mutant Transforming growth factor β3 monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: Y21Z, I22Z, F24Z, Q26Z, L28Z, G29Z, W30Z, W32Z, V33Z, P36Z, G38Z, Y39Z, Y40Z, Y21B, I22B, F24B, Q26B, L28B, G29B, W30B, W32B, V33B, P36B, G38B, Y39B, and Y40B, wherein “Z” is an acidic amino acid and “B” is a basic amino acid.

Mutant transforming growth factor β3 monomers containing mutants in the L3 hairpin loop are also described. These mutants proteins have one or more amino acid substitutions, deletion or insertions, between positions 82 and 102, inclusive, excluding Cys residues, of the L3 hairpin loop, as depicted in FIG. 16 (SEQ ID No: 15). The amino acid substitutions include: D82X, L83X, E84X, P85X, L86X, T87X, I88X, L89X, Y90X, Y91X, V92X, G93X, R94X, T95X, P96X, K97X, V98X, E99X, Q100X, L101X, and S102X, wherein “X” is any amino acid residue, the substitution of which alters the electrostatic character of the L3 loop.

One set of mutations of the L3 hairpin loop includes introducing one or more basic amino acid residues into the transforming growth factor β3 L3 hairpin loop amino acid sequence. For example, when introducing basic residues into L3 loop of the transforming growth factor β3 monomer, the variable “X” of the sequence described above corresponds to a basic amino acid residue. Specific examples of electrostatic charge altering mutations where a basic residue is introduced into the transforming growth factor β3 monomer include one or more of the following: D82B, E84B, and E99B, wherein “B” is a basic amino acid residue.

The invention further contemplates introducing one or more acidic residues into the amino acid sequence of the transforming growth factor β3 L3 hairpin loop. For example, one or more acidic amino acids can be introduced in the sequence of 82-102 described above, wherein the variable “X” corresponds to an acidic amino acid. Specific examples of such mutations include R94Z and K97Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at D82U, E84U, R94U, K97U, and E99U, wherein “U” is a neutral amino acid.

Mutant transforming growth factor β1 proteins are provided containing one or more electrostatic charge altering mutations in the L3 hairpin loop amino acid sequence that convert non-charged or neutral amino acid residues to charged residues. Examples of mutations converting neutral amino acid residues to charged residues include, L83Z, P85Z, L86Z, T87Z, I88Z, L89Z, Y90Z, Y91Z, V92Z, G93Z, T95Z, P96Z, V98Z, Q100Z, L101Z, S102Z, L83B, P85B, L86B, T87B, I88B, L89B, Y90B, Y91B, V92B, G93B, T95B, P96B, V98B, Q100B, L101B, and S102B, wherein “Z” is an acidic amino acid and “B” is a basic amino acid.

The present invention also contemplate transforming growth factor β3 monomers containing mutations outside of said β hairpin loop structures that alter the structure or conformation of those hairpin loops. These structural alterations in turn serve to increase the electrostatic interactions between regions of the β hairpin loop structures of transforming growth factor β3 monomer contained in a dimeric molecule, and a receptor having affinity for the dimeric protein. These mutations are found at positions selected from the group consisting of positions 1-20, 41-81, and 103-112 of the transforming growth factor β3 monomer.

Specific examples of these mutation outside of the β hairpin L1 and L3 loop structures include, A1J, L2J, D3J, T4J, N5J, Y6J, C7J, F8J, R9J, N10J, L11J, E12J, E13J, N14J, C15J, C16J, V17J, R18J, P19J, L20J, A41J, N42J, F43J, C44J, S45J, G46J, P47J, C48J, P49J, Y50J, L51J, R52J, S53J, A54J, D55J, T56J, T57J, H58J, S59J, T60J, V61J, L62J, C63J, L64J, Y665J, N66J, T67J, L68J, N69J, P70J, E71J, A72J, S73J, A74J, S75J, P76J, C77J, C78J, V79J, P80J, Q81J, N103J, M104J, V105J, V106J, K107J, S108J, C109J, K110J, C111J, and S112J. The variable “J” is any amino acid whose introduction results in an increase in the electrostatic interaction between the L1 and L3 β hairpin loop structures of the transforming growth factor β1 and a receptor with affinity for a dimeric protein containing the mutant transforming growth factor β3 monomer.

The invention also contemplates a number of transforming growth factor β3 monomers in modified forms. These modified forms include transforming growth factor β3 monomers linked to another cystine knot growth factor monomer or a fraction of such a monomer.

In specific embodiments, the mutant TGF-β3 heterodimer comprising at least one mutant subunit or the single chain TGF-β3 analog as described above is functionally active, i.e., capable of exhibiting one or more functional activities associated with the wild-type TGF-β3, such as TGF-β3 receptor binding, TGF-β3 protein family receptor signalling and extracellular secretion. Preferably, the mutant TGF-β3 heterodimer or single chain TGF-β3 analog is capable of binding to the TGF-β3 receptor, preferably with affinity greater than the wild type TGF-β3. Also it is preferable that such a mutant TGF-β3 heterodimer or single chain TGF-β3 analog triggers signal transduction. Most preferably, the mutant TGF-β3 heterodimer comprising at least one mutant subunit or the single chain TGF-β3 analog of the present invention has an in vitro bioactivity and/or in vivo bioactivity greater than the wild type TGF-β3 and has a longer serum half-life than wild type TGF-β3. Mutant TGF-β3 heterodimers and single chain TGF-β3 analogs of the invention can be tested for the desired activity by procedures known in the art.

Mutants of the Human Transforming Growth Factor-β4 (TGF-β4)/ebaf Subunit

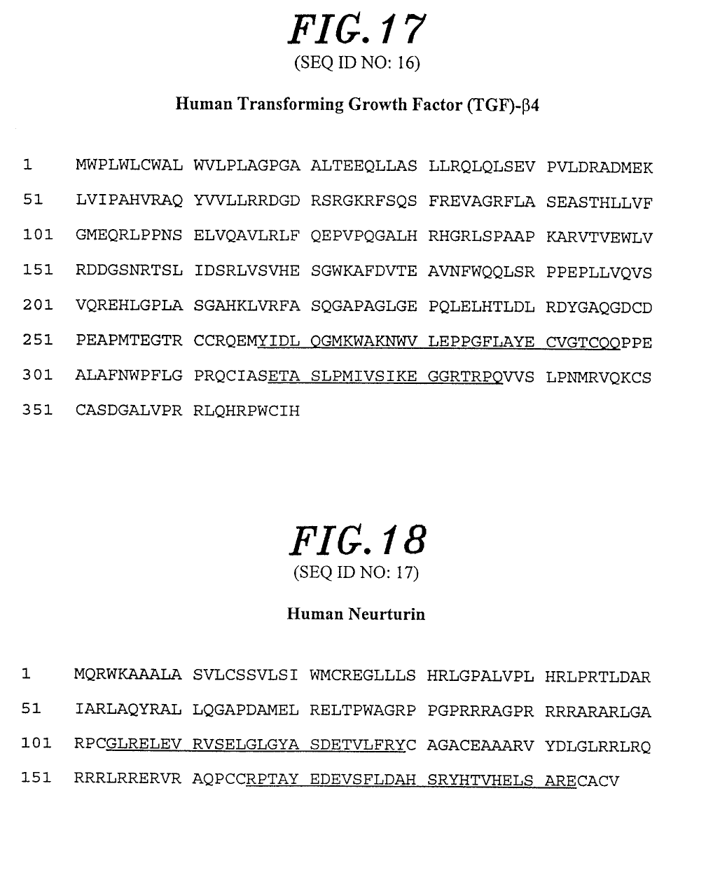

The human transforming growth factor-β4 (TGF-β4)/ebaf subunit contains 370 amino acids as shown in FIG. 17 (SEQ ID No: 16). The invention contemplates mutants of the β4 comprising single or multiple amino acid substitutions, deletions or insertions, of one, two, three, four or more amino acid residues when compared with the wild type monomer. Furthermore, the invention contemplates mutant β4 that are linked to another CKGF protein.

The present invention provides mutant TGF-β4 L1 hairpin loops having one or more amino acid substitutions between positions 267 and 287, inclusive, excluding Cys residues, as depicted in FIG. 17 (SEQ ID NO: 16). The amino acid substitutions include: Y267X, I268X, D269X, L270X, Q271X, G272X, M273X, K274X, W275X, A276X, K277X, N278X, W279X, V280X, L281X, E282X, P283X, P284X, G285X, F286X, and L287X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the TGF-β4 where an acidic residue is present, the variable “X” would correspond to a basic amino acid residue. Specific examples of electrostatic charge altering mutations where a basic residue is introduced into the TGF-β4 include one or more of the following: D269B and E282B, wherein “B” is a basic amino acid residue.

Introducing acidic amino acid residues where basic residues are present in the TGF-β4 sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: K274Z and K277Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D269U, K274U, K277U, and E282U, wherein “U” is a neutral amino acid.

Mutant TGF-β4 proteins are provided containing one or more electrostatic charge altering mutations in the L1 hairpin loop amino acid sequence that convert non-charged or neutral amino acid residues to charged residues. Examples of mutations converting neutral amino acid residues to charged residues include: Y267Z, I268Z, L270Z, Q271Z, G272Z, M273Z, W275Z, A276Z, N278Z, W279Z, V280Z, L281Z, P283Z, P284Z, G285Z, F286Z, L287Z, Y267B, I268B, L270B, Q271B, G272B, M273B, W275B, A276B, N278B, W279B, V280B, L281B, P283B, P284B, G285B, F286B, and L287B, wherein “Z” is an acidic amino acid and “B” is a basic amino acid.

Mutant TGF-β4 containing mutants in the L3 hairpin loop are also described. These mutant proteins have one or more amino acid substitutions, deletion or insertions, between positions 318 and 337, inclusive, excluding Cys residues, of the L3 hairpin loop, as depicted in FIG. 17 (SEQ ID NO: 16). The amino acid substitutions include: E318X, T319X, A320X, S321X, L322X, P323X, M324X, I325X, V326X, S327X, I328X, K329X, E330X, G331X, G332X, R333X, T334X, R335X, P336X, and Q337X, wherein “X” is any amino acid residue, the substitution of which alters the electrostatic character of the L3 loop.

One set of mutations of the L3 hairpin loop includes introducing one or more basic amino acid residues into the TGF-β4 L3 hairpin loop amino acid sequence. For example, when introducing basic residues into the L3 loop of the TGF-β4, the variable “X” of the sequence described above corresponds to a basic amino acid residue. Specific examples of electrostatic charge altering mutations where a basic residue is introduced into the TGF-β4 include one or more of the following: E318B and E330B, wherein “B” is a basic amino acid residue.

The invention further contemplates introducing one or more acidic residues into the amino acid sequence of the TGF-β4 L3 hairpin loop. For example, one or more acidic amino acids can be introduced in the sequence of 318-337 described above, wherein the variable “X” corresponds to an acidic amino acid. Specific examples of such mutations include K329Z, R333Z, and R335Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at E318U, K329U, E330U, R333U, and R335U, wherein “U” is a neutral amino acid.

Mutant TGF-β4proteins are provided containing one or more electrostatic charge altering mutations in the L3 hairpin loop amino acid sequence that convert non-charged or neutral amino acid residues to charged residues. Examples of mutations converting neutral amino acid residues to charged residues include, T319Z, A320Z, S321Z, L322Z, P323Z, M324Z, I325Z, V326Z, S327Z, I328Z, G331Z, G332Z, T334Z, R335Z, P336Z, Q337Z, T319B, A320B, S321B, L322B, P323B, M324B, I325B, V326B, S327B, I328B, G331B, G332B, T334B, R335B, P336B, and Q337B, wherein “Z” is an acidic amino acid and “B” is a basic amino acid.

The present invention also contemplate TGF-β4 containing mutations outside of said β hairpin loop structures that alter the structure or conformation of those hairpin loops. These structural alterations in turn serve to increase the electrostatic interactions between regions of the β hairpin loop structures of TGF-β4 contained in a dimeric molecule, and a receptor having affinity for the dimeric protein. These mutations are found at positions selected from the group consisting of positions 1-266, 288-317, and 338-370 of the TGF-β4.

Specific examples of these mutation outside of the β hairpin L1 and L3 loop structures include, M1J, W2J, P3J, L4J, W5J, L6J, C7J, W8J, A9J, L10J, W11J, V12J, L13J, P14J, L15J, A16J, G17J, P18J, G19J, A20J, A21J, L22J, T23J, E24J, E25J, Q26J, L27J, L28J, A29J, S30J, L31J, L32J, R33J, Q34J, L35J, Q36J, L37J, S38J, E39J, V40J, P41J, V42J, L43J, D44J, R45J, A46J, D47J, M48J, E49J, K50J, L51J, V52J, I53J, P54J, A55J, H56J, V57J, R58J, A59J, Q60J, Y61J, V62J, V63J, L64J, L65J, R66J, R67J, D68J, G69J, D70J, R71J, S72J, R73J, G74J, K75J, R76J, F77J, S78J, Q79J, S80J, F81J, R82J, E83J, V84J, A85J, G86J, R87J, F88J, L89J, A90J, S91J, E92J, A93J, S94J, T95J, H96J, L97J, L98J, V99J, F100J, G101J, M102J, E103J, Q104J, R105J, L106J, P107J, P108J, N109J, S110J, E111J, L112J, V113J, Q114J, A115J, V116J, L117J, R118J, L119J, F120J, Q121J, E122J, P123J, V124J, P125J, Q126J, G127J, A128J, L129J, H130J, R131J, H132J, G133J, R134J, L135J, S136J, P137J, A138J, A139J, P140J, K141J, A142J, R143J, V144J, T145J, V146J, E147J, W148J, L149J, V150J, R151J D152J, D153J, G154J, S155J, N156J, R157J, T158J, S159J, L160J, I161J, D162J, S163J, R164J, L165J, V166J, S167J, V168J, H169J, E170J, S171J, G172J, W173J, K174J, A175J, F176J, D177J, V178J, T179J, E180J, A181J, V182J, N183J, F184J, W185J, Q186J, Q187J, L188J, S189J, R190J, P191, P192J, E193J, P194J, L195J, L196J, V197J, Q198J, V199J, S200J, V201J, Q202J, R203J, E204J, H205J, L206J, G207J, P208J, L209J, A210J, S211J, G212J, A213J, H214J, K215J, L216J, V217J, R218J, F219J, A220J, S221J, Q222J, G223J, A224J, P225J, A226J, G227J, L228J, G229J, E230J, P231J, Q232J, L233J, E234J, L235J, H236J, T237J, L238J, D239J, L240J, R241J, D242J, Y243J, G244J, A245J, Q246J, G247J, D248J, C249J, D250J, P251J, E252J, A253J, P254J, M255J, T256J, E257J, G258J, T259J, R260J, C261J, C262J, R263J, Q264J, E265J, M266J, A288J, Y289J, E290J, C291J, V292J, G293J, T294J, C295J, Q296J, Q297J, P298J, P299J, E300J, A301J, L302J, A303J, F304J, N305J, W306J, P307J, F308J, L309J, G310J, P311J, R312J, Q313J, C314J, I315J, A316J, S317J, V338J, V339J, S340J, L341J, P342J, N343J, M344J, R345J, V346J, Q347J, K348J, C349J, S350J, C351J, A352J, S353J, D354J, G355J, A356J, L357J, V358J, P359J, R360J, R361J, L362J, Q363J, H364J, R365J, P366J, W367J, C368J, I369J, and H370J. The variable “J” is any amino acid whose introduction results in an increase in the electrostatic interaction between the L1 and L3 β hairpin loop structures of the TGF-β4 and a receptor with affinity for a dimeric protein containing the mutant TGF-β4.

The invention also contemplates a number of mutant TGF-β4 subunits in modified forms. These modified forms include mutant TGF-β4 linked to another cystine knot growth factor or a fraction of such a monomer.

In specific embodiments, the mutant TGF-β4 heterodimer comprising at least one mutant subunit or the single chain mutant TGF-β4 subunit analog as described above is functionally active, i.e., capable of exhibiting one or more functional activities associated with the wild-type TGF-β4, such as TGF-β4 receptor binding, TGF-β4 protein family receptor signalling and extracellular secretion. Preferably, the mutant TGF-β4 heterodimer or single chain TGF-β4 analog is capable of binding to the TGF-β4 receptor, preferably with affinity greater than the wild type TGF-β4. Also it is preferable that such a mutant TGF-β4 heterodimer or single chain TGF-β4 analog triggers signal transduction. Most preferably, the mutant TGF-β4 heterodimer comprising at least one mutant subunit or the single chain TGF-β4 analog of the present invention has an in vitro bioactivity and/or in vivo bioactivity greater than the wild type TGF-β4 and has a longer serum half-life than wild type TGF-β4. Mutant TGF-β4 heterodimers and single chain TGF-β4 analogs of the invention can be tested for the desired activity by procedures known in the art.

Mutants of the Human Neurturin

The human neurturin protein contains 197 amino acids as shown in FIG. 18 (SEQ ID No: 17). The invention contemplates mutants of the human neurturin protein comprising single or multiple amino acid substitutions, deletions or insertions, of one, two, three, four or more amino acid residues when compared with the wild type monomer. Furthermore, the invention contemplates mutant human neurturin protein that are linked to another CKGF protein.

The present invention provides mutant neurturin protein L1 hairpin loops having one or more amino acid substitutions between positions 104-129, inclusive, excluding Cys residues, as depicted in FIG. 18 (SEQ ID NO: 17). The amino acid substitutions include G104X, L105X, R106X, E107X, L108X, E109X, V110X, R111X, V112X, S113X, E114X, L115X, G116X, L117X, G118X, Y119X, A120X, S121X, D122X, E123X, T124X, V125X, L126X, F127X, R128X, and Y129X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the neurturin protein where an acidic residue is present, the variable “X” would correspond to a basic amino acid residue. Specific examples of electrostatic charge altering mutations where a basic residue is introduced into the neurturin protein include one or more of the following: E107B, E109B, E114B, D122B, and E123B, wherein “B” is a basic amino acid residue.

Introducing acidic amino acid residues where basic residues are present in the neurturin protein sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following R106Z, R111Z, and R128Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at R106U, E107U, E109U, R111U, E114U, D122U, E123U, and R128U, wherein “U” is a neutral amino acid.

Mutant neurturin protein proteins are provided containing one or more electrostatic charge altering mutations in the L1 hairpin loop amino acid sequence that convert non-charged or neutral amino acid residues to charged residues. Examples of mutations converting neutral amino acid residues to charged residues include: G104Z, L105Z, L108Z, V110Z, V112Z, S113Z, L115Z, G116Z, L117Z, G118Z, Y119Z, A120Z, S121Z, T124Z, V125Z, L126Z, F127Z, Y129Z, G104B, L105B, L108B, V110B, V112B, S113B, L115B, G116B, L117B, G118B, Y119B, A120B, S121B, T124B, V125B, L126B, F127B, and Y129B, wherein “Z” is an acidic amino acid and “B” is a basic amino acid.

Mutant neurturin protein containing mutants in the L3 hairpin loop are also described. These mutant proteins have one or more amino acid substitutions, deletion or insertions, between positions 166 and 193, inclusive, excluding Cys residues, of the L3 hairpin loop, as depicted in FIG. 18 (SEQ ID NO: 17). The amino acid substitutions include: R166X, P167X, T168X, A169X, Y170X, E171X, D172X, E173X, V174X, S175X, F176X, L177X, D178X, A179X, H180X, S181X, R182X, Y183X, H184X, T185X, V186X, H187X, E188X, L189X, S190X, A191X, R192X, and E193X, wherein “X” is any amino acid residue, the substitution of which alters the electrostatic character of the L3 loop.

One set of mutations of the L3 hairpin loop includes introducing one or more basic amino acid residues into the neurturin protein L3 hairpin loop amino acid sequence. For example, when introducing basic residues into the L3 loop of the neurturin protein, the variable “X” of the sequence described above corresponds to a basic amino acid residue. Specific examples of electrostatic charge altering mutations where a basic residue is introduced into the neurturin protein include one or more of the following: E171B, D172B, E173B, E188B, and E193B, wherein “B” is a basic amino acid residue.

The invention further contemplates introducing one or more acidic residues into the amino acid sequence of the neurturin protein L3 hairpin loop. For example, one or more acidic amino acids can be introduced in the sequence of 166-3193 described above, wherein the variable “X” corresponds to an acidic amino acid. Specific examples of such mutations include R166Z, H180Z, R182Z, H184Z, H187Z, and R192Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at R166U, E171U, D172U, E173U, H180U, R182U, H184U, H187U, E188U, R192U, and E193U, wherein “U” is a neutral amino acid.

Mutant neurturin protein proteins are provided containing one or more electrostatic charge altering mutations in the L3 hairpin loop amino acid sequence that convert non-charged or neutral amino acid residues to charged residues. Examples of mutations converting neutral amino acid residues to charged residues include P167Z, T168Z, A169Z, Y170Z, V174Z, S175Z, F176Z, L177Z, A179Z, S181Z, Y183Z, T185Z, V186Z, L189Z, S190Z, A191Z, P167B, T168B, A169B, Y170B, V174B, S175B, F176B, L177B, A179B, S181B, Y183B, T185B, V186B,

L189B, S190B, and A191B, wherein “Z” is an acidic amino acid and “B” is a basic amino acid.

The present invention also contemplate neurturin protein containing mutations outside of said β hairpin loop structures that alter the structure or conformation of those hairpin loops. These structural alterations in turn serve to increase the electrostatic interactions between regions of the β hairpin loop structures of neurturin protein contained in a dimeric molecule, and a receptor having affinity for the dimeric protein. These mutations are found at positions selected from the group consisting of positions 1-103, 130-165, and 194-197 of the neurturin protein.

Specific examples of these mutation outside of the β hairpin L1 and L3 loop structures include, M1J, Q2J, R3J, W4J, K5J, A6J, A7J, A8J, L9J, A10J, S11J, V12J, L13J, C14J, S15J, S16J, V17J, L18J, S19J, I20J, W21J, M22J, C23J, R24J, E25J, G26J, L27J, L28J, L29J, S30J, H31J, R32J, L33J, G34J, P35J, A36J, L37J, V38J, P39J, L40J, H41J, R42J, L43J, P44J, R45J, T46J, L47J, D48J, A49J, R50J, I51J, A52J, R53J, L54J, A55J, Q56J, Y57J, R58J, A59J, L60J, L61J, Q62J, G63J, A64J, P65J, D66J, A67J, M68J, E69J, L70J, R71J, E72J, L73J, T74J, P75J, W76J, A77J, G78J, R79J, P80J, P81J, G82J, P83J, R84J, R85J, R86J, A87J, G88J, P89J, R90J, R91J, R92J, R93J, A94J, R95J, A96J, R97J, L98J, G99J, A100J, R101J, P102J, C103J, C130J, A131J, G132J, A133J, C134J, E135J, A136J, A137J, A138J, R139J, V140J, Y141J, D142J, L143J, G144J, L145J, R146J, R147J, L148J, R149J, Q150J, R151J, R152J, R153J, L154J, R155J, R156J, E157J, R158J, V159J, R160J, A161J, Q162J, P163J, C164J, C165J, C194J, A195J, C196J, and V197J. The variable “J” is any amino acid whose introduction results in an increase in the electrostatic interaction between the L1 and L3 β hairpin loop structures of the neurturin protein and a receptor with affinity for a dimeric protein containing the mutant neurturin protein monomer.

The invention also contemplates a number of neurturin protein in modified forms. These modified forms include neurturin protein linked to another cystine knot growth factor monomer or a fraction of such a monomer.

In specific embodiments, the mutant neurturin protein heterodimer comprising at least one mutant subunit or the single chain neurturin protein analog as described above is functionally active, i.e., capable of exhibiting one or more functional activities associated with the wild-type neurturin protein, such as neurturin protein receptor binding, neurturin protein protein family receptor signalling and extracellular secretion. Preferably, the mutant neurturin protein heterodimer or single chain neurturin protein analog is capable of binding to the neurturin protein receptor, preferably with affinity greater than the wild type neurturin protein. Also it is preferable that such a mutant neurturin protein heterodimer or single chain neurturin protein analog triggers signal transduction. Most preferably, the mutant neurturin protein heterodimer comprising at least one mutant subunit or the single chain neurturin protein analog of the present invention has an in vitro bioactivity and/or in vivo bioactivity greater than the wild type neurtlrin protein and has a longer serum half-life than wild type neurturin protein. Mutant neurturin protein heterodimers and single chain neurturin protein analogs of the invention can be tested for the desired activity by procedures known in the art.

Mutants of the Human Inhibin A α protein

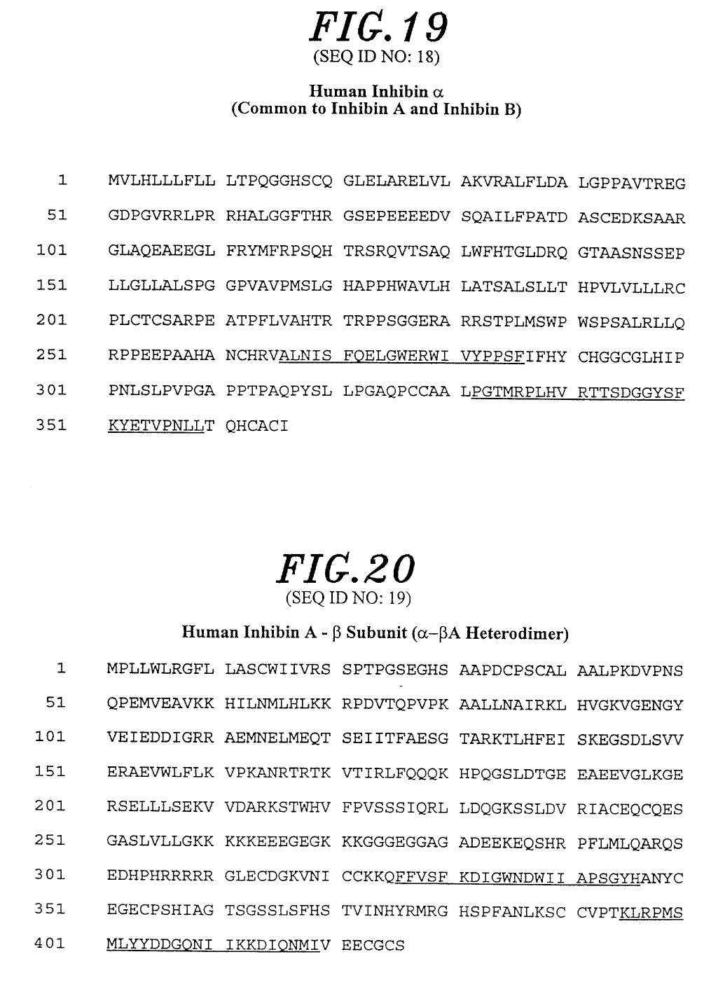

The human inhibin A α protein contains 366 amino acids as shown in FIG. 19 (SEQ ID No: 18). The invention contemplates mutants of the human inhibin A α protein comprising single or multiple amino acid substitutions, deletions or insertions, of one, two, three, four or more amino acid residues when compared with the wild type monomer. Furthermore, the invention contemplates mutant human inhibin A α protein that are linked to another CKGF protein.

The present invention provides mutant inhibin A α protein L1 hairpin loops having one or more amino acid substitutions between positions 266-286, inclusive, excluding Cys residues, as depicted in FIG. 19 (SEQ ID NO: 18). The amino acid substitutions include: A266X, L267X, N268X, I269X, S270X, F271X, Q272X, E273X, L274X, G275X, W276X, E277X, R278X, W279X, I280X, V281X, Y282X, P283X, P284X, S285X, and F286X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the inhibin A α protein where an acidic residue is present, the variable “X” would correspond to a basic amino acid residue. Specific examples of electrostatic charge altering mutations where a basic residue is introduced into the inhibin A α protein include one or more of the following: E273B and E277B, wherein “B” is a basic amino acid residue.

Introducing acidic amino acid residues where basic residues are present in the inhibin A α protein sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following R278Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at E273U, E277U, and R278U, wherein “U” is a neutral amino acid.

Mutant inhibin A α protein proteins are provided containing one or more electrostatic charge altering mutations in the L1 hairpin loop amino acid sequence that convert non-charged or neutral amino acid residues to charged residues. Examples of mutations converting neutral amino acid residues to charged residues include: of A266Z, L267Z, N268Z, I269Z, S270Z, F271Z, Q272Z, L274Z, G275Z, W276Z, W279Z, I280Z, V281Z, Y282Z, P283Z, P284Z, S285Z, F286Z, A266B, L267B, N268B, I269B, S270B, F271B, Q272B, L274B, G275B, W276B, W279B, I280B, V281B, Y282B, P283B, P284B, S285B, and F286B, wherein “Z” is an acidic amino acid and “B” is a basic amino acid.

Mutant inhibin A α protein containing mutants in the L3 hairpin loop are also described. These mutant proteins have one or more amino acid substitutions, deletion or insertions, between positions 332 and 359, inclusive, excluding Cys residues, of the L3 hairpin loop, as depicted in FIG. 19 (SEQ ID NO: 18). The amino acid substitutions include: P332X, G333X, T334X, M335X, R336X, P337X, L338X, H339X, V340X, R341X, T342X, T343X, S344X, D345X, G346X, G347X, Y348X, S349X, F350X, K351X, Y352X, E353X, T354X, V355X, P356X, N357X, L358X, and L359X, wherein “X” is any amino acid residue, the substitution of which alters the electrostatic character of the L3 loop.

One set of mutations of the L3 hairpin loop includes introducing one or more basic amino acid residues into the inhibin A α protein L3 hairpin loop amino acid sequence. For example, when introducing basic residues into the L3 loop of the inhibin A α protein, the variable “X” of the sequence described above corresponds to a basic amino acid residue. Specific examples of electrostatic charge altering mutations where a basic residue is introduced into the inhibin A α protein include one or more of the following: D345B and E353B, wherein “B” is a basic amino acid residue.

The invention further contemplates introducing one or more acidic residues into the amino acid sequence of the inhibin A α protein L3 hairpin loop. For example, one or more acidic amino acids can be introduced in the sequence of 332-359 described above, wherein the variable “X” corresponds to an acidic amino acid. Specific examples of such mutations include R336Z, H339Z, R341Z, and K351Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at R336U, H339U, R341U, D345U, K351U, and E353U, wherein “U” is a neutral amino acid.

Mutant inhibin A α protein proteins are provided containing one or more electrostatic charge altering mutations in the L3 hairpin loop amino acid sequence that convert non-charged or neutral amino acid residues to charged residues. Examples of mutations converting neutral amino acid residues to charged residues include of P332Z, G333Z, T334Z, M335Z, P337Z, L338Z, V340Z, T342Z, T343Z, S344Z, G346Z, G347Z, Y348Z, S349Z, F350Z, Y352Z, T354Z, V355Z, P356Z, N357Z, L358Z, L359Z, P332B, G333B, T334B, M335B, P337B, L338B, V340B, T342B, T343B, S344B, G346B, G347B, Y348B, S349B, F350B, Y352B, T354B, V355B, P356B, N357B, L358B, and L359B, wherein “Z” is an acidic amino acid and “B” is a basic amino acid.

The present invention also contemplate inhibin A α protein containing mutations outside of said β hairpin loop structures that alter the structure or conformation of those hairpin loops. These structural alterations in turn serve to increase the electrostatic interactions between regions of the β hairpin loop structures of inhibin A α protein contained in a dimeric molecule, and a receptor having affinity for the dimeric protein. These mutations are found at positions selected from the group consisting of positions 1-265, 287-331, and 360-366 of the inhibin A α protein.

Specific examples of these mutation outside of the β hairpin L1 and L3 loop structures include, M1J, V2J, L3J, H4J, L5J, L6J, L7J, F8J, L9J, L10J, L11J, T12J, P13J, Q14J, G15J, G16J, H17J, S18J, C19J, Q20J, G21J, L22J, E23J, L24J, A25J, R26J, E27J, L28J, V29J, L30J, A31J, K32J, V33J, R34J, A35J, L36J, F37J, L38J, D39J, A40J, L41J, G42J, P43J, P44J, A45J, V46J, T47J, R48J, E49J, G50J, G51J, D52J, P53J, G54J, V55J, R56J, R57J, L58J, P59J, R60J, R61J, H62J, A63J, L64J, G65J, G66J, F67J, T68J, H69J, R70J, G71J, S72J, E73J, P74J, E75J, E76J, E77J, E78J, D79J, V80J, S81J, Q82J, A83J, I84J, L85J, F86J, P87J, A88J, T89J, D90J, A91J, S92J, C93J, E94J, D95J, K96J, S97J, A98J, A99J, R100J, G101J, L102J, A103J, Q104J, E105J, A106J, E107J, E108J, G109J, L110J, F111J, R112J, Y113J, M114J, F115J, R116J, P117J, S118J, Q119J, H120J, T121J, R122J, S123J, R124J, Q125J, V126J, T127J, S128J, A129J, Q130J, L131J, W132J, F133J, H134J, T135J, G136J, L137J, D138J, R139J, Q140J, G141J, T142J, A143J, A144J, S145J, N146J, S147J, S148J, E149J, P150J, L151J, L152J, G153J, L154J, L155J, A156J, L157J, S158J, P159J, G160J, G161J, P162J, V163J, A164J, V165J, P166J, M167J, S168J, L169J, G170J, H171J, A172J, P173J, P174J, H175J, W176J, A177J, V178J, L179J, H180J, L181J, A182J, T183J, S184J, A185J, L186J, S187J, L188J, L189J, T190J, H191J, P192J, V193J, L194J, V195J, L196J, L197J, L198J, R199J, C200J, P201J, L202J, C203J, T204J, C205J, S206J, A207J, R208J, P209J, E210J, A211J, T212J, P213J, F214J, L215J, V216J, A217J, H218J, T219J, R220J, T221J, R222J, P223J, P224J, S225J, G226J, G227J, E228J, R229J, A230J, R231J, R232J, S233J, T234J, P235J, L236J, M237J, S238J, W239J, P240J, W241J, S242J, P243J, S244J, A245J, L246J, R247J, L248J, L249J, Q250J, R251J, P252J, P253J, E254J, E255J, P256J, A257J, A258J, H259J, A260J, N261J, C262J, H263J, R264J, V265J, I287J, F288J, H289J, Y290J, C291J, H292J, G293J, G294J, C295J, G296J, L297J, H298J, I299J, P300J, P301J, N302J, L303J, S304J, L305J, L305J, P306J, V307J, P308J, G309J, A310J, P311J, P312J, T313J, P314J, A315J, Q316J, P317J, Y318J, S319J, L320J, L321J, P322J, G323J, A324J, Q325J, P326J, C327J, C328J, A329J, A330J, L331J, T360J, Q361J, H362J, C363J, A364J, C365J, and I366J. The variable “J” is any amino acid whose introduction results in an increase in the electrostatic interaction between the L1 and L3 β hairpin loop structures of the inhibin A α protein and a receptor with affinity for a dimeric protein containing the mutant inhibin A α protein monomer.

The invention also contemplates a number of inhibin A α protein in modified forms. These modified forms include inhibin A α protein linked to another cystine knot growth factor monomer or a fraction of such a monomer.

In specific embodiments, the mutant inhibin A α protein heterodimer comprising at least one mutant subunit or the single chain inhibin A α protein analog as described above is functionally active, i.e., capable of exhibiting one or more functional activities associated with the wild-type inhibin A α protein, such as inhibin A α protein receptor binding, inhibin A α protein protein family receptor signalling and extracellular secretion. Preferably, the mutant inhibin A α protein heterodimer or single chain inhlbin A α protein analog is capable of binding to the inhibin A α protein receptor, preferably with affinity greater than the wild type inhibin A α protein. Also it is preferable that such a mutant inhibin A α protein heterodimer or single chain inhibin A α protein analog triggers signal transduction. Most preferably, the mutant inhibin A α protein heterodimer comprising at least one mutant subunit or the single chain inhibin A α protein analog of the present invention has an in vitro bioactivity and/or in vivo bioactivity greater than the wild type inhibin A α protein and has a longer serum half-life than wild type inhibin A α protein. Mutant innibin A α protein heterodimers and single chain inhibin A α protein analogs of the invention can be tested for the desired activity by procedures known in the art.

Mutants of the Human Inhibin A β Subunit

The human human inhibin A β subunit contains 426 amino acids as shown in FIG. 20 (SEQ ID No: 19). The invention contemplates mutants of the human human inhibin A β subunit comprising single or multiple amino acid substitutions, deletions or insertions, of one, two, three, four or more amino acid residues when compared with the wild type monomer. Furthermore, the invention contemplates mutant human human inhibin A β subunit that are linked to another CKGF protein.

The present invention provides mutant human inhibin A β subunit L1 hairpin loops having one or more amino acid substitutions between positions 326 and 346, inclusive, excluding Cys residues, as depicted in FIG. 20 (SEQ ID NO: 19). The amino acid substitutions include: F326X, F327X, V328X, S329X, F330X, K331X, D332X, I333X, G334X, W335X, N336X, D337X, W338X, I339X, I340X, A341X, P342X, S343X, G344X, Y345X, and H346X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the human inhibin A β subunit where an acidic residue is present, the variable “X” would correspond to a basic amino acid residue. Specific examples of electrostatic charge altering mutations where a basic residue is introduced into the human inhibin A β subunit include one or more of the following: D332B and D337B wherein “B” is a basic amino acid residue.

Introducing acidic amino acid residues where basic residues are present in the human inhibin A β subunit sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following K331Z and H346Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at K331U, D332U, D337U, wherein “U” is a neutral amino acid.

Mutant human inhibin A β subunit proteins are provided containing one or more electrostatic charge altering mutations in the L1 hairpin loop amino acid sequence that convert non-charged or neutral amino acid residues to charged residues. Examples of mutations converting neutral amino acid residues to charged residues include F326Z, F327Z, V328Z, S329Z, F330Z, I333Z, G334Z, W335Z, N336Z, W338Z, I339Z, I340Z, A341Z, P342Z, S343Z, G344Z, Y345Z, F326B, F327B, V328B, S329B, F330B, I333B, G334B, W335B, N336B, W338B, I339B, I340B, A341B, P342B, S343B, G344B, and Y345B, wherein “Z” is an acidic amino acid and “B” is a basic amino acid.

Mutant human inhibin A β subunit containing mutants in the L3 hairpin loop are also described. These mutant proteins have one or more amino acid substitutions, deletion or insertions, between positions 395 and 419, inclusive, excluding Cys residues, of the L3 hairpin loop, as depicted in FIG. 20 (SEQ ID NO: 19). The amino acid substitutions include: K395X, L396X, R397X, P398X, M399X, S400X, M401X, L402X, Y403X, Y404X, D405X, D406X, G407X, Q408X, N409X, I410X, I411X, K412X, K413X, D414X, I415X, Q416X, N417X, M418X, and I419X, wherein “X” is any amino acid residue, the substitution of which alters the electrostatic character of the L3 loop.

One set of mutations of the L3 hairpin loop includes introducing one or more basic amino acid residues into the human inhibin A β subunit L3 hairpin loop amino acid sequence. For example, when introducing basic residues into the L3 loop of the human inhibin A β subunit, the variable “X” of the sequence described above corresponds to a basic amino acid residue. Specific examples of electrostatic charge altering mutations where a basic residue is introduced into the human inhibin A β subunit include one or more of the following: D405B, D406B, and D414B, wherein “B” is a basic amino acid residue.

The invention further contemplates introducing one or more acidic residues into the amino acid sequence of the human inhibin A β subunit L3 hairpin loop. For example, one or more acidic amino acids can be introduced in the sequence of 395-419 described above, wherein the variable “X” corresponds to an acidic amino acid. Specific examples of such mutations include K395Z, R397Z, K412Z, and K413Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K395U, R397U, D405, D406, K412U, K413U, and D414U, wherein “U” is a neutral amino acid.

Mutant human inhibin A β subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include L396Z, P398Z, M399Z, S400Z, M401Z, L402Z, Y403Z, Y404Z, G407Z, P408Z, N409Z, I410Z, I411Z, I415Z, Q416Z, N417Z, M418Z, I419Z, L396B, P398B, M399B, S400B, M401B, L402B, Y403B, Y404B, G407B, P408B, N409B, I410B, I411B, I415B, Q416B, N417B, M418B, and i419B, wherein “Z” is an acidic amino acid and “B” is a basic amino acid.

The present invention also contemplate human inhibin A β subunit containing mutations outside of said β hairpin loop structures that alter the structure or confornation of those hairpin loops. These structural alterations in turn serve to increase the electrostatic interactions between regions of the β hairpin loop structures of human inhibin A β subunit contained in a dimeric molecule, and a receptor having affinity for the dimeric protein. These mutations are found at positions selected from the group consisting of positions 1-325, 347-394, and 420-426 of the human inhibin A β subunit monomer.

Specific examples of these mutation outside of the β hairpin L1 and L3 loop structures include, M1J, P2J, L3J, L4J, W5J, L6J, R7J, G8J, F9J, L10J, L11J, A12J, S14J, W15J, I16J, I17J, V18J, R19J, S20J, S21J, P22J, T23J, P24J, G25J, S26J, E27J, G28J, H29J, S30J, A31J, A32J, P33J, D34J, C35J, P36J, S37J, C38J, A39J, L40J, A41J, A42J, L43J, P44J, K45J, D46J, V47J, P48J, N49J, S50J, Q51J, P52J, E53J, M54J, V55J, E56J, A57J, V58J, K59J, K60J, H61J, I62J, L63J, N64J, M65J, L66J, H67J, L68J, K69J, K70J, R71J, P72J, D73J, V74J, T75J, Q76J, P77J, V78J, P79J, K80J, A81J, A82J, L83J, L84J, N85J, A86J, I87J, K89J, L90J, H91J, V92J, G93J, K94J, V95J, G96J, E97J, N98J, G99J, Y100J, V101J, E102J, I103J, E104J, D105J, D106J, I107J, G108J, R109J, R110J, A111J, E112J, M113J, N114J, E115J, L116J, M117J, E118J, Q119J, T120J, S121J, E122J, I123J, I124J, T125J, F126J, A127J, E128J, S129J, G130J, T131J, A132J, R133J, K134J, T135J, L136J, H137J, F138J, E139J, I140J, S141J, K142J, E143J, G144J, S145J, D146J, L147J, S148J, V149J, V150J, E151J, R152J, A153J, E154J, V155J, W156J, L157J, F158J, L159J, K160J, V161J, P162J, K163J, A164J, N165J, R166J, T167J, R168J, T169J, K170J, V171J, T172J, I173J, R174J, L175J, F176J, Q177J, Q178J, Q179J, K180J, H181J, P182J, Q183J, G184J, S185J, L186J, D187J, T188J, G189J, E190J, E191J, A192J, E193J, E194J, V195J, G196J, L197J, K198J, G199J, E200J, R201J, S202J, E203J, L204J, L205J, L206J, S207J, E208J, K209J, V210J, V211J, D212J, A213J, R214J, K215J, S216J, T217J, W218J, H219J, V220J, F221J, P222J, V223J, S224J, S225J, S226J, I227J, Q228J, R229J, L230J, L231J, D232J, Q233J, G234J, K235J, S236J, S237J, L238J, D239J, V240J, R241J, I242J, A243J, C244J, E245J, Q246J, C247J, Q248J, E249J, S250J, G251J, A252J, S253J, L254J, V255J, L256J, L257J, G258J, K259J, K260J, K261J, K262J, K263J, E264J, E265J, E266J, G267J, E268J, G269J, K270J, K271J, K272J, G273J, G274J, G275J, E276J, G277J, G278J, A279J, G280J, A281J, D282J, E283J, E284J, K285J, E286J, Q287J, S288J, H289J, R290J, P291J, F292J, L293J, M294J, L295J, Q296J, A297J, R298J, Q299J, S300J, E301J, D302J, H303J, P304J, H305J, R306J, R307J, R308J, R309J, R310J, G311J, L312J, E313J, C314J, D315J, G316J, K317J, V318J, N319J, I320J, C321J, C322KJ, 323J, K324J, Q325J, A347J, N348J, Y349J, C350J, E351J, G352J, E353J, C354J, P355J, S356J, H357J, I358J, A359J, G360J, T361J, S362J, G363J, S364J, S365J, L366J, S367J, F368J, H369J, S370J, T371J, V372J, I373J, N374J, H375J, Y376J, R377J, M378J, R379GJ, 380J, H381J, S382J, P383J, F384J, A385J, N386J, L387J, K388J, S389J, C390J, C391J, V392J, P393J, T394J, V420J, E421J, E422J, C423J, G424J, C425J, and S426J. The variable “J” is any amino acid whose introduction results in an increase in the electrostatic interaction between the L1 and L3 β hairpin loop structures of the human inhibin A β subunit and a receptor with affinity for a dimeric protein containing the mutant human inhibin A β subunit monomer.

The invention also contemplates a number of human inhibin A β subunit in modified forms. These modified forms include human inhibin A β subunit linked to another cystine knot growth factor or a fraction of such a monomer.

In specific embodiments, the mutant human inhibin A β subunit heterodimer comprising at least one mutant subunit or the single chain human inhibin A β subunit analog as described above is functionally active, i.e., Capable of exhibiting one or more functional activities associated with the wild-type human inhibin A β subunit, such as human inhibin A β subunit receptor binding, human inhibin A β subunit protein family receptor signalling and extracellular secretion. Preferably, the mutant human inhibin A β subunit heterodimer or single chain human inhibin A β subunit analog is capable of binding to the human inhibin A β subunit receptor, preferably with affinity greater than the wild type human inhibin A β subunit. Also it is preferable that such a mutant human inhibin A β subunit heterodimner or single chain human inhibin A β subunit analog triggers signal transduction. Most preferably, the mutant human inhibin A β subunit heterodimer comprising at least one mutant subunit or the single chain human inhibin A β subunit analog of the present invention has an in vitro bioactivity and/or in vivo bioactivity greater than the wild type human inhibin A β subunit and has a longer serum half-life than wild type human inhibin A β subunit. Mutant human inhibin A β subunit heterodimers and single chain human inhibin A β subunit analogs of the invention can be tested for the desired activity by procedures known in the art.

Mutants of the Human Human Inhibin B β Subunit

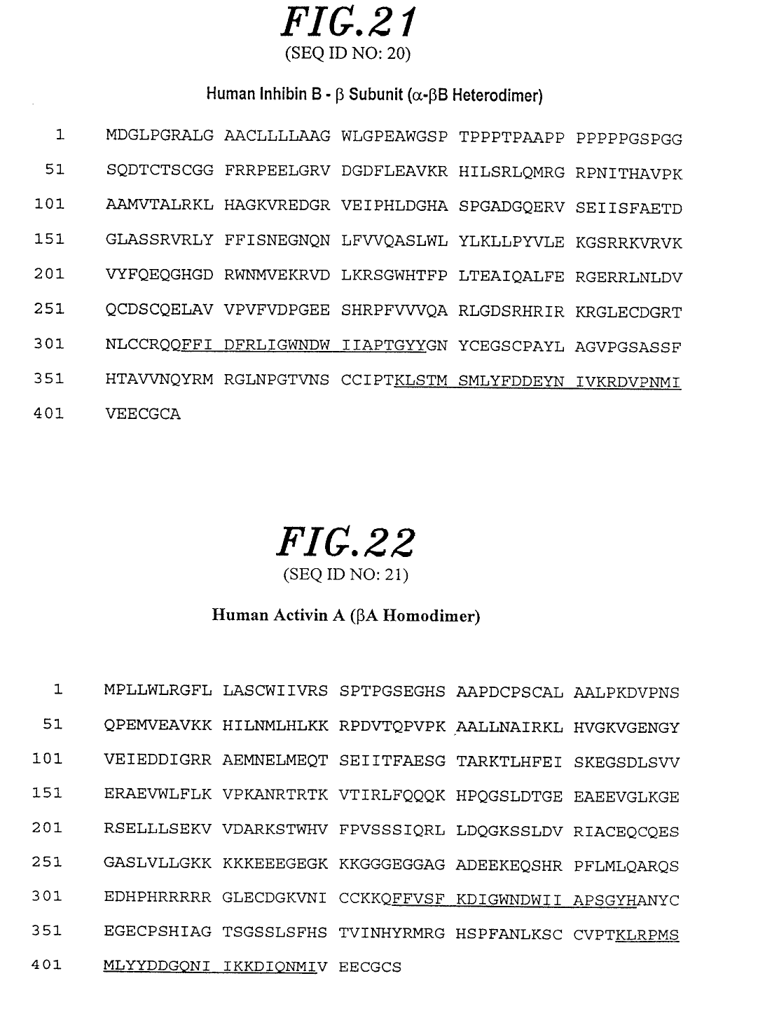

The human human inhibin B β subunit contains 407 amino acids as shown in FIG. 21 (SEQ ID No: 20). The invention contemplates mutants of the human human inhibin B β subunit comprising single or multiple amino acid substitutions, deletions or insertions, of one, two, three, four or more amino acid residues when compared with the wild type monomer. Furthermore, the invention contemplates mutant human human inhibin B β subunit that are linked to another CKGF protein.

The present invention provides mutant human inhibin B β subunit L1 hairpin loops having one or more amino acid substitutions between positions 308 and 328, inclusive, excluding Cys residues, as depicted in FIG. 21 (SEQ ID NO: 20). The amino acid substitutions include: F308X, F309X, I310X, D311X, F312X, R313X, L314X, I315X, G316X, W317X, N318X, D319X, W320X, I321X, I322X, A323X, P324X, T325X, G326X, Y327X, and Y328X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the human inhibin B β subunit where an acidic residue is present, the variable “X” would correspond to a basic amino acid residue. Specific examples of electrostatic charge altering mutations where a basic residue is introduced into the human inhibin B β subunit include one or more of the following: D311B and D319B wherein “B” is a basic amino acid residue.

Introducing acidic amino acid residues where basic residues are present in the human inhibin B β subunit sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following R313Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D311U, R313U, and D319U, wherein “U” is a neutral amino acid.

Mutant human inhibin B β subunit proteins are provided containing one or more electrostatic charge altering mutations in the L1 hairpin loop amino acid sequence that convert non-charged or neutral amino acid residues to charged residues. Examples of mutations converting neutral amino acid residues to charged residues include: F308Z, F309Z, I310Z, F312Z, L314Z, I315Z, G316Z, W317Z, N318Z, W320Z, I321Z, I322Z, A323Z, P324Z, T325Z, G326Z, Y327Z, Y328Z, F308B, F309B, I310B, F312B, L314B, I315B, G316B, W317B, N318B, W320B, I321B, I322B, A323B, P324B, T325B, G326B, Y327B, and Y328B, wherein “Z” is an acidic amino acid and “B” is a basic amino acid.

Mutant human inhibin B β subunit containing mutants in the L3 hairpin loop are also described. These mutant proteins have one or more amino acid substitutions, deletion or insertions, between positions 376 and 400, inclusive, excluding Cys residues, of the L3 hairpin loop, as depicted in FIG. 21 (SEQ ID NO: 20). The amino acid substitutions include: K376X, L377X, S378X, T379X, M380X, S381X, M382X, L383X, Y384X, F385X, D386X, D387X, E388X Y389X, N390X, I391X, V392X, K393X, R394X, D395X, V396X, P397X, N398X, M399X, and I400X, wherein “X” is any amino acid residue, the substitution of which alters the electrostatic character of the L3 loop.

One set of mutations of the L3 hairpin loop includes introducing one or more basic amino acid residues into the human inhibin B β subunit L3 hairpin loop amino acid sequence. For example, when introducing basic residues into the L3 loop of the human inhibin B β subunit, the variable “X” of the sequence described above corresponds to a basic amino acid residue. Specific examples of electrostatic charge altering mutations where a basic residue is introduced into the human inhibin B β subunit include one or more of the following: D386B, D387B, E388B, and D395B, wherein “B” is a basic amino acid residue.

The invention further contemplates introducing one or more acidic residues into the amino acid sequence of the human inhibin B β subunit L3 hairpin loop. For example, one or more acidic amino acids can be introduced in the sequence of 376-400 described above, wherein the variable “X” corresponds to an acidic amino acid. Specific examples of such mutations include K376Z, K393Z, and K394Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K376U, D386U, D387U, E388U, K393U, R394U, and D395U, wherein “U” is a neutral amino acid.

Mutant human inhibin B β subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include, L377Z, S378Z, T379Z, M380Z, S381Z, M382Z, L383Z, Y384Z, F385Z, Y389Z, N390Z, I391Z, V392Z, V396Z, P397Z, N398Z, M399Z, I400Z, L377B, S378B, T379B, M380B, S381B, M382B, L383B, Y384B, F385B, Y389B, N390B, I391B, V392B, V396B, P397B, N398B, M399B, and I400B, wherein “Z” is an acidic amino acid and “B” is a basic amino acid.

The present invention also contemplate human inhibin B β subunit containing mutations outside of said β hairpin loop structures that alter the structure or conformation of those hairpin loops. These structural alterations in turn serve to increase the electrostatic interactions between regions of the β hairpin loop structures of human inhibin B β subunit contained in a dimeric molecule, and a receptor having affinity for the dimeric protein. These mutations are found at positions selected from the group consisting of positions 1-307, 329-375, and 401-407 of the human inhibin B β subunit monomer.

Specific examples of these mutation outside of the β hairpin L1 and L3 loop structures include, M1J, D2J, G3J, L4J, P5J, G6J, R7J, A8J, L9J, G10J, A11J, A12J, C13J, L14J, L15J, L16J, L17J, A18J, A19J, G20J, W21J, L22J, G23J, P24J, E25J, A26J, W27J, G28J, S29J, P30J, T31J, P32J, P33J, P34J, T35J, P36J, A37J, A38J, P39J, P40J, P41J, P42J, P43J, P44J, P45J, G46J, S47J, P48J, G49J, G50J, S51J, Q52J, D53J, T54J, C55J, T56J, S57J, C58J, G59J, G60J, F61J, R62J, R63J, P64J, E65J, E66J, L67J, G68J, R69J, V70J, D71J, G72J, D73J, F74J, L75J, E76J, A77J, V78J, K79J, R80J, H81J, I82J, L83J, S84J, R85J, L86J, Q87J, M88J, R89J, G90J, R91J, P92J, N93J, I94J, T95J, H96J, A97J, V98J, P99J, K100J, A101J, A102J, M103J, V104J, T105J, A106J, L107J, R108J, K109J, L110J, H111J, A112J, G113J, K114J, V115J, R116J, E117J, D118J, G119J, R120J, V121J, E122J, I123J, P124J, H125J, L126J, D127J, G128J, H129J, A130J, S131J, P132J, G133J, A134J, D135J, G136J, Q137J, E138J, R139J, V140J, S141J, E142J, I143J, I144J, S145J, F146J, A147J, E148J, T149J, D150J, G151J, A153J, S154J, S155J, R156J, V157J, R158J, L159J, Y160J, F161J, F162J, I163J, S164J, N165J, E166J, G167J, N168J, Q169J, N170J, L171J, F172J, V173J, V174J, Q175J, A176J, S177J, L178J. W179J, L180J, Y181J, L182J, K183J, L184J, L185J, P186J, Y187J, V188J, L189J, E190J, K191J, G192J, S193J, R194J, R195J, K196J, V197J, R198J, V199J, K200J, V201J, Y202J, F203J, Q204J, E205J, Q206J, G207J, H208J, G209J, D210J, R211J, W212J, N213J, M214J, V215J, E216J, K217J, R218J, V219J, D220J, L221J, K222J, R223J, S224J, G225J, W226J, H227J, T228J, F229J, P230J, L231J, T232J, E233J, A234J, I235J, Q236J, A237J, L238J, F239J, E240J, R241J, G242J, E243J, R244J, R245J, L246J, N247J, L248J, D249J, V250J, Q251J, C252J, D253J, S254J, C255J, Q256J, E257J, L258J, A259J, V260J, V261J, P262J, V263J, F264J, V265J, D266J, P267J, G268J, E269J, E270J, S271J, H272J, R273J, P274J, F275J, V276J, V277J, V278J, Q279J, A280J, R281J, L282J, G283J, D284J, S285J, R286J, H287J, R288J, I289J, R290J, K291J, R292J, G293J, L294EJ, 295CJ, 296J, D297J, G298J, R299J, T300J, N301J, L302J, C303J, C304J, R305J, Q306J, Q307J, G329J, N330J, Y331J, C332J, E333J, G334J, S335J, C336J, P337J, A338J, Y339J, L340J, A341J, G342J, V343J, P344J, G345J, S346J, A347J, S348J, S349J, F350J, H351J, T352J, A353J, V354J, V355J, N356J, Q357J, Y358J, R359J, M360J, R361J, G362J, L363J, N364J, P365J, G366J, T367J, V368J, N369J, S370J, C371J, C372J, I373J, P374J, T375J, V401J, E402J, E403J, C404J, G405J, C406J, and A407J. The variable “J” is any amino acid whose introduction results in an increase in the electrostatic interaction between the L1 and L3 β hairpin loop structures of the human inhibin B β subunit and a receptor with affinity for a dimeric protein containing the mutant human inhibin B β subunit monomer.

The invention also contemplates a number of human inhibin B β subunit in modified forms. These modified forms include human inhibin B β subunit linked to another cystine knot growth factor or a fraction of such a monomer.

In specific embodiments, the mutant human inhibin B β heterodimer comprising at least one mutant subunit or the single chain human inhibin B β analog as described above is functionally active, i.e., capable of exhibiting one or more functional activities associated with the wild-type human inhibin B β, such as human inhibin B β receptor binding, human inhibin B β protein family receptor signalling and extracellular secretion. Preferably, the mutant human inhibin B β heterodimer or single chain human inhibin B β analog is capable of binding to the human inhibin B β receptor, preferably with affinity greater than the wild type human inhibin B β. Also it is preferable that such a mutant human inhibin B β heterodimer or single chain human inhibin B β analog triggers signal transduction. Most preferably, the mutant human inhibin B β heterodimer comprising at least one mutant subunit or the single chain human inhibin B β analog of the present invention has an in vitro bioactivity and/or in vivo bioactivity greater than the wild type human inhibin B β and has a longer serum half-life than wild type human inhibin B β. Mutant human inhibin B β heterodimers and single chain human inhibin B β analogs of the invention can be tested for the desired activity by procedures known in the art.

Mutants of the Human Activin A Subunit

The human activin A subunit contains 426 amino acids as shown in FIG. 22 (SEQ ID No: 21). The invention contemplates mutants of the human activin A subunit comprising single or multiple amino acid substitutions, deletions or insertions, of one, two, three, four or more amino acid residues when compared with the wild type monomer. Furthermore, the invention contemplates mutant human activin A subunit that are linked to another CKGF protein.

The present invention provides mutant human activin A subunit L1 hairpin loops having one or more amino acid substitutions between positions 326 and 346, inclusive, excluding Cys residues, as depicted in FIG. 22 (SEQ ID NO: 21). The amino acid substitutions include: F326X, F327X, V328X, S329X, F330X, K331X, D332X, I333X, G334X, W335X, N336X, D337X, W338X, I339X, I340X, A341X, P342X, S343X, G344X, Y345X, and H346X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the human activin A subunit monomer where an acidic residue is present, the variable “X” would correspond to a basic amino acid residue. Specific examples of electrostatic charge altering mutations where a basic residue is introduced into the human activin A subunit monomer include one or more of the following: K331B and H346B, wherein “B” is a basic amino acid residue.

Introducing acidic amino acid residues where basic residues are present in the human activin A subunit monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: D332Z and D337Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at K331U, D332U, D337U, and H346U, wherein “U” is a neutral amino acid.

Mutant human activin A subunit monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: F326Z, F327Z, V328Z, S329Z, F330Z, I333Z, G334Z, W335Z, N336Z, W338Z, I339Z, I340Z, A341Z, P342Z, S343Z, G344Z, Y345Z, F326B, F327B, V328B, S329B, F330B, I333B, G334B, W335B, N336B, W338B, I339B, I340B, A341B, P342B, S343B, G344B, and Y345B, wherein “Z” is an acidic amino acid and “B” is a basic amino acid.

Mutant human activin A subunit containing mutants in the L3 hairpin loop are also described. These mutant proteins have one or more amino acid substitutions, deletion or insertions, between positions 395 and 419, inclusive, excluding Cys residues, of the L3 hairpin loop, as depicted in FIG. 22 (SEQ ID NO: 21). The amino acid substitutions include: K395X, L396X, R397X, P398X, M399X, S400X, M401X, L402X, Y403X, Y404X, D405X, D406X, G407X, Q408X, N409X, I410X, I411X, K412X, K413X, D414X, I415X, Q416X, N417X, M418X I419X, wherein “X” is any amino acid residue, the substitution of which alters the electrostatic character of the L3 loop.

One set of mutations of the L3 hairpin loop includes introducing one or more basic amino acid residues into the human activin A subunit L3 hairpin loop amino acid sequence. For example, when introducing basic residues into the L3 loop of the human activin A subunit, the variable “X” of the sequence described above corresponds to a basic amino acid residue. Specific examples of electrostatic charge altering mutations where a basic residue is introduced into the human activin A subunit include one or more of the following: D405B, D406B, and D414B, wherein “B” is a basic amino acid residue.

The invention further contemplates introducing one or more acidic residues into the amino acid sequence of the human activin A subunit L3 hairpin loop. For example, one or more acidic amino acids can be introduced in the sequence of 395-419 described above, wherein the variable “X” corresponds to an acidic amino acid. Specific examples of such mutations include K395Z, R397Z, K412Z, and K413Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K395U, R397U, D405U, D406U, K412U, K413U, and D414U, wherein “U” is a neutral amino acid.

Mutant human activin A subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include, L396Z, P398Z, M399Z, S400Z, M401Z, L402Z, Y403Z, Y404Z, G407Z, Q408Z, N409Z, I410Z, I411Z, I415Z, Q416Z, N417Z, M418Z, I419Z, L396B, P398B, M399B, S400B, M401B, L402B, Y403B, Y404B, G407B, Q408B, N409B, I410B, I411B, I415B, Q416B, N417B, M418B, and I419B, wherein “Z” is an acidic amino acid and “B” is a basic amino acid.

The present invention also contemplate human activin A subunit containing mutations outside of said β hairpin loop structures that alter the structure or conformation of those hairpin loops. These structural alterations in turn serve to increase the electrostatic interactions between regions of the β hairpin loop structures of human activin A subunit contained in a dimeric molecule, and a receptor having affinity for the dimeric protein. These mutations are found at positions selected from the group consisting of positions 1-325, 347-394, and 420-426 of the human activin A subunit monomer.

Specific examples of these mutation outside of the β hairpin L1 and L3 loop structures include, M1J, P2J, L3J, L4J, W5J, L6J, R7J, G8J, F9J, L10J, L11J, A12J, S13J, C14J, W15J, I16J, I17J, V18J, R19J, S20J, S21J, P22J, T23J, P24J, G25J, S26J, E27J, G28J, H29J, S30J, A31J, A32J, P33J, D34J, C35J, P36J, S37J, C38J, A39J, L40J, A41J, A42J, L43J, P44J, K45J, D46J, V47J, P48J, N49J, S50J, Q51J, P52J, E53J, M54J, V55J, E56J, A57J, V58J, K59J, K60J, H61J, I62J, L63J, N64J, M65J, L66J, H67J, L68J, K69J, K70J, R71J, P72J, D73J, V74J, T75J, Q76J, P77J, V78J, P79J, K80J, A81J, A82J, L83J, L84J, N85J, A86J, I87J, R88J, K89J, L90J, H91J, V92J, G93J, K94J, V95J, G96J, E97J, N98J, G99J, Y100J, V101J, E102J, I103J, E104J, D105J, D106J, I107J, G108J, R109J, R110J, A111J, E112J, M113J, N114J, E115J, L116J, M117J, E118J, Q119J, T120J, S121J, E122J, I123J, I124J, T125J, F126J, A127J, E128J, S129J, G130J, T131J, A132J, R133J, K134J, T135J, L136J, H137J, F138J, E139J, I140J, S141J, K142J, E143J, G144J, S145J, D146J, L147J, S148J, V149J, V150J, E151J, R152J, A153J, E154J, V155J, W156J, L157J, F158J, L159J, K160J, V161J, P162J, K163J, A164J, N165J, R166J, T167J, R168J, T169J, K170J, V171J, T172J, I173J, R174J, L175J, F176J, Q177J, Q178J, Q179J, K180J, H181J, P182J, Q183J, G184J, S185J, L186J, D187J, T188J, G189J, E190J, E191J, A192J, E193J, E194J, V195J, G196J, L197J, K198J, G199J, E200J, R201J, S202J, E203J, L204J, L205J, L206J, S207J, E208J, K209J, V210J, V211J, D212J, A213J, R214J, K215J, S216J, T217J, W218J, H219J, V220J, F221J, P222J, V223J, S224J, S225J, S226J, I227J, Q228J, R229J, L230J, L231J, D232J, Q233J, G234J, K235J, S236J, S237J, L238J, D239J, V240J, R241J, I242J, A243J, C244J, E245J, Q246J, C247J, Q248J, E249J, S250J, G251J, A252J, S253J, L254J, V255J, L256J, L257J, G258J, K259J, K260J, K261J, K262J, K263J, E264J, E265J, E266J, G267J, E268J, G269J, K270J, K271J, K272J, G273J, G274J, G275J, E276J, G277J, G278J, A279J, G280J, A281J, D282J, E283J, E284J, K285J, E286J, Q287J, S288J, H289J, R290J, P291J, F292J, L293J, M294J, L295J, Q296J, A297J, R298J, Q299J, S300J, E301J, D302J, H303J, P304J, H305J, R306J, R307J, R308J, R309J, R310J, G311J, L312J, E313J, C314J, D315J, G316J, K317J, V318J, N319J, I320J, C321J, C322J, K323J, K324J, Q325J, A347J, N348J, Y349J, C350J, E351J, G352J, E353J, C354J, P355J, S356J, H357J, I358J, A359J, G360J, T361J, S362J, G363J, S364J, S365J, L366J, S367J, F368J, H369J, S370J, T371J, V372J, I373J, N374J, H375J, Y376J, R377J, M378J, R379J, G380J, H381J, S382J, P383J, F384J, A385J, N386J, L387J, K388J, S389J, C390J, C391J, V392J, P393J, T394J, V420J, E421J, E422J, C423J, G424J, C425J, and S426J. The variable “J” is any amino acid whose introduction results in an increase in the electrostatic interaction between the L1 and L3 β hairpin loop structures of the human activin A subunit and a receptor with affinity for a dimeric protein containing the mutant human activin A subunit monomer.

The invention also contemplates a number of human activin A subunit in modified forms. These modified forms include human activin A subunit linked to another cystine knot growth factor or a fraction of such a monomer.

In specific embodiments, the mutant human activin A subunit heterodimer comprising at least one mutant subunit or the single chain human activin A subunit analog as described above is functionally active, i.e., capable of exhibiting one or more functional activities associated with the wild-type human activin A subunit, such as human activin A subunit receptor binding, human activin A subunit protein family receptor signalling and extracellular secretion. Preferably, the mutant human activin A subunit heterodimer or single chain human activin A subunit analog is capable of binding to the human activin A subunit receptor, preferably with affinity greater than the wild type human activin A subunit. Also it is preferable that such a mutant human activin A subunit heterodimer or single chain human activin A subunit analog triggers signal transduction. Most preferably, the mutant human activin A subunit heterodimer comprising at least one mutant subunit or the single chain human activin A subunit analog of the present invention has an in vitro bioactivity and/or in vivo bioactivity greater than the wild type human activin A subunit and has a longer serum half-life than wild type human activin A subunit. Mutant human activin A subunit heterodimers and single chain human activin A subunit analogs of the invention can be tested for the desired activity by procedures known in the art.

Mutants of the Human Activin B Subunit

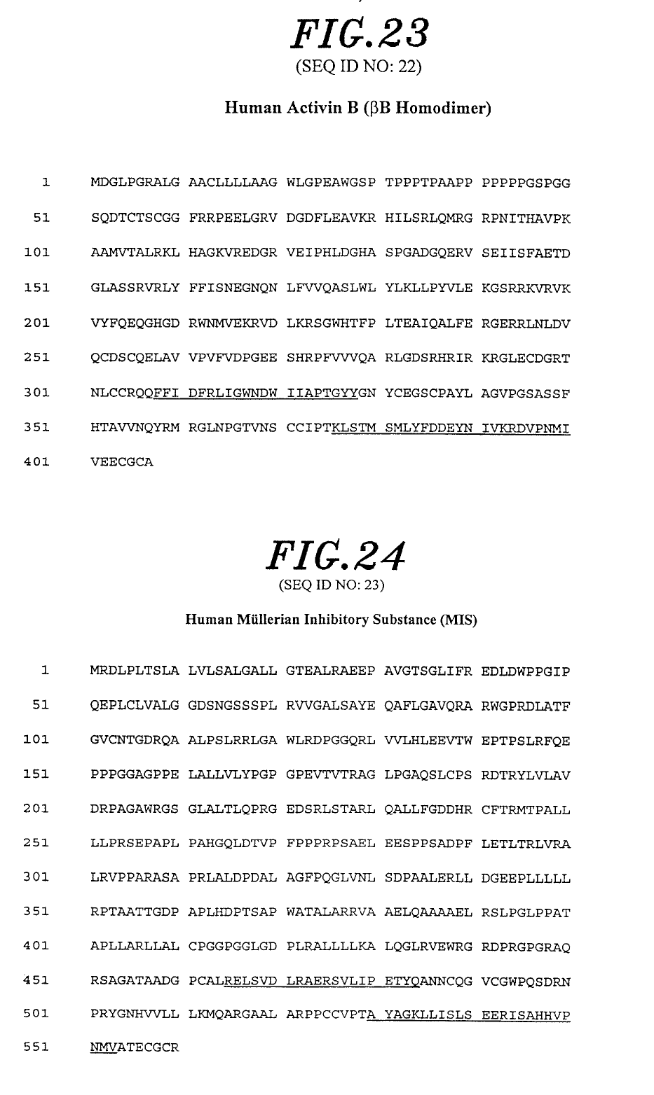

The human activin B subunit contains 407 amino acids as shown in FIG. 23 (SEQ ID No: 22). The invention contemplates mutants of the human activin B subunit comprising single or multiple amino acid substitutions, deletions or insertions, of one, two, three, four or more amino acid residues when compared with the wild type monomer. Furthermore, the invention contemplates mutant human activin B subunit that are linked to another CKGF protein.

The present invention provides mutant human activin B subunit L1 hairpin loops having one or more amino acid substitutions between positions 308 and 328, inclusive, excluding Cys residues, as depicted in FIG. 23 (SEQ ID NO: 22). The amino acid substitutions include: F308X, F309X, I310X, D311X, F312X, R313X, L314X, I315X, G316X, W317X, N318X, D319X, W320X, I321X, I322X, A323X, P324X, T325X, G326X, Y327X, and Y328X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the human activin B subunit monomer where an acidic residue is present, the variable “X” would correspond to a basic amino acid residue. Specific examples of electrostatic charge altering mutations where a basic residue is introduced into the human activin B subunit monomer include one or more of the following: D311B and D319B, wherein “B” is a basic amino acid residue.

Introducing acidic amino acid residues where basic residues are present in the human activin B subunit monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include R313Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D311U, R313U, and D319U, wherein “U” is a neutral amino acid.

Mutant human activin B subunit monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: F308Z, F309Z, I310Z, F312Z, L314Z, I315Z, G316Z, W317Z, N318Z, W320Z, I321Z, I322Z, A323Z, P324Z, T325Z, G326Z, Y327Z, Y328Z, F308B, F309B, I310B, F312B, L314B, I315B, G316B, W317B, N318B, W320B, I321B, I322B, A323B, P324B, T325B, G326B, Y327B, and Y328B, wherein “Z” is an acidic amino acid and “B” is a basic amino acid.

Mutant human activin B subunit containing mutants in the L3 hairpin loop are also described. These mutant proteins have one or more amino acid substitutions, deletion or insertions, between positions 376 and 400, inclusive, excluding Cys residues, of the L3 hairpin loop, as depicted in FIG. 23 (SEQ ID NO: 22). The amino acid substitutions include: K376X, L377X, S378X, T379X, M380X, S381X, M382X, L383X, Y384X, F385X, D386X, D387X, E388X, Y389X, N390X, I391X, V392X, K393X, R394X, D395X, V396X, P397X, N398X, M399X, and I400X, wherein “X” is any amino acid residue, the substitution of which alters the electrostatic character of the L3 loop.

One set of mutations of the L3 hairpin loop includes introducing one or more basic amino acid residues into the human activin B subunit L3 hairpin loop amino acid sequence. For example, when introducing basic residues into the L3 loop of the human activin B subunit, the variable “X” of the sequence described above corresponds to a basic amino acid residue. Specific examples of electrostatic charge altering mutations where a basic residue is introduced into the human activin B subunit include one or more of the following: D386B, D387B, E388B, and D395B, wherein “B” is a basic amino acid residue.

The invention further contemplates introducing one or more acidic residues into the amino acid sequence of the human activin B subunit L3 hairpin loop. For example, one or more acidic amino acids can be introduced in the sequence of 376-400 described above, wherein the variable “X” corresponds to an acidic amino acid. Specific examples of such mutations include K376Z, K393Z, and R394Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K376U, D386U, D387U, E388U, K393U, R394U, and D395U, wherein “U” is a neutral amino acid.

Mutant human activin B subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include, L377Z, S378Z, T279Z, M380Z, S381Z, M382Z, L383Z, Y384Z, F385Z, Y389Z, N390Z, I391Z, V392Z, V396Z, P397Z, N398Z, M399Z, I400Z, L377B, S378B, T279B, M380B, S381B, M382B, L383B, Y384B, F385B, Y389B, N390B, I391B, V392B, V396B, P397B, N398B, M399B, and I400B, wherein “Z” is an acidic amino acid and “B” is a basic amino acid.

1. The present invention also contemplate human activin B subunit containing mutations outside of said β hairpin loop structures that alter the structure or conformation of those hairpin loops. These structural alterations in turn serve to increase the electrostatic interactions between regions of the β hairpin loop structures of human activin B subunit contained in a dimeric molecule, and a receptor having affinity for the dimeric protein. These mutations are found at positions selected from the group consisting of positions 1-307, 329-375, and 401-407 of the human activin B subunit monomer.

Specific examples of these mutation outside of the β hairpin L1 and L3 loop structures include, M1J, D2J, G3J, L4J, P5J, G6J, R7J, A8J, L9J, G10J, A11J, A12J, C13J, L14J, L15J, L16J, L17J, A18J, G20J, W21J, L22J, G23J, P24J, E25J, A26J, W27J, G28J, S20J, P30J, T31J, P32J, P33J, P34J, T35J, P36J, A37J, A38J, P39J, P40J, P41J, P42J, P43J, P44J, P45J, G46J, S47J, P48J, G49J, G50J, S51J, Q52J, D53J, T54J, C55J, T56J, S57J, C58J, G59J, G60J, F61J, R62J, R63J, P64J, E65J, E66J, L67J, G68J, R69J, V70J, D71J, G72J, D73J, F74J, L75J, E76J, A77J, V78J, K79J, R80J, H81J, I82J, L83J, S84J, R85J, L86J, Q87J, M88J, R89J, G90J, R91J, P92J, N93J, I94J, T95J, H96J, A97J, V98J, P99J, K100J, A101J, A102J, M103J, V104J, T105J, A106J, L107J, R108J, K109J, L110J, H111J, A112J, G113J, K114J, V115J, R116J, E117J, D118J, G119J, R120J, V121J, E122J, I123J, P124J, H125J, L126J, D127J, G128J, H129J, A130J, S131J, P132J, G133J, A134J, D135J, G136J, Q137J, E138J, R139J, V140J, S141J, E142J, I143J, I144J, S145J, F146J, A147J, E148J, T149J, D150J, G151J, L152J, A153J, S154J, S155J, R156J, V157J, R158J, L159J, Y160J, F161J, F162J, I163J, S164J, N165J, E166J, G167J, N168J, Q169J, N170J, L171J, F172J, V173J, V174J, Q175J, A176J, S177J, L178J, W179J, L180J, Y181J, L182J, K183J, L184J, L185J, P186J, Y187J, V188J, L189J, E190J, K191J, G192J, S193J, R194J, R195J, K196J, V197J, R198J, V199J, K200J, V201J, Y202J, F203J, Q204J, E205J, Q206J, G207J, H208J, G209J, D210J, R211J, W212J, N213J, M214J, V215J, E216J, K217J, R218J, V219J, D220J, L221J, K222J, R223J, S224J, G225J, W226J, H227J, T228J, F229J, P230J, L231J, T232J, E233J, A234J, I235J, Q236J, A237J, L238J, F239J, E240J, R241J, G242J, E243J, R244J, R245J, L246J, N247J, L248J, D249J, V250J, Q251J, C252J, D253J, S254J, C255J, Q256J, E257J, L258J, A259J, V260J, V261J, P262J, V263J, F264J, V265J, D266J, P267J, G268J, E269J, E270J, S271J, H272J, R273J, P274J, F275J, V276J, V277J, V278J, Q279J, A280J, R281J, L282J, G283J, D284J, S285J, R286J, H287J, R288J, I289J, R290J, K291J, R292J, G293J, L294J, E295J, C296J, D297J, G298J, R299J, T300J, N301J, L302J, C303J, C304J, R305J, Q306J, Q307J, G329J, N330J, Y331J, C332J, E333J, G334J, S335J, C336J, P337J, A338J, Y339J, L340J, A341J, G342J, V343J, P344J, G345J, S346J, A347J, S348J, S349J, F350J, H351J, T352J, A353J, V354J, V35J, 5N356J, Q357J, Y358J, R359J, M360J, R361J, G362J, L363J, N364J, P365J, G366J, T367J, V368J, N369J, S370J, C371J, C372J, I373J, P374J, T375VJ, 401J, E402J, E403J, C404J, G405J, C406J, and A407J. Wherein J is any amino acid that results in an increase in an electrostatic interaction between said β hairpin structure of said human transforming growth factor family protein and a receptor with affinity for said human transforming growth factor family protein. The variable “J” is any amino acid whose introduction results in an increase in the electrostatic interaction between the L1 and L3 β hairpin loop structures of the human activin B subunit and a receptor with affinity for a dimeric protein containing the mutant human activin B subunit monomer.

The invention also contemplates a number of human activin B subunit in modified forms. These modified forms include human activin B subunit linked to another cystine knot growth factor or a fraction of such a monomer.

In specific embodiments, the mutant human activin B subunit heterodimer comprising at least one mutant subunit or the single chain human activin B subunit analog as described above is functionally active, i.e., capable of exhibiting one or more functional activities associated with the wild-type human activin B subunit, such as human activin B subunit receptor binding, human activin B subunit protein family receptor signalling and extracellular secretion. Preferably, the mutant human activin B subunit heterodimer or single chain human activin B subunit analog is capable of binding to the human activin B subunit receptor, preferably with affinity greater than the wild type human activin B subunit. Also it is preferable that such a mutant human activin B subunit heterodimer or single chain human activin B subunit analog triggers signal transduction. Most preferably, the mutant human activin B subunit heterodimer comprising at least one mutant subunit or the single chain human activin B subunit analog of the present invention has an in vitro bioactivity and/or in vivo bioactivity greater than the wild type human activin B subunit and has a longer serum half-life than wild type human activin B subunit. Mutant human activin B subunit heterodimers and single chain human activin B subunit analogs of the invention can be tested for the desired activity by procedures known in the art.

Mutants of the Mullerian Inhibitory Substance

The Mullerian Inhibitory Substance contains 560 amino acids as shown in FIG. 24 (SEQ ID No: 23). The invention contemplates mutants of the mullerian inhibitory substance comprising single or multiple amino acid substitutions, deletions or insertions, of one, two, three, four or more amino acid residues when compared with the wild type monomer. Furthermore, the invention contemplates mutant mullerian inhibitory substance that are linked to another CKGF protein.

The present invention provides mutant mullerian inhibitory substance L1 hairpin loops having one or more amino acid substitutions between positions 21 and 40, inclusive, excluding Cys residues, as depicted in FIG. 24 (SEQ ID NO: 23). The amino acid substitutions include: R465X, E466X, L467X, S468X, V469X, D470X, L471X, R472X, A473X, E474X, R475X, S476X, V477X, L478X, I479X, P480X, E481X, T482X, Y483X, and 484X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the mullerian inhibitory substancemonomer where an acidic residue is present, the variable “X” would correspond to a basic amino acid residue. Specific examples of electrostatic charge altering mutations where a basic residue is introduced into the mullerian inhibitory substancemonomer include one or more of the following: E466B, D470B, E474B, and E481B wherein “B” is a basic amino acid residue.

Introducing acidic amino acid residues where basic residues are present in the mullerian inhibitory substancemonomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: R465, R472, and R475, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at R465U, E466U, D470U, R472U, E474U, R475U, and E481U, wherein “U” is a neutral amino acid.

Mutant mullerian inhibitory substancemonomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: L467Z, S468Z, V469Z, L471Z, A473Z, S476Z, V477Z, L478Z, I479Z, P480Z, T482Z, Y483Z, Q484Z, L467B, S468B, V469B, L471B, A473B, S476B, V477B, L478B, I479B, P480B, T482B, Y483B, and Q484B, wherein “Z” is an acidic amino acid and “B” is a basic amino acid.

Mutant mullerian inhibitory substance containing mutants in the L3 hairpin loop are also described. These mutant proteins have one or more amino acid substitutions, deletion or insertions, between positions 530 and 553, inclusive, excluding Cys residues, of the L3 hairpin loop, as depicted in FIG. 24 (SEQ ID NO: 23). The amino acid substitutions include: A530X, Y531X, A532X, G533X, K534X, L535X, L536X, I537X, S538X, L539X, S540X, E541X, E542X, R543X, I544X, S545X, A546X, H547X, H548X, V549X, P550X, N551X, M552X, and V553X, wherein “X” is any amino acid residue, the substitution of which alters the electrostatic character of the L3 loop.

One set of mutations of the L3 hairpin loop includes introducing one or more basic amino acid residues into the mullerian inhibitory substance L3 hairpin loop amino acid sequence. For example, when introducing basic residues into the L3 loop of the mullerian inhibitory substance, the variable “X” of the sequence described above corresponds to a basic amino acid residue. Specific examples of electrostatic charge altering mutations where a basic residue is introduced into the mullerian inhibitory substance include one or more of the following: E541B and E542B, wherein “B” is a basic amino acid residue.

The invention further contemplates introducing one or more acidic residues into the amino acid sequence of the mullerian inhibitory substance L3 hairpin loop. For example, one or more acidic amino acids can be introduced in the sequence of 530-553 described above, wherein the variable “X” corresponds to an acidic amino acid. Specific examples of such mutations include K534Z, R543Z, H547Z, and H548Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced of K534U, E541U, E542U, R543U, H547U, and H548U, wherein “U” is a neutral amino acid.

Mutant mullerian inhibitory substance proteins are provided containing one or more electrostatic charge altering mutations in the L3 hairpin loop amino acid sequence that convert non-charged or neutral amino acid residues to charged residues. Examples of mutations converting neutral amino acid residues to charged residues include, A530Z, Y531Z, A532Z, G533Z, L535Z, L536Z, I537Z, S538Z, L539Z, S540Z, I544Z, S545Z, A546Z, V549Z, P550Z, N551Z, M552Z, V553Z, A530B, Y531B, A532B, G533B, L535B, L536B, I537B, S538B, L539B, S540B, I544B, S545B, A546B, V549B, P550B, N551B, M552B, and V553B, wherein “Z” is an acidic amino acid and “B” is a basic amino acid.

The present invention also contemplate mullerian inhibitory substance containing mutations outside of said β hairpin loop structures that alter the structure or conformation of those hairpin loops. These structural alterations in turn serve to increase the electrostatic interactions between regions of the β hairpin loop structures of mullerian inhibitory substance contained in a dimeric molecule, and a receptor having affinity for the dimeric protein. These mutations are found at positions selected from the group consisting of positions 1-464, 485-529, and 554-560 of the mullerian inhibitory substance monomer.

Specific examples of these mutation outside of the β hairpin L1 and L3 loop structures include, M1J, R2J, D3J, L4J, P5J, L6J, T7J, S8J, L9J, A10J, L11J, V12J, L13J, S14J, A15J, L16J, G17J, A18J, L19J, L20J, G21J, T22J, E23J, A24J, L25J, R26J, A27J, E28J, E29J, P30J, A31J, V32J, G33J, T34J, S35J, G36J, L37J, I38J, F39J, R40J, E41J, D42J, L43J, D44J, W45J, P46J, P47J, G48J, I49J, P50J, Q51J, E52J, P53J, L54J, C55J, L56J, V57J, A58J, L59J, G60J, G61J, D62J, S63J, N64J, G65J, S66J, S67J, S68J, P69J, L70J, R71J, V72J, V37J, G74J, A75J, L76J, S77J, A78J, Y79J, E80J, Q81J, A82J, F83J, L84J, G85J, A86J, V87J, Q88J, R89J, A90J, R91J, W92J, G93J, P94J, R95J, D96J, L97J, A98J, T99J, F100J, G101J, V102J, C103J, N104J, T105J, G106J, D107J, R108J, Q109J, A110J, A111J, L112J, P113J, S114J, L115J, R116J, R117J, L118J, G119J, A120J, W121J, L122J, R123J, D124J, P125J, G126J, G127J, Q128J, R129J, L130J, V131J, V132J, L133J, H134J, L135J, E136J, E137J, V138J, T139J, W140J, E141J, P142J, T143J, P144J, S145J, L146J, R147J, F148J, Q149J, E150J, P151J, P152J, P153J, G154J, G155J, A156J, G157J, P158J, P159J, E160J, L161J, A162J, L163J, L164J, V165J, L166J, Y167J, P168J, G169J, P170J, G171J, P172J, E173J, V174J, T175J, V176J, T177J, R178J, A179J, G180J, L181J, P182J, G183J, A184J, Q185J, S186J, L187J, C188J, P189J, S190J, R191J, D192J, T193J, R194J, Y195J, L196J, V197J, L198J, A199J, V200J, D201J, R202J, P203J, A204J, G205J, A206J, W207J, R208J, G209J, S210J, G211J, L212J, A213J, L214J, T215J, L216J, Q217J, P218J, R219J, G220J, E221J, D222J, S223J, R224J, L225J, S226J, T227J, A228J, R229J, L230J, Q231J, A232J, L233J, L234J, F235J, G236J, D237J, D238J, H239J, R240J, C241J, F242J, T243J, R244J, M245J, T246J, P247J, A248J, L249J, L250J, L251J, L252J, P253J, R254J, S255J, E256J, P257J, A258J, P259J, L260J, P261J, A262J, H263J, G264J, Q265J, L266J, D267J, T268J, V269J, P270J, F271J, P272J, P273J, P274J, R275J, P276J, S277J, A278J, E279J, L280J, E281J, E282J, S283J, P284J, P285J, S286J, A287J, D288J, P289J, F290J, L291J, E292J, T293J, L294J, T295J, R296J, L297J, V298J, R299J, A300J, L301J, R302J, V303J, P304J, P305J, A306J, R307J, A308J, S309J, A310J, P311J, R312J, L313J, A314J, L315J, D316J, P317J, D318J, A319J, L320J, A321J, G322J, F323J, P324J, Q325J, G326J, L327J, V328J, N329J, L330J, S331J, D332J, P333J, A334J, A335J, L336J, E337J, R338J, L339J, L340J, D341J, G342J, E343J, E344J, P345J, L346J, L347J, L348J, L349J, L350J, R351J, P352J, T353J, A354J, A355J, T356J, T357J, G358J, D359J, P360J, A361J, P362J, L363J, H364J, D365J, P366J, T367J, S368J, A369J, P370J, W371J, A372J, T373J, A374J, L375J, A376J, R377J, R378J, V379J, A380J, A381J, E382J, L383J, Q384J, A385J, A386J, A387J, A388J, E389J, L390J, R391J, S392J, L393J, P394J, G395J, L396J, P397J, P398J, A399J, T400J, A401J, P402J, L403J, L404J, A405J, R406J, L407J, L408J, A409J, L410J, C411J, P412J, G413J, G414J, P415J, G416J, G417J, L418J, G419J, D420J, P421J, L422J, R423J, A424J, L425J, L426J, L427J, L428J, K429J, A430J, L431J, Q432J, G433J, L434J, R435J, V436J, E437J, W438J, R439J, G440J, R441J, D442J, P443J, R444J, G445J, P446J, G447J, R448J, A449J, Q450J, R451J, S452J, A453J, G454J, A455J, T456J, A457J, A458J, D459J, G460J, P461J, C462J, A463J, L464J, A485J, N486J, N487J, C488J, Q489J, G490J, V491J, C492J, G493J, W494J, P495J, Q496J, S497J, D498J, R499J, N500J, P501J, R502J, Y503J, G504J, N505J, H506J, V507J, V508J, L509J, L510J, L511J, K512J, M513J, Q514J, A515J, R516J, G517J, A518J, A519J, L520J, A521J, R522J, P523J, P524J, C525J, C526J, V527J, P528J, T529J, A554J, T555J, E556J, C557J, G558J, C559J, R560J. The variable “J” is any amino acid whose introduction results in an increase in the electrostatic interaction between the L1 and L3 β hairpin loop structures of the mullerian inhibitory substanceand a receptor with affinity for a dimeric protein containing the mutant mullerian inhibitory substance monomer.

The invention also contemplates a number of mullerian inhibitory substance in modified forms. These modified forms include mullerian inhibitory substance linked to another cystine knot growth factor or a fraction of such a monomer.

In specific embodiments, the mutant mullerian inhibitory substance heterodimer comprising at least one mutant subunit or the single chain mullerian inhibitory substance analog as described above is functionally active, i.e., capable of exhibiting one or more functional activities associated with the wild-type mullerian inhibitory substance, such as mullerian inhibitory substance receptor binding, mullerian inhibitory substance protein family receptor signalling and extracellular secretion. Preferably, the mutant mullerian inhibitory substance heterodimer or single chain mullerian inhibitory substance analog is capable of binding to the mullerian inhibitory substance receptor, preferably with affinity greater than the wild type mullerian inhibitory substance. Also it is preferable that such a mutant mullerian inhibitory substance heterodimer or single chain mullerian inhibitory substance analog triggers signal transduction. Most preferably, the mutant mullerian inhibitory substance heterodimer comprising at least one mutant subunit or the single chain mullerian inhibitory substance analog of the present invention has an in vitro bioactivity and/or in vivo bioactivity greater than the wild type mullerian inhibitory substance and has a longer serum half-life than wild type mullerian inhibitory substance. Mutant mullerian inhibitory substance heterodimers and single chain mullerian inhibitory substance analogs of the invention can be tested for the desired activity by procedures known in the art.

Mutants of the Human Bone Morphogenic Protein-2 (BMP-2) Subunit

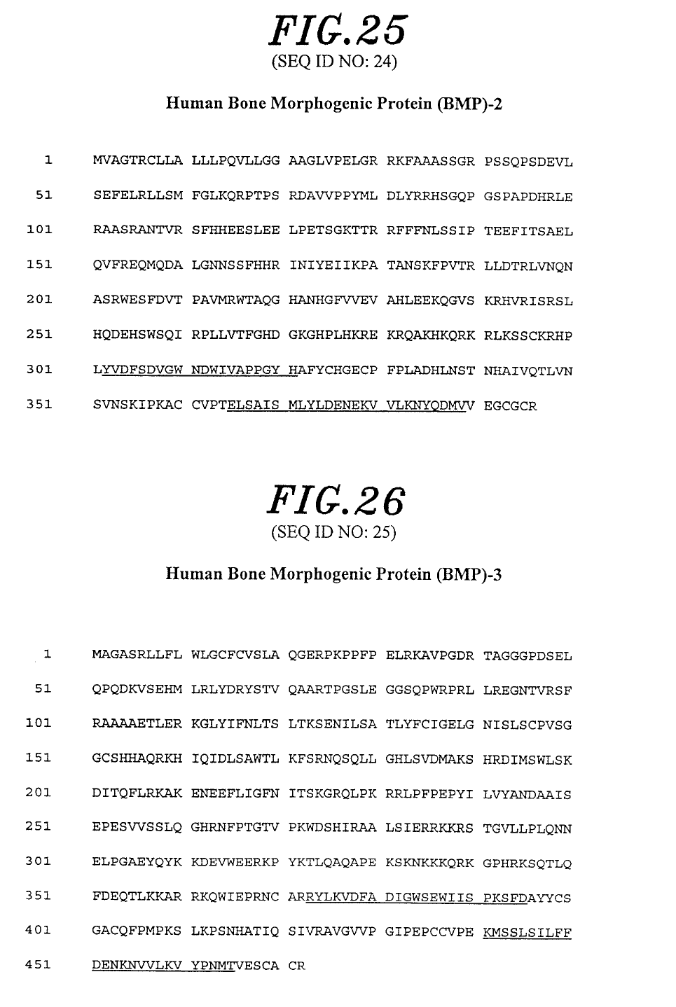

The human bone morphogenic protein-2 (BMP-2) subunit contains 396 amino acids as shown in FIG. 25 (SEQ ID No: 24). The invention contemplates mutants of the BMP-2 subunit comprising single or multiple amino acid substitutions, deletions or insertions, of one, two, three, four or more amino acid residues when compared with the wild type monomer. Furthermore, the invention contemplates mutant BMP-2 subunit that are linked to another CKGF protein.

The present invention provides mutant BMP-2 subunit L1 hairpin loops having one or more amino acid substitutions between positions 302 and 321, inclusive, excluding Cys residues, as depicted in FIG. 25 (SEQ ID NO: 24). The amino acid substitutions include: Y302X, V303X, D304X, F305X, S306X, D307X, V308X, G309X, W310X, N311X, D312X, W313X, I314X, V315X, A316X, P317X, P318X, G319X, Y320X, and H321X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the BMP-2 subunit monomer where an acidic residue is present, the variable “X” would correspond to a basic amino acid residue. Specific examples of electrostatic charge altering mutations where a basic residue is introduced into the BMP-2 subunit monomer include one or more of the following: D304B, D307B, and D312B wherein “B” is a basic amino acid residue.

Introducing acidic amino acid residues where basic residues are present in the BMP-2 subunit monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: H321Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced D304U, D307U, D312U, and H321U, wherein “U” is a neutral amino acid.

Mutant BMP-2 subunit monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: of Y302Z, V303Z, F305Z, S306Z, V308Z, G309Z, W310Z, N311Z, W313Z, I314Z, V315Z, A316Z, P317Z, P318B, G319Z, Y320Z, Y302B, V303B, F305B, S306B, V308B, G309B, W310B, N311B, W313B, I314B, V315B, A316B, P317B, P318B, G319B, and Y320B, wherein “Z” is an acidic amino acid and “B” is a basic amino acid.

Mutant BMP-2 subunit containing mutants in the L3 hairpin loop are also described. These mutant proteins have one or more amino acid substitutions, deletion or insertions, between positions 365 and 389, inclusive, excluding Cys residues, of the L3 hairpin loop, as depicted in FIG. 25 (SEQ ID NO: 24). The amino acid substitutions include: E365X, L366X, S367X, A368X, I369X, S370X, M371X, L372X, Y373X, L374X, D375X, E376X, N377X, E378X, K379X, V380X, V381X, L382X, K383X, N384X, Y385X, Q386X, D387X, M388X, and V389X, wherein “X” is any amino acid residue, the substitution of which alters the electrostatic character of the L3 loop.

One set of mutations of the L3 hairpin loop includes introducing one or more basic amino acid residues into the BMP-2 subunit L3 hairpin loop amino acid sequence. For example, when introducing basic residues into the L3 loop of the BMP-2 subunit, the variable “X” of the sequence described above corresponds to a basic amino acid residue. Specific examples of electrostatic charge altering mutations where a basic residue is introduced into the BMP-2 subunit include one or more of the following: E365B, D375B, E376B, E378B, and D387, wherein “B” is a basic amino acid residue.

The invention further contemplates introducing one or more acidic residues into the amino acid sequence of the BMP-2 subunit L3 hairpin loop. For example, one or more acidic amino acids can be introduced in the sequence of 365-389 described above, wherein the variable “X” corresponds to an acidic amino acid. Specific examples of such mutations include K379Z and K383Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced E365U D375U, E376U E378U, K379U K383U and D387U, wherein “U” is a neutral amino acid.

Mutant BMP-2 subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include L366Z, S367Z, A368Z, I369Z, S370Z, M371Z, L372Z, Y373Z, L374Z, N377Z, V380Z, V381Z, L382Z, N384Z, Y385Z, Q386Z, M388Z, V389Z, L366B, S367B, A368B, I369B, S370B, M371B, L372B, Y373B, L374B, N377B, V380B, V381B, L382B, N384B, Y385B, Q386B, M388B, and V389B, wherein “Z” is an acidic amino acid and “B” is a basic amino acid.

The present invention also contemplate BMP-2 subunit containing mutations outside of said β hairpin loop structures that alter the structure or conformation of those hairpin loops. These structural alterations in turn serve to increase the electrostatic interactions between regions of the β hairpin loop structures of BMP-2 subunit contained in a dimeric molecule, and a receptor having affinity for the dimeric protein. These mutations are found at positions selected from the group consisting of 1-301, 322-364, and 390-396 of the BMP-2 subunit monomer.

Specific examples of these mutation outside of the β hairpin L1 and L3 loop structures include, M1J, V2J, A3J, G4J, T5J, R6J, C7J, L8J, L9J, A10J, L11J, L12J, L13J, P14J, Q15J, V16J, L17J, L18J, G19J, G20J, A21J, A22J, G23J, L24J, V25J, P26J, E27J, L28J, G29J, R30J, R31J, K32J, F33J, A34J, A35J, A36J, S37J, S38J, G39J, R40J, P41J, S42J, S43J, Q44J, P45J, S46J, D47J, E48J, V49J, L50J, S51J, E52J, F53J, E54J, L55J, R56J, L57J, L58J, S59J, M60J, F61J, G62J, L63J, K64J, Q65J, R66J, P67J, T68J, P69J, S70J, R71J, D72J, A73J, V74J, V75J, P76J, P77J, Y78J, M79J, L80J, D81J, L82J, Y83J, R84J, R85J, H86J, S87J, G88J, Q89J, P90J, G91J, S92J, P93J, A94J, P95J, D96J, H97J, R98J, L99J, E100J, R101J, A102J, A103J, S104J, R105J, A106J, N107J, T108J, V109J, R110J, S111J, F112J, H113J, H114J, E115J, E116J, S117J, L118J, E119J, E120J, L121J, P122J, E123J, T124J, S125J, G126J, K127J, T128J, T129J, R130J, R131J, F132J, F133J, F134J, N135J, L136J, S137J, S138J, I139J, P140J, T141J, E142J, E143J, F144J, I145J, T146J, S147J, A148J, E149J, L150J, Q151J, V152J, F153J, R154J, E155J, Q156J, M157J, Q158J, D159J, A160J, L161J, G162J, N163J, N164J, S165J, S166J, F167J, H168J, H169J, R170J, I171J, N172J, I173J, Y174J, E175J, I176J, I177J, K178J, P179J, A180J, T181J, A182J, N183J, S184J, K185J, F186J, P187J, V188J, T189J, R190J, L191J, L192J, D193J, T194J, R195J, L196J, V197J, N198J, Q199J, N200J, A201J, S202J, R203J, W204J, E205J, S206J, F207J, D208J, V209J, T210J, P211J, A212J, V213J, M214J, R215J, W216J, T217J, A218J, Q219J, G220J, H221J, A222J, N223J, H224J, G225J, F226J, V227J, V228J, E229J, V230J, A231J, H232J, L233J, E234J, E235J, K236J, Q237J, G238J, V239J, S240J, K241J, R242J, H243J, V244J, R245J, I256J, S247J, R248J, S249J, L250J, H251J, Q252J, D253J, E254J, H255J, S256J, W257J, S258J, Q259J, I260J, R261J, P262J, L263J, L264J, V265J, T266J, F267J, G268J, H269J, D270J, G271J, K272J, G273J, H274J, P275J, L276J, H277J, K278J, R279J, E280J, K281J, R282J, Q283J, A284J, K285J, H286J, K287J, Q288J, R289J, K290J, R291J, L292J, K293J, S294J, S295J, C296J, K297J, R298J, H299J, P300J, L301J, A322J, F323J, Y324J, C325J, H326J, G327J, E328J, C329J, P330J, F331J, P332J, L333J, A334J, D335J, H336J, L337J, N338J, S339J, T340J, N341J, H342J, A343J, I344J, V345J, Q346J, T347J, L348J, V349J, N350J, S351J, V352J, N353J, S354J, K355J, I356J, P357J, K358J, A359J, C360J, C361J, V362J, P363J, T364J, V390J, E391J, G392J, C393J, G394J, C395J, and R396J. The variable “J” is any amino acid whose introduction results in an increase in the electrostatic interaction between the L1 and L3 β hairpin loop structures of the BMP-2 subunit and a receptor with affinity for a dimeric protein containing the mutant BMP-2 subunit monomer.

The invention also contemplates a number of BMP-2 subunit in modified forms. These modified forms include BMP-2 subunit linked to another cystine knot growth factor or a fraction of such a monomer.

In specific embodiments, the mutant BMP-2 subunit heterodimer comprising at least one mutant subunit or the single chain BMP-2 subunit analog as described above is functionally active, i.e., capable of exhibiting one or more functional activities associated with the wild-type BMP-2 subunit, such as BMP-2 subunit receptor binding, BMP-2 subunit protein family receptor signalling and extracellular secretion. Preferably, the mutant BMP-2 subunit heterodimer or single chain BMP-2 subunit analog is capable of binding to the BMP-2 subunit receptor, preferably with affinity greater than the wild type BMP-2 subunit. Also it is preferable that such a mutant BMP-2 subunit heterodimer or single chain BMP-2 subunit analog triggers signal transduction. Most preferably, the mutant BMP-2 subunit heterodimer comprising at least one mutant subunit or the single chain BMP-2 subunit analog of the present invention has an in vitro bioactivity and/or in vivo bioactivity greater than the wild type BMP-2 subunit and has a longer serum half-life than wild type BMP-2 subunit. Mutant BMP-2 subunit heterodimers and single chain BMP-2 subunit analogs of the invention can be tested for the desired activity by procedures known in the art.

Mutants of the Human Bone Morphogenic Protein-3 (BMP-3) Subunit

The human bone morphogenic protein-3 (BMP-3) subunit contains 472 amino acids as shown in FIG. 26 (SEQ ID No: 25). The invention contemplates mutants of the BMP-3 comprising single or multiple amino acid substitutions, deletions or insertions, of one, two, three, four or more amino acid residues when compared with the wild type monomer. Furthermore, the invention contemplates mutant BMP-3 that are linked to another CKGF protein.

The present invention provides mutant BMP-3 L1 hairpin loops having one or more amino acid substitutions between positions 373 and 395, inclusive, excluding Cys residues, as depicted in FIG. 26 (SEQ ID NO: 25). The amino acid substitutions R373, Y374X, L375X, K376X, V377X, D378X, F379X, A380X, D381X, I382X, G383X, W384X, S385X, E386X, I387X, I388X, S389X, P390X, K391X, S392X, F393X, and D394X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the BMP-3monomer where an acidic residue is present, the variable “X” would correspond to a basic amino acid residue. Specific examples of electrostatic charge altering mutations where a basic residue is introduced into the BMP-3monomer include one or more of the following: D378B, D381B, E386B, and D395B, wherein “B” is a basic amino acid residue.

Introducing acidic amino acid residues where basic residues are present in the BMP-3 sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: R373Z, K376Z, and K392Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at R373U, K376U, D378U, D381U, E386U, K392U, and D395U, wherein “U” is a neutral amino acid.

Mutant BMP-3monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: Y374Z, L375Z, V377Z, F379Z, A380Z, I382Z, G383Z, W384Z, S385Z, W387Z, I388Z, I389Z, S390Z, P391Z, S393Z, F394Z, Y374B, L375B, V377B, F379B, A380B, I382B, G383B, W384B, S385B, W387B, I388B, I389B, S390B, P391B, S393B, and F394B, wherein “Z” is an acidic amino acid and “B” is a basic amino acid.

Mutant BMP-3 containing mutants in the L3 hairpin loop are also described. These mutant proteins have one or more amino acid substitutions, deletion or insertions, between positions 441 and 465, inclusive, excluding Cys residues, of the L3 hairpin loop, as depicted in FIG. 26 (SEQ ID NO: 25). The amino acid substitutions include K441X, M442X, S443X, S444X, L445X, S446X, I447X, L448X, F449X, F450X, D451X, E452X, N453X, K454X, N455X, V456X, V457X, L458X, K459X, V460X, Y461X, P462X, N463X, M464X, and T465X, wherein “X” is any amino acid residue, the substitution of which alters the electrostatic character of the L3 loop.

One set of mutations of the L3 hairpin loop includes introducing one or more basic amino acid residues into the BMP-3 L3 hairpin loop amino acid sequence. For example, when introducing basic residues into the L3 loop of the BMP-3, the variable “X” of the sequence described above corresponds to a basic amino acid residue. Specific examples of electrostatic charge altering mutations where a basic residue is introduced into the BMP-3 include one or more of the following: D451B and E452B, wherein “B” is a basic amino acid residue.

The invention further contemplates introducing one or more acidic residues into the amino acid sequence of the BMP-3 L3 hairpin loop. For example, one or more acidic amino acids can be introduced in the sequence of 441-465 described above, wherein the variable “X” corresponds to an acidic amino acid. Specific examples of such mutations include K441Z, K454Z and K459Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K441U, D451U, E452U, K454U, and K459U, wherein “U” is a neutral amino acid.

Mutant BMP-3 proteins are provided containing one or more electrostatic charge altering mutations in the L3 hairpin loop amino acid sequence that convert non-charged or neutral amino acid residues to charged residues. Examples of mutations converting neutral amino acid residues to charged residues include, M442Z, S443Z, S444Z, L445Z, S446Z, I447Z, L448Z, F449Z, F450Z, N453Z, N455Z, V456Z, V457Z, L458Z, V460Z, Y461Z, P462Z, N463Z, M464Z, T465Z, M442B, S443B, S444B, L445B, S446B, I447B, L448B, F449B, F450B, N453B, N455B, V456B, V457B, L458B, V460B, Y461B, P462B, N463B, M464B, and T465B, wherein “Z” is an acidic amino acid and “B” is a basic amino acid.

The present invention also contemplate BMP-3 containing mutations outside of said β hairpin loop structures that alter the structure or conformation of those hairpin loops. These structural alterations in turn serve to increase the electrostatic interactions between regions of the β hairpin loop structures of BMP-3 contained in a dimeric molecule, and a receptor having affinity for the dimeric protein. These mutations are found at positions selected from the group consisting of positions 1-372, 396-440, and 466-472of the BMP-3.

Specific examples of these mutation outside of the β hairpin L1 and L3 loop structures include, M1J, A2J, G3J, A4J, S5J, R6J, L7J, L8J, F9J, L10J, W11J, L12J, G13J, C14J, F15J, C16J, V17J, S18J, L19J, A20J, Q21J, G22J, E23J, R24J, P25J, K26J, P27J, P28J, F29J, P30J, E31J, L32J, R33J, K34J, A35J, V36J, P37J, G38J, D39J, R40J, T41J, A42J, G43J, G44J, G45J, P46J, D47J, S48J, E49J, L50J, Q51J, P52J, Q53J, D54J, K55J, V56J, S57J, E58J, H59J, M60J, L61J, R62J, L63J, Y64J, D65J, R66J, Y67J, S68J, T69J, V70J, Q71J, A72J, A73J, R74J, T75J, P76J, G77J, S78J, L79J, E80J, G81J, G82J, S83J, Q84J, P85J, W86J, R87J, P88J, R89J, L90J, L91J, R92J, E93J, G94J, N95J, T96J, V97J, R98J, S99J, F100J, R101J, A102J, A103J, A104J, A105J, E106J, T107J, L108J, E109J, R110J, K111J, G112J, L113J, Y114J, I115J, F116J, N117J, L118J, T119J, S120J, L121J, T122J, K123J, S124J, E125J, N126J, I127J, L128J, S129J, A130J, T131J, L132J, Y133J, F134J, C135J, I136J, G137J, E138J, L139J, G140J, N141J, I142J, S143J, L144J, S145J, C146J, P147J, V148J, S149J, G150J, G151J, C152J, S153J, H154J, H155J, A156J, Q157J, R158J, K159J, H160J, I161J, Q162J, I163J, D164J, L165J, S166J, A167J, W168J, T169J, L170J, K171J, F172J, S173J, R174J, N175J, Q176J, S177J, Q178J, L179J, L180J, G181J, H182J, L183J, S184J, V185J, D186J, M187J, A188J, K189J, S190J, H191J, R192J, D193J, I194J, M195J, S196J, W197J, L198J, S199J, K200J, D201J, I202J, T203J, Q204J, F205J, L206J, R207J, K208J, A209J, K210J, E211J, N212J, E213J, E214J, F215J, L216J, I217J, G218J, F219J, N220J, I221J, T222J, S223J, K224J, G225J, R226J, Q227J, L228J, P229J, K230J, R231J, R232J, L233J, P234J, F235J, P236J, E237J, P238J, Y239J, I240J, L241J, V242J, Y243J, A244J, N245J, D246J, A247J, A248J, I249J, S250J, E251J, P252J, E253J, S254J, V255J, V256J, S257J, S258J, L259J, Q260J, G261J, H262J, R263J, N264J, F265J, P266J, T267J, G268J, T269J, V270J, P271J, K272J, W273J, D274J, S275J, H276J, I277J, R278J, A279J, A280J, L281J, S282J, I283J, E284J, R285J, R286J, K287J, K288J, R289J, S290J, T291J, G292J, V293J, L294J, L295J, P296J, L297J, Q298J, N299J, N300J, E301J, L302J, P303J, G304J, A305J, E306J, Y307J, Q308J, Y309J, K310J, K311J, D312J, E313J, V314J, W315J, E316J, E317J, R318J, K319J, P320J, Y321J, K322J, T323J, L324J, Q325J, A326J, Q327J, A328J, P329J, E330J, K331J, S332J, K333J, N334J, K335J, K336J, K337J, Q338J, R339J, K340J, G341J, P342J, H343J, R344J, K345J, S346J, Q347J, T348J, L349J, Q350J, F351J, D352J, E353J, Q354J, T355J, L356J, K357J, K358J, A359J, R360J, R361J, K362J, Q363J, W364J, I365J, E366J, P367J, R368J, N369J, C370J, A371J, R372J, A396J, Y397J, Y398J, C399J, S400J, G401J, A402J, C403J, Q404J, F405J, P406J, M407J, P408J, K409J, S410J, L411J, K412J, P413J, S414J, N415J, H416J, A417J, T418J, I419J, Q420J, S421J, I422J, V423J, R424J, A425J, V426J, G427J, V428J, V429J, P430J, G431J, I432J, P433J, E434J, P435J, C436J, C437J, V438J, P439J, E440J, V466J, E467J, S468J, C469J, A470J, C471J, and R472J. The variable “J” is any amino acid whose introduction results in an increase in the electrostatic interaction between the L1 and L3 β hairpin loop structures of the BMP-3 and a receptor with affinity for a dimeric protein containing the mutant BMP-3monomer.

The invention also contemplates a number of BMP-3 in modified forms. These modified forms include BMP-3 linked to another cystine knot growth factor or a fraction of such a monomer.

In specific embodiments, the mutant BMP-3 heterodimer comprising at least one mutant subunit or the single chain BMP-3 analog as described above is functionally active, i.e., capable of exhibiting one or more functional activities associated with the wild-type BMP-3, such as BMP-3 receptor binding, BMP-3 protein family receptor signalling and extracellular secretion. Preferably, the mutant BMP-3 heterodimer or single chain BMP-3 analog is capable of binding to the BMP-3 receptor, preferably with affinity greater than the wild type BMP-3. Also it is preferable that such a mutant BMP-3 heterodimer or single chain BMP-3 analog triggers signal transduction. Most preferably, the mutant BMP-3 heterodimer comprising at least one mutant subunit or the single chain BMP-3 analog of the present invention has an in vitro bioactivity and/or in vivo bioactivity greater than the wild type BMP-3 and has a longer serum half-life than wild type BMP-3. Mutant BMP-3 heterodimers and single chain BMP-3 analogs of the invention can be tested for the desired activity by procedures known in the art.

Mutants of the Human Bone Morphogenic Protein-3b (BMP-3b) Subunit

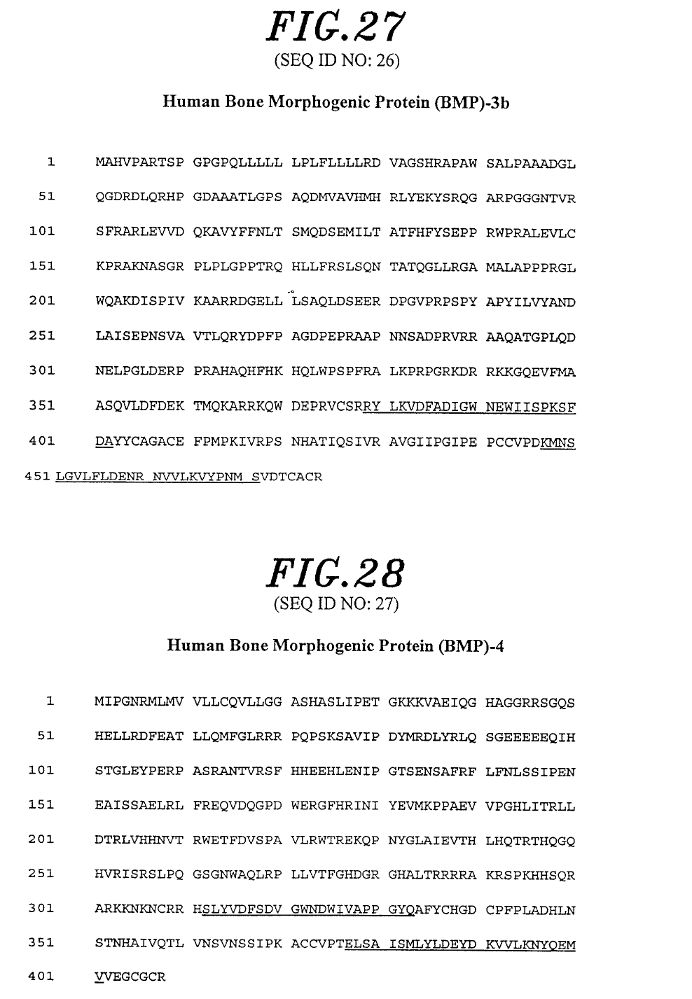

The human bone morphogenic protein-3b (BMP-3b) subunit contains 478 amino acids as shown in FIG. 27 (SEQ ID No: 26). The invention contemplates mutants of the BMP-3b subunit comprising single or multiple amino acid substitutions, deletions or insertions, of one, two, three, four or more amino acid residues when compared with the wild type monomer. Furthermore, the invention contemplates mutant BMP-3b subunit that are linked to another CKGF protein.

The present invention provides mutant BMP-3b subunit L1 hairpin loops having one or more amino acid substitutions between positions 379 to 402, inclusive, excluding Cys residues, as depicted in FIG. 27 (SEQ ID NO: 26). The amino acid substitutions include: R379X, Y380X, L381X, K382X, V383X, D384X, F385X, A386X, D387X, I388X, G389X, W390X, N391X, E392X, W393X, I394X, I395X, S396X, P397X, K398X, S399X, F400X, D401X, and A402X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the BMP-3b subunit monomer where an acidic residue is present, the variable “X” would correspond to a basic amino acid residue. Specific examples of electrostatic charge altering mutations where a basic residue is introduced into the BMP-3b subunit monomer include one or more of the following: D384B, D387B, E392B, and D401 wherein “B” is a basic amino acid residue.

Introducing acidic amino acid residues where basic residues are present in the BMP-3b subunit monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following R379Z, K382Z, and K398Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at R379U, K382U, D384U, D387U, E392U, K398U, and D401U, wherein “U” is a neutral amino acid.

Mutant BMP-3b subunit monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: Y380Z, L381Z, V383Z, F385Z, A386Z, I388Z, G389Z, W390Z, N391Z, W393Z, I394Z, I395Z, S396Z, P397Z, S399Z, F400Z, A402Z, Y380B, L381B, V383B, F385B, A386B, I388B, G389B, W390B, N391B, W393B, I394B, I395B, S396B, P397B, S399B, F400B, and A402B, wherein “Z” is an acidic amino acid and “B” is a basic amino acid.

Mutant BMP-3b subunit containing mutants in the L3 hairpin loop are also described. These mutant proteins have one or more amino acid substitutions, deletion or insertions, between positions 447 and 471, inclusive, excluding Cys residues, of the L3 hairpin loop, as depicted in FIG. 27 (SEQ ID NO: 26). The amino acid substitutions include: K447X, M448X, N449X, S450X, L451X, G452X, V453X, L454X, F455X, L456X, D457X, E458X, N459X, R460X, N461X, V462X, V463X, L464X, K465X, V466X, Y467X, P468X, N469X, M470X, and S471X, wherein “X” is any amino acid residue, the substitution of which alters the electrostatic character of the L3 loop.

One set of mutations of the L3 hairpin loop includes introducing one or more basic amino acid residues into the BMP-3b subunit L3 hairpin loop amino acid sequence. For example, when introducing basic residues into the L3 loop of the BMP-3b subunit, the variable “X” of the sequence described above corresponds to a basic amino acid residue. Specific examples of electrostatic charge altering mutations where a basic residue is introduced into the BMP-3b subunit include one or more of the following: D457B and E458B, wherein “B” is a basic amino acid residue.

The invention further contemplates introducing one or more acidic residues into the amino acid sequence of the BMP-3b subunit L3 hairpin loop. For example, one or more acidic amino acids can be introduced in the sequence of 447-471described above, wherein the variable “X” corresponds to an acidic amino acid. Specific examples of such mutations include K447Z, R460Z,and K465Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced of K447U, D457U, E458U, R460U, and K465, wherein “U” is a neutral amino acid.

Mutant BMP-3b subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include, M448Z, N449Z, S450Z, L451Z, G452Z, V453Z, L454Z, F455Z, L456Z, N459Z, N461Z, V462Z, V463Z, L464Z, V466Z, Y467Z, P468Z, N469Z, M470Z, S471Z, M448B, N449B, S450B, L451B, G452B, V453B, L454B, F455B, L456B, N459B, N461B, V462B, V463B, L464B, V466B, Y467B, P468B, N469B, M470B, and S471B, wherein “Z” is an acidic amino acid and “B” is a basic amino acid.

The present invention also contemplate BMP-3b subunit containing mutations outside of said β hairpin loop structures that alter the structure or conformation of those hairpin loops. These structural alterations in turn serve to increase the electrostatic interactions between regions of the β hairpin loop structures of BMP-3b subunit contained in a dimeric molecule, and a receptor having affinity for the dimeric protein. These mutations are found at positions selected from the group consisting of positions 1-378, 403-446, and 472-478 of the BMP-3b subunit monomer.

Specific examples of these mutation outside of the β hairpin L1 and L3 loop structures include, M1J, A2J, H3J, V4J, P5J, A6J, R7J, T8J, S9J, P10J, G11J, P12J, G13J, P14J, Q15J, L16J, L17J, L18J, L19J, L20J, L21J, P22J, L23J, F24J, L25J, L26J, L27J, L28J, R29J, D30J, V31J, A32J, G33J, S34J, H35J, R36J, A37J, P38J, A39J, W40J, S41J, A42J, L43J, P44J, A45J, A46J, A47J, D48J, G49J, L50J, Q51J, G52J, D53J, R54J, D55J, L56J, Q57J, R58J, H59J, P60J, G61J, D62J, A63J, A64J, A65J, T66J, L67J, G68J, P69J, S70J, A71J, Q72J, D73J, M74J, V75J, A76J, V77J, H78J, M79J, H80J, R81J, L82J, Y83J, E84J, K85J, Y86J, S87J, R88J, Q89J, G90J, A91J, R92J, P93J, G94J, G95J, G96J, N97J, T98J, V99J, R100J, S101J, F102J, R103J, A104J, R105J, L106J, E107J, V108J, V109J, D110J, Q111J, K112J, A113J, V114J, Y115J, F116J, F117J, N118J, L119J, T120J, S121J, M122J, Q123J, D124J, S125J, E126J, M127J, I128J, L129J, T130J, A131J, T132J, F133J, H134J, F135J, Y136J, S137J, E138J, P139J, P140J, R141J, W142J, P143J, R144J, A145J, L146J, E147J, V148J, L149J, C150J, K151J, P152J, R153J, A154J, K155J, N156J, A157J, S158J, G159J, R160J, P161J, L162J, P163J, L164J, G165J, P166J, P167J, T168J, R169J, Q170J, H171J, L172J, L173J, F174J, R175J, S176J, L177J, S178J, Q179J, N180J, T181J, A182J, T183J, Q184J, G185J, L186J, L187J, R188J, G189J, A190J, M191J, A192J, L193J, A194J, P195J, P196J, P197J, R198J, G199J, L200J, W201J, Q202J, A203J, K204J, D205J, I206J, S207J, P208J, I209J, V210J, K211J, A212J, A213J, R214J, R215J, D216J, G217J, E218J, L219J, L220J, L221J, S222J, A223J, Q224J, L225J, D226J, S227J, E228J, E229J, R230J, D231J, P232J, G233J, V234J, P235J, R236J, P237J, S238J, P239J, Y240J, A241J, P242J, Y243J, 1244J, L245J, V246J, Y247J, A248J, N249J, D250J, L251J, A252J, I253J, S254J, E255J, P256J, N257J, S258J, V259J, A260J, V261J, T262J, L263J, Q264J, R265J, Y266J, D267J, P268J, F269J, P270J, A271J, G272J, D273J, P274J, E275J, P276J, R277J, A278J, A279PJ, 280J, N281J, N282J, S283J, A284J, D285J, P286J, R287J, V288J, R289J, R290J, A291J, A292J, Q293J, A294J, T295J, G296J, P297J, L298J, Q299J, D300J, N301J, E302J, L303J, P304J, G305J, L306J, D307J, E308J, R309J, P310J, P311J, R312J, A313J, H314J, A315J, Q316J, H317J, F318J, H319J, K320J, H321J, Q322J, L323J, W324J, P325J, S326J, P327J, F328J, R329J, A330J, L331J, K332J, P333J, R334J, P335J, G336J, R337J, K338J, D339J, R340J, R341J, K342J, K343J, G344J, Q345J, E346J, V347J, F348J, M349J, A350J, A351J, S352J, Q353J, V354J, L355J, D356J, F357J, D358J, E359J, K360J, T361J, M362J, Q363J, K364J, A365J, R366J, R367J, K368J, Q369J, W370J, D371J, E372J, P373J, R374J, V375J, C376J, S377J, R378J, Y403J, Y404J, C405J, A406J, G407J, A408J, C409J, E410J, F411J, P412J, M413J, P414J, K415J, I416J, V417J, R418J, P419J, S420J, N421J, H422J, A423J, T424J, I425J, Q426J, S427J, I428J, V429J, R430J, A431J, V432J, G433J, I434J, I435J, P436J, G437J, I438J, P439J, E440J, P441J, C442J, C443J, V444J, P445J, D446J, V472J, D473J, T474J, C475J, A476J, C477J, and R478J. The variable “J” is any amino acid whose introduction results in an increase in the electrostatic interaction between the L1 and L3 β hairpin loop structures of the BMP-3b subunit and a receptor with affinity for a dimeric protein containing the mutant BMP-3b subunit monomer.

The invention also contemplates a number of BMP-3b subunit in modified forms. These modified forms include BMP-3b subunit linked to another cystine knot growth factor or a fraction of such a monomer.

In specific embodiments, the mutant BMP-3b subunit heterodimer comprising at least one mutant subunit or the single chain BMP-3b subunit analog as described above is functionally active, i.e., capable of exhibiting one or more functional activities associated with the wild-type BMP-3b subunit, such as BMP-3b subunit receptor binding, BMP-3b subunit protein family receptor signalling and extracellular secretion. Preferably, the mutant BMP-3b subunit heterodimer or single chain BMP-3b subunit analog is capable of binding to the BMP-3b subunit receptor, preferably with affinity greater than the wild type BMP-3b subunit. Also it is preferable that such a mutant BMP-3b subunit heterodimer or single chain BMP-3b subunit analog triggers signal transduction. Most preferably, the mutant BMP-3b subunit heterodimer comprising at least one mutant subunit or the single chain BMP-3b subunit analog of the present invention has an in vitro bioactivity and/or in vivo bioactivity greater than the wild type BMP-3b subunit and has a longer serum half-life than wild type BMP-3b subunit. Mutant BMP-3b subunit heterodimers and single chain BMP-3b subunit analogs of the invention can be tested for the desired activity by procedures known in the art.

Mutants of the Human Bone Morphogenic Protein-4 (BMP-4) Subunit

The human bone morphogenic protein-4 (BMP-4) subunit contains 408 amino acids as shown in FIG. 28 (SEQ ID No: 27). The invention contemplates mutants of the BMP-4 subunit comprising single or multiple amino acid substitutions, deletions or insertions, of one, two, three, four or more amino acid residues when compared with the wild type monomer. Furthermore, the invention contemplates mutant BMP-4 subunit that are linked to another CKGF protein.

The present invention provides mutant BMP-4 subunit L1 hairpin loops having one or more amino acid substitutions between positions 312 and 33, inclusive, excluding Cys residues, as depicted in FIG. 28 (SEQ ID NO: 27). The amino acid substitutions include: S312X, L313X, Y314X, V315X, D316X, F317X, S318X, D139X, V320X, G321X, W322X, N323X, D324X, W325X, I326X, V327X, A328X, P329X, P330X, G331X, Y332X, and Q333X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the BMP-4 subunit monomer where an acidic residue is present, the variable “X” would correspond to a basic amino acid residue. Specific examples of electrostatic charge altering mutations where a basic residue is introduced into the BMP-4 subunit monomer include one or more of the following: D316B, D319B, and D324B wherein “B” is a basic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced D316U, D319U, and D324U, wherein “U” is a neutral amino acid.

Mutant BMP-4 subunit proteins are provided containing one or more electrostatic charge altering mutations in the L1 hairpin loop amino acid sequence that convert non-charged or neutral amino acid residues to charged residues. Examples of mutations converting neutral amino acid residues to charged residues include: S312Z, L313Z, Y314Z, V315Z, F317Z, S318Z, V320Z, G321Z, W322Z, N323Z, W325Z, I326Z, V327Z, A328Z, P329Z, P330Z, G331Z, Y332Z, Q333Z, S312B, L313B, Y314B, V315B, F317B, S318B, V320B, G321B, W322B, N323B, W325B, I326B, V327B, A328B, P329B, P330B, G331B, Y332B, and Q333B, wherein “Z” is an acidic amino acid and “B” is a basic amino acid.

Mutant BMP-4 subunit containing mutants in the L3 hairpin loop are also described. These mutant proteins have one or more amino acid substitutions, deletion or insertions, between positions 377 and 401, inclusive, excluding Cys residues, of the L3 hairpin loop, as depicted in FIG. 28 (SEQ ID NO: 27). The amino acid substitutions include E377X, L378X, S379X, A380X, I381X, S382X, M383X, L384X, Y385X, L386X, D387X, E388X, Y389X, D390X, K391X, V392X, V393X, L394X, K395X, N396X, Y397X, Q398X, E399X, M400X, and V401X, wherein “X” is any amino acid residue, the substitution of which alters the electrostatic character of the L3 loop.

One set of mutations of the L3 hairpin loop includes introducing one or more basic amino acid residues into the BMP-4 subunit L3 hairpin loop amino acid sequence. For example, when introducing basic residues into the L3 loop of the BMP-4 subunit, the variable “X” of the sequence described above corresponds to a basic amino acid residue. Specific examples of electrostatic charge altering mutations where a basic residue is introduced into the BMP-4 subunit include one or more of the following: E377B, D387B, E388B, D390B, and E399B, wherein “B” is a basic amino acid residue.

The invention further contemplates introducing one or more acidic residues into the amino acid sequence of the BMP-4 subunit L3 hairpin loop. For example, one or more acidic amino acids can be introduced in the sequence of 377-401described above, wherein the variable “X” corresponds to an acidic amino acid. Specific examples of such mutations include K391Z and K395Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at E377U, D387U, E388U, D390U, K391U, K395U, and E399U, wherein “U” is a neutral amino acid.

Mutant BMP-4 subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include, L378Z, S379Z, A380Z, I381Z, S382Z, M383Z, L384Z, Y385Z, L386Z, Y389Z, V392Z, V393Z, L394Z, N396Z, Y397Z, Q398Z, M400Z, V401Z, L378B, S379B, A380B, I381B, S382B, M383B, L384B, Y385B, L386B, Y389B, V392B, V393B, L394B, N396B, Y397B, Q398B, M400B, and V401B, wherein “Z” is an acidic amino acid and “B” is a basic amino acid.

The present invention also contemplate BMP-4 subunit containing mutations outside of said β hairpin loop structures that alter the structure or conformation of those hairpin loops. These structural alterations in turn serve to increase the electrostatic interactions between regions of the β hairpin loop structures of BMP-4 subunit contained in a dimeric molecule, and a receptor having affinity for the dimeric protein. These mutations are found at positions selected from the group consisting of positions 1-311, 334-376, and 402-408 of the BMP-4 subunit monomer.

Specific examples of these mutation outside of the β hairpin L1 and L3 loop structures include, M1J, I2J, P3J, G4J, N5J, R6J, M7J, L8J, M9J, V10J, V11J, L12J, L13J, C14J, Q15J, V16J, L17J, L18J, G19J, G20J, A21J, S22J, H23J, A24J, S25J, L26J, I27J, P28J, E29J, T30J, G31J, K32J, K33J, K34J, V35J, A36J, E37J, I38J, Q39J, G40J, H41J, A42J, G43J, G44J, R45J, R46J, S47J, G48J, Q49J, S50J, H51J, E52J, L53J, L54J, R55J, D56J, F57J, E58J, A59J, T60J, L61J, L62J, Q63J, M64J, F65J, G66J, L67J, R68J, R69J, R70J, P71J, Q72J, P73J, S74J, K75J, S76J, A77J, V78J, I79J, P80J, D81J, Y82J, M83J, R84J, D85J, L86J, Y87J, R88J, L89J, Q90J, S91J, G92J, E93J, E94J, E95J, E96J, E97J, Q98J, I99J, H100J, S101J, T102J, G103J, L104J, E105J, Y106J, P107J, E108J, R109J, P110J, A111J, S112J, R113J, A114J, N115J, T116J, V117J, R118J, S119J, F120J, H121J, H122J, E123J, E124J, H125J, L126J, E127J, N128J, I129J, P130J, G131J, T132J, S133J, E134J, N135J, S136J, A137J, F138J, R139J, F140J, L141J, F142J, N143J, L144J, S145J, S146J, I147J, P148J, E149J, N150J, E151J, A152J, I153J, S154J, S155J, A156J, E157J, L158J, R159J, L160J, F161J, R162J, E163J, Q164J, V165J, D166J, Q167J, G168J, P169J, D107J, W171J, E172J, R173J, G174J, F175J, H176J, R177J, I178J, N179J, I180J, Y181J, E182J, V183J, M184J, K185J, P186J, P187J, A188J, E189J, V190J, V191J, P192J, G193J, H194J, L195J, I196J, T197J, R198J, L199J, L200J, D201J, T202J, R203J, L204J, V205J, H206J, H207J, N208J, V209J, T210J, R211J, W212J, E213J, T214J, F215J, D216J, V217J, S218J, P219J, A220J, V221J, L222J, R223J, W224J, T225J, R226J, E227J, K228J, Q229J, P230J, N231J, Y232J, G233J, L234J, A235J, I236J, E237J, V238J, T239J, H240J, L241J, H242J, Q243J, T244J, R245J, T246J, H247J, Q248J, G249J, Q250J, H251J, V252J, R253J, I254J, S255J, R256J, S257J, L258J, P259J, Q260J, G261J, S262J, G263J, N264J, W265J, A266J, Q267J, L268J, R269J, P270J, L271J, L272J, V273J, T274J, F275J, G276J, H277J, D278J, G279J, R280J, G281J, H282J, A283J, L284J, T285J, R286J, R287J, R288J, R289J, A290J, K291J, R292J, S293J, P294J, K295J, H296J, H297J, S298J, Q299J, R300J, A301J, R302J, K303J, K304J, N305J, K306J, N307J, C308J, R309J, R310J, H311J, A334J, F335J, Y336J, C337J, H338J, G339J, D340J, C341J, P342J, F343J, P344J, L345J, A346J, D347J, H348J, L349J, N350J, S351J, T352J, N353J, H354J, A355J, I356J, V357J, Q358J, T359J, L360J, V361J, N362J, S363J, V364J, N365J, S366J, S367J, I368J, P369J, K370J, A371J, C372J, C373J, V374J, P375J, T376J, V402J, E403J, G404J, C405J, G406J, C407J, and R408J, The variable “J” is any amino acid whose introduction results in an increase in the electrostatic interaction between the L1 and L3 β hairpin loop structures of the BMP-4 subunit and a receptor with affinity for a dimeric protein containing the mutant BMP-4 subunit monomer.

The invention also contemplates a number of BMP-4 subunit in modified forms. These modified forms include BMP-4 subunit linked to another cystine knot growth factor or a fraction of such a monomer.

In specific embodiments, the mutant BMP-4 subunit heterodimer comprising at least one mutant subunit or the single chain BMP-4 subunit analog as described above is functionally active, i.e., capable of exhibiting one or more functional activities associated with the wild-type BMP-4 subunit, such as BMP-4 subunit receptor binding, BMP-4 subunit protein family receptor signalling and extracellular secretion. Preferably, the mutant BMP-4 subunit heterodimer or single chain BMP-4 subunit analog is capable of binding to the BMP-4 subunit receptor, preferably with affinity greater than the wild type BMP-4 subunit. Also it is preferable that such a mutant BMP-4 subunit heterodimer or single chain BMP-4 subunit analog triggers signal transduction. Most preferably, the mutant BMP-4 subunit heterodimer comprising at least one mutant subunit or the single chain BMP-4 subunit analog of the present invention has an in vitro bioactivity and/or in vivo bioactivity greater than the wild type BMP-4 subunit and has a longer serum half-life than wild type BMP-4 subunit. Mutant BMP-4 subunit heterodimers and single chain BMP-4 subunit analogs of the invention can be tested for the desired activity by procedures known in the art.

Mutants of the Human Bone Morohogenic Protein-5 (BMP-5) Precusor Subunit

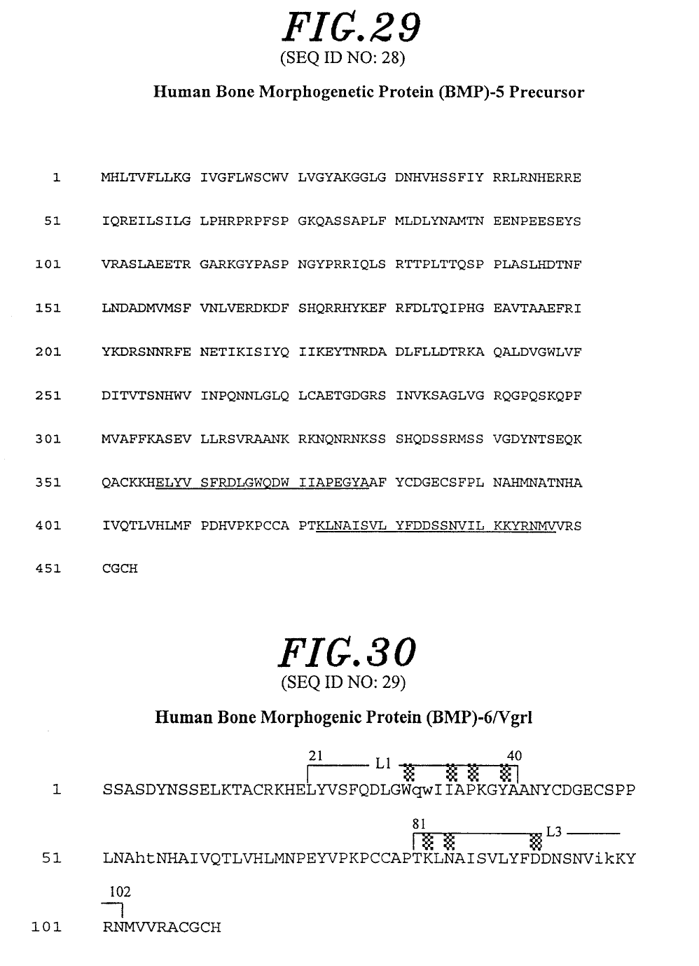

The human bone morphogenic protein-5 (BMP-5) precusor subunit contains 112 amino acids as shown in FIG. 29 (SEQ ID No: 28). The invention contemplates mutants of the BMP-5 precusor subunit comprising single or multiple amino acid substitutions, deletions or insertions, of one, two, three, four or more amino acid residues when compared with the wild type monomer. Furthermore, the invention contemplates mutant BMP-5 precusor subunit that are linked to another CKGF protein.

The present invention provides mutant BMP-5 precusor subunit L1 hairpin loops having one or more amino acid substitutions between positions 357 and 378, inclusive, excluding Cys residues, as depicted in FIG. 29 (SEQ ID NO: 28). The amino acid substitutions include: E357X, L358X, Y359X, V360X, S361X, F362X, R363X, D364X, L365X, G366X, W367X, Q368X, D369X, W370X, I371X, I372X, A373X, P374X, E375X, G376X, Y377X, and A378X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the BMP-5 precusor subunit monomer where an acidic residue is present, the variable “X” would correspond to a basic amino acid residue. Specific examples of electrostatic charge altering mutations where a basic residue is introduced into the BMP-5 precusor subunit monomer include one or more of the following: E357B, D364B, D369B, and E375B wherein “B” is a basic amino acid residue.

Introducing acidic amino acid residues where basic residues are present in the BMP-5 precusor subunit monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include R363Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced of E357U, R363U, D364U, D369U, and E375U, wherein “U” is a neutral amino acid.

Mutant BMP-5 precusor subunit monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include, L358Z, Y359Z, V360Z, S361Z, F362Z, L365Z, G366Z, W367Z, Q368Z, W370Z, I371Z, I372Z, A373Z, P374Z, G376Z, Y377Z, A378Z, L358B, Y359B, V360B, S361B, F362B, L365B, G366B, W367B, Q368B, W370B, I371B, I372B, A373B, P374B, G376B, Y377B, and A378B, wherein “Z” is an acidic amino acid and “B” is a basic amino acid.

Mutant BMP-5 precusor subunit containing mutants in the L3 hairpin loop are also described. These mutant proteins have one or more amino acid substitutions, deletion or insertions, between positions 423 and 447, inclusive, excluding Cys residues, of the L3 hairpin loop, as depicted in FIG. 29 (SEQ ID NO: 28). The amino acid substitutions include: K423X, L424X, N425X, A426X, I427X, S428X, V429X, L430X, Y431X, F432X, D433X, D434X, S435X, S436X, N437X, V438X, I439X, L440X, K441X, K442X, Y443X, R444X, N445X, M446X, and V447X, wherein “X” is any amino acid residue, the substitution of which alters the electrostatic character of the L3 loop.

One set of mutations of the L3 hairpin loop includes introducing one or more basic amino acid residues into the BMP-5 precusor subunit L3 hairpin loop amino acid sequence. For example, when introducing basic residues into the L3 loop of the BMP-5 precusor subunit, the variable “X” of the sequence described above corresponds to a basic amino acid residue. Specific examples of electrostatic charge altering mutations where a basic residue is introduced into the BMP-5 precusor subunit include one or more of the following: D433B and D434B, wherein “B” is a basic amino acid residue.

The invention further contemplates introducing one or more acidic residues into the amino acid sequence of the BMP-5 precusor subunit L3 hairpin loop. For example, one or more acidic amino acids can be introduced in the sequence of 423-447described above, wherein the variable “X” corresponds to an acidic amino acid. Specific examples of such mutations include K423Z, K441Z, K442Z, and R444Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where ffie variable “X” corresponds to a neutral amino acid. For example, one or more neutral residues can be introduced at K423U, D433U, D434U, K441U, K442U, and R444U, wherein “U” is a neutral amino acid.

Mutant BMP-5 precusor subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include, L424Z, N425Z, A426Z, I427Z, S428Z, V429Z, L430Z, Y431Z, F432Z, S435Z, S436Z, N437Z, V438Z, I439Z, L440Z, Y443Z, R444Z, N445Z, M446Z, V447Z, L424B, N425B, A426B, I427B, S428B, V429B, L430B, Y431B, F432B, S435B, S436B, N437B, V438B, I439B, L440B, Y443B, N445B, M446B, and V447B, wherein “Z” is an acidic amino acid and “B” is a basic amino acid.

The present invention also contemplate BMP-5 precusor subunit containing mutations outside of said β hairpin loop structures that alter the structure or conformation of those hairpin loops. These structural alterations in turn serve to increase the electrostatic interactions between regions of the β hairpin loop structures of BMP-5 precusor subunit contained in a dimeric molecule, and a receptor having affinity for the dimeric protein. These mutations are found at positions selected from the group consisting of positions 1-356, 379-422, and 448-454 of the BMP-5 precusor subunit monomer.

Specific examples of these mutation outside of the β hairpin L1 and L3 loop structures include, M1J, H2J, L3J, T4J, V5J, F6J, L7J, L8J, K9J, G10J, I11J, V12J, G13J, F14J, L15J, W16J, S17J, C18J, W19J, V20J, L21J, V22J, G23J, Y24J, A25J, K26J, G27J, G28J, L29J, G30J, D31J, N32J, H33J, V34J, H35J, S36J, S37J, F38J, I39J, Y40J, R41J, R42J, L43J, R44J, N45J, H46J, E47J, R48J, R49J, E50J, I51J, Q52J, R53J, E54J, I55J, L56J, S57J, I58J, L59J, G60J, L61J, P62J, H63J, R64J, P65J, R66J, P67J, F68J, S69J, P70J, G71J, K72J, Q73J, A74J, S75J, S76J, A77J, P78J, L79J, F80J, M81J, L82J, D83J, L84J, Y85J, N86J, A87J, M88J, T89J, N90J, E91J, E92J, N93J, P94J, E95J, E96J, S97J, E98J, Y99J, S100J, V101J, R102J, A103J, S104J, L105J, A106J, E107J, E108J, T109J, R110J, G111J, A112J, R113J, K114J, G115J, Y116J, P117J, A118J, S119J, P120J, N121J, G122J, Y123J, P124J, R125J, R126J, I127J, Q128J, L129J, S130J, R131J, T132J, T133J, P134J, L135J, T136J, T137J, Q138J, S139J, P140J, P141J, L142J, A143J, S144J, L145J, H146J, D147J, T148J, N149J, F150J, L151J, N152J, D153J, A154J, D155J, M156J, V157J, M158J, S159J, F160J, V161J, N162J, L163J, V164J, E165J, R166J, D167J, K168J, D169J, F170J, S171J, H172J, Q173J, R174J, R175J, H176J, Y177J, K178J, E179J, F180J, R181J, F182J, D183J, L184J, T185J, Q186J, I187J, P188J, H189J, G190J, E191J, A192J, V193J, T194J, A195J, A196J, E197J, F198J, R199J, I200J, Y201J, K202J, D203J, R204J, S205J, N206J, N207J, R208J, F209J, E210J, N211J, E212J, T213J, I214J, K215J, I216J, S217J, I218J, Y219J, Q220J, I221J, I222J, K223J, E224J, Y225J, T226J, N227J, R228J, D229J, A230J, D231J, L232J, F233J, L234J, L235J, D236J, T237J, R238J, K239J, A240J, Q241J, A242J, L243J, D244J, V245J, G246J, W247J, L248J, V249J, F250J, D251J, I252J, T253J, V254J, T255J, S256J, N257J, H258J, W259J, V260J, I261J, N262J, P263J, Q264J, N265J, N266J, L267J, G268J, L269J, Q270J, L271J, C272J, A273J, E274J, T275J, G276J, D277J, G278J, R279J, S280J, I281J, N282J, V283J, K284J, S285J, A286J, G287J, L288J, V289J, G290J, R291J, Q292J, G293J, P294J, Q295J, S296J, K297J, Q298J, P299J, F300J, M301J, V302J, A303J, F304J, F305J, K306J, A307J, S308J, E309J, V310J, L311J, L312J, R313J, S314J, V315J, R316J, A317J, A318J, N319J, K320J, R321J, K322J, N323J, Q324J, N325J, R326J, N327J, K328J, S329J, S330J, S331J, H332J, Q333J, D334J, S335J, S336J, R337J, M338J, S339J, S340J, V341J, G342J, D343J, Y344J, N345J, T346J, S347J, E348J, Q349J, K350J, Q351J, A352J, C353J, K354J, K355J, H356J, A379J, F380J, Y381J, C382J, D283J, G384J, E385J, C386J, S387J, F388J, P389J, L390J, N391J, A392J, H393J, M394J, N395J, A396J, T397J, N398J, H399J, A400J, I401J, V402J, Q403J, T404J, L405J, V406J, H407J, L408J, M409J, F410J, P411J, D412J, H413J, V414J, P415J, K416J, P417J, C418J, C419J, A420J, P421J, T422J, V448J, R449J, S450J, C451J, G452J, C453J, and H454J. The variable “J” is any amino acid whose introduction results in an increase in the electrostatic interaction between the L1 and L3 β hairpin loop structures of the BMP-5 precusor subunit and a receptor with affinity for a dimeric protein containing the mutant BMP-5 precusor subunit monomer.

The invention also contemplates a number of BMP-5 precusor subunit in modified forms. These modified forms include BMP-5 precusor subunit linked to another cystine knot growth factor or a fraction of such a monomer.

In specific embodiments, the mutant BMP-5 precusor subunit heterodimer comprising at least one mutant subunit or the single chain BMP-5 precusor subunit analog as described above is functionally active, i.e., capable of exhibiting one or more functional activities associated with the wild-type BMP-5 precusor subunit, such as BMP-5 precusor subunit receptor binding, BMP-5 precusor subunit protein family receptor signalling and extracellular secretion. Preferably, the mutant BMP-5 precusor subunit heterodimer or single chain BMP-5 precusor subunit analog is capable of binding to the BMP-5 precusor subunit receptor, preferably with affinity greater than the wild type BMP-5 precusor subunit. Also it is preferable that such a mutant BMP-5 precusor subunit heterodimer or single chain BMP-5 precusor subunit analog triggers signal transduction. Most preferably, the mutant BMP-5 precusor subunit heterodimer comprising at least one mutant subunit or the single chain BMP-5 precusor subunit analog of the present invention has an in vitro bioactivity and/or in vivo bioactivity greater than the wild type BMP-5 precusor subunit and has a longer serum half-life than wild type BMP-5 precusor subunit. Mutant BMP-5 precusor subunit heterodimers and single chain BMP-5 precusor subunit analogs of the invention can be tested for the desired activity by procedures known in the art.

Mutants of the Human Bone Morphogenic Protein-6/Vgrl Growth Factor Monomer

The human contains 111 amino acids as shown in FIG. 30 (SEQ ID No: 29). The invention contemplates mutants of the human bone morphogenic protein-6/Vgrl growth factor monomer comprising single or multiple amino acid substitutions, deletions or insertions, of one, two, three, four or more amino acid residues when compared with the wild type monomer. Furthermore, the invention contemplates mutant human bone morphogenic protein-6/Vgrl growth factor monomers that are linked to another CKGF protein.

The present invention provides mutant bone morphogenic protein-6/Vgrl growth factor monomer L1 hairpin loops having one or more amino acid substitutions between positions 21 and 40, inclusive, excluding Cys residues, as depicted in FIG. 30 (SEQ ID No: 29). The amino acid substitutions include Y21X, V22X, S23X, F24X, Q25X, D26X, L27X, G28X, W29X, Q30X, W31X, I32X, I33X, A34X, P35X, K36X, G37X, Y38X, A39X, and A40X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the bone morphogenic protein-6/Vgrl growth factor monomer, the variable “X” would correspond to a basic amino acid residue. Specific examples of electrostatic charge altering mutations where a basic residue is introduced into the bone morphogenic protein-6/Vgrl growth factor monomer at D26B, wherein “B” is a basic amino acid residue.

Introducing acidic amino acid residues where basic residues are present in the bone morphogenic protein-6/Vgrl growth factor monomer sequence is also contemplated. In this embodiment, the variable “X” corresponds to an acidic amino acid. The introduction of these amino acids serves to alter the electrostatic character of the L1 hairpin loops to a more negative state. An example of such an amino acid substitution is K36Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced of D26U and K36U, wherein “U” is a neutral amino acid.

Mutant bone morphogenic protein-6/Vgrl growth factor monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include of Y21Z, V22Z, S23Z, F24Z, Q25Z, L27Z, G28Z, W29Z, Q30Z, W31Z, I32Z, I33Z, A34Z, P35Z, G37Z, Y38Z, A39Z, A40Z, Y21B, V22B, S23B, F24B, Q25B, L27B, G28B, W29B, Q30B, W31B, I32B, I33B, A34B, P35B, G37B, Y38B, A39B, and A40B, wherein “Z” is an acidic amino acid and “B” is a basic amino acid.

Mutant transforming growth factor β3 monomers containing mutants in the L3 hairpin loop are also described. These mutant proteins have one or more amino acid substitutions, deletion or insertions, between positions 81 and 102, inclusive, excluding Cys residues, of the L3 hairpin loop, as depicted in FIG. 30 (SEQ ID No: 29). The amino acid substitutions include: K81X, L82X, N83X, A84X, I85X, S86X, V87X, L88X, Y89X, F90X, D91X, D92X, N93X, S94X, N95X, V96X, I97X, K98X, K99X, Y100X, R101X, and N102X, wherein “X” is any amino acid residue, the substitution of which alters the electrostatic character of the L3 loop.

One set of mutations of the L3 hairpin loop includes introducing one or more basic amino acid residues into the transforming growth factor β1 L3 hairpin loop amino acid sequence. For example, when introducing basic residues into the L3 loop of the transforming growth factor β3 monomer, the variable “X” of the sequence described above corresponds to a basic amino acid residue. Specific examples of electrostatic charge altering mutations where a basic residue is introduced into the bone morphogenic protein-6/Vgrl growth factor monomer include one or more of the following: D91B and D92B, wherein “B” is a basic amino acid residue.

The invention further contemplates introducing one or more acidic residues into the amino acid sequence of the bone morphogenic protein-6/Vgrl growth factor L3 hairpin loop. For example, one or more acidic amino acids can be introduced in the sequence of 81-102 described above, wherein the variable “X” corresponds to an acidic amino acid. Specific examples of such mutations include, K81Z, K98Z, K99Z, and R101Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K81U, D91U, D92U, K98U, K99U, and R101U, wherein “U” is a neutral amino acid.

Mutant transforming growth factor β1 proteins are provided containing one or more electrostatic charge altering mutations in the L3 hairpin loop amino acid sequence that convert non-charged or neutral amino acid residues to charged residues. Examples of mutations converting neutral amino acid residues to charged residues include, L82Z, N83Z, A84Z, I85Z, S86Z, V87Z, L88Z, Y89Z, F90Z, N93Z, S94Z, N95Z, V96Z, I97Z, Y100Z, N102Z, L82B, N83B, A84B, I85B, S86B, V87B, L88B, Y89B, F90B, N93B, S94B, N95B, V96B, I97B, Y100B, and N102B, wherein “Z” is an acidic amino acid and “B” is a basic amino acid.

The present invention also contemplates transforming growth factor β3 monomers containing mutations outside of said β hairpin loop structures that alter the structure or conformation of those hairpin loops. These structural alterations in turn serve to increase the electrostatic interactions between regions of the β hairpin loop structures of a bone morphogenic protein-6/Vgrl growth factor monomer contained in a dimeric molecule, and a receptor having affinity for the dimeric protein. These mutations are found at positions selected from the group consisting of positions 1-20, 41-81, and 103-111 of the bone morphogenic protein-6/Vgrl growth factor monomer.

Specific examples of these mutation outside of the β hairpin L1 and L3 loop structures include, S1J, S2J, A3J, S4J, D5J, Y6J, N7J, S8J, S9J, E10J, L11J, K12J, T13J, A14J, C15J, R16J, K17J, H18J, E19J, L20J, N41J, Y42J, C43J, D44J, G45J, E46J, C47J, S48J, P49J, P50J, L51J, N52J, A53J, H54J, T55J, N56J, H57J, A58J, I59J, V60J, Q61J, T62J, L63J, V64J, H65J, L66J, M67J, N68J, P69J, E70J, Y71J, V72J, P73J, K74J, P75J, C76J, C77J, A78J, P79J, T80J, M103J, V104J, V105J, R106J, A107J, C108J, G109J, C110J, and H111J, The variable “J” is any amino acid whose introduction results in an increase in the electrostatic interaction between the L1 and L3 β hairpin loop structures of the bone morphogenic protein-6/Vgrl growth factor and a receptor with affinity for a dimeric protein containing the mutant bone morphogenic protein-6/Vgrl growth factor monomer.

The invention also contemplates a number of bone morphogenic protein-6/Vgrl growth factor monomers in modified forms. These modified forms include bone morphogenic protein-6/Vgrl growth factor monomers linked to another cystine knot growth factor monomer or a fraction of such a monomer.

In specific embodiments, the mutant bone morphogenic protein-6/Vgrl growth factor heterodimer comprising at least one mutant subunit or the single chain bone morphogenic protein-6/Vgrl growth factor analog as described above is functionally active, i.e., capable of exhibiting one or more functional activities associated with the wild-type bone morphogenic protein-6/Vgrl growth factor, such as bone morphogenic protein-6/Vgrl growth factor receptor binding, bone morphogenic protein-6/Vgrl growth factor receptor signalling and extracellular secretion. Preferably, the mutant bone morphogenic protein-6/Vgrl growth factor heterodimer or single chain bone morphogenic protein-6/Vgrl growth factor analog is capable of binding to the bone morphogenic protein-6/Vgrl growth factor receptor, preferably with affinity greater than the wild type bone morphogenic protein-6/Vgrl growth factor. Also it is preferable that such a mutant bone morphogenic protein-6/Vgrl growth factor heterodimer or single chain bone morphogenic protein-6/Vgrl growth factor analog triggers signal transduction. Most preferably, the mutant bone morphogenic protein-6/Vgrl growth factor heterodimer comprising at least one mutant subunit or the single chain bone morphogenic protein-6/Vgrl growth factor analog of the present invention has an in vitro bioactivity and/or in vivo bioactivity greater than the wild type bone morphogenic protein-6/Vgrl growth factor and has a longer serum half-life than wild type bone morphogenic protein-6/Vgrl growth factor. Mutant bone morphogenic protein-6/Vgrl growth factor heterodimers and single chain bone morphogenic protein-6/Vgrl growth factor analogs of the invention can be tested for the desired activity by procedures known in the art.

Mutants of the Human Bone Morohogenic Protein-7/Osteogenic Protein-1 Monomer

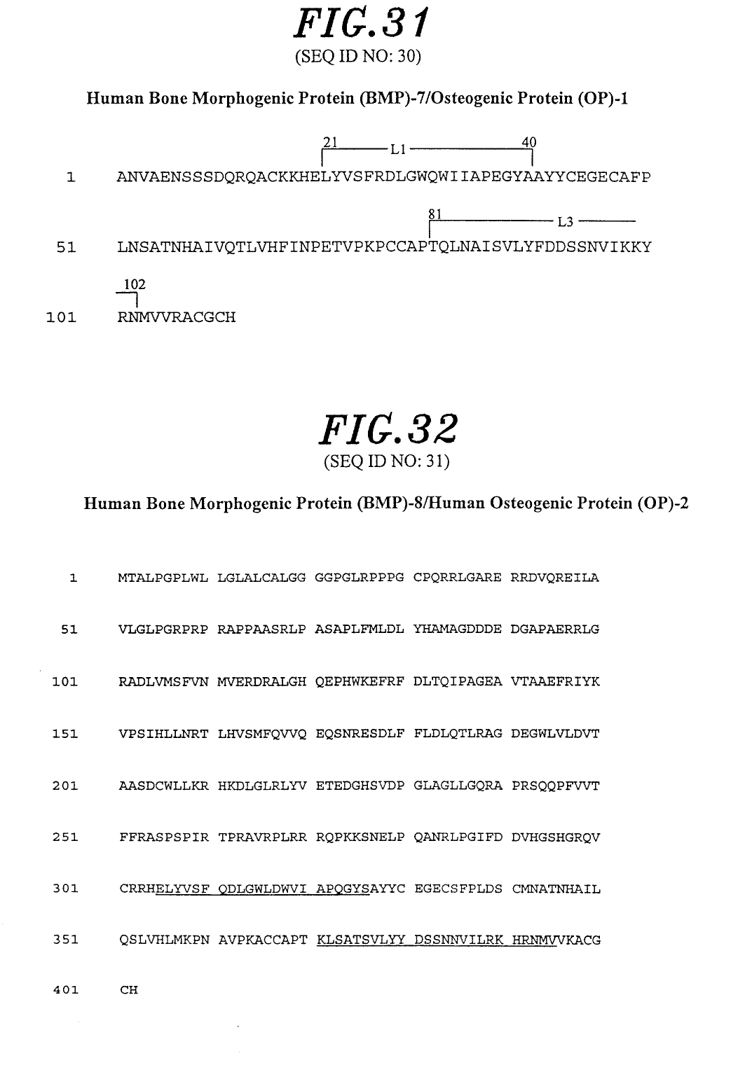

The human contains 111 amino acids as shown in FIG. 31 (SEQ ID No: 30). The invention contemplates mutants of the human bone morphogenic protein-7/osteogenic protein-1 monomer comprising single or multiple amino acid substitutions, deletions or insertions, of one, two, three, four or more amino acid residues when compared with the wild type monomer. Furthermore, the invention contemplates mutant human bone morphogenic protein-7/osteogenic protein-1 monomers that are linked to another CKGF protein.

The present invention provides mutant bone morphogenic protein-7/osteogenic protein-1 monomer L1 hairpin loops having one or more amino acid substitutions between positions 21 and 40, inclusive, excluding Cys residues, as depicted in FIG. 31 (SEQ ID NO: 30). The amino acid substitutions include: Y21X, V22X, S23X, F24X, R25X, D26X, L27X, G28X, W29X, Q30X, W31X, I32X, I33X, A34X, P35X, E36X, G37X, Y38X, A39X, and A40X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the bone morphogenic protein-7/osteogenic protein-1 monomer, the variable “X” would correspond to a basic amino acid residue. Specific examples of electrostatic charge altering mutations where a basic residue is introduced into the bone morphogenic protein-7/osteogenic protein-1 monomer include one or more of the following: D26B and E36B, wherein “B” is a basic amino acid residue.

Introducing acidic amino acid residues where basic residues are present in the bone morphogenic protein-7/osteogenic protein-1 monomer sequence is also contemplated. In this embodiment, the variable “X” corresponds to an acidic amino acid. The introduction of these amino acids serves to alter the electrostatic character of the L1 hairpin loops to a more negative state. An example of such an amino acid substitution is R25Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced of R25U, D26U and E36U, wherein “U” is a neutral amino acid.

Mutant bone morphogenic protein-7/osteogenic protein-1 monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include of Y21Z, V22Z, S23Z, F24Z, L27Z, G28Z, W29Z, Q30Z, W31Z, I32Z, I33Z, A34Z, P35Z, G37Z, Y38Z, A39Z, and A40Z, wherein “Z” is an acidic amino acid and “B” is a basic amino acid.

Mutant bone morphogenic protein-7/osteogenic protein-1 monomers containing mutants in the L3 hairpin loop are also described. These mutant proteins have one or more amino acid substitutions, deletion or insertions, between positions 81 and 102, inclusive, excluding Cys residues, of the L3 hairpin loop, as depicted in FIG. 31 (SEQ ID NO: 30). The amino acid substitutions include: Q81X, L82X, N83X, A84X, I85X, S86X, V87X, L88X, Y89X, F90X, D91X, D92X, S93X, S94X, N95X, V96X, I97X, K98X, K99X, Y100X, R101X, and N102X, wherein “X” is any amino acid residue, the substitution of which alters the electrostatic character of the L3 loop.

One set of mutations of the L3 hairpin loop includes introducing one or more basic amino acid residues into the transforming growth factor β1 L3 hairpin loop amino acid sequence. For example, when introducing basic residues into the L3 loop of the transforming growth factor β3 monomer, the variable “X” of the sequence described above corresponds to a basic amino acid residue. Specific examples of electrostatic charge altering mutations where a basic residue is introduced into the bone morphogenic protein-7/osteogenic protein-1 monomer include one or more of the following: D91B and D92B, wherein “B” is a basic amino acid residue.

The invention further contemplates introducing one or more acidic residues into the amino acid sequence of the bone morphogenic protein-7/osteogenic protein-1 L3 hairpin loop. For example, one or more acidic amino acids can be introduced in the sequence of 81-102 described above, wherein the variable “X” corresponds to an acidic amino acid. Specific examples of such mutations include of K98Z, K99Z, and R101Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at D91U, D92U, K98U, K99U, and R101U, wherein “U” is a neutral amino acid.

Mutant bone morphogenic protein-7/osteogenic protein-1 monomers are provided containing one or more electrostatic charge altering mutations in the L3 hairpin loop amino acid sequence that convert non-charged or neutral amino acid residues to charged residues. Examples of mutations converting neutral amino acid residues to charged residues include, Q81Z, L82Z, N83Z, A84Z, I85Z, S86Z, V87Z, L88Z, Y89Z, F90Z, N93Z, S94Z, N95Z, V96Z, I97Z, Y100Z, N102B, Q81B, L82B, N83B, A84B, I85B, S86B, V87B, L88B, Y89B, F90B, N93B, S94B, N95B, V96B, I97B, Y100B, and N102B, wherein “Z” is an acidic amino acid and “B” is a basic amino acid.

The present invention also contemplate bone morphogenic protein-7/osteogenic protein-1 monomers containing mutations outside of said β hairpin loop structures that alter the structure or conformation of those hairpin loops. These structural alterations in turn serve to increase the electrostatic interactions between regions of the β hairpin loop structures of bone morphogenic protein-7/osteogenic protein-1 monomer contained in a dimeric molecule, and a receptor having affinity for the dimeric protein. These mutations are found at positions selected from the group consisting of positions 1-20, 41-81, and 103-111 of bone morphogenic protein-7/osteogenic protein-1 monomer.

Specific examples of these mutation outside of the β hairpin L1 and L3 loop structures include, A1J, N2J, V3J, A4J, E5J, N6J, S7J, S8J, S9J, D10J, Q11J, R12J, Q13J, A14J, C15J, K16J, K17J, H18J, E19J, L20J, Y41J, Y42J, C43J, E44J, G45J, E46J, C47J, A48J, F49J, P50J, L51J, N52J, S53J, A54J, T55J, N56J, H57J, A58J, I59J, V60J, Q61J, T62J, L63J, V64J, H65J, F66J, I67J, N68J, P69J, E70J, T71J, V72J, P73J, K74J, P75J, C76J, C77J, A78J, P79J, T80J, M103J, V104J, V105J, R106J, A107J, C108J, G109J, C110J, and H111J. The variable “J” is any amino acid whose introduction results in an increase in the electrostatic interaction between the L1 and L3 β hairpin loop structures of the bone morphogenic protein-7/osteogenic protein-1 and a receptor with affinity for a dimeric protein containing the mutant bone morphogenic protein-7/osteogenic protein-1 monomer.

The invention also contemplates a number of bone morphogenic protein-7/osteogenic protein-1 monomers in modified forms. These modified forms include bone morphogenic protein-7/osteogenic protein-1 monomers linked to another cystine knot growth factor monomer or a fraction of such a monomer.

In specific embodiments, the mutant bone morphogenic protein-7/osteogenic protein-1 growth factor heterodimer comprising at least one mutant subunit or the single chain bone morphogenic protein-7/osteogenic protein-1 growth factor analog as described above is functionally active, i.e., capable of exhibiting one or more functional activities associated with the wild-type bone morphogenic protein-7/osteogenic protein-1 growth factor, such as bone morphogenic protein-7/osteogenic protein-1 growth factor receptor binding, bone morphogenic protein-7/osteogenic protein-1 growth factor receptor signalling and extracellular secretion. Preferably, the mutant bone morphogenic protein-7/osteogenic protein-1 growth factor heterodimer or single chain bone morphogenic protein-7/osteogenic protein-1 growth factor analog is capable of binding to the bone morphogenic protein-7/osteogenic protein-1 growth factor receptor, preferably with affinity greater than the wild type bone morphogenic protein-7/osteogenic protein-1 growth factor. Also it is preferable that such a mutant bone morphogenic protein-7/osteogenic protein-1 growth factor heterodimer or single chain bone morphogenic protein-7/osteogenic protein-1 growth factor analog triggers signal transduction. Most preferably, the mutant bone morphogenic protein-7/osteogenic protein-1 growth factor heterodimer comprising at least one mutant subunit or the single chain bone morphogenic protein-7/osteogenic protein-1 growth factor analog of the present invention has an in vitro bioactivity and/or in vivo bioactivity greater than the wild type bone morphogenic protein-7/osteogenic protein-1 growth factor and has a longer serum half-life than wild type bone morphogenic protein-7/osteogenic protein-1 growth factor. Mutant bone morphogenic protein-7/osteogenic protein-1 growth factor heterodimers and single chain bone morphogenic protein-7/osteogenic protein-1 growth factor analogs of the invention can be tested for the desired activity by procedures known in the art.

Mutants of the Human Bone Morphogenic Protein-8 (BMP-8) Subunit

The human bone morphogenic protein-8 (BMP-8) subunit contains 402 amino acids as shown in FIG. 32 (SEQ ID No: 31). The invention contemplates mutants of the BMP-8 subunit comprising single or multiple amino acid substitutions, deletions or insertions, of one, two, three, four or more amino acid residues when compared with the wild type monomer. Furthermore, the invention contemplates mutant BMP-8 subunit that are linked to another CKGF protein.

The present invention provides mutant BMP-8 subunit L1 hairpin loops having one or more amino acid substitutions between positions 305 and 326, inclusive, excluding Cys residues, as depicted in FIG. 32 (SEQ ID NO: 31). The amino acid substitutions include: E305X, L306X, Y307X, V308X, S309X, F310X, Q311X, D312X, L313X, G314X, W315X, L316X, D317X, W318X, V319X, I320X, A321X, P322X, Q323X, G324X, Y325X, and S326X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the BMP-8 subunit monomer where an acidic residue is present, the variable “X” would correspond to a basic amino acid residue. Specific examples of electrostatic charge altering mutations where a basic residue is introduced into the BMP-8 subunit monomer include one or more of the following: D332B and D337B wherein “B” is a basic amino acid residue.

Introducing acidic amino acid residues where basic residues are present in the BMP-8 subunit monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following K331Z and H346Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at K331U, D332U, D337U, and H346U, wherein “U” is a neutral amino acid.

Mutant BMP-8 subunit monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: F326Z, F327Z, V328Z, S329Z, F330Z, I333Z, G334Z, W335Z, N336Z, W338Z, I339Z, I340Z, A341Z, P342Z, S343Z, G344Z, Y345Z, F326B, F327B, V328B, S329B, F330B, I333B, G334B, W335B, N336B, W338B, I339B, I340B, A341B, P342B, S343B, G344B, and Y345B, wherein “Z” is an acidic amino acid and “B” is a basic amino acid.

Mutant BMP-8 subunit containing mutants in the L3 hairpin loop are also described. These mutant proteins have one or more amino acid substitutions, deletion or insertions, between positions 371 and 395, inclusive, excluding Cys residues, of the L3 hairpin loop, as depicted in FIG. 32 (SEQ ID NO: 31). The amino acid substitutions include K371X, L372X, S373X, A374X, T375X, S376X, V377X, L378X, Y379X, Y380X, D381X, S382X, S383X, N384X, N385X, V386X, I387X, L388X, R389X, K390X, H391X, R392X, N393X, M394X, and V395X, wherein “X” is any amino acid residue, the substitution of which alters the electrostatic character of the L3 loop.

One set of mutations of the L3 hairpin loop includes introducing one or more basic amino acid residues into the BMP-8 subunit L3 hairpin loop amino acid sequence. For example, when introducing basic residues into the L3 loop of the BMP-8 subunit, the variable “X” of the sequence described above corresponds to a basic amino acid residue. Specific examples of electrostatic charge altering mutations where a basic residue is introduced into the BMP-8 subunit include one or more of the following: D405B, D406B, and D414B, wherein “B” is a basic amino acid residue.

The invention further contemplates introducing one or more acidic residues into the amino acid sequence of the BMP-8 subunit L3 hairpin loop. For example, one or more acidic amino acids can be introduced in the sequence of 395-419 described above, wherein the variable “X” corresponds to an acidic amino acid. Specific examples of such mutations include K395Z, K412Z, and K413Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K395U, D405U, D406U, K412U, K413U, and D414U, wherein “U” is a neutral amino acid.

Mutant BMP-8 subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include, L396Z, R397Z, P398Z, M399Z, S400Z, M401Z, L402Z, Y403Z, Y404Z, G407Z, Q408Z, N409Z, I410Z, I411Z, I415Z, Q416Z, N417Z, M418Z, I419Z, L396B, R397B, P398B, M399B, S400B, M401B, L402B, Y403B, Y404B, G407B, Q408B, N409B, I410B, I411B, I415B, Q416B, N417B, M418B, and I419B, wherein “Z” is an acidic amino acid and “B” is a basic amino acid.

The present invention also contemplate BMP-8 subunit containing mutations outside of said β hairpin loop structures that alter the structure or conformation of those hairpin loops. These structural alterations in turn serve to increase the electrostatic interactions between regions of the β hairpin loop structures of BMP-8 subunit contained in a dimeric molecule, and a receptor having affinity for the dimeric protein. These mutations are found at positions selected from the group consisting of positions 1-325, 347-394, and 420-426 of the BMP-8 subunit monomer.

Specific examples of these mutation outside of the β hairpin L1 and L3 loop structures include, M1J, P2J, L3J, L4J, W5J, L6J, R7J, G8J, F9J, L10J, L11J, A12J, S13J, C14J, W15J, I16J, I17J, V18J, R19J, S20J, S21J, P22J, T23J, P24J, G25J, S26J, E27J, G28J, H29J, S30J, A31J, A32J, P33J, D34J, C35J, P36J, S37J, C38J, A39J, L40J, A41J, A42J, L43J, P44J, K45J, D46J, V47J, P48J, N49J, S50J, Q51J, P52J, E53J, M54J, V55J, E56J, A57J, V58J, K59J, K60J, H61J, I62J, L63J, N64J, M65J, L66J, H67J, L68J, K69J, K70J, R71J, P72J, D73J, V74J, T75J, Q76J, P77J, V78J, P79J, K80J, A81J, A82J, L83J, L84J, N85J, A86J, I87J, R88J, L89J, L90J, H91J, V92J, G93J, K94J, V95J, G96J, E97J, N98J, G99J, Y100J, V101J, E102J, I103J, E104J, D105J, D106J, I107J, G108J, R109J, R110J, A111J, E112J, M113J, N114J, E115J, L116J, M117J, E118J, Q119J, T120J, S121J, E122J, I123J, I124J, T125J, F126J, A127J, E128J, S129J, G130J, T131J, A132J, R133J, K134J, T135J, L136J, H137J, F138J, E139J, I140J, S141J, K142J, E143J, G144J, S145J, D146J, L147J, S148J, V149J, V150J, E151J, R152J, A153J, E154J, V155J, W156J, L157J, F158J, L159J, K160J, V161J, P162J, K163J, A164J, N165J, R166J, T167J, R168J, T169J, K170J, V171J, T172J, I173J, R174J, L175J, F176J, Q177J, Q178J, Q179J, K180J, H181J, P182J, Q183J, G184J, S185J, L186J, D187J, T188J, G189J, E190J, E191J, A192J, E193J, E194J, V195J, G196J, L197J, K198J, G199J, E200J, R201J, S202J, E203J, L204J, L205J, L206J, S207J, E208J, K209J, V210J, V211J, D212J, A213J, R214J, K215J, S216J, T217J, W218J, H219J, V220J, F221J, P222J, V223J, S224J, S225J, S226J, I227J, Q228J, R229J, L230J, L231J, D232J, Q233J, G234J, K235J, S236J, S237J, L238J, D39J, V240J, R241J, I242J, A243J, C244J, E245J, Q246J, C247J, Q248J, E249J, S250J, G251J, A252J, S253J, L254J, V255J, L256J, L257J, G258J, K259J, K260J, K261J, K262J, K263J, E264J, E265J, E266J, G267J, E268J, G269J, K270J, K271J, K272J, G273J, G274J, G275J, E276J, G277J, G278J, A279J, G280J, A281J, D282J, E283J, E284J, K285J, E286J, Q287J, S288J, H289J, R290J, P291J, F292J, L293J, M294J, L295J, Q296J, A297J, R298J, Q299J, S300J, E301J, D302J, H303J, P304J, H305J, R306J, R307J, R308J, R309J, R310J, G311J, L312J, E313J, C314J, D315J, G316J, K317J, V318J, N319J, I320J, C321J, C322J, K323J, K324J, Q325J, A347J, N348J, Y349J, C350J, E351J, G352J, E353J, C354J, P355J, S356J, H357J, I358J, A359J, G360J, T361J, S362J, G363J, S364J, S365J, L366J, S367F368J, H369J, S370J, T371J, V372J, I373J, N374J, H375J, Y376J, R377J, M378J, R379J, G380J, H381J, S382J, P383J, F384J, A385J, N386J, L387J, K388J, S389J, C390J, C391J, V392J, P393J, T394J, V420J, E421J, E422J, C423J, G424J, C425J, and S426J. The variable “J” is any amino acid whose introduction results in an increase in the electrostatic interaction between the L1 and L3 β hairpin loop structures of the BMP-8 subunit and a receptor with affinity for a dimeric protein containing the mutant BMP-8 subunit monomer.

The invention also contemplates a number of BMP-8 subunit in modified forms. These modified forms include BMP-8 subunit linked to another cystine knot growth factor or a fraction of such a monomer.

In specific embodiments, the mutant BMP-8 subunit heterodimer comprising at least one mutant subunit or the single chain BMP-8 subunit analog as described above is functionally active, i.e., capable of exhibiting one or more functional activities associated with the wild-type BMP-8 subunit, such as BMP-8 subunit receptor binding, BMP-8 subunit protein family receptor signalling and extracellular secretion. Preferably, the mutant BMP-8 subunit heterodimer or single chain BMP-8 subunit analog is capable of binding to the BMP-8 subunit receptor, preferably with affinity greater than the wild type BMP-8 subunit. Also it is preferable that such a mutant BMP-8 subunit heterodimer or single chain BMP-8 subunit analog triggers signal transduction. Most preferably, the mutant BMP-8 subunit heterodimer comprising at least one mutant subunit or the single chain BMP-8 subunit analog of the present invention has an in vitro bioactivity and/or in vivo bioactivity greater than the wild type BMP-8 subunit and has a longer serum half-life than wild type BMP-8 subunit. Mutant BMP-8 subunit heterodimers and single chain BMP-8 subunit analogs of the invention can be tested for the desired activity by procedures known in the art.

Mutants of the Human Bone Morphogenic Protein-10 (BMP-10)

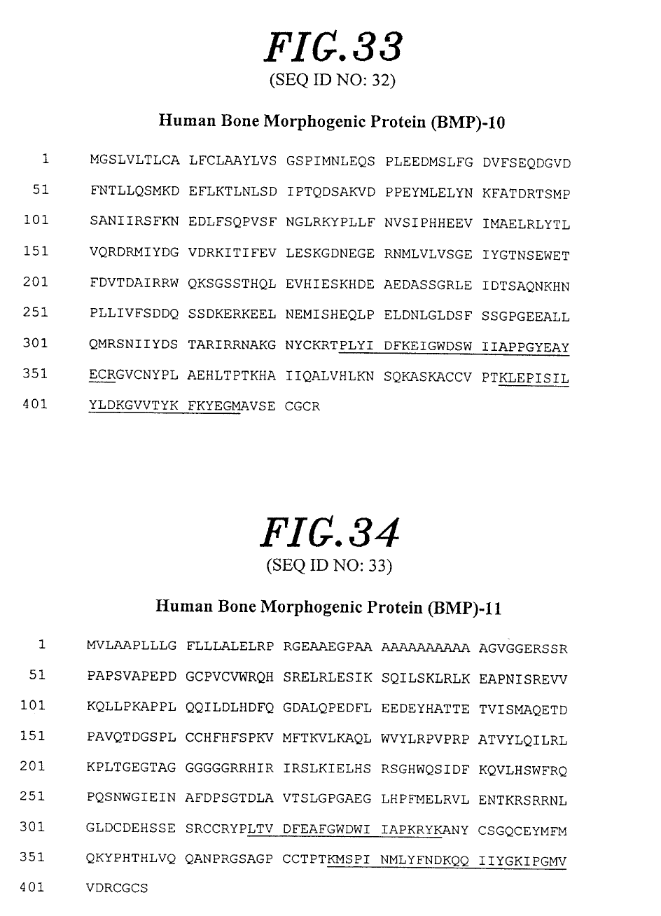

The human bone morphogenic protein-10 (BMP-10) contains 424 amino acids as shown in FIG. 33 (SEQ ID No: 32). The invention contemplates mutants of the BMP-10 comprising single or multiple amino acid substitutions, deletions or insertions, of one, two, three, four or more amino acid residues when compared with the wild type monomer. Furthermore, the invention contemplates mutant BMP-10 that are linked to another CKGF protein.

The present invention provides mutant BMP-10 L1 hairpin loops having one or more amino acid substitutions between positions 327 and 353, inclusive, excluding Cys residues, as depicted in FIG. 33 (SEQ ID NO: 32). The amino acid substitutions include: P327X, L328X, Y329X, I330X, D331X, F332X, K333X, E334X, I335X, G336X, W337X, D338X, S339X, W340X, I341X, I342X, A343X, P344X, P345X, G346X, Y347X, E348X, A349X, Y350X, E351X, C352X, and R353X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the BMP-10 monomer where an acidic residue is present, the variable “X” would correspond to a basic amino acid residue. Specific examples of electrostatic charge altering mutations where a basic residue is introduced into the BMP-10 include one or more of the following D331B, E334B, D338B, E348B, and E351B, wherein “B” is a basic amino acid residue.

Introducing acidic amino acid residues where basic residues are present in the BMP-10 monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following K333Z and R353Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D331U, K333U, E334U, D338U, E348U, E351U, and R353U, wherein “U” is a neutral amino acid.

Mutant BMP-10 monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: P327Z, L328Z, Y329Z, I330Z, F332Z, I335Z, G336Z, W337Z, S339Z, W340Z, I341Z, I342Z, A343Z, P344Z, P345Z, G346Z, Y347Z, A349Z, Y350Z, C352Z, P327B, L328B, Y329B, I330B, F332B, I335B, G336B, W337B, S339B, W340B, I341B, I342B, A343B, P344B, P345B, G346B, Y347B, A349B, Y350B, and C352B, wherein “Z” is an acidic amino acid and “B” is a basic amino acid.

Mutant BMP-10 containing mutants in the L3 hairpin loop are also described. These mutant proteins have one or more amino acid substitutions, deletion or insertions, between positions 327 and 353, inclusive, excluding Cys residues, of the L3 hairpin loop, as depicted in FIG. 33 (SEQ ID NO: 32). The amino acid substitutions include K393X, L394X, E395X, P396X, I397X, S398X, I399X, L400X, Y401X, L402X, D403X, K404X, G405X, V406X, V407X, T408X, Y409X, K410X, F411X, K412X, Y413X, E414X, G415X, and M416X, wherein “X” is any amino acid residue, the substitution of which alters the electrostatic character of the L3 loop.

One set of mutations of the L3 hairpin loop includes introducing one or more basic amino acid residues into the BMP-10 L3 hairpin loop amino acid sequence. For example, when introducing basic residues into the L3 loop of the BMP-10, the variable “X” of the sequence described above corresponds to a basic amino acid residue. Specific examples of electrostatic charge altering mutations where a basic residue is introduced into the BMP-10 include one or more of the following: E395B, D403B, and E414B, wherein “B” is a basic amino acid residue.

The invention further contemplates introducing one or more acidic residues into the amino acid sequence of the BMP-10 L3 hairpin loop. For example, one or more acidic amino acids can be introduced in the sequence of 393-416described above, wherein the variable “X” corresponds to an acidic amino acid. Specific examples of such mutations include K393Z, K404Z, K410Z, and K412Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced of K393U, E395U, D403U, K404U, K410U, K412U, and E414U, wherein “U” is a neutral amino acid.

Mutant BMP-10 proteins are provided containing one or more electrostatic charge altering mutations in the L3 hairpin loop amino acid sequence that convert non-charged or neutral amino acid residues to charged residues. Examples of mutations converting neutral amino acid residues to charged residues include, L394Z, P396Z, I397Z, S398Z, I399Z, L400Z, Y401Z, L402Z, G405Z, V406Z, V407Z, T408Z, Y409Z, F411Z, Y413Z, G415Z, M416Z, L394B, P396B, I397B, S398B, I399B, L400B, Y401B, L402B, G405B, V406B, V407B, T408B, Y409B, F411B, Y413B, G415B, and M416B, wherein “Z” is an acidic amino acid and “B” is a basic amino acid.

The present invention also contemplate BMP-10 containing mutations outside of said β hairpin loop structures that alter the structure or conformation of those hairpin loops. These structural alterations in turn serve to increase the electrostatic interactions between regions of the β hairpin loop structures of BMP-10 contained in a dimeric molecule, and a receptor having affinity for the dimeric protein. These mutations are found at positions selected from the group consisting of positions 1-326, 354-392, and 417-424 of the BMP-10.

Specific examples of these mutation outside of the β hairpin L1 and L3 loop structures include, M1J, G2J, S3J, L4J, V5J, L6J, T7J, L8J, C9J, A10J, L11J, F12J, C13J, L14J, A15J, A16J, Y17J, L18J, V19J, S20J, G21J, S22J, P23J, I24J, M25J, N26J, L27J, E28J, Q29J, S30J, P31J, L32J, E33J, E34J, D35J, M36J, S37J, L38J, F39J, G40J, D41J, V42J, F43J, S44J, E45J, Q46J, D47J, G48J, V49J, D50J, F51J, N52J, T53J, L54J, L55J, Q56J, S57J, M58J, K59J, D60J, E61J, F62J, L63J, K64J, T65J, L66J, N67J, L68J, S69J, D70J, 171J, P72J, T73J, Q74J, D75J, S76J, A7J, K78J, V79J, D80J, P81J, P82J, E83J, Y84J, M85J, L86J, E87J, L88J, Y89J, N90J, K91J, F92J, A93J, T94J, D95J, R96J, T9J, S98J, M99J, P100J, S101J, A102J, N103J, I104J, I105J, R106J, S107J, F108J, K109J, N110J, E111J, D112J, L113J, F114J, S115J, Q116J, P117J, V118J, S119J, F120J, N121J, G122J, L123J, R124J, K125J, Y126J, P127J, L128J, L129J, F130J, N131J, V132J, S133J, I134J, P135J, H136J, H137J, E138J, E139J, V140J, I141J, M142J, A143J, E144J, L145J, R146J, L147J, Y148J, T149J, L150J, V151J, Q152J, R153J, D154J, R155J, M156J, I157J, Y158J, D159J, G160J, V161J, D162J, R163J, K164J, I165J, T166J, I167J, F168J, E169J, V170J, L171J, E172J, S173J, K174J, G175J, D176J, N177J, E178J, G179J, E180J, R181J, N182J, M183J, L184J, V185J, L186J, V187J, S188J, G189J, E190J, I191J, Y192J, G193J, T194J, N195J, S196J, E197J, W198J, E199J, T200J, F201J, D202J, V203J, T204J, D205J, A206J, I207J, R208J, R209J, W210J, Q211J, K212J, S213J, G214J, S215J, S216J, T217J, H218J, Q219J, L220J, E221J, V222J, H223J, I224J, E225J, S226J, K227J, H228J, D229J, E230J, A231J, E232J, D233J, A234J, S235J, S236J, G237J, R238J, L239J, E240J, I241J, D242J, T243J, S244J, A245J, Q246J, N247J, K248J, H249J, N250J, P251J, L252J, L253J, I254J, V255J, F256J, S257J, D258J, D259J, Q260J, S261J, S262J, D263J, K264J, E265J, R266J, K267J, E268J, E269J, L270J, N271J, E272J, M273J, I274J, S275J, H276J, E277J, Q278J, L279J, P280J, E281J, L282J, D283J, N284J, L285J, G286J, L287J, D288J, S289J, F290J, S291J, S292J, G293J, P294J, G295J, E296J, E297J, A298J, L299J, L300J, Q301J, M302J, R303J, S304J, N305J, I306J, I307J, Y308J, D309J, S310J, T311J, A312J, R313J, I314J, R315J, R316J, N317J, A318J, K319J, G320J, N321J, Y322J, C323J, K324J, R325J, T326J, G354J, V355J, C356J, N357J, Y358J, P359J, L360J, A361J, E362J, H363J, L364J, T365J, P366J, T367J, K368J, H369J, A370J, I371J, I372J, Q373J, A374J, L375J, V376J, H377J, L378J, K379J, N380J, S381J, Q382J, K383J, A384J, S385J, K386J, A387J, C388J, C389J, V390J, P391J, T392J, A417J, V418J, S419J, E420J, C421J, G422J, C423J, and R424J. The variable “J” is any amino acid whose introduction results in an increase in the electrostatic interaction between the L1 and L3 β hairpin loop structures of the BMP-10and a receptor with affinity for a dimeric protein containing the mutant BMP-10 monomer.

The invention also contemplates a number of BMP-10 in modified forms. These modified forms include BMP-10 linked to another cystine knot growth factor or a fraction of such a monomer.

In specific embodiments, the mutant BMP-10 heterodimer comprising at least one mutant subunit or the single chain BMP-10 analog as described above is functionally active, i.e., capable of exhibiting one or more functional activities associated with the wild-type BMP-10, such as BMP-10 receptor binding, BMP-10 protein family receptor signalling and extracellular secretion. Preferably, the mutant BMP-10 heterodimer or single chain BMP-10 analog is capable of binding to the BMP-10 receptor, preferably with affinity greater than the wild type BMP-10. Also it is preferable that such a mutant BMP-10 heterodimer or single chain BMP-10 analog triggers signal transduction. Most preferably, the mutant BMP-10 heterodimer comprising at least one mutant subunit or the single chain BMP-10 analog of the present invention has an in vitro bioactivity and/or in vivo bioactivity greater than the wild type BMP-10 and has a longer serum half-life than wild type BMP-10. Mutant BMP-10 heterodimers and single chain BMP-10 analogs of the invention can be tested for the desired activity by procedures known in the art.

Mutants of the Human Bone Morphogenic Protein-11 (BMP-11)

The human bone morphogenic protein-11 (BMP-11) contains 407 amino acids as shown in FIG. 34 (SEQ ID No: 33). The invention contemplates mutants of the BMP-11 comprising single or multiple amino acid substitutions, deletions or insertions, of one, two, three, four or more amino acid residues when compared with the wild type monomer. Furthermore, the invention contemplates mutant BMP-11 that are linked to another CKGF protein.

The present invention provides mutant BMP-11 L1 hairpin loops having one or more amino acid substitutions between positions 318 and 337, inclusive, excluding Cys residues, as depicted in FIG. 34 (SEQ ID NO: 33). The amino acid substitutions include: L318X, T319X, V320X, D321X, F322X, E323X, A324X, F325X, G326X, W327X, D328X, W329X, I330X, I331X, A332X, P333X, K334X, R335X, Y336X, and K337X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the BMP-11 monomer where an acidic residue is present, the variable “X” would correspond to a basic amino acid residue. Specific examples of electrostatic charge altering mutations where a basic residue is introduced into the BMP-11 monomer include one or more of the following: D321B, E323B, and D328B, wherein “B” is a basic amino acid residue.

Introducing acidic amino acid residues where basic residues are present in the BMP-11 monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following K334Z, R335Z, and K337Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D321U, E323U, D328U, K334U, R335U, and K337U, wherein “U” is a neutral amino acid.

Mutant BMP-11 monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include L318Z, T319Z, V320Z, F322Z, A324Z, F325Z, G326Z, W327Z, W329Z, I330Z, I331Z, A332Z, P333Z, Y336Z, L318B, T319B, V320B, F322B, A324B, F325B, G326B, W327B, W329B, I330B, I331B, A332B, P333B, and Y336B, wherein “Z” is an acidic amino acid and “B” is a basic amino acid.

Mutant BMP-11 containing mutants in the L3 hairpin loop are also described. These mutant proteins have one or more amino acid substitutions, deletion or insertions, between positions 376 and 400, inclusive, excluding Cys residues, of the L3 hairpin loop, as depicted in FIG. 34 (SEQ ID NO: 33). The amino acid substitutions include: K376X, M377X, S378X, P379X, I380X, N381X, M382X, L383X, Y384X, F385X, N386X, D387X, K388X, Q389X, Q390X, I391X, I392X, Y393X, G394X, K395X, I396X, P397X, G398X, M399X, and V400X, wherein “X” is any amino acid residue, the substitution of which alters the electrostatic character of the L3 loop.

One set of mutations of the L3 hairpin loop includes introducing one or more basic amino acid residues into the BMP-11 L3 hairpin loop amino acid sequence. For example, when introducing basic residues into the L3 loop of the BMP-11, the variable “X” of the sequence described above corresponds to a basic amino acid residue. Specific examples of electrostatic charge altering mutations where a basic residue is introduced into the BMP-11 include one or more of the following: D387B, wherein “B” is a basic amino acid residue.

The invention further contemplates introducing one or more acidic residues into the amino acid sequence of the BMP-11 L3 hairpin loop. For example, one or more acidic amino acids can be introduced in the sequence of 376-400 described above, wherein the variable “X” corresponds to an acidic amino acid. Specific examples of such mutations include K376Z, K388Z, and K395Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K376U, D387U, K388U, and K395U, wherein “U” is a neutral amino acid.

Mutant BMP-11 proteins are provided containing one or more electrostatic charge altering mutations in the L3 hairpin loop amino acid sequence that convert non-charged or neutral amino acid residues to charged residues. Examples of mutations converting neutral amino acid residues to charged residues include, M377Z, S378Z, P379Z, I380Z, N381Z, M382Z, L383Z, Y384Z, F385Z, N386Z, Q389Z, Q390Z, I391Z, I392Z, Y393Z, G394Z, I396Z, P397Z, G398Z, M399Z, V400Z, M377B, S378B, P379B, I380B, N381B, M382B, L383B, Y384B, F385B, N386B, Q389B, Q390B, I391B, I392B, Y393B, G394B, I396B, P397B, G398B, M399B, and V400B, wherein “Z” is an acidic amino acid and “B” is a basic amino acid.

The present invention also contemplate BMP-11 containing mutations outside of said β hairpin loop structures that alter the structure or conformation of those hairpin loops. These structural alterations in turn serve to increase the electrostatic interactions between regions of the β hairpin loop structures of BMP-11 contained in a dimeric molecule, and a receptor having affinity for the dimeric protein. These mutations are found at positions selected from the group consisting of positions 1-317, 338-375, and 401-407 of the BMP-11 monomer.

Specific examples of these mutation outside of the β hairpin L1 and L3 loop structures include, M1J, V2J, L3J, A4J, A5J, P6J, L7J, L8J, L9J, G10J, F11J, L12J, L23J, L24J, A25J, L26J, E27J, L28J, R19J, P20J, R21J, G22J, E23J, A24J, A25J, E26J, G27J, P28J, A29J, A30J, A31J, A32J, A33J, A34J, A35J, A36J, A37J, A38J, A39J, A40J, A41J, G42J, V43J, G44J, G45J, E46J, R47J, S48J, S49J, R50J, P51J, A52J, P53J, S54J, V55J, A56J, P57J, E58J, P59J, D60J, G61J, C62J, P63J, V64J, C65J, V66J, W67J, R68J, Q69J, H70J, S71J, R72J, L74J, R75J, L76J, E77J, S78J, I79J, K80J, S81J, Q82J, I83J, L84J, S85J, K86J, L87J, R88J, L89J, K90J, E91J, A92J, P93J, N94J, I95J, S96J, R97J, E98J, V99J, V100J, K101J, Q102J, L103J, L104J, P105J, K106J, A107J, P108J, P109J, L110J, Q111J, Q112J, I113J, L114J, D115J, L116J, H117J, D118J, F119J, Q120J, G121J, D122J, A123J, L124J, Q125J, P126J, E127J, D128J, F129J, L130J, E131J, E132J, D133J, E134J, Y135J, H136J, A137J, T138J, T139J, E140J, T141J, V142J, I143J, S144J, M145J, A146J, Q147J, E148J, T149J, D150J, P151J, A152J, V153J, Q154J, T155J, D156J, G157J, S158J, P159J, L160J, C161J, C162J, H163J, F164J, H165J, F166J, S167J, P168J, K169J, V170J, M171J, F172J, T173J, K174J, V175J, L176J, K177J, A178J, Q179J, L180J, W181J, V182J, Y183J, L184J, R185J, P186J, V187J, P188J, R189J, P190J, A191J, T192J, V193J, Y194J, L195J, Q196J, I197J, L198J, R199J, L200J, K201J, P202J, L203J, T204J, G205J, E206J, G207J, T208J, A209J, G210J, G211J, G212J, G213J, G214J, G215J, R216J, R217J, H218J, I219J, R220J, I221J, R222J, S223J, L224J, K225J, I226J, E227J, L228J, H229J, S230J, R231J, S232J, G233J, H234J, W235J, Q236J, S237J, I238J, D239J, F240J, K241J, Q242J, V243J, L244J, H245J, S246J, W247J, F248J, R249J, Q250J, P251J, Q252J, S253J, N254J, W255J, G256J, I257J, E258J, I259J, N260J, A261J, F262J, D263J, P264J, S265J, G266J, T267J, D268J, L269J, A270J, V271J, T272J, S273J, L274J, G275J, P276J, G277J, A278J, E279J, G280J, L281J, H282J, P283J, F284J, M285J, E286J, L287J, R288J, V289J, L290J, E291J, N292J, T293J, K294J, R295J, S296J, R297J, R298J, N299J, L300J, G301J, L302J, D303J, C304J, D305J, E306J, H307J, S308J, S309J, E310J, S311J, R312J, C313J, C314J, R315J, Y316J, P317J, A338J, N339J, Y340J, C341J, S342J, G343J, Q344J, C345J, E346J, Y347J, M348J, F349J, M350J, Q351J, K352J, Y353J, P354J, H355J, T356J, H357J, L358J, V359J, Q360J, Q361J, A362J, N363J, P364J, R365J, G366J, S367J, A368J, G369J, P370J, C371J, C372J, T373J, P374J, T375J, V401J, D402J, R403J, C404J, G405J, C406J, and S407J. The variable “J” is any amino acid whose introduction results in an increase in the electrostatic interaction between the L1 and L3 β hairpin loop structures of the BMP-11 and a receptor with affinity for a dimeric protein containing the mutant BMP-11 monomer.

The invention also contemplates a number of BMP-11 in modified forms. These modified forms include BMP-11 linked to another cystine knot growth factor or a fraction of such a monomer.

In specific embodiments, the mutant BMP-11 heterodimer comprising at least one mutant subunit or the single chain BMP-11 analog as described above is functionally active, i.e., capable of exhibiting one or more functional activities associated with the wild-type BMP-11, such as BMP-11 receptor binding, BMP-11 protein family receptor signalling and extracellular secretion. Preferably, the mutant BMP-11 heterodimer or single chain BMP-11 analog is capable of binding to the BMP-11 receptor, preferably with affinity greater than the wild type BMP-11. Also it is preferable that such a mutant BMP-11 heterodimer or single chain BMP-11 analog triggers signal transduction. Most preferably, the mutant BMP-11 heterodimer comprising at least one mutant subunit or the single chain BMP-11 analog of the present invention has an in vitro bioactivity and/or in vivo bioactivity greater than the wild type BMP-11 and has a longer serum half-life than wild type BMP-11. Mutant BMP-11 heterodimers and single chain BMP-11 analogs of the invention can be tested for the desired activity by procedures known in the art.

Mutants of the Human Bone Morphogenic Protein-15 (BMP-15)

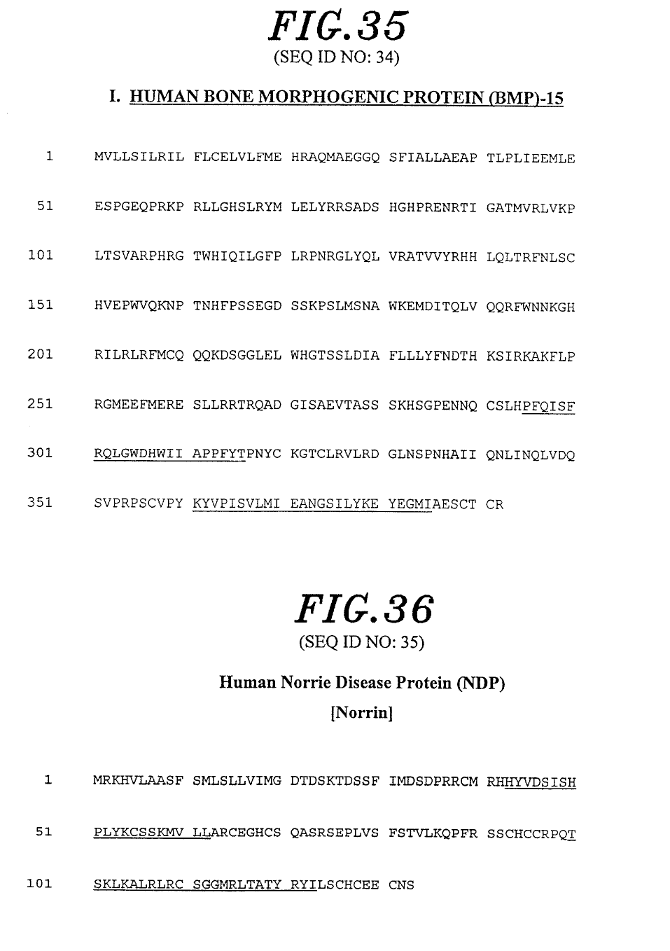

The human bone morphogenic protein-15 (BMP-15) contains 392 amino acids as shown in FIG. 35 (SEQ ID No: 34). The invention contemplates mutants of the BMP-15 comprising single or multiple amino acid substitutions, deletions or insertions, of one, two, three, four or more amino acid residues when compared with the wild type monomer. Furthermore, the invention contemplates mutant BMP-15 that are linked to another CKGF protein.

The present invention provides mutant BMP-15 L1 hairpin loops having one or more amino acid substitutions between positions 295 and 316, inclusive, excluding Cys residues, as depicted in FIG. 35 (SEQ ID NO: 34). The amino acid substitutions include: P295X, F296X, Q297X, I298X, S299X, F300X, R301X, Q302X, L303X, G304X, W305X, D306X, H307X, W308X, I309X, I310X, A311X, P312X, P313X, F314X, Y315X, and T316X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the BMP-15 monomer where an acidic residue is present, the variable “X” would correspond to a basic amino acid residue. Specific examples of electrostatic charge altering mutations where a basic residue is introduced into the BMP-15 monomer include one or more of the following: D306B, wherein “B” is a basic amino acid residue.

Introducing acidic amino acid residues where basic residues are present in the BMP-15 monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: R301Z and H307Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at R301U, D306U, and H307U, wherein “U” is a neutral amino acid.

Mutant BMP-15 monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: P295Z, F296Z, Q297Z, I298Z, S299Z, F300Z, Q302Z, L303Z, G304Z, W305Z, W308Z, I309Z, I310Z, A311Z, P312Z, P313Z, F314Z, Y315Z, T316Z, P295B, F296B, Q297B, I298B, S299B, F300B, Q302B, L303B, G304B, W305B, W308B, I309B, I310B, A311B, P312B, P313B, F314B, Y315B, and T316B, wherein “Z” is an acidic amino acid and “B” is a basic amino acid.

Mutant BMP-15 containing mutants in the L3 hairpin loop are also described. These mutant proteins have one or more amino acid substitutions, deletion or insertions, between positions 361 and 385, inclusive, excluding Cys residues, of the L3 hairpin loop, as depicted in FIG. 35 (SEQ ID NO: 34). The amino acid substitutions include: K361X, Y362X, V363X, P364X, I365X, S366X, V367X, L368X, M369X, I370X, E371X, A372X, N373X, G374X, S375X, I376X, L377X, Y378X, K379X, E380X, Y381X, E382X, G383X, M384X, and I385X, wherein “X” is any amino acid residue, the substitution of which alters the electrostatic character of the L3 loop.

One set of mutations of the L3 hairpin loop includes introducing one or more basic amino acid residues into the BMP-15 L3 hairpin loop amino acid sequence. For example, when introducing basic residues into the L3 loop of the BMP-15, the variable “X” of the sequence described above corresponds to a basic amino acid residue. Specific examples of electrostatic charge altering mutations where a basic residue is introduced into the BMP-15 include one or more of the following: E371B, E380B, and E382B, wherein “B” is a basic amino acid residue.

The invention further contemplates introducing one or more acidic residues into the amino acid sequence of the BMP-15 L3 hairpin loop. For example, one or more acidic amino acids can be introduced in the sequence of 361-385 described above, wherein the variable “X” corresponds to an acidic amino acid. Specific examples of such mutations include K361Z and K379Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K361U, E371U, K379U, E380U, and E382U, wherein “U” is a neutral amino acid.

Mutant BMP-15 proteins are provided containing one or more electrostatic charge altering mutations in the L3 hairpin loop amino acid sequence that convert non-charged or neutral amino acid residues to charged residues. Examples of mutations converting neutral amino acid residues to charged residues include, Y362Z, V363Z, P364Z, 1365Z, S366Z, V367Z, L368Z, M369Z, I370Z, A372Z, N373Z, G374Z, S375Z, I376Z, L377Z, Y378Z, Y381Z, G383Z, M384Z, I385Z, Y362B, V363B, P364B, I365B, S366B, V367B, L368B, M369B, I370B, A372B, N373B, G374B, S375B, I376B, L377B, Y378B, Y381B, G383B, M384B, and I385B, wherein “Z” is an acidic amino acid and “B” is a basic amino acid.

The present invention also contemplate BMP-15 containing mutations outside of said β hairpin loop structures that alter the structure or conformation of those hairpin loops. These structural alterations in turn serve to increase the electrostatic interactions between regions of the β hairpin loop structures of BMP-15 contained in a dimeric molecule, and a receptor having affinity for the dimeric protein. These mutations are found at positions selected from the group consisting of positions 1-294, 317-360, and 386-392 of the BMP-15 monomer.

Specific examples of these mutation outside of the β hairpin L1 and L3 loop structures include, M1J, V2J, L3J, L4J, S5J, I6J, L7J, R8J, I9J, L10J, F11J, L12J, C13J, E14J, L15J, V16J, L17J, F18J, M19J, E20J, H21J, R22J, A23J, Q24J, M25J, A26J, E27J, G28J, G29J, Q30J, S31J, F32J, I33J, A34J, L35J, L36J, A37J, E38J, A39J, P40J, T41J, L42J, P43J, L44J, I45J, E46J, E47J, M48J, L49J, E50J, E51J, S52J, P53J, G54J, E55J, Q56J, P57J, R58J, K59J, P60J, R61J, L62J, L63J, G64J, H65J, S66J, L67J, R68J, Y69J, M70J, L71J, E72J, L73J, Y74J, R75J, R76J, S77J, A78J, D79J, S80J, H81J, G82J, H83J, P84J, R85J, E86J, N87J, R88J, T89J, I90J, G91J, A92J, T93J, M94J, V95J, R96J, L97J, V98J, K99J, P100J, L101J, T102J, S103J, V104J, A105J, R106J, P107J, H108J, R109J, G110J, T111J, W112J, H113J, I114J, Q115J, I116J, L117J, G118J, F119J, P120J, L121J, R122J, P123J, N124J, R125J, G126J, L127J, Y128J, Q129J, L130J, V131J, R132J, A133J, T134J, V135J, V136J, Y137J, R138J, H139J, H140J, L141J, Q142J, L143J, T144J, R145J, F146J, N147J, L148J, S149J, C150J, H151J, V152J, E153J, P154J, W155J, V156J, Q157J, K158J, N159J, P160J, T161J, N162J, H163J, F164J, P165J, S166J, S167J, E168J, G169J, D170J, S171J, S172J, K173J, P174J, S175J, L176J, M177J, S178J, N179J, A180J, W181J, K182J, E183J, M184J, D185J, I186J, T187J, Q188J, L189J, V190J, Q191J, Q192J, R193J, F194J, W195J, N196J, N197J, K198J, G199J, H200J, R201J, I202J, L203J, R204J, L205J, R206J, F207J, M208J, C209J, Q210J, Q211J, Q212J, K213J, D214J, S215J, G216J, G217J, L218J, E219J, L220J, W221J, H222J, G223TJ, 224J, S225J, S226J, L227J, D228J, I229J, A230J, F231J, L232J, L233J, L234J, Y235J, F236J, N237J, D238J, T239J, H240J, K241J, S242J, I243J, R244J, K245J, A246J, K247J, F248J, L249J, P250J, R251J, G252J, M253J, E254J, E255J, F256J, M257J, E258J, R259J, E260J, S261J, L262J, L264J, R264J, R265J, T266J, R267J, Q268J, A269J, D270J, G271J, I272J, S273J, A274J, E275J, V276J, T277J, A278J, S279J, S280J, S281J, K282J, H283J, S284J, G285J, P286J, E287J, N288J, N289J, Q290J, C291J, S292J, L293J, H294J, P317J, N318J, Y319J, C320J, K321J, G322J, T323J, C324J, L325J, R326J, V327J, L328J, R329J, D330J, G331J, L332J, N333J, S334J, P335J, N336J, H337J, A338J, I339J, I340J, Q341J, N342J, L343J, I344J, N345J, Q346J, L347J, V348J, D349J, Q350J, S351J, V352J, P353J, R354J, P355J, S356J, C357J, V358J, P359J, Y360J, A386J, E387J, S388J, C389J, T390J, C391J, and R392J. The variable “J” is any amino acid whose introduction results in an increase in the electrostatic interaction between the L1 and L3 β hairpin loop structures of the BMP-15 and a receptor with affinity for a dimeric protein containing the mutant BMP-15 monomer.

The invention also contemplates a number of BMP-15 in modified forms. These modified forms include BMP-15 linked to another cystine knot growth factor or a fraction of such a monomer.

In specific embodiments, the mutant BMP-15 heterodimer comprising at least one mutant subunit or the single chain BMP-15 analog as described above is functionally active, i.e., capable of exhibiting one or more functional activities associated with the wild-type BMP-15, such as BMP-15 receptor binding, BMP-15 protein family receptor signalling and extracellular secretion. Preferably, the mutant BMP-15 heterodimer or single chain BMP-15 analog is capable of binding to the BMP-15 receptor, preferably with affinity greater than the wild type BMP-15. Also it is preferable that such a mutant BMP-15 heterodimer or single chain BMP-15 analog triggers signal transduction. Most preferably, the mutant BMP-15 heterodimer comprising at least one mutant subunit or the single chain BMP-15 analog of the present invention has an in vitro bioactivity and/or in vivo bioactivity greater than the wild type BMP-15 and has a longer serum half-life than wild type BMP-15. Mutant BMP-15 heterodimers and single chain BMP-15 analogs of the invention can be tested for the desired activity by procedures known in the art.

Mutants of the Human Norrie Disease Protein

The Human Norrie Disease Protein (NDP) contains 133 amino acids as shown in FIG. 36 (SEQ ID No: 35). The invention contemplates mutants of the NDP comprising single or multiple amino acid substitutions, deletions or insertions, of one, two, three, four or more amino acid residues when compared with the wild type monomer. Furthermore, the invention contemplates mutant NDP that are linked to another CKGF protein.

The present invention provides mutant NDP L1 hairpin loops having one or more amino acid substitutions between positions 43 and 62, inclusive, excluding Cys residues, as depicted in FIG. 36 (SEQ ID NO: 35). The amino acid substitutions include: H43X, Y44X, V45X, D46X, S47X, I48X, S49X, H50X, P51X, L52X, Y53X, K54X, C55X, S56X, S57X, K58X, M59X, V60X, L61X, and L62X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the NDP monomer where an acidic residue is present, the variable “X” would correspond to a basic amino acid residue. Specific examples of electrostatic charge altering mutations where a basic residue is introduced into the NDP monomer include one or more of the following: D46B, wherein “B” is a basic amino acid residue.

Introducing acidic amino acid residues where basic residues are present in the NDP monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: H43Z, H50Z, K54Z, and K58Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at H43U, D46U, H50U, K54U, and K58U, wherein “U” is a neutral amino acid.

Mutant NDP monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: Y44Z, V45Z, S47Z, I48Z, S49Z, P51Z, L52Z, Y53Z, C55Z, S56Z, S57Z, M59Z, V60Z, L61Z, L62Z, Y44B, V45B, S47B, I48B, S49B, P51B, L52B, Y53B, C55B, S56B, S57B, M59B, V60B, L61B, and L62B, wherein “Z” is an acidic amino acid and “B” is a basic amino acid.

Mutant NDP containing mutants in the L3 hairpin loop are also described. These mutant proteins have one or more amino acid substitutions, deletion or insertions, between positions 100 and 123, inclusive, excluding Cys residues, of the L3 hairpin loop, as depicted in FIG. 36 (SEQ ID NO: 35). The amino acid substitutions include: T100X, S101X, K102X, L103X, K104X, A105X, L106X, R107X, L108X, R109X, C110X, S111X, G112X, G113X, M114X, R115X, L116X, T117X, A118X, T119X, Y120X, R121X, Y122X, and I123X, wherein “X” is any amino acid residue, the substitution of which alters the electrostatic character of the L3 loop.

The invention further contemplates introducing one or more acidic residues into the amino acid sequence of the NDP L3 hairpin loop. For example, one or more acidic amino acids can be introduced in the sequence of 100-123 described above, wherein the variable “X” corresponds to an acidic amino acid. Specific examples of such mutations include of K102Z, K104Z, R107Z, R109Z, R115Z, and R121Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at K102U, K104U, R107U, R109U, R115U, and R121U, wherein “U” is a neutral amino acid.

Mutant NDP proteins are provided containing one or more electrostatic charge altering mutations in the L3 hairpin loop amino acid sequence that convert non-charged or neutral amino acid residues to charged residues. Examples of mutations converting neutral amino acid residues to charged residues include, T100Z, S101Z, L103Z, A105Z, L106Z, L108Z, C110Z, S111Z, G112Z, G113Z, M114Z, L116Z, T117Z, A118Z, T119Z, Y120Z, Y122Z, I123Z, T100B, S101B, L103B, A105B, L106B, L108B, C110B, S111B, G112B, G113B, M114B, L116B, T117B, A118B, T119B, Y120B, Y122B, and I123B, wherein “Z” is an acidic amino acid and “B” is a basic amino acid.

The present invention also contemplate NDP containing mutations outside of said β hairpin loop structures that alter the structure or conformation of those hairpin loops. These structural alterations in turn serve to increase the electrostatic interactions between regions of the β hairpin loop structures of NDP contained in a dimeric molecule, and a receptor having affinity for the dimeric protein. These mutations are found at positions selected from the group consisting of positions 1-42, 63-99, 124-133 of the NDP monomer.

Specific examples of these mutation outside of the β hairpin L1 and L3 loop structures include, M1J, R2J, K3J, H4J, V5J, L6J, A7J, A8J, S9J, F10J, S11J, M12J, L13J, S14J, L15J, L16J, V17J, I18J, M19J, G20J, D21J, T22J, D23J, S24J, K25J, T26J, D27J, S28J, S29J, F30J, I31J, M32J, D33J, S34J, D35J, P36J, R37J, R38J, C39J, M40J, R41J, H42J, A63J, R64J, C65J, E66J, G67J, H68J, C69J, S70J, Q71J, A72J, S73J, R74J, S75J, E76J, P77J, L78J, V79J, S80J, F81J, S82J, T83J, V84J, L85J, K86J, Q87J, P88J, F89J, R90J, S91J, S92J, C93J, H94J, C95J, C96J, R97J, P98J, Q99J, L124J, S125J, C126J, H127J, C128J, E129J, E130J, C131J, N132J, and S133J. The variable “J” is any amino acid whose introduction results in an increase in the electrostatic interaction between the L1 and L3 β hairpin loop structures of the NDP and a receptor with affinity for a dimeric protein containing the mutant NDP monomer.

The invention also contemplates a number of NDP in modified forms. These modified forms include NDP linked to another cystine knot growth factor or a fraction of such a monomer.

In specific embodiments, the mutant NDP heterodimer comprising at least one mutant subunit or the single chain NDP analog as described above is functionally active, i.e., capable of exhibiting one or more functional activities associated with the wild-type NDP, such as NDP receptor binding, NDP protein family receptor signalling and extracellular secretion. Preferably, the mutant NDP heterodimer or single chain NDP analog is capable of binding to the NDP receptor, preferably with affinity greater than the wild type NDP. Also it is preferable that such a mutant NDP heterodimer or single chain NDP analog triggers signal transduction. Most preferably, the mutant NDP heterodimer comprising at least one mutant subunit or the single chain NDP analog of the present invention has an in vitro bioactivity and/or in vivo bioactivity greater than the wild type NDP and has a longer serum half-life than wild type NDP. Mutant NDP heterodimers and single chain NDP analogs of the invention can be tested for the desired activity by procedures known in the art.

Mutants of the Human Growth Differentiation Factor-1 (GDF-1)

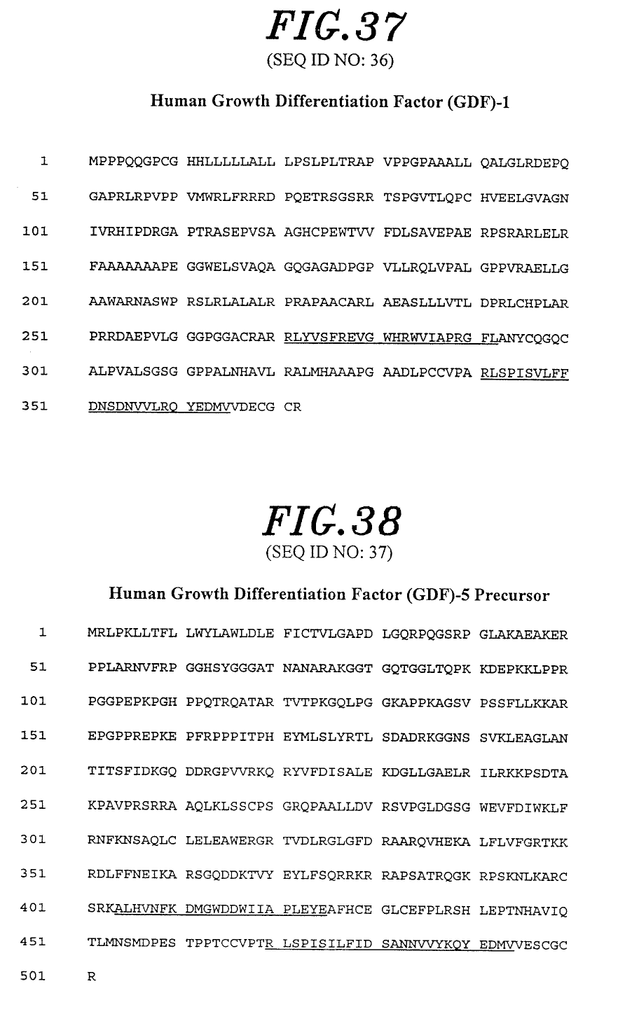

The human growth differentiation factor-1 (GDF-1) contains 372 amino acids as shown in FIG. 37 (SEQ ID No: 36). The invention contemplates mutants of the GDF-1 comprising single or multiple amino acid substitutions, deletions or insertions, of one, two, three, four or more amino acid residues when compared with the wild type monomer. Furthermore, the invention contemplates mutant GDF-1 that are linked to another CKGF protein.

The present invention provides mutant GDF-1 L1 hairpin loops having one or more amino acid substitutions between positions 271 and 292, inclusive, excluding Cys residues, as depicted in FIG. 37 (SEQ ID NO: 36). The amino acid substitutions include R271X, L272X, Y273X, V274X, S275X, F276X, R277X, E278X, V279X, G280X, W281X, H282X, R283X, W284X, V285X, I286X, A287X, P288X, R289X, G290X, F291X, and L292X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the GDF-1 monomer where an acidic residue is present, the variable “X” would correspond to a basic amino acid residue. Specific examples of electrostatic charge altering mutations where a basic residue is introduced into the GDF-1 monomer include E278B wherein “B” is a basic amino acid residue.

Introducing acidic amino acid residues where basic residues are present in the GDF-1 monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following R271Z, R277Z, H282Z, R283Z, and R289Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced of R271U, R277U, E278U, H282U, R283U, and R289U, wherein “U” is a neutral amino acid.

Mutant GDF-1 monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: of L272Z, Y273Z, V274Z, S275Z, F276Z, V279Z, G280Z, W281Z, W284Z, V285Z, I286Z, A287Z, P288Z, G290Z, F291Z, L292Z, L272B, Y273B, V274B, S275B, F276B, V279B, G280B, W281B, W284B, V285B, I286B, A287B, P288B, G290B, F291B, and L292B, wherein “Z” is an acidic amino acid and “B” is a basic amino acid.

Mutant GDF-1 containing mutants in the L3 hairpin loop are also described. These mutant proteins have one or more amino acid substitutions, deletion or insertions, between positions 341 and 365, inclusive, excluding Cys residues, of the L3 hairpin loop, as depicted in FIG. 37 (SEQ ID NO: 36). The amino acid substitutions include: R341X, L342X, S343X, P344X, I345X, S346X, V347X, L348X, F349X, F350X, D351X, N352X, S353X, D354X, N355X, V356X, V357X, L358X, R359X, Q360X, Y361X, E362X, D363X, M364X, and V365X, wherein “X” is any amino acid residue, the substitution of which alters the electrostatic character of the L3 loop.

One set of mutations of the L3 hairpin loop includes introducing one or more basic amino acid residues into the GDF-1 L3 hairpin loop amino acid sequence. For example, when introducing basic residues into the L3 loop of the GDF-1, the variable “X” of the sequence described above corresponds to a basic amino acid residue. Specific examples of electrostatic charge altering mutations where a basic residue is introduced into the GDF-1 include one or more of the following: D351B, D354B, E362B, and D363B, wherein “B” is a basic amino acid residue.

The invention further contemplates introducing one or more acidic residues into the amino acid sequence of the GDF-1 L3 hairpin loop. For example, one or more acidic amino acids can be introduced in the sequence of 341-365 described above, wherein the variable “X” corresponds to an acidic amino acid. Specific examples of such mutations include R341Z and R359Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced of R341U, D351U, D354U, R359U, E362U, and D363U, wherein “U” is a neutral amino acid.

Mutant GDF-1 proteins are provided containing one or more electrostatic charge altering mutations in the L3 hairpin loop amino acid sequence that convert non-charged or neutral amino acid residues to charged residues. Examples of mutations converting neutral amino acid residues to charged residues include, L342Z, S343Z, P344Z, I345Z, S346Z, V347Z, L348Z, F349Z, F350Z, N352Z, S353Z, N355Z, V356Z, V357Z, L358Z, Q360Z, Y361Z, M36Z, V365Z, L342B, S343B, P344B, I345B, S346B, V347B, L348B, F349B, F350B, N352B, S353B, N355B, V356J, V357B, L358B, Q360B, Y361B, M36B, and V365B, wherein “Z” is an acidic amino acid and “B” is a basic amino acid.

The present invention also contemplate GDF-1 containing mutations outside of said β hairpin loop structures that alter the structure or conformation of those hairpin loops. These structural alterations in turn serve to increase the electrostatic interactions between regions of the β hairpin loop structures of GDF-1 contained in a dimeric molecule, and a receptor having affinity for the dimeric protein. These mutations are found at positions selected from the group consisting of 1-270, 293-340, and 366-372 of the GDF-1.

Specific examples of these mutation outside of the β hairpin L1 and L3 loop structures include, M1J, P2J, P3J, P4J, Q5J, Q6J, G7J, P8J, C9J, G10J, H11J, H12J, L13J, L14J, L15J, L16J, L17J, A18J, L19J, L20J, L21J, P22J, S23J, L24J, P25J, L26J, T27J, R28J, A29J, P30J, V31J, P32J, P33J, G34J, P35J, A36J, A37J, A38J, L39J, L40J, Q41J, A42J, L43J, G44J, L45J, R46J, D47J, E48J, P49J, Q50J, G51J, A52J, P53J, R54J, L55J, R56J, P57J, V58J, P59J, P60J, V61J, M62J, W63J, R64J, L65J, F66J, R67J, R68J, R69J, D70J, P71J, Q72J, E73J, T74J, R75J, S76J, G77J, S78J, R79J, R80J, T81J, S82J, P83J, G84J, V85J, T86J, L87J, Q88J, P89J, C90J, H91J, V92J, E93J, E94J, L95J, G96J, V97J, A98J, G9J, N100J, I101J, V102J, R103J, H104J, I105J, P106J, D107J, R108J, G109J, A110J, P111J, T112J, R113J, A114J, S115J, E116J, P117J, V118J, S119J, A120J, A121J, G122J, H123J, C12J, P125J, E126J, W127J, T128J, V129J, V130J, F131J, D132J, L133J, S134J, A135J, V136J, E137J, P138J, A139J, E140J, R141J, P142J, S143J, R144J, A145J, R146J, L147J, E148J, L149J, R150J, F151J, A152J, A153J, A154J, A155J, A156J, A157J, A158J, P159J, E160J, G161J, G162J, W163J, E164J, L165J, S166J, V167J, A168J, Q169J, A170J, G171J, Q172J, G173J, A174J, G175J, A176J, D177J, P178J, G179J, P180J, V181J, L182J, L183J, R184J, Q185J, L186J, V187J, P188J, A189J, L190J, G191J, P192J, P193J, V194J, R195J, A196J, E197J, L198J, L199J, G200J, A201J, A202J, W203J, A204J, R205J, N206J, A207J, S208J, W209J, P210J, R211J, S212J, L213J, R214J, L215J, A216J, L217J, A218J, L219J, R220J, P221J, R222J, A223J, P224J, A225J, A226J, C227J, A228J, R229J, L230J, A231J, E232J, A233J, S234J, L235J, L236J, L237J, V238J, T239J, L240J, D241J, P242J, R243J, L244J, C245J, H246J, P247J, L248J, A249J, R250J, P251J, R252J, R253J, D254J, A255J, E256J, P257J, V258J, L52J, G260J, G261J, G262J, P263J, G264J, G265J, A266J, C267J, R268J, A269J, R270J, A293J, N294J, Y295J, C296J, Q297J, G298J, Q299J, C300J, A301J, L302J, P303J, V304J, A305J, L306J, S307J, G308J, S309J, G310J, G311J, P312J, P313J, A314J, L315J, N316J, H317J, A318J, V319J, L320J, R321J, A322J, L323J, M324J, H325J, A326J, A327J, A328J, P329J, G330J, A331J, A332J, D333J, L334J, P335J, C336J, C337J, V338J, P339J, A340J, V366J, D367J, E368J, C369J, G370J, C371J, and R372J. The variable “J” is any amino acid whose introduction results in an increase in the electrostatic interaction between the L1 and L3 β hairpin loop structures of the GDF-1 and a receptor with affinity for a dimeric protein containing the mutant GDF-1 monomer.

The invention also contemplates a number of GDF-1 in modified forms. These modified forms include GDF-1 linked to another cystine knot growth factor or a fraction of such a monomer.

In specific embodiments, the mutant GDF-1 heterodimer comprising at least one mutant subunit or the single chain GDF-1 analog as described above is functionally active, i.e., capable of exhibiting one or more functional activities associated with the wild-type GDF-1, such as GDF-1 receptor binding, GDF-1 protein family receptor signalling and extracellular secretion. Preferably, the mutant GDF-1 heterodimer or single chain GDF-1 analog is capable of binding to the GDF-1 receptor, preferably with affinity greater than the wild type GDF-1. Also it is preferable that such a mutant GDF-1 heterodimer or single chain GDF-1 analog triggers signal transduction. Most preferably, the mutant GDF-1 heterodimer comprising at least one mutant subunit or the single chain GDF-1 analog of the present invention has an in vitro bioactivity and/or in vivo bioactivity greater than the wild type GDF-1 and has a longer serum half-life than wild type GDF-1. Mutant GDF-1 heterodimers and single chain GDF-1 analogs of the invention can be tested for the desired activity by procedures known in the art.

Mutants of the Human Growth Differentiation Factor-5 Precursor (GDF-5 Precursor)

The human growth differentiation factor-5 Precursor (GDF-5 Precursor)contains 501 amino acids as shown in FIG. 38 (SEQ ID No: 37). The invention contemplates mutants of the GDF-5 precursor comprising single or multiple amino acid substitutions, deletions or insertions, of one, two, three, four or more amino acid residues when compared with the wild type GDF-5. Furthermore, the invention contemplates mutant GDF-5 precursor that are linked to another CKGF protein.

The present invention provides mutant GDF-5 precursor L1 hairpin loops having one or more amino acid substitutions between positions 404 and 425, inclusive, excluding Cys residues, as depicted in FIG. 38 (SEQ ID NO: 37). The amino acid substitutions include: A404X, L405X, H406X, V407X, N408X, F409X, K410X, D411X, M412X, G413X, W414X, D415X, D416X, W417X, I418X, I419X, A420X, P421X, L422X, E423X, Y424X, and E425X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the GDF-5 precursor where an acidic residue is present, the variable “X” would correspond to a basic amino acid residue. Specific examples of electrostatic charge altering mutations where a basic residue is introduced into the GDF-5 precursor sequence include one or more of the following: D411B, D415B, D416B, E423B, and E425B, wherein “B” is a basic amino acid residue.

Introducing acidic amino acid residues where basic residues are present in the GDF-5 precursor sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following H406Z and K410Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at H406U, K410U, D411U, D415U, D416U, E423U, and E425U, wherein “U” is a neutral amino acid.

Mutant GDF-5 precursor proteins are provided containing one or more electrostatic charge altering mutations in the L1 hairpin loop amino acid sequence that convert non-charged or neutral amino acid residues to charged residues. Examples of mutations converting neutral amino acid residues to charged residues include: A404Z, L405Z, V407Z, N408Z, F409Z, M412Z, G413Z, W414Z, W417Z, I418Z, I419Z, A420Z, P421Z, L422Z, Y424Z, A404B, L405B, V407B, N408B, F409B, M412B, G413B, W414B, W417B, I418B, I419B, A420B, P421B, L422B, and Y424B, wherein “Z” is an acidic amino acid and “B” is a basic amino acid.

Mutant GDF-5 precursor containing mutants in the L3 hairpin loop are also described. These mutant proteins have one or more amino acid substitutions, deletion or insertions, between positions 470 and 494, inclusive, excluding Cys residues, of the L3 hairpin loop, as depicted in FIG. 38 (SEQ ID NO: 37). The amino acid substitutions include: T469X, R470X, L471X, S472X, P473X, I474X, S475X, I476X, L477X, F478X, 1479X, D480X, S481X, A482X, N483X, N484X, V485X, V486X, Y487X, K488X, Q489X, Y490X, E491X, D492X, M493X, and V494X, wherein “X” is any amino acid residue, the substitution of which alters the electrostatic character of the L3 loop.

One set of mutations of the L3 hairpin loop includes introducing one or more basic amino acid residues into the GDF-5 precursor L3 hairpin loop amino acid sequence. For example, when introducing basic residues into the L3 loop of the GDF-5 precursor, the variable “X” of the sequence described above corresponds to a basic amino acid residue. Specific examples of electrostatic charge altering mutations where a basic residue is introduced into the GDF-5 precursor include one or more of the following: D480B, E491B, and D492B, wherein “B” is a basic amino acid residue.

The invention further contemplates introducing one or more acidic residues into the amino acid sequence of the GDF-5 precursor L3 hairpin loop. For example, one or more acidic amino acids can be introduced in the sequence of 470-494 described above, wherein the variable “X” corresponds to an acidic amino acid. Specific examples of such mutations include R470Z and K488Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at R470U, D480U, K488U, E491U, and D492U, wherein “U” is a neutral amino acid.

Mutant GDF-5 precursor proteins are provided containing one or more electrostatic charge altering mutations in the L3 hairpin loop amino acid sequence that convert non-charged or neutral amino acid residues to charged residues. Examples of mutations converting neutral amino acid residues to charged residues include L471Z, S472Z, P473Z, I474Z, S475Z, I476Z, L477Z, F478Z, I479Z, S481Z, A482Z, N483Z, N484Z, V485Z, V486Z, Y487Z, Q489Z, Y490Z, M493Z, V494Z, L471B, S472B, P473B, I474B, S475B, I476B, L477B, F478B, I479B, S481B, A482B, N483B, N484B, V485B, V486B, Y487B, Q489B, Y490B, M493B, and V494B, wherein “Z” is an acidic amino acid and “B” is a basic amino acid.

The present invention also contemplate GDF-5 precursor containing mutations outside of said β hairpin loop structures that alter the structure or conformation of those hairpin loops. These structural alterations in turn serve to increase the electrostatic interactions between regions of the β hairpin loop structures of GDF-5 precursor contained in a dimeric molecule, and a receptor having affinity for the dimeric protein. These mutations are found at positions selected from the group consisting of 1-403, 426-469, and 495-501 of the GDF-5 precursor

Specific examples of these mutation outside of the β hairpin L1 and L3 loop structures include, M1J, R2J, L3J, P4J, K5J, L6J, L7J, T8J, F9J, L10J, L11J, W12J, Y13J, L14J, A15J, W16J, L17J, D18J, L19J, E20J, F21J, I22J, C23J, T24J, V25J, L26J, G27J, A28J, P29J, D30J, L31J, G32J, Q33J, R34J, P35J, Q36J, G37J, S38J, R39J, P40J, G41J, L42J, A43J, K44J, A45J, E46J, A47J, K48J, E49J, R50J, P51J, P52J, L53J, A54J, R55J, N56J, V57J, F58J, R59J, P60J, G61J, G62J, H63J, S64J, Y65J, G66J, G67J, G68J, A69J, T70J, N71J, A72J, N73J, A74J, R75J, A76J, K77J, G78J, G79J, T80J, G81J, Q82J, T83J, G84J, G85J, L86J, T87J, Q88J, p89J, K90J, K91J, D92J, E93J, P94J, K95J, K96J, L97J, P98J, P99J, R100J, P101J, G102J, G103J, P104J, E105J, P106J, K107J, P108J, G109J, H110J, P111J, P112J, Q113J, T114J, R115J, Q116J, A117J, T118J, A119J, R120J, T121J, V122J, T123J, P124J, K125J, G126J, Q127J, L128J, P129J, G130J, G131J, K132J, A133J, P134J, P135J, K136J, A137J, G138J, S139J, V140J, P141J, S142J, S143J, F144J, L145J, L146J, K147J, K148J, A149J, R150J, E151J, P152J, G153J, P154J, P155J, R156J, E157J, P158J, K159J, E160J, P161J, F162J, R163J, P164J, P165J, P166J, I167J, T168J, P169J, H170J, E171J, Y172J, M173J, L174J, S175J, L176J, Y177J, R178J, T179J, L180J, S181J, D182J, A183J, D184J, R185J, K186J, G187J, G188J, N189J, S190J, S191J, V192J, K193J, L194J, E195J, A196J, G197J, L198J, A199J, N200J, T201J, I202J, T203J, S204J, F205J, I206J, D207J, K208J, G209J, Q210J, D211J, D212J, R213J, G214J, P215J, V21J, V217J, R218J, K219J, Q220J, R221J, Y222J, V223J, F224J, D225J, I226J, S227J, A228J, L229J, E230J, K231J, D232J, G233J, L234J, L235J, G236J, A237J, E238J, L239J, R240J, I241J, L242J, R243J, K244J, K245J, P246J, S247J, D248J, T249J, A250J, K251J, P252J, A253J, V254J, P255J, R256J, S257J, R258J, R259J, A260J, A261J, Q262J, L263J, K264J, L265J, S266J, S267J, C268J, P269J, S270J, G271J, R272J, Q273J, P274J, A275J, A276J, L277J, L278J, D279J, V280J, R281J, S282J, V283J, P284J, G285J, L286J, D287J, G288J, S289J, G290J, W291J, E292J, V293J, F294J, D295J, I296J, W297J, K298J, L299J, F300J, R301J, N302J, F303J, K304J, N305J, S306J, A307J, Q308J, L309J, C310J, L311J, E312J, L313J, E314J, A315J, W316J, E317J, R318J, G319J, R320J, T321J, V322J, D323J, L324J, R325J, G326J, L327J, G328J, F329J, D330J, R331J, A332J, A333J, R334J, Q33J, 5J, V336J, H337J, E338J, K339J, A340J, L341J, F342J, L343J, V344J, F345J, G346J, R347J, T348J, K349J, K350J, R351J, D352J, L353J, F354J, F355J, N356J, E357J, I358J, K359J, A360J, R361J, S362J, G363J, Q364J, D365J, D366J, K367J, T368J, V369J, Y370J, E371J, Y372J, L373J, F374J, S375J, Q376J, R377J, R378J, K379J, R380J, R381J, A382J, P383J, S384J, A385J, T386J, R387J, Q388J, G389J, K390J, R391J, P392J, S393J, K394J, N395J, L396J, K397J, A398J, R399J, C400J, S401J, R402J, K403J, A426J, F427J, H428J, C429J, E430J, G431J, L432J, C433J, E434J, F435J, P436J, L437J, R438J, S439J, H440J, L441J, E442J, P443J, T444J, N445J, H446J, A447J, V448J, I449J, Q450J, T451J, L452J, M453J, N454J, S455J, M456J, D457J, P458J, E459J, S460J, T461J, P462J, P463J, T464J, C465J, C466J, V467J, P468J, T469J, V495J, E496J, S497J, C498J, G499J, C500J, and R501J. The variable “J” is any amino acid whose introduction results in an increase in the electrostatic interaction between the L1 and L3 β hairpin loop structures of the GDF-5 precursor and a receptor with affinity for a dimeric protein containing the mutant GDF-5 precursor.

The invention also contemplates a number of GDF-5 precursor in modified forms. These modified forms include GDF-5 precursor linked to another cystine knot growth factor or a fraction of such a.

In specific embodiments, the mutant GDF-5 precursor heterodimer comprising at least one mutant subunit or the single chain GDF-5 precursor analog as described above is functionally active, i.e., capable of exhibiting one or more functional activities associated with the wild-type GDF-5 precursor, such as GDF-5 precursor receptor binding, GDF-5 precursor protein family receptor signalling and extracellular secretion. Preferably, the mutant GDF-5 precursor heterodimer or single chain GDF-5 precursor analog is capable of binding to the GDF-5 precursor receptor, preferably with affinity greater than the wild type GDF-5 precursor. Also it is preferable that such a mutant GDF-5 precursor heterodimer or single chain GDF-5 precursor analog triggers signal transduction. Most preferably, the mutant GDF-5 precursor heterodimer comprising at least one mutant subunit or the single chain GDF-5 precursor analog of the present invention has an in vitro bioactivity and/or in vivo bioactivity greater than the wild type GDF-5 precursor and has a longer serum half-life than wild type GDF-5 precursor. Mutant GDF-5 precursor heterodimers and single chain GDF-5 precursor analogs of the invention can be tested for the desired activity by procedures known in the art.

Mutants of the Human Growth Differentiation Factor-8 (GDF-8) Subunit

The human growth differentiation factor-8 (GDF-8) subunit contains 375 amino acids as shown in FIG. 39 (SEQ ID No: 38). The invention contemplates mutants of the GDF-8 subunit comprising single or multiple amino acid substitutions, deletions or insertions, of one, two, three, four or more amino acid residues when compared with the wild type monomer. Furthermore, the invention contemplates mutant GDF-8 subunit that are linked to another CKGF protein.

The present invention provides mutant GDF-8 subunit L1 hairpin loops having one or more amino acid substitutions between positions 286 and 305, inclusive, excluding Cys residues, as depicted in FIG. 39 (SEQ ID NO: 38). The amino acid substitutions include: L286X, T287X, V288X, D289X, F290X, E291X, A292X, F293X, G294X, W295X, D296X, W297X, I298X, I299X, A300X, P301X, K302X, R303X, Y304X, and K305X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the GDF-8 subunit monomer where an acidic residue is present, the variable “X” would correspond to a basic amino acid residue. Specific examples of electrostatic charge altering mutations where a basic residue is introduced into the GDF-8 subunit monomer include one or more of the following: D289B, E291B, and D296B, wherein “B” is a basic amino acid residue.

Introducing acidic amino acid residues where basic residues are present in the GDF-8 subunit monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following K302Z, R303Z, and K305Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D289U, E291U, D296U, K302U, R303U, and K305U, wherein “U” is a neutral amino acid.

Mutant GDF-8 subunit monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: L286Z, T287Z, V288Z, F290Z, A292Z, F293Z, G294Z, W295Z, W297Z, I298Z, I299Z, A300Z, P301Z, Y304Z, L286B, T287B, V288B, F290B, A292B, F293B, G294B, W295B, W297B, I298B, I299B, A300B, P301B, and Y304B, wherein “Z” is an acidic amino acid and “B” is a basic amino acid.

Mutant GDF-8 subunit containing mutants in the L3 hairpin loop are also described. These mutant proteins have one or more amino acid substitutions, deletion or insertions, between positions 344 and 368, inclusive, excluding Cys residues, of the L3 hairpin loop, as depicted in FIG. 39 (SEQ ID NO: 38). The amino acid substitutions include: K344X, M345X, S346X, P347X, I348X, N349X, M350X, L351X, Y352X, F353X, N354X, G355X, K356X, E357X, Q358X, I359X, I360X, Y361X, G362X, K363X, I364X, P365X, A366X, M367X, and V368X, wherein “X” is any amino acid residue, the substitution of which alters the electrostatic character of the L3 loop.

One set of mutations of the L3 hairpin loop includes introducing one or more basic amino acid residues into the GDF-8 subunit L3 hairpin loop amino acid sequence. For example, when introducing basic residues into the L3 loop of the GDF-8 subunit, the variable “X” of the sequence described above corresponds to a basic amino acid residue. Specific examples of electrostatic charge altering mutations where a basic residue is introduced into the GDF-8 subunit include E357B, wherein “B” is a basic amino acid residue.

The invention further contemplates introducing one or more acidic residues into the amino acid sequence of the GDF-8 subunit L3 hairpin loop. For example, one or more acidic amino acids can be introduced in the sequence of 344 and 368 described above, wherein the variable “X” corresponds to an acidic amino acid. Specific examples of such mutations include K344Z, K356Z, and K363Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced K344U, K356U, E357U, and K363U, wherein “U” is a neutral amino acid.

Mutant GDF-8 subunit proteins are provided containing one or more electrostatic charge altering mutations in the L3 hairpin loop amino acid sequence that convert non-charged or neutral amino acid residues to charged residues. Examples of mutations converting neutral amino acid residues to charged residues include, M345Z, S346Z, P347Z, 1348Z, N349Z, M350Z, L351Z, Y352Z, F353Z, N354Z, G355Z, Q358Z, I359Z, I360Z, Y361Z, G362Z, I364Z, P365J, A366Z, M367Z, V368Z, M345B, S346B, P347B, 1348B, N349B, M350B, L351B, Y352B, F353B, N354B, G355B, Q358B, 1359B, I360B, Y361B, G362B, I364B, P365B, A366B, M367B, and V368B, wherein “Z” is an acidic amino acid and “B” is a basic amino acid.

The present invention also contemplate GDF-8 subunit containing mutations outside of said β hairpin loop structures that alter the structure or conformation of those hairpin loops. These structural alterations in turn serve to increase the electrostatic interactions between regions of the β hairpin loop structures of GDF-8 subunit contained in a dimeric molecule, and a receptor having affinity for the dimeric protein. These mutations are found at positions selected from the group consisting of positions 1-285, 306-343, and 369-375 of the GDF-8 subunit monomer.

Specific examples of these mutation outside of the β hairpin L1 and L3 loop structures include, M1J, Q2J, K3J, L4J, Q5J, L6J, C7J, V8J, Y9J, I10J, Y11J, L12J, F13J, M14J, L15J, I16J, V17J, A18J, G19J, P20J, V21J, D22J, L23J, N24J, E25J, N26J, S27J, E28J, Q29J, K30J, E31J, N32J, V33J, E34J, K35J, E36J, G37J, L38J, C39J, N40J, A41J, C42J, T43J, W44J, R45J, Q46J, N47J, T48J, K49J, S50J, S51J, R52J, I53J, E54J, A55J, I56J, K57J, I58J, Q59J, I60J, L61J, S62J, K63J, L64J, R65J, L66J, E67J, T68J, A69J, P70J, N71J, I72J, S73J, K74J, D75J, V76J, I77J, R78J, Q79J, L80J, L81J, P82J, K83J, A84J, P85J, P86J, L87J, R88J, E89J, I90J, I91J, D92J, Q93J, Y94J, D95J, V96J, Q97J, R98J, D99J, D100J, S101J, S102J, D103J, G104J, S105J, L106J, E107J, D108J, D109J, D110J, Y111J, H112J, A113J, T114J, T115J, E116J, T117J, I118J, I119J, T120J, M121J, P122J, T123J, E124J, S125J, D126J, F127J, L128J, M129J, Q130J, V131J, D132J, G133J, K134J, P135J, K136J, C137J, C138J, F139J, F140J, K141J, F142J, S143J, S144J, K145J, I146J, Q147J, Y148J, N149J, K150J, V151J, V152J, K153J, A154J, Q155J, L156J, W157J, I158J, Y159J, L160J, R161J, P162J, V163J, E164J, T165J, P166J, T167J, T168J, V169J, F170J, V171J, Q172J, I173J, L174J, R175J, L176J, I177J, K178J, P179J, M180J, K181J, D182J, G183J, T184J, R185J, Y186J, T187J, G188J, I189J, R190J, S191J, L192J, K193J, L194J, D195J, M196J, N197J, P198J, G199J, T200J, G201J, I202J, W203J, Q204J, S205J, I206J, D207J, V208J, K209J, T210J, V211J, L212J, Q213J, N214J, W215J, L216J, K217J, Q218J, P219J, E220J, S221J, N222J, L223J, G224J, I225J, E226J, I227J, K228J, A229J, L230J, D231J, E232J, N233J, G234J, H235J, D236J, L237J, A238J, V239J, T240J, F241J, P242J, G243J, P244J, G245J, E246J, D247J, G248J, L249J, N250J, P251J, F252J, L253J, E254J, V255J, K256J, V257J, T258J, D259J, T260J, P261J, K262J, R263J, S264J, R265J, R266J, D267J, F268J, G269J, L270J, D271J, C272J, D273J, E274J, H275J, S276J, T277J, E278J, S279J, R280J, C281J, C282J, R283J, Y284J, P285J, A306J, N307J, Y308J, C309J, S310J, G311J, E312J, C313J, E314J, F315J, V316J, F317J, L318J, Q319J, K320J, Y321J, P322J, H323J, T324J, H325J, L326J, V327J, H328J, Q329J, A330J, Q331j, P332J, R333J, G334J, S335J, A336J, G337J, P338J, C339J, C340J, T341J, P342J, T343J, V369J, D370J, R371J, C372J, G373J, C374J, and S375J. The variable “J” is any amino acid whose introduction results in an increase in the electrostatic interaction between the L1 and L3 β hairpin loop structures of the GDF-8 subunit and a receptor with affinity for a dimeric protein containing the mutant GDF-8 subunit monomer.

The invention also contemplates a number of GDF-8 subunit in modified forms. These modified forms include GDF-8 subunit linked to another cystine knot growth factor or a fraction of such a monomer.

In specific embodiments, the mutant GDF-8 subunit heterodimer comprising at least one mutant subunit or the single chain GDF-8 subunit analog as described above is functionally active, i.e., capable of exhibiting one or more functional activities associated with the wild-type GDF-8 subunit, such as GDF-8 subunit receptor binding, GDF-8 subunit protein family receptor signalling and extracellular secretion. Preferably, the mutant GDF-8 subunit heterodimer or single chain GDF-8 subunit analog is capable of binding to the GDF-8 subunit receptor, preferably with affinity greater than the wild type GDF-8 subunit. Also it is preferable that such a mutant GDF-8 subunit heterodimer or single chain GDF-8 subunit analog triggers signal transduction. Most preferably, the mutant GDF-8 subunit heterodimer comprising at least one mutant subunit or the single chain GDF-8 subunit analog of the present invention has an in vitro bioactivity and/or in vivo bioactivity greater than the wild type GDF-8 subunit and has a longer serum half-life than wild type GDF-8 subunit. Mutant GDF-8 subunit heterodimers and single chain GDF-8 subunit analogs of the invention can be tested for the desired activity by procedures known in the art.

Mutants of the Human Growth Differentiation Factor-9 (GDF-9) Subunit

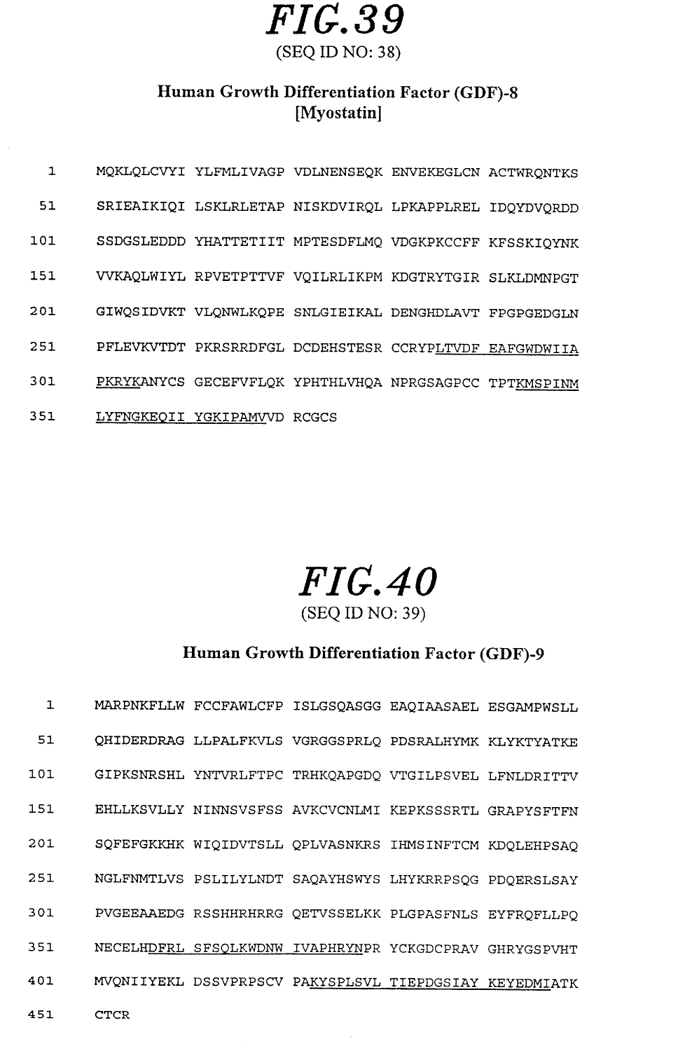

The human growth differentiation factor-9 (GDF-9) subunit contains 454 amino acids as shown in FIG. 40 (SEQ ID No: 39). The invention contemplates mutants of the GDF-9 comprising single or multiple amino acid substitutions, deletions or insertions, of one, two, three, four or more amino acid residues when compared with the wild type monomer. Furthermore, the invention contemplates mutant GDF-9 that are linked to another CKGF protein.

The present invention provides mutant GDF-9 L1 hairpin loops having one or more amino acid substitutions between positions 357 and 378, inclusive, excluding Cys residues, as depicted in FIG. 40 (SEQ ID NO: 39). The amino acid substitutions include: D357X, F358X, R359X, L360X, S361X, F362X, S363X, Q364X, L365X, K366X, W367X, D368X, N369X, W370X, I371X, V372X, A373X, P374X, H375X, R376X, Y377X, and N378X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the GDF-9 monomer where an acidic residue is present, the variable “X” would correspond to a basic amino acid residue. Specific examples of electrostatic charge altering mutations where a basic residue is introduced into the GDF-9 monomer include one or more of the following: D357B and D368B wherein “B” is a basic amino acid residue.

Introducing acidic amino acid residues where basic residues are present in the GDF-9 monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following R359Z, K366Z, H375Z, and R376Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at D357U, R359U, K366U, D368U, H375U, and R376U, wherein “U” is a neutral amino acid.

Mutant GDF-9 monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: F358Z, L360Z, S361Z, F362Z, S363Z, Q364Z, L365Z, W367Z, N369Z, W370Z, I371Z, V372Z, A373Z, P374Z, Y377Z, N378Z, F358B, L360B, S361B, F362B, S363B, Q364B, L365B, W367B, N369B, W370B, I371B, V372B, A373B, P374B, Y377B, and N378B, wherein “Z” is an acidic amino acid and “B” is a basic amino acid.

Mutant GDF-9 containing mutants in the L3 hairpin loop are also described. These mutant proteins have one or more amino acid substitutions, deletion or insertions, between positions 423 and 447, inclusive, excluding Cys residues, of the L3 hairpin loop, as depicted in FIG. 40 (SEQ ID NO: 39). The amino acid substitutions include: K423X, Y424X, S425X, P426X, L427X, S428X, V429X, L430X, T431X, I432X, E433X, P434X, X, D435X, G436X, S437X, I438A439X, Y440X, K441X, E442X, Y443X, E444X, D445X, M446X, and I447X, wherein “X” is any amino acid residue, the substitution of which alters the electrostatic character of the L3 loop.

One set of mutations of the L3 hairpin loop includes introducing one or more basic amino acid residues into the GDF-9 L3 hairpin loop amino acid sequence. For example, when introducing basic residues into the L3 loop of the GDF-9, the variable “X” of the sequence described above corresponds to a basic amino acid residue. Specific examples of electrostatic charge altering mutations where a basic residue is introduced into the GDF-9 include one or more of the following: E433B, D435B, E442B, and E444B, wherein “B” is a basic amino acid residue.

The invention further contemplates introducing one or more acidic residues into the amino acid sequence of the GDF-9 L3 hairpin loop. For example, one or more acidic amino acids can be introduced in the sequence of 423-447 described above, wherein the variable “X” corresponds to an acidic amino acid. Specific examples of such mutations include K423Z and K441Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced K423U, E433U, D435U, K441U, E442U, E444U, and D445U, wherein “U” is a neutral amino acid.

Mutant GDF-9 proteins are provided containing one or more electrostatic charge altering mutations in the L3 hairpin loop amino acid sequence that convert non-charged or neutral amino acid residues to charged residues. Examples of mutations converting neutral amino acid residues to charged residues include, Y424Z, S425Z, P426Z, L427Z, S428Z, V429Z, L430Z, T431Z, I432Z, P434Z, G436Z, S437Z, I438Z, A439Z, Y440Z, Y443Z, M446Z, I447Z, Y424B, S425B, P426B, L427B, S428B, V429B, L430B, T431B, I432B, P434B, G436B, S437B, I438B, A439B, Y440B, Y443B, M446B, and I447B, wherein “Z” is an acidic amino acid and “B” is a basic amino acid.

The present invention also contemplate GDF-9 containing mutations outside of said β hairpin loop structures that alter the structure or conformation of those hairpin loops. These structural alterations in turn serve to increase the electrostatic interactions between regions of the β hairpin loop structures of GDF-9 contained in a dimeric molecule, and a receptor having affinity for the dimeric protein. These mutations are found at positions selected from the group consisting of positions 1-356, 379-422, and 448-454 of the GDF-9 monomer.

Specific examples of these mutation outside of the β hairpin L1 and L3 loop structures include, M1J, A2J, R3J, P4J, N5J, K6J, F7J, L8J, L9J, W10J, F11J, C12J, C13J, F14J, A15J, W16J, L17J, C18J, F19J, P20J, I21J, S22J, L23J, G24J, S25J, Q26J, A27J, S28J, G29J, G30J, E31J, A32J, Q33J, I34J, A35J, A36J, S37J, A38J, E39J, L40J, E41J, S42J, G43J, A44J, M45J, P46J, W47J, S48J, L49J, L50J, Q51J, H52J, I53J, D54J, E55J, R56J, D57J, R58J, A59J, G60J, L61J, L62J, P63J, A64J, L65J, F66J, K67J, V68J, L69J, S70J, V71J, G72J, R73J, G74J, G75J, S76J, P77J, R78J, L79J, Q80J, P81J, D82J, S83J, R84J, A85J, L86J, H87J, Y88J, M89J, K90J, K91J, L92J, Y93J, K94J, T95J, Y96J, A97J, T98J, K99J, E100J, G101J, I102J, P103J, K104J, S105J, N106J, R107J, S108J, H109J, L110J, Y111J, N112J, T113J, V114J, R115J, L116J, F117J, T118J, P119J, C120J, T121J, R122J, H123J, K124J, Q125J, A126J, P127J, G128J, D129J, Q130J, V131J, T132J, G133J, I134J, L135J, P136J, S137J, V138J, E140J, L141J, F142J, N143J, L144J, D145J, R146J, I147J, T148J, T149J, V150J, E151J, H152J, L153J, L154J, K155J, S156J, V157J, L158J, L159J, Y160J, N161J, I162J, N163J, N164J, S165J, V166J, S167J, F168J, S169J, S170J, A171J, V172J, K173J, C174J, V175J, C176J, N177J, L178J, M179J, I180J, K181J, E182J, P183J, K184J, S185J, S186J, S187J, R188J, T189J, L190J, G191J, R192J, A193J, P194J, Y195J, S196J, F197J, T198J, F199J, N200J, S201J, Q202J, F203J, E204J, F205J, G206J, K207J, K208J, H209J, K210J, W211J, 1212J, Q213J, 1214J, D215J, V216J, T217J, S218J, L219J, L220J, Q221J, P222J, L223J, V224AJ, 225J, S226J, N227J, K228J, R229J, S230J, I231J, H232J, M233J, S234J, 1235J, N236J, F237J, T238J, C239J, M240J, K241J, D242J, Q243J, L244J, E245J, H246J, P247J, S248J, A249J, Q250J, N251J, G252J, L253J, F254J, N255J, M256J, T257J, L258VJ, 259J, S260J, P261J, S262J, L263J, I264J, L265J, Y266J, L267J, N268J, D269J, T270J, S271J, A272J, Q273J, A274J, Y275J, H276J, S277J, W278J, Y279J, S280J, L281J, H282J, Y283J, K284J, R285J, R286J, P287J, S288J, Q289J, G290J, P291J, D292J, Q293J, E294J, R295J, S296J, L297J, S298J, A299J, Y300J, P301J, V302J, G303J, E304J, E305J, A306J, A307J, E308J, D309J, G310J, R311J, S312J, S313j, H314J, H315J, R316J, H317J, R318J, R319J, G320J, Q321J, E322J, T323J, V324J, S325J, S326J, E327J, L328J, K329J, K330J, P331J, L332J, G333J, P334J, A335J, S336J, F337J, N338J, L339J, S340J, E341J, Y342J, F343J, R344J, Q345J, F346J, L347J, L348J, P349J, Q350J, N351J, E352J, C353J, E354J, L355J, H356J, P379J, R380J, Y381J, C382J, K383J, G384J, D385J, C386J, P387J, R388J, A389J, V390J, G391J, H392J, R393J, Y394J, G395J, S396J, P397J, V398J, H399J, T400J, M401J, V402J, Q403J, N404J, 1405J, I406J, Y407J, E408J, K409J, L410J, D411J, S412J, S413J, V414J, P415J, R416J, P417J, S418J, C419J, V420J, P421J, A422J, A448J, T449J, K450J, C451J, T452J, C453J, and R454J. The variable “J” is any amino acid whose introduction results in an increase in the electrostatic interaction between the L1 and L3 β hairpin loop structures of the GDF-9 and a receptor with affinity for a dimeric protein containing the mutant GDF-9 monomer.

The invention also contemplates a number of GDF-9 in modified forms. These modified forms include GDF-9 linked to another cystine knot growth factor or a fraction of such a monomer.

In specific embodiments, the mutant GDF-9 heterodimer comprising at least one mutant subunit or the single chain GDF-9 analog as described above is functionally active, i.e., capable of exhibiting one or more functional activities associated with the wild-type GDF-9, such as GDF-9 receptor binding, GDF-9 protein family receptor signalling and extracellular secretion. Preferably, the mutant GDF-9 heterodimer or single chain GDF-9 analog is capable of binding to the GDF-9 receptor, preferably with affinity greater than the wild type GDF-9. Also it is preferable that such a mutant GDF-9 heterodimer or single chain GDF-9 analog triggers signal transduction. Most preferably, the mutant GDF-9 heterodimer comprising at least one mutant subunit or the single chain GDF-9 analog of the present invention has an in vitro bioactivity and/or in vivo bioactivity greater than the wild type GDF-9 and has a longer serum half-life than wild type GDF-9. Mutant GDF-9 heterodimers and single chain GDF-9 analogs of the invention can be tested for the desired activity by procedures known in the art.

Mutants of the Human Artemin/Glial-Cell Derived Neurotrophic Factor (GDNF)

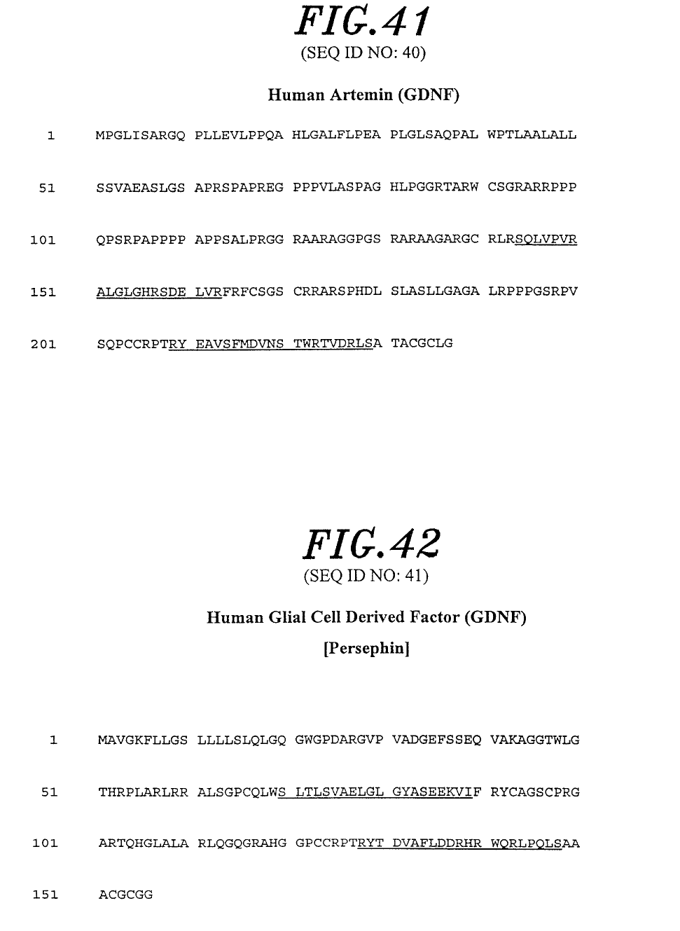

The human artemin/Glial-Cell Derived Neurotrophic Factor (GDNF) contains 337 amino acids as shown in FIG. 41 (SEQ ID No: 40). The invention contemplates mutants of the human artemin (GDNF) comprising single or multiple amino acid substitutions, deletions or insertions, of one, two, three, four or more amino acid residues when compared with the wild type monomer. Furthermore, the invention contemplates mutant human artemin (GDNF) that are linked to another CKGF protein.

The present invention provides mutant human artemin (GDNF) L1 hairpin loops having one or more amino acid substitutions between positions 144 and 163, inclusive, excluding Cys residues, as depicted in FIG. 41 (SEQ ID NO: 40). The amino acid substitutions include: S144X, Q145X, L146X, V147X, P148X, V149X, R150X, A151X, L152X, G153X, L154X, G155X, H156X, R157X, S158X, D159X, E160X, L161X, V162X, and R163X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the human artemin (GDNF) monomer where an acidic residue is present, the variable “X” would correspond to a basic amino acid residue. Specific examples of electrostatic charge altering mutations where a basic residue is introduced into the human artemin (GDNF) monomer include one or more of the following: D159B and E160B, wherein “B” is a basic amino acid residue.

Introducing acidic amino acid residues where basic residues are present in the human artemin (GDNF) monomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: R150Z, H156Z, R157Z, and R163Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at R150U, H156U, R157U, D159U, E160U, and R163U, wherein “U” is a neutral amino acid.

Mutant human artemin (GDNF) monomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include: S144Z, Q145Z, L146Z, V147Z, P148Z, V149Z, A151Z, L152Z, G153Z, L154Z, G155Z, S518Z, L161Z, V162Z, S144B, Q145B, L146B, V147B, P148B, V149B, A151B, L152B, G153B, L154B, G155B, S518B, L161B, and V162B, wherein “Z” is an acidic amino acid and “B” is a basic amino acid.

Mutant human artemin (GDNF) containing mutants in the L3 hairpin loop are also described. These mutant proteins have one or more amino acid substitutions, deletion or insertions, between positions 209 and 229, inclusive, excluding Cys residues, of the L3 hairpin loop, as depicted in FIG. 41 (SEQ ID NO: 40). The amino acid substitutions include: R209X, Y210X, E211X, A212X, V213X, S214X, F215X, M216X, D217X, V218X, N219X, S220X, T221X, W222X, R223X, T224X, V225X, D226X, R227X, L228X, and S229X, wherein “X” is any amino acid residue, the substitution of which alters the electrostatic character of the L3 loop.

One set of mutations of the L3 hairpin loop includes introducing one or more basic amino acid residues into the human artemin (GDNF) L3 hairpin loop amino acid sequence. For example, when introducing basic residues into the L3 loop of the human artemin (GDNF), the variable “X” of the sequence described above corresponds to a basic amino acid residue. Specific examples of electrostatic charge altering mutations where a basic residue is introduced into the human artemin (GDNF) include one or more of the following: E211B, D217B, and D226B, wherein “B” is a basic amino acid residue.

The invention further contemplates introducing one or more acidic residues into the amino acid sequence of the human artemin (GDNF) L3 hairpin loop. For example, one or more acidic amino acids can be introduced in the sequence of 209-229 described above, wherein the variable “X” corresponds to an acidic amino acid. Specific examples of such mutations include R209Z, R223Z, and R227Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at R209U, E211U, D217U, R223U, D226U, and R227U, wherein “U” is a neutral amino acid.

Mutant human artemin (GDNF) proteins are provided containing one or more electrostatic charge altering mutations in the L3 hairpin loop amino acid sequence that convert non-charged or neutral amino acid residues to charged residues. Examples of mutations converting neutral amino acid residues to charged residues include, of Y210Z, A212Z, V213Z, S214Z, F215Z, M216Z, V218Z, N219Z, S220Z, T221Z, W222Z, T224Z, V225Z, L228Z, S229Z, Y210B, A212B, V213B, S214B, F215B, M216B, V218B, N219B, S220B, T221B, W222B, T224B, V225B, L228B, and S229B, wherein “Z” is an acidic amino acid and “B” is a basic amino acid.

The present invention also contemplate human artemin (GDNF) containing mutations outside of said β hairpin loop structures that alter the structure or conformation of those hairpin loops. These structural alterations in turn serve to increase the electrostatic interactions between regions of the β hairpin loop structures of human artemin (GDNF) contained in a dimeric molecule, and a receptor having affinity for the dimeric protein. These mutations are found at positions selected from the group consisting of positions 1-143, 164-208, and 230-237 of the human artemin (GDNF) monomer.

Specific examples of these mutation outside of the β hairpin L1 and L3 loop structures include, M1J, P2J, G3J, L4J, 15J, S6J, A7J, R8J, G9J, Q10J, P11J, L12J, L13J, E14J, V15J, L16J, P17J, P18J, Q19J, A20J, H21J, L22J, G23J, A24J, L25J, F26J, L27J, P28J, E29J, A30J, P31J, L32J, G33J, L34J, S35J, A36J, Q37J, P38J, A39J, L40J, W41J, P42J, T43J, L44J, A45J, A46J, L47J, A48J, L49J, L50J, S51J, S52J, V53J, A54J, E55J, A56J, S57J, L58J, G59J, S60J, A61J, P62J, R63J, S64J, P65J, A66J, P67J, R68J, E69J, G70J, P71J, P72J, P73J, V74J, L75J, A76J, S77J, P78J, A79J, G80J, H81J, L82J, P83J, G84J, G85J, R86J, T87J, A88J, R89J, W90J, C91J, S92J, G93J, R94J, A95J, R96J, R97J, P98J, P99J, P100J, Q101J, P102J, S103J, R104J, P105J, A106J, P107J, P108J, P109J, P110J, A111J, P112J, P113J, S114J, A115J, L116J, P117J, R118J, G119J, G120J, R121J, A122J, A123J, R124J, A125J, G126J, G127J, P128J, G129J, S130J, R131J, A132J, R133J, A134J, A135J, G136J, A137J, R138J, G139J, C140J, R141J, L142J, R143J, F164J, R165J, F166J, C167J, S168J, G169J, S170J, C171J, R172J, R173J, A174J, R175J, S176J, P177J, H178J, D179J, L180J, S181J, L182J, A183J, S184J, L185J, L186J, G187J, A188J, G189J, A190J, L191J, R192J, P193J, P194J, P195J, G196J, S197J, R198J, P199J, V200J, S201J, Q202J, P203J, C204J, C205J, R206J, P207J, T208J, A230J, T231J, A232J, C233J, G234J, C235J, L236J, and G237J. The variable “J” is any amino acid whose introduction results in an increase in the electrostatic interaction between the L1 and L3 β hairpin loop structures of the human artemin (GDNF) and a receptor with affinity for a dimeric protein containing the mutant human artemin (GDNF) monomer.

The invention also contemplates a number of human artemin (GDNF) in modified forms. These modified forms include human artemin (GDNF) linked to another cystine knot growth factor or a fraction of such a monomer.

In specific embodiments, the mutant human artemin (GDNF) heterodimer comprising at least one mutant subunit or the single chain human artemin (GDNF) analog as described above is functionally active, i.e., capable of exhibiting one or more functional activities associated with the wild-type human artemin (GDNF), such as human artemin (GDNF) receptor binding, human artemin (GDNF) protein family receptor signalling and extracellular secretion. Preferably, the mutant human artemin (GDNF) heterodimer or single chain human artemin (GDNF) analog is capable of binding to the human artemin (GDNF) receptor, preferably with affinity greater than the wild type human artemin (GDNF). Also it is preferable that such a mutant human artemin (GDNF) heterodimer or single chain human artemin (GDNF) analog triggers signal transduction. Most preferably, the mutant human artemin (GDNF) heterodimer comprising at least one mutant subunit or the single chain human artemin (GDNF) analog of the present invention has an in vitro bioactivity and/or in vivo bioactivity greater than the wild type human artemin (GDNF) and has a longer serum half-life than wild type human artemin (GDNF). Mutant human artemin (GDNF) heterodimers and single chain human artemin (GDNF) analogs of the invention can be tested for the desired activity by procedures known in the art.

Mutants of the Human Glial Cell Derived Factor (GDNF)/Persephin Subunit

The human glial-cell derived neurotrophic factor (GDNF)/Persephin subunit contains 156 amino acids as shown in FIG. 42 (SEQ ID No: 41). The invention contemplates mutants of the human glial cell derived factor (GDNF)/Persephin subunit comprising single or multiple amino acid substitutions, deletions or insertions, of one, two, three, four or more amino acid residues when compared with the wild type monomer. Furthermore, the invention contemplates mutant human glial cell derived factor (GDNF)/Persephin subunit that are linked to another CKGF protein.

The present invention provides mutant human glial cell derived factor (GDNF)/Persephin subunit L1 hairpin loops having one or more amino acid substitutions between positions 70 and 89, inclusive, excluding Cys residues, as depicted in FIG. 42 (SEQ ID NO: 41). The amino acid substitutions include: S70X, L71X, T72X, L73X, S74X, V75X, A76X, E77X, L78X, G79X, L80X, G81X, Y82X, A83X, S84X, E85X, E86X, K87X, V88X, and I89X. “X” is any amino acid residue, the substitution with which alters the electrostatic character of the hairpin loop.

Specific examples of the mutagenesis regime of the present invention include the introduction of basic amino acid residues where acidic residues are present. For example, when introducing basic residues into the L1 loop of the human glial cell derived factor (GDNF)/Persephin subunitmonomer where an acidic residue is present, the variable “X” would correspond to a basic amino acid residue. Specific examples of electrostatic charge altering mutations where a basic residue is introduced into the human glial cell derived factor (GDNF)/Persephin subunitmonomer include one or more of the following: E77B, E85B, and E86B, wherein “B” is a basic amino acid residue.

Introducing acidic amino acid residues where basic residues are present in the human glial cell derived factor (GDNF)/Persephin subunitmonomer sequence is also contemplated. In this embodiment, the variable “X” corresponds to an acidic amino acid. The introduction of these amino acids serves to alter the electrostatic character of the L1 hairpin loops to a more negative state. Examples of such amino acid substitutions include one or more of the following: K87Z, wherein “Z” is an acidic amino acid residue.

The invention also contemplates reducing a positive or negative charge in the L1 hairpin loop by mutating a charged residue to a neutral residue. For example, one or more neutral amino acids can be introduced into the L1 sequence described above where the variable “X” corresponds to a neutral amino acid. In another example, one or more neutral residues can be introduced at E77U, E85U, E86U, and K87U, wherein “U” is a neutral amino acid.

Mutant human glial cell derived factor (GDNF)/Persephin subunitmonomer proteins are provided containing one or more electrostatic charge altering mutations in the L1 hairpin loop amino acid sequence that convert non-charged or neutral amino acid residues to charged residues. Examples of mutations converting neutral amino acid residues to charged residues include S70Z, L71Z, T72Z, L73Z, S74Z, V75Z, A76Z, L78Z, G79Z, L80Z, G81Z, Y82Z, A83Z, S84Z, V88Z, I89Z, S70B, L71B, T72B, L73B, S74B, V75B, A76B, L78B, G79B, L80B, G81B, Y82B, A83B, S84B, V88B, and I89B, wherein “Z” is an acidic amino acid and “B” is a basic amino acid.

Mutant human glial cell derived factor (GDNF)/Persephin subunit containing mutants in the L3 hairpin loop are also described. These mutant proteins have one or more amino acid substitutions, deletion or insertions, between positions 128 and 148, inclusive, excluding Cys residues, of the L3 hairpin loop, as depicted in FIG. 42 (SEQ ID NO: 41). The amino acid substitutions include: R128X, Y129X, T130X, D131X, V132X, A133X, F134X, L135X, D136X, D137X, R138X, H139X, R140X, W141X, Q142X, R143X, L144X, P145X, Q146X, L147X, and S148X, wherein “X” is any amino acid residue, the substitution of which alters the electrostatic character of the L3 loop.

One set of mutations of the L3 hairpin loop includes introducing one or more basic amino acid residues into the human glial cell derived factor (GDNF)/Persephin subunitL3 hairpin loop amino acid sequence. For example, when introducing basic residues into the L3 loop of the human glial cell derived factor (GDNF)/Persephin subunit, the variable “X” of the sequence described above corresponds to a basic amino acid residue. Specific examples of electrostatic charge altering mutations where a basic residue is introduced into the human glial cell derived factor (GDNF)/Persephin subunit include one or more of the following: D131B, D136B, and D137B, wherein “B” is a basic amino acid residue.

The invention further contemplates introducing one or more acidic residues into the amino acid sequence of the human glial cell derived factor (GDNF)/Persephin subunit L3 hairpin loop. For example, one or more acidic amino acids can be introduced in the sequence of 128-148 described above, wherein the variable “X” corresponds to an acidic amino acid. Specific examples of such mutations include R128Z, R138Z, H139Z, R14OZ, and R143Z, wherein “Z” is an acidic amino acid residue.

The invention also contemplates reducing a positive or negative electrostatic charge in the L3 hairpin loop by mutating a charged residue to a neutral residue in this region. For example, one or more neutral amino acids can be introduced into the L3 hairpin loop amino acid sequence described above where the variable “X” corresponds to a neutral amino acid. For example, one or more neutral residues can be introduced at R128U, D131U, D136U, D137U, R138U, H139U, R140U, and R143U, wherein “U” is a neutral amino acid.

Mutant human glial cell derived factor (GDNF)/Persephin subunitproteins are provided containing one or more electrostatic charge altering mutations in the L3 hairpin loop amino acid sequence that convert non-charged or neutral amino acid residues to charged residues. Examples of mutations converting neutral amino acid residues to charged residues include, Y129Z, T130Z, V132Z, A133Z, F134Z, L135Z, W141Z, Q142Z, L144Z, P145Z, Q146Z, L147Z, S148Z, Y129B, T130B, V132B, A133B, F134B, L135B, W141B, Q142B, L144B, P145B, Q146B, L147B, and S148B, wherein “Z” is an acidic amino acid and “B” is a basic amino acid.

The present invention also contemplate human glial cell derived factor (GDNF)/Persephin subunit containing mutations outside of said β hairpin loop structures that alter the structure or conformation of those hairpin loops. These structural alterations in turn serve to increase the electrostatic interactions between regions of the β hairpin loop structures of human glial cell derived factor (GDNF)/Persephin subunit contained in a dimeric molecule, and a receptor having affinity for the dimeric protein. These mutations are found at positions selected from the group consisting of positions 1-69, 90-127, and 149-156 of the human glial cell derived factor (GDNF)/Persephin subunitmonomer.

Specific examples of these mutation outside of the β hairpin L1 and L3 loop structures include, M1J, A2J, V3J, G4J, K5J, F6J, L7J, L8J, G9J, S10J, L11J, L12J, L13J, L14J, S15J, L16J, Q17J, L18J, G19J, Q20J, G21J, W22J, G23J, P24J, D25J, A26J, R27J, G28J, V29J, P30J, V31J, A32J, D33J, G34J, E35J, F36J, S37J, S38J, E39J, Q40J, V41J, A42J, K43J, A44J, G45J, G46J, T47J, W48J, L49J, G50J, T51J, H52J, R53J, P54J, L55J, A56J, R57J, L58J, R59J, R60J, A61J, L62J, S63J, G64J, P65J, C66J, Q67J, L68J, W69J, F9OJ, R91J, Y92J, C93J, A94J, G95J, S96J, C97J, P98J, R99J, G100J, A101J, R102J, T103J, Q104J, H105J, G106J, L107J, A108J, L109J, A110J, R111J, L112J, Q113J, G114J, Q115J, G116J, R117J, A118J, H119J, G120J, G121J, P122J, C123J, C124J, R125J, P126J, T127J, A149J, A150J, A151J, C152J, G153J, C154J, G155J, and G156J. The variable “J” is any amino acid whose introduction results in an increase in the electrostatic interaction between the L1 and L3 β hairpin loop structures of the human glial cell derived factor (GDNF)/Persephin subunitand a receptor with affinity for a dimeric protein containing the mutant human glial cell derived factor (GDNF)/Persephin subunitmonomer.

The invention also contemplates a number of human glial cell derived factor (GDNF)/Persephin subunit in modified forms. These modified forms include human glial cell derived factor (GDNF)/Persephin subunit linked to another cystine knot growth factor or a fraction of such a monomer.

In specific embodiments, the mutant human glial cell derived factor (GDNF)/Persephin subunit heterodimer comprising at least one mutant subunit or the single chain human glial cell derived factor (GDNF)/Persephin subunit analog as described above is functionally active, i.e., capable of exhibiting one or more functional activities associated with the wild-type human glial cell derived factor (GDNF)/Persephin subunit, such as human glial cell derived factor (GDNF)/Persephin subunit receptor binding, human glial cell derived factor (GDNF)/Persephin subunit protein family receptor signalling and extracellular secretion. Preferably, the mutant human glial cell derived factor (GDNF)/Persephin subunit heterodimer or single chain human glial cell derived factor (GDNF)/Persephin subunit analog is capable of binding to the human glial cell derived factor (GDNF)/Persephin subunit receptor, preferably with affinity greater than the wild type human glial cell derived factor (GDNF)/Persephin subunit. Also it is preferable that such a mutant human glial cell derived factor (GDNF)/Persephin subunit heterodimer or single chain human glial cell derived factor (GDNF)/Persephin subunit analog triggers signal transduction. Most preferably, the mutant human glial cell derived factor (GDNF)/Persephin subunit heterodimer comprising at least one mutant subunit or the single chain human glial cell derived factor (GDNF)/Persephin subunit analog of the present invention has an in vitro bioactivity and/or in vivo bioactivity greater than the wild type human glial cell derived factor (GDNF)/Persephin subunit and has a longer serum half-life than wild type human glial cell derived factor (GDNF)/Persephin subunit. Mutant human glial cell derived factor (GDNF)/Persephin subunit heterodimers and single chain human glial cell derived factor (GDNF)/Persephin subunit analogs of the invention can be tested for the desired activity by procedures known in the art.

Polynucleotides Encoding Mutant Tumor Growth Factor β Family Proteins and Analogs

The present invention also relates to nucleic acids molecules comprising sequences encoding mutant subunits of human tumor growth factor-β (TGF-β) family protein and TGF-β family protein analogs of the invention, wherein the sequences contain at least one base insertion, deletion or substitution, or combinations thereof that results in single or multiple amino acid additions, deletions and substitutions relative to the wild type protein. Base mutations that do not alter the reading frame of the coding region are preferred. As used herein, when two coding regions are said to be fused, the 3′ end of one nucleic acid molecule is ligated to the 5′ (or through a nucleic acid encoding a peptide linker) end of the other nucleic acid molecule such that translation proceeds from the coding region of one nucleic acid molecule into the other without a frameshift.

Due to the degeneracy of the genetic code, any other DNA sequences that encode the same amino acid sequence for a mutant subunit or monomer may be used in the practice of the present invention. These include but are not limited to nucleotide sequences comprising all or portions of the coding region of the subunit or monomer that are altered by the substitution of different codons that encode the same amino acid residue within the sequence, thus producing a silent change.

In one embodiment, the present invention provides nucleic acid molecules comprising sequences encoding mutant TGF-β family protein subunits, wherein the mutant TGF-β family protein subunits comprise single or multiple amino acid substitutions, preferably located in or near the β hairpin L1 and/or L3 loops of the target protein. The invention also provides nucleic acids molecules encoding mutant TGF-β family protein subunits having an amino acid substitution outside of the L1 and/or L3 loops such that the electrostatic interaction between those loops and the cognate receptor of the TGF-β family protein dimer are increased. The present invention further provides nucleic acids molecules comprising sequences encoding mutant TGF-β family protein subunits comprising single or multiple amino acid substitutions, preferably located in or near the β hairpin L1 and/or L3 loops of the TGF-β family protein subunit, and/or covalently joined to another CKGF protein.

In yet another embodiment, the invention provides nucleic acid molecules comprising sequences encoding TGF-β family protein analogs, wherein the coding region of a mutant TGF-β family protein subunit comprising single or multiple amino acid substitutions, is fused with the coding region of its corresponding dimeric unit, which can be a wild type subunit or another mutagenized monomeric subunit. Also provided are nucleic acid molecules encoding a single chain TGF-β family protein analog wherein the carboxyl terminus of the mutant TGF-β family protein monomer is linked to the amino terminus of another CKGF protein. In still another embodiment, the nucleic acid molecule encodes a single chain TGF-β family protein analog, wherein the carboxyl terminus of the mutant TGF-β family protein monomer is covalently bound to the amino terminus another CKGF protein such as the amino terminus of CTEP, and the carboxyl terminus of bound amino acid sequence is covalently bound to the amino terminus of a mutant TGF-β family protein monomer without the signal peptide.

The single chain analogs of the invention can be made by ligating the nucleic acid sequences encoding monomeric subunits of TGF-β family protein to each other by methods known in the art, in the proper coding frame, and expressing the fusion protein by methods commonly known in the art. Alternatively, such a fusion protein may be made by protein synthetic techniques, e.g., by use of a peptide synthesizer.

Preparation of Mutant TGF-β Family Protein Subunits and Analogs

The production and use of the mutant TGF-β family protein, mutant TGF-β family protein heterodimers, TGF-β family protein analogs, single chain analogs, derivatives and fragments thereof of the invention are within the scope of the present invention. In specific embodiments, the mutant subunit or TGF-β family protein analog is a fusion protein either comprising, for example, but not limited to, a mutant TGF-β family protein subunit and another CKGF, in whole or in part, two mutant nerve growth subunits. In one embodiment, such a fusion protein is produced by recombinant expression of a nucleic acid encoding a mutant or wild type subunit joined in-frame to the coding sequence for another protein, such as but not limited to toxins, such as ricin or diphtheria toxin. Such a fusion protein can be made by ligating the appropriate nucleic acid sequences encoding the desired amino acid sequences to each other by methods known in the art, in the proper coding frame, and expressing the fusion protein by methods commonly known in the art. Alternatively, such a fusion protein may be made by protein synthetic techniques, e.g. by use of a peptide synthesizer. Chimeric genes comprising portions of mutant TGF-β family protein subunits fused to any heterologous protein-encoding sequences may be constructed. A specific embodiment relates to a single chain analog comprising a mutant TGF-β family protein subunit fused to another mutant TGF-β family protein subunit, preferably with a peptide linker between the two mutant.

Structure and Function Analysis of Mutant TGF-β Family Protein Subunits

Described herein are methods for determining the structure of mutant TGF-β family protein subunits, mutant heterodimers and TGF-β family protein analogs, and for analyzing the in vitro activities and in vivo biological functions of the foregoing.

Once a mutant TGF-β family protein subunit is identified, it may be isolated and purified by standard methods including chromatography (e.g., ion exchange, affinity, and sizing column chromatography), centrifugation, differential solubility, or by any other standard technique for the purification of protein. The functional properties may be evaluated using any suitable assay (including immunoassays as described infra).

Alternatively, once a mutant TGF-β family protein subunit produced by a recombinant host cell is identified, the amino acid sequence of the subunit(s) can be determined by standard techniques for protein sequencing, e.g., with an automated amino acid sequencer.

The mutant subunit sequence can be characterized by a hydrophilicity analysis (Hopp, T. and Woods, K., 1981, Proc. Natl. Acad. Sci. U.S.A. 78:3824). A hydrophilicity profile can be used to identify the hydrophobic and hydrophilic regions of the subunit and the corresponding regions of the gene sequence which encode such regions.

Secondary structural analysis (Chou, P. and Fasman, G., 1974, Biochemistry 13:222) can also be done, to identify regions of the subunit that assume specific secondary structures.

Other methods of structural analysis can also be employed. These include but are not limited to X-ray crystallography (Engstom, A., 1974, Biochem. Exp. Biol. 11:7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.). Structure prediction, analysis of crystallographic data, sequence alignment, as well as homology modelling, can also be accomplished using computer software programs available in the art, such as BLAST, CHARMM release 21.2 for the Convex, and QUANTA v.3.3, (Molecular Simulations, Inc., York, United Kingdom).

The functional activity of mutant TGF-β family protein subunits, mutant TGF-β family protein heterodimers, TGF-β family protein analogs, single chain analogs, derivatives and fragments thereof can be assayed by various methods known in the art.

For example, where one is assaying for the ability of a mutant subunit or mutant TGF-β family protein to bind or compete with wild-type TGF-β family protein or its subunits for binding to an antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. Antibody binding can be detected by detecting a label on the primary antibody. Alternatively, the primary antibody is detected by detecting binding of a secondary antibody or reagent to the primary antibody, particularly where the secondary antibody is labeled. Many means are known in the art for detecting binding in an immunoassay and are within the scope of the present invention.

The binding of mutant TGF-β family protein subunits, mutant TGF-β family protein heterodimers, TGF-β family protein analogs, single chain analogs, derivatives and fragments thereof, to the TGF-β family protein receptor can be determined by methods well-known in the art, such as but not limited to in vitro assays based on displacement from the TGF-β family protein receptor of a radiolabeled TGF-β family protein of another species, such as bovine TGF-β family protein. The bioactivity of mutant TGF-β family protein heterodimers, TGF-β family protein analogs, single chain analogs, derivatives and fragments thereof, can also be measured, by a variety of bioassays are known in the art to determine the functionality of mutant TGF-β protein. For example, the androgen metabolism bioassay described above can also be used to test mutant TGF-β proteins. Additional assays are described below.

TGF-β Radioreceptor Assay

TGF-β radioreceptor assays are performed to compare mutant TGF-β protein bioactivity to that of the wild type protein. The assays are performed using AKR-2B (clone 84A) cells as previously described by Taylor, et al., Biochim. Biophys. Acta, 442:324-330 (1976). Briefly, mutant and wild type TGF-β proteins are radiolabeled (specific activity, 2.3×108 cpm/μg) using a modified chloramine-T method described by Frolik et al., J. Biol. Chem., 259:10995-11000 (1984). Nonspecific binding is determined in the presence of 150-fold excess of unlabeled TGF-β wild type protein.

Soft Agar Assays

Soft agar assays are performed using concentrations of medium containing either mutant or wild type TGF-β proteins to stimulate soft agar colony growth of AKR-2B (clone 84A) cells to estimate the bioactivity of the mutant TGF family proteins as compared to the wild type form of the molecules. Colonies are allowed to grow for 2 weeks, and colonies greater than 50 μm diameter are quantitated on a Bausch and Lomb Omnicon (Rochester, N.Y.) colony counter. The nontransformed AKR-2B (clone 84A) cells are from a mouse fibroblast cell line of embryonic mesenchymal origin as described in Moses, et al., Cancer Res., 38:2807-2812 (1978). These cells are used as indicator cells in both soft agar and radioreceptor assays.

[³H]Thymidine Incorporation Assay

The thymidine incorporation assay is performed as previously described by Shipley, et al., Can Res., 44:710-716 (1984). This assay uses serum-starved, quiescent AKR-2B cells under various restimulation conditions. These conditions include the growth of the AKR-2B cells in the presence of [3H]thymidine and various wild type and mutant TGF-β proteins. Incorporation of the labeled bases is determined using standard techniques well known in the art and reflects DNA synthesis as a result of TGF-β stimulation.

Endothelial Cell Growth

Bovine pulmonary artery endothelial cells are grown in a basal medium of 1:1 mixture of Mediun 199 and Dulbecco's modified essential medium supplemented with 5% FBS (GIBCO), 5% Nu-serum (Collaborative Research, Inc., Lexington, Mass.), 1% L-glutamine, 100 units/ml penicillin, and 100 μg/ml streptomycin using methods previously described by Ryan et al., Tissue Cell, 10:535-554 (1984) and Meyrick et al., J. Cell. Physiol., 138:165-174 (1988). The cells are verified as being endothelial cells by their morphology, the presence of angiotensin-converting enzyme activity, binding of acetylated low-density lipoprotein, and the presence of factor VIII-associated antigen. Cells between passages 5 and 20 are used in the assay.

Endothelial cells are removed with gentle trypsinization and seeded into 24-well plates at a density of 5,000-10,000 cells/well in medium 199 containing 10% FBS. After 24 hours, medium was removed and experimental media is added to the cells. The experimental media contains wild type and mutant TGF-β proteins in various concentrations. Cells are counted with a Courter counter after trypsinization of cells from the wells. Cell number is determined prior to the addition of the experimental media and at 2- and 3-day intervals. The number of cells is compared between wild type and mutant TGF-β stimulated samples.

Neurturin Bioassay Systems

Neurturin is known to promote the formation of ganglia and interconnected neuronal and glial processes. The assays described below exploit this and other bioactivities of wild type Neurturin to analyze the bioactivity of mutant neurturin proteins described by the present invention. This assay also has utility in analyzing the bioactivity of glial derived neurotrophic factor (GDNF) mutants.

In one embodiment, the assay for neurturin bioactivity consists of treating primary cultures of cells with wild type neurturin or mutant neurturin proteins of the present invention and determining the effect these proteins have on cell growth. Primary cultures are prepared according to the method of Heuckeroth, et al., Dev. Biol., 200:116-129 (1998). Briefly, embryos are obtained from pregnant Spraque-Dawley rats and embryonic gut samples including the small and large bowel, but excluding stomach and pancreas, are dissected from the embryos. The gut samples are then digested with dispase (1 mg/ml) and collagenase (1 mg/ml). Single cell suspensions are obtained by trituration with a polished glass pipet. Incubation of the triturated cells for 10 minutes at 37° C. followed by gentle mixing allows dead cells to break open and aggregate. Cell suspensions are filtered through nylon mesh, and trypan blue-excluding cells are counted on a hemocytometer. Cells are then grown in a modified N2 medium containing 50% DME, 50% F12, bovine insulin (5 μg/ml), rat transferin (10 μg/ml), 20 nM progesterone, sodium selenite (Na2SeO3, 30 nM), putrescine dihydrochloride (100 μM), bovine serum albumin fraction V (1 mg/ml) and fetuin (0.1 mg/ml). Cultures are grown on 8-well chamber slides coated with poly-D-lysine (0.1 mg/ml) and then with mouse laminin (20 μg/ml). The slides are then washed with L15 medium with 10% fetal bovine serum and allowed to dry. Typically 10,000 trypan-excluding cells are plated into single wells (1 cm²) of an 8-well chamber slide. Care is taken to ensure uniform distribution of cells. For Brdu/Ret double labeling studies, 30,000 trypan blue-excluding cells are plated per well to increase the number of Ret-expressing cells in the untreated and persephin-treated cultures to at least 100 per well. After allowing cells to adhere to the slide for 30 minutes, 200 μl medium is added with the wild type or mutant neurturin proteins. Cells are grown in a humidified tissue culture incubator containing 5% CO₂ at 37° C. Medium is changed every 2-3 days by withdrawing half of the medium and adding new medium.

Cell counts are obtained manually on DAB-stained slides using a counting grid and a 20× objective. Slides were numerically coded so that the individual counting cells was not aware of the treatment conditions. All of the immunostained cells in an individual well are counted. To determine the percentage of Ret-positive cells per well, all Ret-expressing and total cells are counted in individual wells of an 8-well chamber slide.

Bromodeoxyuridine/Ret Double Immunofluorescence

Cells from rat gut are plated onto 8-well chamber slides as described above. Bromodeoxyuridine (10 μmol/L final concentration) are added to cells in culture at 3, 24, 48 or 72 hours or 5 days after plating. After 26 hours, exposure to bromodeoxyuridine, cultures are washed three times with PBS and fixed (70% ethanol.30% 50 mM glycine, pH 2, for 20 minutes at −20° C.). Ret immunofluorescent signal is detected by incubation with Ret antibody overnight at 4° C., followed by a biotin-conjugated goat anti-rabbit secondary antibody and amplification of signal with a TSA indirect kit per manufacture's instructions. Bromodeoxyurdine (Brdu) incorporation is detected on the same slides with a mouse anti-bromodeoxyuridine primary and goat anti-mouse Cy3 secondary antibody. To determine Brdu incorporation in to c-Ret expressing cells, Ret was detected as fluorescein isothiocyanate (FITC) signal. For each Ret-expressing cell, Cy3 staining in the nucleus is determined to calculate the percentage of Ret+ cells that have incorporated Brdu during the 26 hour labeling period. One hundred cells per well are examined.

Bromodeoxyuridine/GFAP Double Immunofluorescence

Cells from cultures are grown for 5 days in 8-well chamber slides either with or without added factors or with 100 ng/ml of GDNF, neurturin, or persephin. Medium was changed after 48-72 hours by removing half of the medium and adding fresh medium. On the fifth day of culture, Brdu (10 μmol/L final concentration) is added and culture is continued for an additional 26 hours before fixation (70% ethanol/30% 50 mM glycine, pH 2, 20 min, −20° C.). GFAP staining is detected after amplification using a TSA indirect kit per manufacturer's instructions. Streptavidin-FITC is used to detect the biotin deposited on GFAP-expressing cells. Brdu incorporation is detected above with a Cy3-conjugated secondary antibody. Cells are first examined for GFAP expression under the fluorescent microscope. Brdu incorporation into GFAP-expressing cells is determined for 800 cells total for each condition (100 cells per well, 8 wells, 2 separate experiments.)

Bisbenzimide/Ret Double Staining and Guantitation of Condensed Nuclei

Enteric neuron cultures are grown for 72 hours as described above in the presence or absence of 100 ng/mL GDNF. Cells are then fixed with 4% paraformaldehyde in PBS for 30 minutes at 25° C. Slides are incubated with Ret antibody followed by Cy3-conjugated secondary antibody as described above. After being washed with PBS, slides are incubated with 1 μg/ml of 2′-(4-hydroxyophenyl)-5-(4-methyl-1-piperazinyl)-2,5′-bi-1H-bisbenzimidazole trihydrochloride pentahydrate (bisbenzimide, Hoecht 33258; Molecular Probes, Eugene Oreg.) in PBS for 1 hour at 25° C. Slides are washed with PBS, mounted, and examined for Cy3 fluorescence to identify Ret-expressing cells and with ultra-violet illumination to see bisbenzimide staining of the nucleus. Ret-expressing cells in 130 randomly selected high-power fields (24 separate culture wells, 3 separate experiments) with and without GDNF are examined for nuclear condensation characteristic of dying cells. Examples of each of these assays are found in Heuckeroth, et al., Dev. Biol., 200:116-129 (1998).

Inhibins and Activins

The TGF-β family encompasses the inhibin family (e.g., inhibin A and inhibin B) and activin family (e.g., activin A, activin B, activin AB, and activin BB) of proteins. Human scrotal skin fibroblasts in primary culture have been used to measure the bioactivity of TGF-β proteins that are potent inducers of 5α-reductase (5αR). This system can also be used to measure the bioactivity of the inhibins and activins, as these protein are also 5αR inducers.

To perform the assay, human scrotal skin is obtained from healthy male individuals undergoing bilateral vasectomy. The biopsy specimens of human scrotal skin are cleaned from subcutaneous fat and minced to approximately 1 mm cubes and spread on 100 mm Falcon dishes. RPMI 1640 medium containing 10% fetal bovine serum (FBS) and 100 units/ml penicillin and 100 μg/ml streptomycin buffered with NaHCO₃ and 25 mM HEPES are added to each dish and incubated at 37° C. in the presence of 5% CO₂ in a humidified atmosphere in a Stericult 200 Forma Scientific incubator (Marietta, Ohio). When cells reach confluence, they are sub-cultured after trypsin dissociation. these cells are plated in 6-well dishes and used between 3 and 7 passages for the assay of 5α-reductase activity.

Prior to the assay, cells (200,000 cells/well) are made quiescent by serum starvation for 48 hours in RPMI-1640 medium containing 0.2% BSA. Cells are then treated with the wild type or mutant inhibins or activins and DHT in serum depleted RPMI media with 0.2% BSA for 2 days. After 48 hours, the medium is removed and the cells are again incubated with serum free medium containing [3H]testosterone (200,000 cpm, 4.8 pmol) at 37° C. in a 5% CO2 incubator for 4 hours. At the end of incubation, the cells are rapidly cooled on ice and the medium is transferred into ice cold tubes containing diethyl ether and 14 C standards to monitor recovery. Each well is rinsed with 1 ml phosphate buffered saline (PBS), and the rinse is added to the medium for extraction. The separation of [³H]DHT is achieved by celite and paper chromatography. Results are expressed at % conversion in 4 hours/2×10⁵ cells. Cell number in each well is determined by counting an aliquot in a hemocytometer before and after the 2 day treatment period with the test substances.

3α-reductase activity is also measure of inhibin and activin bioactivity. 3α-reductase enzyme activity is measured in the same manner as 5αR activity except that [³H]DHT is added (200,000 cpm) with the 14 C standards. [³H]DHT and [³H]androstane-3,17-diol (3α-diol) are purified by celite and paper chromatography.

Cells (10⁵) are incubated in serum-free RPMI medium with 0.2% BSA for 48 hours. They are then treated with mutant or wild type activins or inhibins at 2.4×10-9 M for 48 hours as described above, followed by incubation with [³H]thymidine (1 μCi/well). Six hours later cells are washed twice with 1 ml PBS, twice with 10% ice cold trichloroacetic acid solution, followed by a wash with PBS. The cells are then solubilized with 1% sodium dodecyl sulfate in 0.3N NaOH. An aliquot is then counted in a scintillation counter. The levels of reductase activity generated for wild type and mutant proteins are determined and compared to assess the bioactivity of the mutant proteins. Examples of this assay system are found in Antonipillai, et al., Mole. Cell. Endo., 107:99-104 (1995).

Mullerian Inhibiting Substance: MIS

Mullerian inhibiting substance (MIS) is the gonadal hormone that causes recession of the Mullerian ducts, the anlagen of the female internal reproductive structures, during male embryogenesis. MIS is a member of the TGF-β family of proteins that are involved in the regulation of growth and differentiation.

MIS Organ Culture Assay System

An organ culture assay system has been developed to establish a graded bioassay in which 14.5 day female rat embryonic urogenital ridges are incubated with the mutant MIS proteins to be tested. To facilitate morphological comparison, testosterone is added to the media at 10-9M to enhance the effect of MIS and stimulate growth of the Wolffian duct. After 72 hours of incubation in humidified 5% CO2, the specimen is sectioned and stained with hematoxylin and eosin. Regression of the Mullerian duct is graded from 0 (no regression) to 5 (complete regression) by at least two independent observers. The organ culture bioassay requires 1.5-2 μg/ml of recombinant holoMIS for full ductal regression. The amount of ductal regression is compared between wild type MIS and mutant MIS proteins disclosed in the present invention. An example of this assay is described in Donahoe, et al., J. Surg. Res., 23:141-148 (1977).

MIS Granulosa-Luteal Cell Proliferation Inhibition Assay

Granulosa-luteal cells have been used to measure the inhibitory effect of MIS exposure. In this assay, granulosa-luteal cells are harvested transvaginally from preovulatory follicles of women under the age of 40 with tubal factor infertility undergoing ovum retrieval for in vitro fertilization/embryo transfer. Follicular development is initiated with clomiphene citrate (50-100 mg/day) beginning days 3 to 5 of the follicular phase for a total of 5 days. On treatment day 5, 150 or 225 IU of human menopausal gonadotropin is administered intramuscularly daily until 3 or more follicles greater than or equal to 20 mm in diameter are seen, and serum estradiol levels reached 200 pg per follicle. Human chorionic gonadotropin (hCG) 5,000 IU was given to each patient 34 hours before oocyte retrieval.

Oocytes are identified visually and isolated for insemination and culture. The remaining follicular contents are centrifuged at 600×g at room temperature for 10 minutes, and the supernatant discarded. The granulosa-luteal cells in the pellets are combined, washed twice in 2 ml Ham's F-10 (GIBCO, Grand Island, N.Y.) in 10% female fetal calf serum (FFCS, Metrix Co., Dubuque, N.Y.) determined to be MIS-free by bioassay and immunoassay, and dispersed with gentle shaling in 2 ml of Ham's F-10 containing 0.1% collagenase/dispase (Boehringer Mannheim GmbH, Germany) for 30 minutes at 37° C. in 5% CO₂. After centrifugation at 600×g for 10 minutes and resuspension in 1 ml of culture medium, cells are layered over 5 ml 50% percoll (Sigma Chemical Co., St. Louis, Mo.) and centrifuged at 300×g for 30 minutes to remove erythrocytes. The purified granulosa-luteal cells are aspirated from the interface, washed once, resuspended and counted in a hemocytometer. Cell viability should be greater than 95% as determined by the exclusion of trypan blue (0.4%).

Approximately 30,000 viable granulosa-luteal cells are plated per well in triplicate in 24 multiwell dishes with 1 ml culture medium consisting of Ham's F-10 with 10% MIS-free FFCS, 2 mmol L-glutamine (Sigma), 2.5 μg/ml Fungizone (GIBCO), and 100 IU/ml penicillin and 100 μg/ml streptomycin sulfate (Sigma). Cells were cultured at 37° C. in 95% air and 5% CO₂ environment.

Before initiating the assays, granulosa-luteal cells are incubated at 37° C. for 4 days in Ham's F-10 enriched with 10% MIS-free FFCS, with media changes every 48 hours to minimize the effect of hCG given to patients 34 hours before oocyte retrieval. Thereafter, control or test compound containing media are added to the cells. The test compounds are the mutant and wild type MIS proteins that are diluted in Ham's F-10 with 10% MIS-free FFCS culture to a final concentration of 0.2, 2, or 20 ng/ml. The growth modulator EGF is also diluted in Ham's F-10 with 10% MIS-free FFCS culture to a fmal concentration of 0.2, 2, or 20 ng/ml. EGF at 20 ng/ml is mixed with the wild type and mutant MIS proteins at 0.2, 2, or 20 ng/ml just prior to addition to the incubation. The cells are divided into three subgroups, one for each concentration of hormone. The control media is the diluent without MIS added.

Two pools of cells from two or three subjects are used in the assays. Three subgroups consisting of 12 wells each were cultured in 0.2, 2, and 20 ng/ml of MIS containing media with or without EGF at the beginning of culture day 4. Media were changed every 48 hours with the spent media saved for analysis. Three wells from each of the groups are used for either cell counts or DNA contents on days 4, 8, 12, and 16 of culture. In addition, a number of 12-well subgroups determined by the number of mutant MIS proteins being tested are cultured in EGF 20 ng/ml plus the mutant MIS protein at 0.2, 2, or 20 ng/ml beginning on culture day 4.

The amount of growth in a particular well is determnined by DNA assay of the cells. DNA content is determined fluormetrically using the Hoechst 33258 dye (Sigma). Cells harvested in assay buffer (2.0 mol NaCl, 0.05 mol Na2HPO4, and 2 mmol ethylenediaminetetraacetate are transferred into disposable culture tubes (10×75 mm, VWR, San Francisco, Calif.). DNA standards are prepared from 1) calf thymus DNA in DPBS with 2 mol ethylenediaminetetraacetate and 2) known concentrations of human spermatozoa. The DNA stock solution is diluted in assay buffer and 0 to 2500 ng were aliquoted into microcentrifuge tubes and handled in a similar manner as cells to generate a standard curve of DNA (ng) vs. cell number (spermatozoa standards) for each assay. One ml dye (100 ng/ml, in assay buffer) was added to each tube and cells are incubated in the dark at room temperature for 2 hours. Fluorescence is measured on a fluorometer (model A-4, Farrand Optical, New York, N.Y.) with an excitation maximum at 360 nm and an emission maximum at 492 nm. The assay should be linear over the range of 10-1000 ng (˜10³-10⁵ cells). An example of this as is found in Kim et al., J. Clin. Endocrinol. Metab., 75:911-917 (1992).

BMP

The bone morphogenetic protein (BMP) family is a member of the TGF-β superfamily of proteins. Members of the BMP family have been implicated in several aspects of neural crest progenitor differentiation, including neuronal lineage commitment and the acquisition of the adrenergic phenotype. The present invention contemplates numerous mutations to the various BMP family members to alter their bioactivity as compared to the wild type forms of the family members.

A number of bioassays are known that permit one of ordinary skill in the art to determine which mutations to the various BMP family proteins result in an enhanced bio activity. One such assay system measures the differentiation of astroglial progenitor cells (O-2As) into astrocytes in response to BMP stimulation. O-2A progenitor cells undergo progressive oligodendroglial differentiation when cultured in serum-free medium (as measured by the appearance of galactocerebroside in immunochemical assays), but differentiate into astrocytes in medium containing BMPs (as measured by the appearance of the cellular maker glial fibrillary acidic protein (GFAP)). Accordingly, in one embodiment of the present invention, the appearance of cellular makers that indicate the phenotype of the progenitor cell line O-2A are measured to compare the bioactivity of mutant and wild type BMP proteins of O-2A cell differentiation.

To make this comparison, culture of O-2A cells are obtained from rats postnatal day 2 (P2) cortex samples. Cortex samples are dissected and dissociated mechanically by repeated trituration in DMEM/F12 1:1 supplemented with 10% FBS, glucose (6 mg/ml), and glutamine (2 mM), and then filtered through a 60 μM Nytex filter. Cells are then pelleted, resuspended, and plated onto poly-D-lysine (PDL, 20 μg/ml for 1 hour)-coated T75 flasks at 1.5 brains per flask. Cultures are fed twice per week, and ˜1 days after reaching confluence (total of 9-10 days in vitro), flasks are shaken for three hours at 250 rpm to remove microglia, refed, and then shaken overnight at 300 rpm to remove O-2As. Collected O-2As are finther purified by passing through a 60 μM Nytex filter and preplating on uncoated plastic dishes for 2 hours to remoOve contaminating microglia. Cells are then pelleted, resuspended in serum-free medium (SFM), counted and plated at ˜104 cells per well in PDL-coated 24-well plates. SDM consisted of DMEM/F12 (1:1) with glucose (6 ng/ml), glutamine (2 mM), BSA (0.1 mg/ml), transferrin (50 μg/ml), triiodothyronine (30 nM), hydrocortisone (20 nM), progesterone (20 nM), biotin (10 nM), selenium (30 nM), and insulin (5 μg/ml). For the forty-eight hours before experimental manipulation, bFGF (2.5 ng/ml) and PDGF AA (2.5 ng/ml) are added. Cells are maintained in a humidified incubator with 5% CO₂ at 37° C. Control cultures are fed every 2 days, and BMP-treated cultures received fresh medium and growth factors every 4 days. O-2A cultures analyzed at the beginning of the assay should contain at least 95% cells immunoreactive the O-2A-associated antibodies GD3 (J. Goldman, Columbia University) and A2B5 and O4 (S. Pfeiffer, University of Connecticut). The anti-galactocerebroside (GC) antibody GC/01 is also made by S. Pfeiffer, University of Connecticut. See Raff et al., Science, 243:1450-1455 (1989) and Levison and Goldman, Neuron, 10:201-212 (1993), for discussions of these antibodies.

The presence or absence of particular cellular markers is determined using standard immunochemical techniques. For example, at designated times, SFM is withdrawn and cells are fixed with ice-cold absolute methanol for 10 minutes. For the anti-O-2A or GC antibodies, cells are incubated with antibodies for 30 minutes at 4° C., followed by washing and fixing. After treatments with 0.3% H2O2 for 20 minutes and blocking serum (5% goat serum) for 30 minutes, primary antibodies to cellular antigens are applied for 2 hours at room temperature. Appropriate biotinylated N secondary antibodies (Vector Laboratories, Burlingame, Calif.) are applied at 1:200 dilution for 30 minutes, followed by application of the ABC reagent (Vector) for 1 hour. The peroxidase reaction is performed with visualization of label using diaminobenzidine 0.5 mg/ml as substrate in 50 mM Tris, pH 7.6, containing 0.01% H₂O₂ for 5 minutes. All steps are followed by washes in PBS, pH 7.4, except the blocking serum step.

Cell counts per well are calculated by counting representative fields of view making up one quarter of the total culture well area and multiplying by 4. The number of GFAP-immunoreactive cells per well that result from wild type or mutant BMP stimulation are compared to determine the mutant proteins bioactivity relative to the wild type protein. An example of this assay is found in Mabie, et al., J. Neurosci., 17(11): 4112-4120 (1997).

In another embodiment, humane bone marrow osteoprogenitor cells are treated with BMP wild type and mutant protein to stimulate differentiation. This treatment also inhibits DNA synthesis of the treated osteoprogenitor cells. BMP proteins effect on osteoprogenitor cells is determined by measuring cell growth as reflected by DNA synthesis, and cell differentiation by measuring alkaline phosphatase activity and the synthesis of osteocalcin, osteonectin and type I collagen response to 1, 25 (OH)₂D₃ human parathyroid hormone.

To analyze the effects of various wild type and mutant BMP proteins, human bone marrow is obtained by iliac aspiration from normal donors (aged 20-30 years) undergoing hip prosthesis surgery after trauma. Cells are separated into a single suspension by sequential passage through syringes fitted with a 16-, 18- and 21-guage needle. Cells are then counted and plated into 35-mm dishes in BGJb medium (GIBCO, Grand Island, N.Y.) supplemented with 10% (v/v) FCS, at 105 cells/cm2 and incubated in a humidified atmosphere of 95% (v/v) air and 5% (v/v) CO² at 37° C. The initial medium change is performed 3 days later and thereafter the medium is changed every 2 days. Confluence is obtained 3 weeks later, and cells are cloned by limiting dilution followed by successive subculturing, performed until the highest intracellular alkaline phosphatase activity is reached.

At confluence, the medium is replaced with fresh BGJb medium containing 0.2% (e/v) BSA for 24 hours. Thereafter, wild type and mutant BMP dilutions (1, 2.5, and 10 ng/ml) are added to each well. Controls are assessed using 5 mM HCl and 0.2% (w/v) BSA. Cells are treated for three days as described above.

The effect of the BMP proteins of cell proliferation is determined by examining DNA synthesis and cellular proliferation. DNA synthesis is determined by incorporation of [³H]-thymidine according to the method of Hauscka, et al., J. Biol. Chem., 261:12665-12674 (1986). Briefly, human bone marrow derived cells are grown to confluence (104 cells/cm2) in 96-well culture plates. Cells are deprived of FCS for 24 hours and then treated with the various BMP solutions. At 24 hours before the end of the incubation period, cells are incubated with [³H]-thymidine (5 μCi/ml) in medium containing 0.2% (w/v) BSA. Material precipitable with trichloroacetic acid is solubilized in 0.2 ml 0.3 N NaOH, and the radioactivity of the material is determined in a liquid scintillation counter. Proliferation analysis is performed by plating bone marrow stromal cells at 5×10³ cells/cm² with 2.5 ng/ml of either a wild type or mutant BMP protein containing solution. Cell number per well is calculated at different times (days 1, 2, 3, and 6) and the numbers of cells in the wild type BMP containing wells are compared to the cells contained in the mutant BMP containing wells to determine the bioactivity of those mutant proteins.

Cellular differentiation induced by the various BMP solutions is measured by alkaline phosphatase activity, osteocalcin synthesis, and osteonectin synthesis. To measure alkaline phosphatase activity, cells are scraped and sonicated as described in Majeska, et al., J. Biol. Chem., 257:866-872 (1989). The effect of BMP exposure on osteocalcin synthesis is measured by a specific radioimmunoassay with an antibody raised in rabbit against bovine osteocalcin. The detection limit for the assay is 1 ng/ml. Following exposure to the BMP solutions being tested, at the concentrations of 2.5 and 10 ng/ml, and 1,25 (OH)₂D₃ at 10-8 M for 3 days, the medium is removed, and the cell layer is scraped in PBS. Cells are then sonicated and proteins are precipitated with 50% (v/v) aminonium sulfate. Osteocalcin in the cell layer and secreted in the culture medium is then determined by radioimmunoassay. The concentration of osteocalcin is determined for the wild type BMP containing wells and for the mutant BMP containing wells to determine the bioactivity of the mutant proteins.

Osteonectin synthesis induced by BMP stimulation is measured by plating cells at 104 cells/cm2 in chamber slides and growing them for 8 days. At confluence, cells are treated for 3 days with 2.5 and 10 ng/ml of the various BMP solutions being tested for bioactivity. Controls are performed using cells treated for 3 days with the same amount of buffer that is used to solubilize the BMP proteins. Thereafter, medium is collected, the cell layer is fixed using 100% methanol for 10 minutes at 4° C., and incubated overnight at 26° C. with a polyclonal antibody specific to bovine osteonectin diluted at 1/200 in 0.1 M PBS pH 7.4. Fixed immunoglobulins are revealed using [125I]-protein A (1 μCi/μg) diluted at 105 cpm/well. After extensive washings, the radioactivity in ten wells is determined in a γ counter. The concentration of osteonectin is determined for the wild type BMP containing wells and for the mutant BMP containing wells to determine the bioactivity of the mutant proteins. An example of this assay is found in Amédée, et al., Differentiation, 58:157-164 (1994).

In another embodiment, the effects of BMP application of cellular growth are used to determine the bioactivity of BMP mutants described by the present invention as compared to their wild type counterparts. To compare the bioactivity of wild type and mutant proteins, wounds through the alveolar bone and periodontal ligament are made in male Wistar rates. Defects are filled with either a collagen implant or collagen plus a BMP protein, either wild type or mutant, or were left unfilled (controls). Three animals per time period are killed on days 2, 5, 10, 21 and 60 after surgery for each wound type. Cellular proliferation and clonal growth in periodontal tissues are assessed by [3H]-thymidine labeling one hour before death, followed by radioautography. Cellular differentiation of soft and mineralizing connective tissue cell populations is determined by immunohistochemical staining of α-smooth muscle actin, osteopontin and bone sialoprotein, all techniques well known in the art. Wild type BMP-7 is known to induce abundant bone formation by 21 days and so the amount of bone growth generated by a mutant BMP-7 protein would be compared to the wild type levels of bone growth to determine if the mutant protein possessed enhanced bioactivity. Cellular proliferation and α-smooth muscle actin staining patterns are also evaluated to determine the bioactivity of a mutant BMP protein. An example of this assay is described in Rajsjankar, et al., Cell Tissue Res., 294:475-483 (1998).

In another embodiment, BMP-9 binding to liver cells is used to compare the bioactivity of wild type and mutant BMP-9 proteins. To examine BMP-9 bind, HepG2 cells are grown to confluence in Dulbecco's modified Eagle's medium (DMEM) containing 10% heat-inactivated FCS on gelatinized 6-well plates. The cells are incubated with 2 ng/ml [¹²⁵I] labeled wild type or mutant BMP-9 and increasing concentrations of unlabeled wild type BMP-9 in binding buffer (136.9 mM NaCl, 5.37 mM KCl, 1.26 mM CaCl₂, 0.64 mM MgSO₄, 0.34 mM Na₂HPO₄, 0.44 mM KH₂PO₄, 0.49 mM MgCl₂, 25 mM HEPES, and 0.5% BSA, pH 7.4) for 20 hours at 4° C. following a one hour preincubation at 37° C. in binding buffer alone. Cells are washed twice in ice-cold binding buffer and bound BMP-9 is extracted and quantified. The amounts of wild type and mutant BMP-9 are compared.

Cellular proliferation induced by exposure to wild type and mutant BMP-9 proteins is determined by plating HepG2 cells at 105 cells per well in a 96-well plates and culturing the plates for 48 hours in DMEM/0.1% FCS to synchronize the cell cycle. The confluent cells are then treated for 24 hours with or without mutant or wild type BMP-9 in the presence of 0.1% FCS. For [³H]-thymidine incorporation assays, [³H] -thymnidine is included in the last 4 hours of the treatment period, and cellular DNA is collected with a 96-well plate cell harvester. Incorporation of [³H]-thymidine is measured by liquid scintillation counting. For cell counting assays, cells are trypsinized and counted using a hemacytometer.

Primary rat hepatocytes are plated on collagen-coated plates at subconfluence (5000-10000 cells/cm²) in serum-free media and treated with the wild type or a mutant BMP-9 for 36 hours. [³H]-thymidine is included throughout the treatment period, and incorporated [³H]-thymidine is quantified using techniques well known in the art. An example of this assay is found in Song, et al., Endocrinology, 136:4293-4297 (1995).

GDF Mediated Inhibition of Epithelial Cell Proliferation

One assay to test the bioactivity of the GDF family of proteins is the cell clonal growth proliferation assay. In these assays, cell growth, proliferation, and mRNA production is measured in response to GDF stimulation. In this assay, the ability of mutant GDF proteins are to stimulate cell activity is measured and compared to the ability of the corresponding wild type GDF protein to stimulate the test cells. One of skill in the art would be able to use this assay to determine which mutations in the GDF family of proteins results in enhanced or decreased bioactivity as compared to the wild type protein. An example of such an assay is found at You, L., et al., Invest. Ophthalmol. Vis. Sci., 40(2):296-311 (1999).

The half life of a protein is a measurement of protein stability and indicates the time necessary for a one-half reduction in the concentration of the protein. The half life of a mutant TGF-β family protein can be determined by any method for measuring TGF-β family protein levels in samples from a subject over a period of time, for example but not limited to, immunoassays using anti-TGF-β family protein antibodies to measure the mutant TGF-β family protein levels in samples taken over a period of time after administration of the mutant TGF-β family protein or detection of radiolabelled mutant TGF-β family protein in samples taken from a subject after administration of the radiolabelled mutant TGF-β family protein.

Other methods will be known to the skilled artisan and are within the scope of the invention.

Diagnostic and Therapeutic Uses

The invention provides for treatment or prevention of various diseases and disorders by administration of therapeutic compound (termed herein “Therapeutic”) of the invention. Such Therapeutics include TGF-β family protein heterodimers having a mutant α subunit and either a mutant or wild type β subunit; TGF-β family protein heterodimers having a mutant α subunit and a mutant β subunit and covalently bound to another CKGF protein, in whole or in part, such as the CTEP of the β subunit of hLH; TGF-β family protein heterodimers having a mutant α subunit and a mutant β subunit, where the mutant α subunit and the mutant β subunit are covalently bound to form a single chain analog, including a TGF-β family protein heterodimer where the mutant α subunit and the mutant β subunit and the CKGF protein or fragment are covalently bound in a single chain analog, other derivatives, analogs and fragments thereof (e.g. as described hereinabove) and nucleic acids encoding the mutant TGF-β family protein heterodimers of the invention, and derivatives, analogs, and fragments thereof.

The subject to which the Therapeutic is administered is preferably an animal, including but not limited to animals such as cows, pigs, horses, chickens, cats, dogs, etc., and is preferably a mamnmal. In a preferred embodiment, the subject is a human. Generally, administration of products of a species origin that is the same species as that of the subject is preferred. Thus, in a preferred embodiment, a human mutant and/or modified TGF-β family protein heterodimer, derivative or analog, or nucleic acid, is therapeutically or prophylactically or diagnostically administered to a human patient.

In a preferred aspect, the Therapeutic of the invention is substantially purified.

In specific embodiments, mutant PDGF family protein heterodimers or PDGF family protein analogs with bioactivity are administered therapeutically, including prophylactically to treat a number of cellular growth and development conditions, including promoting wound healing. For example, mutant TGF-β proteins of the present invention will inhibit proliferation of epithelial cells and tumor cells.

The absence of or a decrease in PDGF family protein or function, or PDGF family protein receptor and function can be readily detected, e.g., by obtaining a patient tissue sample (e.g., from biopsy tissue) and assaying it in vitro for RNA or protein levels, structure and/or activity of the expressed RNA or protein of PDGF family protein or PDGF family protein receptor. Many methods standard in the art can be thus employed, including but not limited to immunoassays to detect and/or visualize PDGF family protein or PDGF family protein receptor protein (e.g., Western blot, immunoprecipitation followed by sodium dodecyl sulfate polyacrylamide gel electrophoresis, immunocytochemistry, etc.) and/or hybridization assays to detect PDGF family protein or PDGF family protein receptor expression by detecting and/or visualizing PDGF family protein or PDGF family protein receptor mRNA (e.g., Northern assays, dot blots, in situ hybridization, etc.), etc.

A number of disorders which manifest as infertility or sexual disfunction can be treated by the methods of the invention. Disorders in which TGF-β family protein is absent or decreased relative to normal or desired levels are treated or prevented by administration of a mutant TGF-β family protein heterodimer or TGF-β family protein analog of the invention. Disorders in which TGF-β family protein receptor is absent or decreased relative to normal levels or unresponsive or less responsive than normal TGF-β family protein receptor to wild type TGF-β family protein, can also be treated by administration of a mutant TGF-β family protein heterodimer or TGF-β family protein analog. Mutant TGF-β family protein heterodimers and TGF-β family protein analogs for use as antagonists are contemplated by the present invention.

In specific embodiments, mutant TGF-β family protein heterodimers or TGF-β family protein analogs with bioactivity are administered therapeutically, including prophylactically to treat ovulatory dysfunction, luteal phase defect, unexplained infertility, time-limited conception, and in assisted reproduction.

The absence of or a decrease in TGF-β family protein protein or function, or TGF-β family protein receptor protein and function can be readily detected, e.g., by obtaining a patient tissue sample (e.g., from biopsy tissue) and assaying it in vitro for RNA or protein levels, structure and/or activity of the expressed RNA or protein of TGF-β family protein or TGF-β family protein receptor. Many methods standard in the art can be thus employed, including but not limited to immunoassays to detect and/or visualize TGF-β family protein or TGF-β family protein receptor protein (e.g., Western blot, immunoprecipitation followed by sodium dodecyl sulfate polyacrylamide gel electrophoresis, immunocytochemistry, etc.) and/or hybridization assays to detect TGF-β family protein or TGF-β family protein receptor expression by detecting and/or visualizing TGF-β family protein or TGF-β family protein receptor mRNA (e.g., Northern assays, dot blots, in situ hybridization, etc.), etc.

Experiments

The following Experiments demonstrate that mutations introduced into different CKGF subunits advantageously produced hormones having elevated bioactivity. For purposes of illustration, the glycoprotein common α-subunit and the β-subunits specific for TSH and hCG have been mutagenized, expressed as mutant heterodimers and these mutant heterodimers tested in biological assays. In the context of the invention it is to be understood that a mutagenized protein differs in polypeptide sequence from the wild type counterpart protein. Below there is provided a description of the materials and methods used to conduct the procedures that confirmed CKGF mutants exhibited modified biological activities.

Materials

Restriction enzymes, DNA markers and other molecular biological reagents were purchased from either Gibco BRL (Gaithersburg, Md.) or from Boehringer-Mannheim (Indianapolis, Ind.). Cell culture media, fetal bovine serum and LIPOFECTAMINE reagents were purchased from New England Biolabs (Beverly, Mass.). The full-length human α cDNA (840 bp) subdloned into BamHI/XhoI sites of the pcDNA I/Neo vector (Invitrogen, San Diego, Calif.) and hCG-β gene were obtained from T. H. Ji (University of Wyoming, Laramie, Wyo.). The α cDNA sequence encoded the wild type protein sequence shown as SEQ ID NO:1. The hCG-β polynucleotide encoded the wild type protein sequence shown as SEQ ID NO:4. The hTSH-β minigene without the first intron but including the nontranslated first exon and authentic translation initiation site was constructed by the inventors and encoded the protein identified by SEQ ID NO:2. Recombinant human TSH employed as a hormone standard was from Genzyme (Framingham, Mass.). Chinese Hamster Ovary (CHO) cells stably expressing the human TSH receptor (CHO-hTSHR clone JP09 and clone JP26) were provided by G. Vassart (University of Brussels, Brussels, Belgium). ¹²⁵I cAMP, ¹²⁵I-human TSH, and ¹²⁵I-bovine TSH radiolabelled to a specific activity of 40-60 μCi/μg were obtained from Hazleton Biologicals (Vienna, Va.).

Methods

Site-Directed Mutagenesis

Site-directed mutagenesis of the human α-subunit cDNA, the human TSH minigene and the hCG-β subunit cDNA was carried out using the PCR-based megaprimer method described by Sarkar et al., in BioTechniques 8:404 (1990). Polynucleotide amplification was optimized using VENT DNA polymerase (New England Biolabs). Amplification products were digested with BamHI and XhoI and then ligated into the pcDNA I/Neo vector (Invitrogen) from which the BamHI/HhoI fragment had been excised. MC1061/p3 E. coli host cells were transformed using an ULTRACOMP E. coli Transformation Kit (Invitrogen). The QIAPREP 8 plasmid kit (Qiagen) was used for multiple plasmid DNA preparations. Qiagen Mega and Maxi Purification Protocols were used to purify larger quantities of plasmids containing the mutant subunit with single or multiple mutations as a template for further mutagenesis. Construction of the mutant TSH-β subunit fusion with the CTEP is described by Joshi et al., in Endocrinology 136:3839 (1995). Successful mutagenesis was confirmed by double-stranded DNA sequencing using a standars dideoxynucleotide chain termination protocol.

Expression of Recombinant Hormones

CHO-K1 Cells (ATCC, Rockville, Md.) were maintained in Ham's F-12 medium containing glutamine, 10% FBS, penicillin (50 units/ml) and streptomycin (50 μg/ml). Plates of cells (100-mm culture dishes) were cotransfected with wild type or mutant α-subunit cDNA in the pcDNA I/Neo vector and mutant hTSH-β minigene ligated into the p(LB)CMV vector, or the pcDNA I/Neo vector containing the hCG-β cDNA insert, using LIPOFECTAMINE (Gibco BRL) according to manufacturer's instructions. Transfected cells were transferred to CHO-serum free medium (CHO-SFM-II, Gibco BRL) after 24 hours. The media, including control medium from mock transfections using the expression plasmids without gene inserts, were harvested 72 hours after transfection, concentrated and centrifuged. Aliquots of the cleared culture supernatant containing the recombinant hormones were stored at −20EC and thawed only once immediately prior to the hormone assay. Wild type and mutant hTSH were quantitated and verified using standard bioactivity and immunoassays. Concentrations of wild type and mutant hCG were measured using a commercially obtained chemiluminescence assay kit (Nichols Institute, San Juan Capistrano, Calif.) and an hCG immunoradiometric assay kit (ICN, Costa Mesa, Calif.).

cAMP Stimulation in Mammalian Cells Expressing the Human TSH Receptor

CHO-K1 cells stably transfected with an hTSH receptor cDNA expression vector (JP09 or JP26) were propagated and incubated with serial dilutions of wild type and mutant TSH. cAMP released into the culture medium was determined by radioimmunoassay. Equivalent amounts of total media protein were used as the negative control.

Progesterone Production in MA-10 Cells

Transformed murine Leydig cells (MA-10) propagated in 96-well culture plates were incubated with wild type and mutant hCG for 6 hours in the assay medium as described in Ascoli et al., in Endocrinol. 108:88 (1981). Progesterone released into the medium was quantitated by radioimmunoassay using a CT PROGESTERONE KIT (ICN, Costa Mesa, Calif.).

Results

The results from this experiment support the conclusion that CKGF mutated in accordance with the invention exhibited enhanced biological activity when compared with corresponding wild type CKGFs. More particularly, the results indicated that single or multiple mutations within the exemplary glycoprotein subunits in the above-described procedures could be incorporated into the CKGF structure to result in recombinant molecules having enhanced activity. This was true for several different mutations and combinations thereof, and so illustrates the principal underlying the present invention.

In a first example, a mutation in the αL1 loop of the common human α-subunit increased hormone activity of heterodimers that included the mutant α-subunit and a wild type TSH-β subunit. In this instance, the glycine residue ordinarily present at position 22 of the sequence of SEQ ID NO:1 was substituted by an arginine residue (αG22R). The mutant αG22R/TSH-β hormone bound the TSH receptor and stimulated a higher level of cyclic AMP production than did the wild type TSH.

In second and third experiments, four different mutations (αQ13K+αE14K+αP16K+αQ20K) were introduced into the structure of the same α-subunit to form the mutant α4K subunit. When the α4K subunit was expressed in combination with either the wild type human TSH-β subunit or the human TSH-β subunit fusion with CTEP of hCG, the resulting mutant heterodimers were produced at levels sufficient to provide recombinant material in useful quantities despite the substantially changed structure of the mutant heterodimers. More particularly, the results shown in Table 3 indicate that TSH hormones incorporating either the α4K subunit or the α4K in combination with the TSH-β-CTEP fusion could be recovered efficiently (in Table 3 100% expression corresponds to 47 ng of wild type TSH per ml). The presence of the CTEP component in the TSH-β-CTEP fusion served to extend the half-life and increase the stability of the mutant heterodimer that included this protein fusion. As indicated by the results presented in FIG. 6, both the α4K/TSH-β and α4K/TSH-β-CTEP mutant hormones stimulated higher levels of cyclic AMP production than did the wild type TSH. This determination was based on the ability of wild type and mutant TSH heterodimers to bind the TSHR was assessed by the stimulation of cyclic AMP production in CHO-JPO9 that stably express a transfected TSHR. The α4K/TSH-β-CTEP heterodimer showed 200 fold increase of potency and 1.5 fold increase in Vmax (see FIG. 6) compared to wild type TSH. It was surprising that the inclusion of CTEP, which is expected to prolong the in vivo half life of the α4K/TSH-β-CTEP heterodimer, also increased its in vitro activity a further 3-4 fold over that of a α4K/TSH-β wild type heterodimer. This showed that mutations which increase the bioactivity of a mutant TSH advantageously can be combined with a modification that prolongs the circulatory half-life of the molecule to create mutant hormones possessing superior in vitro and in vivo characteristics.

TABLE 3
Production of Recombinant TSH Heterodimers
Incorporating Multiple Mutations

| | Expression | |
|---|---|---|
| Hormone Construct | (% WT) | SEM |
| hTSH Wild Type | 100 | 6 |
| hTSH α4K/TSH-β Wild Type | 26 | 5 |
| hTSH α4K/TSH-β-CTEP | 20 | 3 |

In addtional experiments, mutations in the β hairpin L3 loop of the common human α-subunit also increased hormone activity. One of the mutations was a substitution of the alanine residue at position 81 with a lysine residue (αA81K). The other mutation was a substitution of the asparagine residue at position 66 with a lysine residue (αN66K). Each of the mutant human α-subunits was transiently expressed in CHO-K1 cells in combination with wild type human TSH-β subunits to produce mutant TSH heterodimers. Each of these mutant TSH heterodimers was tested in a bioactivity assay using CHO-JP09 cells that expressed the human TSH receptor. The results indicated that both mutant hormones stimulated higher levels of cAMP production than did the wild type hormone. Substitution of alanine 81 to lysine (αA81K) in the α-subunit represents the first demonstration of introduction of a lysine residue, which is not present in other homologous sequences, into a β hairpin loop.

In a sixth example, a mutation near the β hairpin L1 loop of the human TSH β subunit increased the hormone activity of a heterodimer that included this mutant subunit. The mutation was a substitution of the glutamate residue at position 6 with an asparagine residue (βE6N) which eliminates a negatively charged residue in the periphery of the β hairpin L1 loop. The mutant human TSH-β subunit was transiently expressed in CHO-K1 cells in combination with a wild type human common α-subunit to produce a mutant TSH heterodimer. The mutant TSH heterodimer was then tested in a bioactivity assay using CHO-JP09 cells that expressed the TSH receptor. This mutant TSH hormone bound the receptor and induced higher levels of cAMP production than did the wild type TSH.

In seventh and eighth experiments, two novel mutations in the β hairpin L3 loop of the hCG-β subunit, when expressed in combination with an α-subunit, increased the bioactivity of the resulting mutant hCG hormone. One mutation was a substitution of the glycine residue at position 75 with an arginine residue (hCG-βG75R). The other mutation is a substitution of the asparagine residue at position 77 with an aspartate residue (hCG-βN77D). Each of the mutant hCG β-subunits was transiently expressed in CHO-K1 cells with a wild type common αsubunit to produce mutant hCG heterodimers. Each of the mutant hCG heterodimers was then tested in a bioactivity assay using the murine Leydig cell line (MA-10) that produced progesterone following hCG stimulation. Both mutant hCG hormones induced higher levels of cAMP and progesterone production than did the wild type hCG. Substitution of asparagine 77 by aspartate in the human hCG β-subunit (hCG-βN77D) is the first example that introduction of negatively charged residues into the peripheral β hairpin loops based on sequence alignments, and resulted in increased hormone binding and activity.

The results presented above confirm that mutation of the CKGFs in accordance with the teaching provided herein advantageously could be used to make and use CKGFs having enhanced biological activities.

It will be appreciated that certain variations to this invention may suggest themselves to those skilled in the art. The foregoing detailed description is to be clearly understood as given by way of illustration, the spirit and scope of this invention being interpreted upon reference to the appended claims.

What is claimed is:

1. A human glycoprotein hormone family protein comprising at least one electrostatic charge altering mutation in a β hairpin loop structure, wherein said mutation results in said human glycoprotein hormone family protein having increased bioactivity.

2. The human glycoprotein hormone family protein of claim 1, wherein the protein is the human chorionic gonadotropin (CG) β subunit.

3. The human glycoprotein hormone family protein of claim 2, wherein the at least one electrostatic charge altering mutation is in the L1 β hairpin loop at a position selected from the group consisting of positions 1-37.

4. The human glycoprotein hormone family protein of claim 3, wherein the at least one electrostatic charge altering mutation comprises at least one basic residue introducing mutation selected from the group consisting of S1B, P4B, L5B, P7B, R8B, R10B, P11B, I12B, N13B, A14B, T15B, L16B, A17B, V18B, G22B, P24B, V25B, I27B, T28B, V29B, N30B, T31B, T32B, I33B, A35B, G36B, and Y37B, wherein B is a basic amino acid residue.

5. The human glycoprotein hormone family protein of claim 2, wherein the at least one electrostatic charge altering mutation is in the L3 β hairpin loop at a position selected from the group consisting of positions 58-87.

6. The human glycoprotein hormone family protein of claim 5, wherein the at least one electrostatic charge altering mutation comprises at least one basic residue introducing mutation selected from the group consisting of N58B, Y59B, V62B, F64B, S66B, I67B, L69B, P70B, G71B, P73B, G75B, V76B, N77B, P78B, G79B, V80B, S81B, Y82B, A83B, V84B, A85B, L86B, and S87B, wherein B is a basic amino acid residue.

7. The human glycoprotein hormone family protein of claim 2, wherein the subunit is linked to another cystine knot growth factor monomer.

8. The human glycoprotein hormone family protein of claim 1, wherein the protein is the human luteinizing hormone (LH) β subunit.

9. The human glycoprotein hormone family protein of claim 8, wherein the at least one electrostatic charge altering mutation is in the L1 β hairpin loop at a position selected from the group consisting of positions 1-33.

10. The human glycoprotein hormone family protein of claim 9, wherein the at least one electrostatic charge altering mutation comprises at least one basic residue introducing mutation selected from the group consisting of W8B, P11B, I12B, N13B, A14B, I15B, L16B, A17B, V18B, G22B, P24B, V25B, I27B, T28B, V29B, N30B, T31B, T32B, and I33B, wherein B is a basic amino acid residue.

11. The human glycoprotein hormone family protein of claim 8, wherein the at least one electrostatic charge altering mutation is in the L3 β hairpin loop at a position selected from the group consisting of positions 58-87.

12. The human glycoprotein hormone family protein of claim 8, wherein the at least one electrostatic charge altering mutation comprises at least one basic residue introducing mutation selected from the group consisting of N58B, Y59B, V62B, F64B, S66B, I67B, L69B, P70B, G71B, P73B, G75B, V76B, N77B, P78B, G79B, V79B, V80B, S81B, Y82B, A83B, V84B, A85B, L86B, and S87B, wherein B is a basic amino acid residue.

13. The human glycoprotein hormone family protein of claim 8, wherein the subunit is linked to another cystine knot growth factor monomer.

14. The human glycoprotein hormone family protein of claim 1, wherein the protein is the human follicle stimulating hormone (FSH) β subunit.

15. The human glycoprotein hormone family protein of claim 14, wherein the at least one electrostatic charge altering mutation is in the L1 β hairpin loop at a position selected from the group consisting of positions 4-27.

16. The human glycoprotein hormone family protein of claim 15, wherein the at least one electrostatic charge altering mutation comprises at least one basic residue introducing mutation selected from the group consisting of L5B, T6B, N7B, I8B, T9B, I10B, A11B, I12B, F19B, I21B, S22B, I23B, N24B, T25B, T26B, and W27B, wherein B is a basic amino acid residue.

17. The human glycoprotein hormone family protein of claim 14, wherein the at least one electrostatic charge altering mutation is in the L3 β hairpin loop at a position selected from the group consisting of positions 65-81.

18. The human glycoprotein hormone family protein of claim 17, wherein the at least one electrostatic charge altering mutation comprises at least one basic residue introducing mutation selected from the group consisting of A65B, A68B, S70B, L71B, Y72B, T73B, Y74B, P75B, V76B, A77B, T78B, and Q79B, wherein B is a basic amino acid residue.

19. The human glycoprotein hormone family protein of claim 14, wherein the subunit is linked to another cystine knot growth factor monomer.
